(19) United States
(12) Patent Application Publication
(10) Pub. No.: US 2018/0133337 A1
(43) Pub. Date: May 17, 2018

(54) CALICHEAMICIN CONSTRUCTS AND METHODS OF USE

(71) Applicant: AbbVie Stemcentrx LLC, North Chicago, IL (US)

(72) Inventors: Julia Gavrilyuk, South San Francisco, CA (US); Vikram Natwarsinhji Sisodiya, San Francisco, CA (US)

(21) Appl. No.: 15/567,904

(22) PCT Filed: Apr. 21, 2016
(86) PCT No.: PCT/US16/28530

§ 371 Date: Oct. 19, 2017

Related U.S. Application Data
(60) Provisional application No. 62/150,693, filed on Apr. 21, 2015.

(51) Int. Cl. A61K 47/68; C07K 16/30

(52) U.S. Cl. CPC A61K 47/6809; A61K 47/6851; A61K 2039/505; C07K 16/30; A61K 47/6889

(57) ABSTRACT

Provided herein are antibody drug conjugates (ADCs) comprising calicheamicin and methods of using the same to treat proliferative disorders.

### CROSS-REFERENCE TO RELATED APPLICATIONS

This application claims the benefit of U.S. Provisional Application No. 62/150,693, filed Apr. 21, 2015, which is hereby incorporated by reference in its entirety for all purposes.

### FIELD OF THE INVENTION

This application generally relates to novel compounds including calicheamicin linked to targeting agents (also referred to herein as calicheamicin-linker constructs). The targeting agent may be an antibody thereby providing antibody drug conjugates (ADCs). The ADCs may be used, for example, for the treatment, diagnosis or prophylaxis of cancer and any recurrence or metastasis thereof.

### BACKGROUND OF THE INVENTION

Differentiation and proliferation of stem cells and progenitor cells are normal ongoing processes that act in concert to support tissue growth during organogenesis, cell repair and cell replacement. The system is tightly regulated to ensure that only appropriate signals are generated based on the needs of the organism. Cell proliferation and differentiation normally occur only as necessary for the replacement of damaged or dying cells or for growth. However, disruption of these processes can be triggered by many factors including the under- or overabundance of various signaling chemicals, the presence of altered microenvironments, genetic mutations or a combination thereof. Disruption of normal cellular proliferation and/or differentiation can lead to various disorders including proliferative diseases such as cancer.

Conventional therapeutic treatments for cancer include chemotherapy, radiotherapy and immunotherapy. Often these treatments are ineffective and surgical resection may not provide a viable clinical alternative. Limitations in the current standard of care are particularly evident in those cases where patients undergo first line treatments and subsequently relapse. In such cases refractory tumors, often aggressive and incurable, frequently arise. The overall survival rates for many solid tumors have remained largely unchanged over the years due, at least in part, to the failure of existing therapies to prevent relapse, tumor recurrence and metastasis. There remains therefore a great need to develop more targeted and potent therapies for proliferative disorders. The current invention addresses this need.

### BRIEF SUMMARY OF THE INVENTION

In a first aspect, there is provided a compound, such as an antibody drug conjugate, or a pharmaceutically acceptable salt thereof, having the Formula (I):

Ab-[W-(L³)_{z1}-M-(L⁴)_{z2}-P-D]_{z3} (I).

In the antibody drug conjugate of Formula I, Ab is a targeting agent. W is a connecting group. M is a cleavable moiety. L³ and L⁴ are independently a linker (e.g. a spacer). P is a disulfide protecting group. D is a calicheamicin or analog thereof. The symbols z1, z2 and z3 are independently an integer from 0 to 10. In embodiments, z3 is an integer from 1 to 10.

In an embodiment of Formula I, there is provided a compound, such as an antibody drug conjugate, or a pharmaceutically acceptable salt thereof, of Formula (Ia):

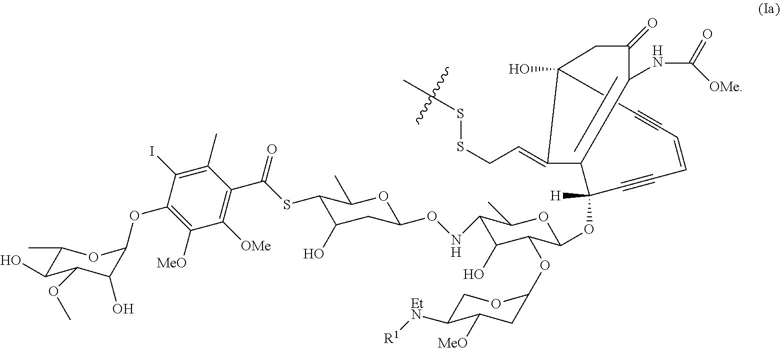

In the compound of Formula (Ia), R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —CI₃, —CN, —C(O)R^{1E}, —OR^{1A}, —NR^{1B}R^{1C}, —C(O)OR^{1A}, —C(O)NR^{1B}R^{1C}, —SR^{1D}, —SOₙ₁R^{1B} or —SOᵥ₁NR^{1B}R^{1C}, R^{1A}, R^{1B}, R^{1C}, R^{1D} and R^{1E} are independently hydrogen, halogen, —CF₃, —CCl₃, —CBr₃, —CI₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{1B} and R^{1C} substituents bonded to the same nitrogen atom may optionally be joined to form a substituted or unsubstituted heterocycloalkyl or substituted or unsubstituted heteroaryl. The symbol n1 is independently an integer from 0 or 4. The symbol v1 is independently 1 or 2. The symbol [symbol] represents the point of attachment to P in Formula I.

In an embodiment of Formula I, there is provided a compound, such as an antibody drug conjugate, or a pharmaceutically acceptable salt thereof, of Formula (II):

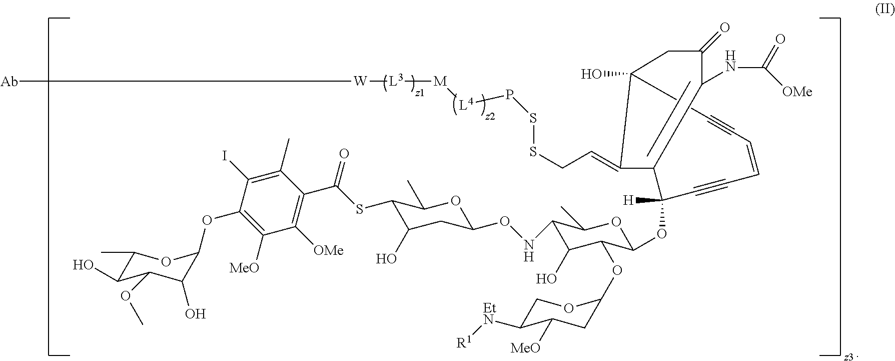

In the compound of Formula (II), Ab is a targeting moiety, such as an antibody. L³ is a bond, —O—, —S—, —NR^{3B}—, —C(O)—, —C(O)O—, —S(O)—, —S(O)₂, —C(O)NR^{3B}—, —NR^{3B}C(O)—, —NR^{3B}C(O)NH—, —NHC(O)NR^{3B}—, substituted or unsubstituted alkylene or substituted or unsubstituted heteroalkylene. L⁴ is a bond, —O—, —S—, —NR^{4B}—, —C(O)—, —C(O)O—, —S(O)—, —S(O)₂—, —C(O)NR^{4B}—, —NR^{4B}C(O)—, —NR^{4B}C(O)NH—, —NHC(O)NR^{4B}—, substituted or unsubstituted alkylene or substituted or unsubstituted heteroalkylene. R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —CI₃, —CN, —C(O)R^{1E}, —OR^{1A}, —NR^{1B}R^{1C}, —C(O)OR^{1A}, —C(O)NR^{1B}R^{1C}, —SR^{1D}, —SOₙ₁R^{1B} or —SOᵥ₁NR^{1B}R^{1C}. P is —O—, —S—, —NR^{2B}—, —C(O)—, —C(O)O—, —S(O)—, —S(O)₂—, —C(O)NR^{2B}—, —NR²BC(O)—, —NR²BC(O)NH—, —NHC(O)NR^{2B}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. M is —O—, —S—, —NR^{5B}—, —C(O)—, —C(O)O—, —S(O)—, —S(O)₂—, —C(O)NR^{5B}—, —NR^{5B}C(O)—, —NR^{5B}C(O)NH—, —NHC(O)NR^{5B}—, —[NR^{5B}C(R^{5E})(R^{5F})C(O)]ₙ₂—, substituted or unsubstituted alkylene, substituted or unsubstituted heteroalkylene, substituted or unsubstituted cycloalkylene, substituted or unsubstituted heterocycloalkylene, substituted or unsubstituted arylene substituted or unsubstituted heteroarylene or M^{1A}-M^{1B}-M^{1C}. W is —O—, —S—, —NR^{6B}, —C(O)—, —C(O)O—, —S(O)—, —S(O)₂—, —C(O)NR^{6B}—, —NR^{6B}C(O)—, —NR^{6B}C(O)NH—, —NHC(O)NR^{6B}—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene or W^{1A}—W^{1B}—W^{1C}. M^{1A} is bonded to L³. M^{1C} is bonded to L⁴. M^{1A} is a bond, —O—, —S—, —NR^{5AB}—, —C(O)—, —C(O)O—, —S(O)—, —S(O)₂—, —C(O)NR^{5AB}—, —NR^{5AB}C(O)—, —NR^{5AB}C(O)NH—, —NHC(O)NR^{5AB}—, —[NR^{5AB}CR^{5AE}R^{5AF}C(O)]ₙ₃—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. M^{1B} is a bond, —O—, —S—, —NR^{5BB}—, —C(O)—, —C(O)O—, —S(O)—, —S(O)₂—, —C(O)NR^{5BB}—, —NR^{5BB}C(O)—, —NR^{5BB}C(O)NH—, —NHC(O)NR^{5BB}—, —[NR^{5BB}C(R^{5BE})(R^{5BF})C(O)]ₙ₄—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. M^{1C} is a bond, —O—, —S—, —NR^{5CB-}, —C(O)—, —C(O)O—, —S(O), —S(O)₂—, —C(O)NR^{5CB}—, —NR^{5CB}C(O)—, —NR^{5CB}C(O)NH—, —NHC(O)NR^{5CB}—, —[NR^{5CB}CR^{5CE}R^{5CF}C(O)]ₙ₅—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. W^{1A} is bonded to Ab. W^{1C} is bonded to L³. W^{1A} is a bond, —O—, —S—, —NR^{6BA}—, —C(O)—, C(O)O—, —S(O)—, —S(O)₂—, —C(O)NR^{6BA-}, —NR^{6BA}C(O)—, —NR^{6BA}C(O)NH—, —NHC(O)NR^{6BA}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. W^{1B} is a bond, —O—, —S—, —NR^{6BB}—, —C(O)—, —C(O)O—, —S(O)—, —S(O)₂—, —C(O)NR^{6BB}—, —NR^{6BB}C(O)—, —NR^{6BB}C(O)NH—, —NHC(O)NR^{6BB}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. W^{1C} is a bond, —O—, —S—, —NR^{6BC-}, —C(O)—, —C(O)O—, —S(O)—, —S(O)₂—, —C(O)NR^{6BC}—, —NR^{6BC}C(O)—, —NR^{6BC}C(O)NH—, —NHC(O)NR^{6BC-}, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R^{1A}, R^{1B}, R^{1C}, R^{1D}, R^{1E}, R^{2B}, R^{3B}, R^{4B}, R^{5B}, R^{5E}, R^{5F}, R^{5AB}, R^{5AE}, R^{5AF}, R^{5BB}, R^{5BE}, R^{5BF}, R^{5CB}, R^{5CE}, R^{5CF}, R^{6B}, R^{6BA}, R^{6BB} and R^{6BC} are independently hydrogen, halogen, —CF₃, —CCl₃, —CBr₃, —CI₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{1B} and R^{1C} substituents bonded to the same nitrogen atom may optionally be joined to form a substituted or unsubstituted heterocycloalkyl or substituted or unsubstituted heteroaryl. The symbol n1 is an integer from 0 to 4. The symbol v1 is 1 or 2. The symbols n2, n3, n4 and n5 are independently and integer from 1 to 10. The symbols z1 and z2 are independently an integer from 0 to 10. The symbol z3 is independently an integer from 1 to 10.

In another aspect, there is provided a compound of Formula (IV):

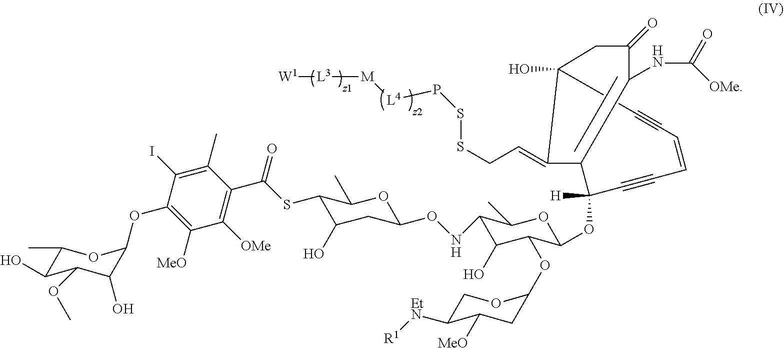

In the compound of Formula (IV), L³ is a bond, —O—, —S—, —NR^{3B}—, —C(O)—, —C(O)O—, —S(O)—, —S(O)₂—, —C(O)NR³B—, —NR³BC(O)—, —NR³BC(O)NH—, —NHC(O)NR³B—, substituted or unsubstituted alkylene or substituted or unsubstituted heteroalkylene. L⁴ is a bond, —O—, —S—, —NR⁴B—, —C(O)—, —C(O)O—, —S(O)—, —S(O)₂—, —C(O)NR⁴B—, —NR⁴BC(O)—, —NR⁴BC(O)NH—, —NHC(O)NR^{4B}—, substituted or unsubstituted alkylene or substituted or unsubstituted heteroalkylene. R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —CI₃, —CN, —C(O)R^{1E}, —OR^{1A}, —NR^{1B}R^{1C}, —C(O)OR^{1A}, —C(O)NR^{1B}R^{1C}, —SR^{1D}, —SOₙ₁R^{1B} or —SOᵥ₁NR^{1B}R^{1C}. P is —O—, —S—, —NR^{2B}—, —C(O)—, —C(O)O—, —S(O)—, —S(O)₂—, —C(O)NR^{2B}—, —NR^{2B}C(O)—, —NR^{2B}C(O)NH—, —NHC(O)NR^{2B}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. M is —O—, —S—, —NR^{5B-}, —C(O)—, —C(O)O—, —S(O)—, —S(O)₂—, —C(O)NR^{5B}—, —NR⁵BC(O)—, —NR⁵BC(O)NH—, —NHC(O)NR^{5B}, [NR^{5B}C(R^{5E})(R^{5F})C(O)]ₙ₂—, substituted or unsubstituted alkylene, substituted or unsubstituted heteroalkylene, substituted or unsubstituted cycloalkylene, substituted or unsubstituted heterocycloalkylene, substituted or unsubstituted arylene substituted or unsubstituted heteroarylene or M^{1A}-M^{1B}-M¹. W¹ is a reactive moiety, hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —N₃, —CF₃, —CCl₃, —CBr₃, —CI₃, —CN, —C(O)R^{7E}, —OR^{7A}, —NR^{7B}R^{7C}, —C(O)OR^{7A}, —C(O)NR^{7B}R^{7C}, —NO₂, —SR^{7D}, —SOᵥ₇R^{7B}, —SOᵥ₇NR^{7B}R^{7C}, —NHNR^{7B}R^{7C}, —ONR^{7B}R^{7C} or —NHC(O)NHNR^{7B}R^{7C}. M^{1A} is bonded to L³. M^{1C} is bonded to L⁴. M^{1A} is a bond, —O—, —S—, —NR^{5AB}—, —C(O)—, —C(O)O—, —S(O)—, —S(O)₂—, —C(O)NR^{5AB}—, —NR^{5AB}C(O)—, —NR^{5AB}C(O)NH—, —NHC(O)NR^{5AB}—, —[NR^{5AB}CR^{5AE}R^{5AF}C(O)]ₙ₃—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. M^{1B} is a bond, —O—, —S—, —NR^{5BB}—, —C(O)—, —C(O)O—, —S(O)—, —S(O)₂—, —C(O)NR^{5BB}—, —NR^{5BB}C(O)—, —NR^{5BB}C(O)NH—, —NHC(O)NR^{5BB}—, [NR^{5BB}C(R^{5BE})(R^{5BF})C(O)]ₙ₄—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. M^{1C} is a bond, —O—, —S—, —NR^{5CB}—, —C(O)—, —C(O)O—, —S(O)—, —S(O)₂—, —C(O)NR^{5CB}—, —NR^{5CB}C(O)—, —NR^{5CB}C(O)NH—, —NHC(O)NR^{5CB}—, —[NR^{5CB}CR^{5CE}R^{5CF}C(O)]ₙ₅—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R^{1A}, R^{1B}, R^{1C}, R^{1D}, R^{1E}, R^{2B}R^{3B}, R^{4B}, R^{5B}, R^{5E}, R^{5F}, R^{5AB}, R^{5AE}, R^{5AF}, R^{5BB}, R^{5BE}, R^{5BF}, R^{5CB}CB, R^{5CE}, R^{5CF}, R^{6B}, R^{7A}, R^{7B}, R^{7C}, R^{7D}, R^{7E}, are independently hydrogen, halogen, —CF₃, —CCl₃, —CBr₃, —CI₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{1B} and R^{1C} substituents bonded to the same nitrogen atom may optionally be joined to form a substituted or unsubstituted heterocycloalkyl or substituted or unsubstituted heteroaryl. The symbols n1 and n7 are independently an integer from 0 to 4. The symbols n7 and v7 are independently 1 or 2. The symbols n2, n3, n4 and n5 are independently and integer from 1 to 10.

In another aspect, there is provided a method of preparing an antibody drug conjugate. The method includes contacting a calicheamicin construct with an amino acid of an antibody such as cysteine or lysine, the calicheanicin construct having formula W¹-(L³)_{z1}-M-(L⁴)_{z2}-P-D as defined herein. W¹ is a functional group reactive with an amino acid such as lysine side chain or cysteine side chain. M is a cleavable moiety. L³ and L⁴ are independently a linker. P is a disulfide protecting group. D is a calicheamicin or analog thereof. The symbols z1 and z2 are independently an integer from 0 to 10. The symbol z3 is independently an integer from 1 to 10.

Also provided herein are pharmaceutical compositions. In one aspect is a pharmaceutical composition that includes a compound described herein and a pharmaceutically acceptable excipient. In another aspect is a pharmaceutical composition that includes an antibody drug conjugate described herein and a pharmaceutically acceptable excipient.

Also provided herein is a method of treating cancer in a subject in need thereof. The method includes administering to the subject a therapeutically effective amount of the pharmaceutical compositions or compounds (e.g. antibody drug conjugates) described herein.

### BRIEF DESCRIPTION OF THE DRAWINGS

FIG. 1 shows the chemical structure of an exemplary calicheamicin-linker construct fabricated in accordance with the instant invention annotated to show certain components of the construct.

FIGS. 2A-2C provide data demonstrating that disclosed calicheamicin-linker constructs of the invention are effectively cleaved to provide active calicheamicin.

FIGS. 3A-3D show that calicheamicin (FIG. 3A) and exemplary calicheamicin-linker constructs (FIGS. 3B-3D) effectively kill cells in vitro while derived IC50 values.

FIGS. 4A and 4B provide mass spectrometry data confirming that calicheamicin-linker constructs of the invention are efficiently conjugated to exemplary antibodies using the disclosed procedures.

FIG. 5 shows conjugation percentages of two exemplary site-specific antibodies light and heavy chains conjugated to two different calicheamicin-linker constructs as determined using RP-HPLC. hSC17ss1-vc is Formula 4′ attached to hSC17 antibody at the indicated point of attachment in Formula 4′; the antibody is hSC17ss1 (an IgG antibody) connected to the remainder of the conjugate/molecule through cysteine side chain(s). hSC17ss1-va is Formula 5′ attached to hSC17 antibody at the indicated point of attachment in Formula 5′; the antibody is hSC17ss1 (an IgG antibody) connected to the remainder of the conjugate/molecule through cysteine side chain(s). hSC1ss1-vc is Formula 4′ attached to to hSC1 antibody at the indicated point of attachment in Formula 4′; the antibody is hSC1ss 1 (an IgG antibody) connected to the remainder of the conjugate/molecule through cysteine side chain(s).

FIG. 6 provides a graphical representation showing the DAR distribution of exemplary site-specific antibody constructs conjugated using procedures disclosed herein as determined using HIC.

FIGS. 7A-7C demonstrate the ability of exemplary antibody drug conjugates comprising a calicheamicin-vc linker (FIG. 7A), a calicheamicin-va linker (FIG. 7B) or a calicheamicin-oxime linker (FIG. 7C) to kill cells in vitro.

FIGS. 8A-8C provide data showing that exemplary antibody drug conjugates of the instant invention can effectively kill tumor cells in vivo. hSC17ss1-ox is Formula 14′ attached to hSC17 antibody at the indicated point of attachment in Formula 14′; the antibody is hSC17ss1 (an IgG antibody) connected to the remainder of the conjugate/molecule through cysteine side chain(s).

FIG. 9 provides data showing exemplary antibody drug conjugates of the instant invention can effectively kill tumor cells in vivo.

FIG. 10 provides pharmacokinetic data showing of exemplary antibody drug conjugates in cynomolgus monkey. hSC27ss1-vc is Formula 4′ attached to hSC27 antibody at the indicated point of attachment in Formula 4′; the antibody is hSC27ss1 (an IgG antibody) connected to the remainder of the conjugate/molecule through cysteine side chain(s).

### DETAILED DESCRIPTION OF THE INVENTION

The invention may be embodied in many different forms. Disclosed herein are non-limiting, illustrative embodiments of the invention that exemplify the principles thereof. Any section headings used herein are for organizational purposes only and are not to be construed as limiting the subject matter described. Generally, the nomenclature used herein and the laboratory procedures in cell culture, molecular genetics, organic chemistry and nucleic acid chemistry and hybridization described below are those well-known and commonly employed in the art. Standard techniques are used for nucleic acid and peptide synthesis. Generally, enzymatic reactions and purification steps are performed according to the manufacturer's specifications. For the purposes of the instant disclosure all identifying sequence accession numbers may be found in the NCBI Reference Sequence (RefSeq) database and/or the NCBI GenBank® archival sequence database unless otherwise noted. The nomenclature used herein and the laboratory procedures in analytical chemistry, and organic synthetic described below are those well known and commonly employed in the art. Standard techniques, or modifications thereof, are used for chemical syntheses and chemical analyses.

### I. Definitions

The term “cleavable moiety” is intended to mean a moiety that is subject to cleavage at the selected target site. Preferably the “cleavable moiety” allows for separation and/or activation of the calicheamicin by cleaving or separating it from the targeting agent. Operatively defined, the linker (as defined below) is preferably cleaved through bifurcation of the cleavable moiety at the target site by physiological effectors. The cleavage may come from any process without limitation, e.g., enzymatic, reduction, pH, etc. Preferably, the cleavable moiety is selected and integrated in the linker so that activation occurs at the desired site of action, which preferably is a site in or near the target cells (e.g., carcinoma cells) or tissue. In selected embodiments cleavable moieties may comprise peptide bonds, hydrazone moieties, oxime moieties, ester linkages and disulfide linkages. In particularly preferred embodiments such cleavage is enzymatic where exemplary enzymatically cleavable groups include natural amino acids or peptide sequences that end with a natural amino acid, and are incorporated in the linker. Preferably the incorporated cleavable moieties are those in which at least about 10% of the calicheamicin is activated and released within 24 hours of administration and more preferably 25% is released.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. These terms also encompass the term “antibody.”

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but functions in a manner similar to a naturally occurring amino acid. The term “unnatural amino acid” is intended to represent the “D” stereochemical form of the twenty naturally occurring amino acids described above. It is further understood that the term unnatural amino acid includes homologues of the natural amino acids, and synthetically modified forms of the natural amino acids. The synthetically modified forms include, but are not limited to, amino acids having alkylene chains shortened or lengthened by up to two carbon atoms, amino acids comprising optionally substituted aryl groups, and amino acids comprised halogenated groups, preferably halogenated alkyl and aryl groups. When attached to a linker or conjugate of the invention, the amino acid is in the form of an “amino acid side chain”, where the carboxylic acid group of the amino acid has been replaced with a keto (C(O)) group. Thus, for example, an alanine side chain is —C(O)—CH(NH₂)—CH₃, and so forth.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

“Aliphatic” means a straight- or branched-chain, saturated or unsaturated, non-aromatic hydrocarbon moiety having the specified number of carbon atoms (e.g., as in “C₃ aliphatic,” “C₁-C₅ aliphatic,” or “C₁ to C₅ aliphatic,” the latter two phrases being synonymous for an aliphatic moiety having from 1 to 5 carbon atoms) or, where the number of carbon atoms is not explicitly specified, from 1 to 4 carbon atoms (2 to 4 carbons in the instance of unsaturated aliphatic moieties).

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). The term “alkyl,” unless otherwise noted, is also meant to include those derivatives of alkyl defined in more detail below, such as “heteroalkyl.” Alkyl groups, which are limited to hydrocarbon groups are termed “homoalkyl”. In embodiments, alkyl does not include cyclic hydrocarbon radicals. In embodiments, the term “alkyl” as used herein refers to a saturated linear or branched-chain monovalent hydrocarbon radical of one to twenty carbon atoms. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. “Monovalent” means that alkyl has one point of attachment to the remainder of the molecule. Examples of alkyl groups include, but are not limited to, methyl, ethyl, 1-propyl, 2-propyl, 1-butyl, 2-methyl-1-propyl, —CH₂CH(CH₃)₂, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. Specifically, the alkyl group has one to ten carbon atoms. More specifically, the alkyl group has one to four carbon atoms.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen, carbon and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). The terms “heteroalkyl” and “heteroalkylene” encompass poly(ethylene glycol) and its derivatives. Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The term “lower” in combination with the terms “alkyl” or “heteroalkyl” refers to a moiety having from 1 to 6 carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of substituted or unsubstituted “alkyl” and substituted or unsubstituted “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The heteroatoms and carbon atoms of the cyclic structures are optionally oxidized.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” (abbrev. Ar) means, unless otherwise stated, a substituted or unsubstituted polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen, carbon and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. “Aryl” and “heteroaryl” also encompass ring systems in which one or more non-aromatic ring systems are fused, or otherwise bound, to an aryl or heteroaryl system.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents,” respectively, and they can be one or more of a variety of groups selected from, but not limited to: —O′, ═O, ═NR′, ═N—OR′, —NR′R″, —S′, -halogen, —SiR′R″R′″, —OC(O)′, —C(O)′, —CO₂′, —CONR′R″, —OC(O)NR′R″, —NR″C(O)′, —NR′—C(O)NR″R′″, NR″C(O)₂′, —NR—C(NR′R″R′″)═NR″″, —NR—C(NR′R″)═NR′″, —S(O)′, —S(O)₂′, —S(O)₂NR′R″, —NRSO₂′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, OC(O)NR′R″, —NR″C(O)R′, —NR′—C(O)NR″R′″, —NR″C(O)₂R′, —NR—C(NR′R″)═NR′″, S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R′, R′″′ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₙ—X—(CR″R′″)_{d}—, where s and d are independently integers of from 0 to 3, and X is —O—, —NR′—, S—, —S(O)—, —S(O)₂—, or —S(O)₂NR′—. The substituents R, R′, R″ and R′″ are preferably independently selected from hydrogen or substituted or unsubstituted (C₁-C₆) alkyl.

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

“Alkylene” as used herein refers to a saturated linear or branched-chain divalent hydrocarbon radical of one to twenty carbon atoms, examples of which include, but are not limited to, those having the same core structures of the alkyl groups as exemplified above. “Divalent” means that the alkylene has two points of attachment to the remainder of the molecule. Specifically, the alkylene group has one to ten carbon atoms. More specifically, the alkylene group has one to four carbon atoms.

The terms “carbocycle,” “carbocyclyl,” carbocyclic and “carbocyclic ring” refer to a monovalent non-aromatic, saturated or partially unsaturated ring having 3 to 12 carbon atoms as a monocyclic ring or 7 to 12 carbon atoms as a bicyclic ring. Bicyclic carbocycles having 7 to 12 atoms can be arranged, for example, as a bicyclo [4,5], [5,5], [5,6] or [6,6] system, and bicyclic carbocycles having 9 or 10 ring atoms can be arranged as a bicyclo [5,6] or [6,6] system, or as bridged systems such as bicyclo[2.2.1]heptane, bicyclo[2.2.2]octane and bicyclo[3.2.2]nonane. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like.

The term “cycloalkylalkyl” refers to a cycloalkyl group that is connected to another group by an alkylene group. Examples of cycloalkylalkyls include, but are not limited to, cyclohexylmethyl, cyclohexylethyl, cyclopentylmethyl, cyclopentylethyl, and the like.

If a group is described as being “optionally substituted,” the group may be either (1) not substituted, or (2) substituted. If a carbon of a group is described as being optionally substituted with one or more of a list of substituents, one or more of the hydrogen atoms on the carbon (to the extent there are any) may separately and/or together be replaced with an independently selected optional substituent.

The terms “targeting agent” and “cell binding agent” may be used interchangeably and are intended to mean a moiety that is (1) able to direct the entity to which it is attached (e.g., calicheamicin) to a target cell, for example to a specific type of tumor cell or (2) is preferentially activated at a target tissue, for example a tumor. The targeting agent can be a small molecule, which is intended to include both non-peptides and peptides. The targeting agent can also be a macromolecule, which includes saccharides, lectins, receptors, ligand for receptors, proteins such as BSA, antibodies, and so forth. Most preferably the targeting agent shall comprise an antibody or immunoreactive fragment thereof. In embodiments, the targeting agent is an antibody or immunoreactive fragment thereof.

The term “salt” as used herein refers to organic or inorganic salts of a compound of the invention. Specifically, a salt is a pharmaceutically acceptable salt. Other non-pharmaceutically acceptable salts are also included in the present invention (e.g. molecule or macromolecule). The salts include salts, formed by reacting a compound of the invention, which comprises a basic group, with an inorganic acid or organic acid (such as a carboxylic acid), and salts, formed by reacting a compound of the invention, which comprises an acidic group, with an inorganic base or organic base (such as an amine). Exemplary salts include those pharmaceutically acceptable salts described immediately below.

When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent.

Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “pharmaceutically acceptable salt” means organic or inorganic salts of a molecule or macromolecule. Pharmaceutically acceptable salts include salts of the active compounds which are prepared with relatively nontoxic acids or bases, depending on the particular substituents found on the compounds described herein. Acid addition salts can be formed with amino groups. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′ methylene bis-(2-hydroxy 3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Where multiple charged atoms are part of the pharmaceutically acceptable salt, the salt can have multiple counter ions. Hence, a pharmaceutically acceptable salt can have one or more charged atoms and/or one or more counterion.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Pharmaceutically acceptable solvate” or “solvate” refers to an association of one or more solvent molecules and a molecule or macromolecule. Examples of solvents that form pharmaceutically acceptable solvates include, but are not limited to, water, isopropanol, ethanol, methanol, DMSO, ethyl acetate, acetic acid, and ethanolamine.

The term “linker,” “bioconjugate linker,” and “spacer” are used interchangeably and as used herein describe a divalent chemical group that covalently joins one chemical moiety to another. Specific examples of linkers are described herein. Linkers may be polyethylene (PEG) linkers or bioconjugate linkers or a combination thereof.

The term “connecting group,” or “bioconjugation moiety” refers to a moiety, which allows for attachment of a targeting agent to the linker. As discussed in more detail below, exemplary connecting groups include, by way of illustration and not limitation, alkyl, aminoalkyl, aminocarbonylalkyl, carboxyalkyl, hydroxyalkyl, alkyl-maleimide, alkyl-N-hydroxylsuccinimide, poly(ethylene glycol)-maleimide and poly(ethylene glycol)-N-hydroxylsuccinimide, all of which may be further substituted. The linker can also have the attaching moiety be actually appended to the targeting group.

“Reactive functional group,” “reactive moiety,” “reactive group,” as used herein refers to groups that react to form linkers between chemical moieties. The reactive groups described include reactive functional groups commonly employed in bioconjugate techniques, as described herein. In embodiments, the reactive moiety may be a functional group reactive with an amino acid (e.g. amino acid side chain) such as a lysine side chain or cysteine side chain. Reactive groups include but are not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application to or modification for a particular purpose is within the ability of one of skill in the art.

As used herein, the term “conjugate” refers to the association between atoms or molecules. The association can be direct or indirect. For example, a conjugate between a nucleic acid (e.g., ribonucleic acid) and a compound moiety as provided herein can be direct, e.g., by covalent bond, or indirect, e.g., by non-covalent bond. Optionally, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. Thus, the nucleic acid acids can be attached to a compound moiety through its backbone. Optionally, the ribonucleic acid includes one or more reactive moieties, e.g., an amino acid reactive moiety, that facilitates the interaction of the ribonucleic acid with the compound moiety.

Useful reactive moieties or reactive functional groups used for conjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds;

(k) phosphoramidites and other standard functional groups useful in nucleic acid synthesis;

(l) metal silicon oxide bonding;

(m) metal bonding to reactive phosphorus groups (e.g. phosphines) to form, for example, phosphate diester bonds; and

(n) sulfones, for example, vinyl sulfone.

Chemical synthesis of compositions by joining small modular units using conjugate (“click”) chemistry is well known in the art and described, for example, in H. C. Kolb, M. G. Finn and K. B. Sharpless ((2001). “Click Chemistry: Diverse Chemical Function from a Few Good Reactions”. Angewandte Chemie International Edition 40 (11): 2004-2021); R. A. Evans ((2007). “The Rise of Azide-Alkyne 1,3-Dipolar ‘Click’ Cycloaddition and its Application to Polymer Science and Surface Modification”. Australian Journal of Chemistry 60 (6): 384-395; W. C. Guida et al. Med. Res. Rev. p 3 1996; Spiteri, Christian and Moses, John E. ((2010). “Copper-Catalyzed Azide-Alkyne Cycloaddition: Regioselective Synthesis of 1,4,5-Trisubstituted 1,2,3-Triazoles”. Angewandte Chemie International Edition 49 (1): 31-33); Hoyle, Charles E. and Bowman, Christopher N. ((2010). “Thiol-Ene Click Chemistry”. Angewandte Chemie International Edition 49 (9): 1540-1573); Blackman, Melissa L. and Royzen, Maksim and Fox, Joseph M. ((2008). “Tetrazine Ligation: Fast Bioconjugation Based on Inverse-Electron-Demand Diels-Alder Reactivity”. Journal of the American Chemical Society 130 (41): 13518-13519); Devaraj, Neal K. and Weissleder, Ralph and Hilderbrand, Scott A. ((2008). “Tetrazine Based Cycloadditions: Application to Pretargeted Live Cell Labeling”. Bioconjugate Chemistry 19 (12): 2297-2299); Stöckmann, Henning; Neves, Andre; Stairs, Shaun; Brindle, Kevin; Leeper, Finian ((2011). “Exploring isonitrile-based click chemistry for ligation with biomolecules”. Organic & Biomolecular Chemistry), all of which are hereby incorporated by reference in their entirety and for all purposes.

The reactive functional groups can be chosen such that they do not participate in, or interfere with, the chemical stability of the proteins described herein. By way of example, the nucleic acids can include a vinyl sulfone or other reactive moiety. Optionally, the nucleic acids can include a reactive moiety having the formula S—S—R. R can be, for example, a protecting group. Optionally, R is hexanol. As used herein, the term hexanol includes compounds with the formula C₆H₁₃OH and includes, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, and 2-ethyl-1-butanol. Optionally, R is 1-hexanol.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The term “therapeutically effective amount” means that amount of active calicheamicin or antibody drug conjugate that elicits the desired biological response in a subject. Such response includes alleviation of the symptoms of the disease or disorder being treated, prevention, inhibition or a delay in the recurrence of symptom of the disease or of the disease itself, an increase in the longevity of the subject compared with the absence of the treatment, or prevention, inhibition or delay in the progression of symptom of the disease or of the disease itself. Determination of the effective amount is well within the capability of those skilled in the art, especially in light of the detailed disclosure provided herein. Toxicity and therapeutic efficacy of the disclosed compounds can be determined by standard pharmaceutical procedures in cell cultures and in experimental animals. The effective amount of compound or conjugate of the present invention or other therapeutic agent to be administered to a subject will depend on the stage, category and status of the multiple myeloma and characteristics of the subject, such as general health, age, sex, body weight and drug tolerance. The effective amount of compound or conjugate of the present invention or other therapeutic agent to be administered will also depend on administration route and dosage form. Dosage amount and interval can be adjusted individually to provide plasma levels of the active compound that are sufficient to maintain desired therapeutic effects.

Provided herein, inter alia, are novel methods, compounds, compositions and articles of manufacture that provide calicheamicin-linker constructs that exhibit favorable pharmacokinetic and pharmacodynamic characteristics. The benefits provided herein may be broadly applicable in the field of antibody drug conjugates and may be used in conjunction with antibodies that react with a variety of targets. In embodiments, the disclosed compounds (e.g. antibody drug conjugates) include novel calicheamicin-linker constructs having a cleavable moiety that allows for efficient presentation of a cytotoxic calicheamicin species at the target site with reduced non-specific toxicity. Moreover, in embodiments the disclosed calicheamicin-linker constructs are used to provide site-specific conjugate preparations that are relatively stable when compared with conventional conjugated preparations and substantially homogenous as to average DAR distribution and payload position. As shown in the appended Examples, the stability and homogeneity of such site-specific calicheamicin conjugates (regarding both average DAR distribution and calicheamicin positioning) provide for a favorable toxicity profile that contributes to an improved therapeutic index.

In one embodiment the invention is directed to calicheamicin-linker constructs comprising one or more cleavable moieties. Those of skill in the art will appreciate that the cleavable calicheamicin payloads allow for the selective and controlled delivery of the activated warhead to the target site (e.g., a tumor cell).

In embodiments the disclosed compounds will immunospecifically react with an antigenic determinant present on tumorigenic cells. Accordingly, in particularly preferred embodiments the present invention is directed to an antibody drug conjugate comprising a cleavable calicheamicin payload wherein the antibody immunospecifically reacts with a SEZ6 determinant which is known to be associated with various tumors.

### II. Compositions

Provided herein are compounds (e.g. antibody drug conjugates) of Formula 2, or a pharmaceutically acceptable salt thereof:

Ab-[W—(X1)ₐ-CM-(X2)_{b}—P-D]ₙ (Formula 2).

Ab is a targeting agent. W is a connecting group or a linker. CM is a cleavable moiety. P is a disulfide protective group. X1 and X2 comprise optional spacer or linker moieties. D is calicheamicin. The symbols a and b are independently 0 or 1. The symbol n is 1, 2, 3, 4, 5, 6, 7, 8, 9 or 10.

In one aspect, there is provided a compound (e.g. an antibody drug conjugate), or a pharmaceutically acceptable salt thereof of Formula (I):

Ab-[W-(L³)_{z1}-M-(L⁴)_{z2}-P-D]_{z3} (I).

Ab is a targeting agent. W is a connecting group or linker group. M is a cleavable moiety. L³ and L⁴ are independently a linker or spacer. P is a disulfide protecting group. D is a calicheamicin or analog thereof. The symbols z1, z2 and z3 are independently an integer from 0 to 10. In embodiments, the symbol z3 is an integer from 1 to 10.

Where D is calicheamicin or analog thereof in any of the formulae provided herein, it is understood that D (the calicheamicin or analog) includes any member of the class of calicheamicin as known in the art wherein the terminal —S—S—S—CH₃ moiety is replaced with —S—S- [symbol] , wherein the symbol [symbol] represents the point of attachment to P. Calicheamicins are a class of enediyne antitumor antibiotics derived from the bacterium Micromonospora echinospora, including but not limited to calicheamicin γ^{I}, calicheamicin β₁^{Br}, calicheamicin γ₁^{Br}, calicheamicin α₂^{I}, calicheamicin α3^{I}, calicheamicin β₁ⁱ and calicheamicin δ₁ⁱ.

In embodiments, the targeting agent is an antibody.

In embodiments, D is of Formula (Ia):

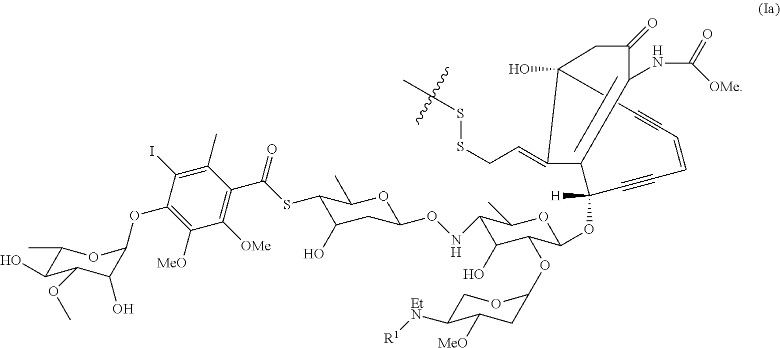

R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —CI₃, —CN, —C(O)R^{1E}, —OR^{1A}, —NR^{1B}R^{1C}, —C(O)OR^{1A}, —C(O)NR^{1B}R^{1C}, —SR^{1D}, —SOₙ₁R^{1B} or —SOᵥ₁NR^{1B}R^{1C}, R^{1A}, R^{1B}, R^{1C}C, R^{1D} and R^{1E} are independently hydrogen, halogen, —CF₃, —CCl₃, —CBr₃, —CI₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1B} and R^{1C} substituents bonded to the same nitrogen atom may optionally be joined to form a substituted or unsubstituted heterocycloalkyl or substituted or unsubstituted heteroaryl. The symbol n1 is independently an integer from 0 to 4. The symbol v1 is independently 1 or 2.

In another aspect, there is provided a compound (e.g., an antibody drug conjugate) of Formula (II):

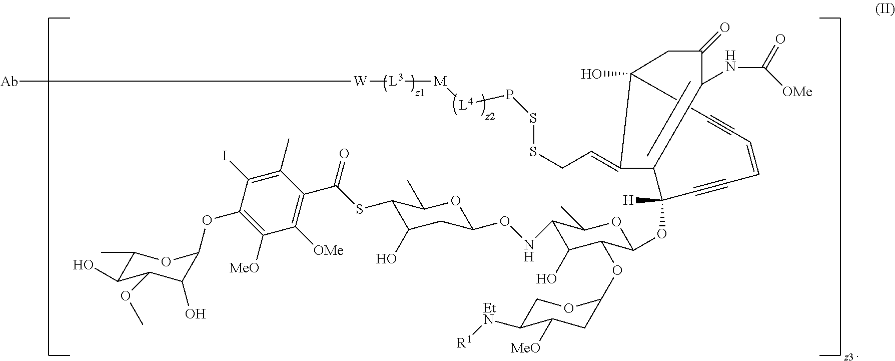

Ab is a targeting agent such as an antibody. In embodiments, the antibody is a chimeric antibody, a CDR grafted antibody, a humanized antibody or a human antibody or an immunoreactive fragment thereof. In embodiments, the antibody is an anti-SEZ6 antibody.

L³ is a bond, —O—, —S—, —NR^{3B}—, —C(O)—, —C(O)O—, —S(O)—, —S(O)₂, —C(O)NR^{3B}—, —NR^{3B}C(O)—, —NR^{3B}C(O)NH—, —NHC(O)NR^{3B}—, substituted or unsubstituted alkylene or substituted or unsubstituted heteroalkylene.

L⁴ is a bond, —O—, —S—, —NR^{4B}—, —C(O)—, —C(O)O—, —S(O)—, —S(O)₂—, —C(O)NR^{4B}—, —NR^{4B}C(O)—, —NR^{4B}C(O)NH—, —NHC(O)NR^{4B}—, substituted or unsubstituted alkylene or substituted or unsubstituted heteroalkylene.

R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —CI₃, —CN, —C(O)R^{1E}, —OR^{1A}, —NR^{1B}R^{1C}, —C(O)OR^{1A}, —C(O)NR^{1B}R^{1C}, —SR^{1D}, —SOᵥ₁R^{1B} or —SOᵥ₁NR^{1B}R^{1C}.

P is —O—, —S—, —NR^{2B}—, —C(O)—, —C(O)O—, —S(O)—, —S(O)₂—, —C(O)NR^{2B}—, —NR^{2B}C(O)—, —NR^{2B}C(O)NH—, —NHC(O)NR^{2B}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

M is —O—, —S—, —NR^{5B}—, —C(O)—, —C(O)O—, —S(O)—, —S(O)₂—, —C(O)NR^{5B}—, —NR^{5B}C(O)—, —NR^{5B}C(O)NH—, —NHC(O)NR^{5B}—, —[NR^{5B}C(R^{5E})(R^{5F})C(O)]ₙ₂—, substituted or unsubstituted alkylene, substituted or unsubstituted heteroalkylene, substituted or unsubstituted cycloalkylene, substituted or unsubstituted heterocycloalkylene, substituted or unsubstituted arylene substituted or unsubstituted heteroarylene or M^{1A}-M^{1B}-M^{1C}.

W is —O—, —S—, —NR^{6B}—, —C(O)—, —C(O)O—, —S(O), —S(O)₂—, —C(O)NR^{6B}—, —NR^{6B}C(O)—, —NR^{6B}C(O)NH—, —NHC(O)NR^{6B}—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene or W^{1A}—W^{1B}—W^{1C}.

M^{1A} is bonded to L³. M^{1C} is bonded to L⁴.

M^{1A} is a bond, —O—, —S—, —NR^{5AB}—, —C(O)—, —C(O)O—, —S(O), —S(O)₂—, —C(O)NR^{5AB}, —NR^{5AB}C(O)—, —NR ABC(O)NH—, —NHC(O)NR^{5AB}—, —[NR^{5AB}CR^{5AE}R^{5AF}C(O)]ₙ₃—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

M^{1B} is a bond, —O—, —S—, —NR^{BB}—, —C(O)—, —C(O)O—, —S(O)—, —S(O)₂—, —C(O)NR^{5BB}—, —NR^{5BB}C(O)—, —NR^{5BB}C(O)NH—, —NHC(O)NR^{5BB}—, —[NR^{5BB}C(R^{5BE})(R^{5BF})C(O)]ₙ₄—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

M^{1C} is a bond, —O—, —S—, —NR^{5CB}—, —C(O)—, —C(O)O—, —S(O)—, —S(O)₂—, —C(O)NR^{5CB}, —NR^{5CB}C(O)—, —NR^{5CB}C(O)NH—, —NHC(O)NR^{5CB}—, —[NR^{5CB}CR^{5CE}R^{5CF}C(O)]ₙ₅—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

W^{1A} is bonded to Ab. W^{1C} is bonded to L³.

W^{1A} is a bond, —O—, —S—, —NR^{6BA}—, —C(O)—, C(O)O—, —S(O)—, —S(O)₂—, —C(O)NR^{6BA}—, —NR^{6BA}C(O)—, —NR^{6BA}C(O)NH—, —NHC(O)NR^{6BA}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

W^{1B} is a bond, —O—, —S—, —NR^{6BB}—, —C(O)—, —C(O)O—, —S(O)—, —S(O)₂—, —C(O)NR^{6BB}—, —NR^{6BB}C(O)—, —NR^{6BB}C(O)NH—, —NHC(O)NR^{6BB}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

W^{1C} is a bond, —O—, —S—, —NR^{6BC}—, —C(O)—, —C(O)O—, —S(O)—, —S(O)₂—, —C(O)NR^{6BC}, —NR^{6BC}C(O)—, —NR^{6BC}C(O)NH—, —NHC(O)NR^{6BC}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

R^{1A}, R^{1B}, R^{1C}, R^{1D}, R^{1E}, R^{2B}, R^{3B}, R^{4B}, R^{5B}, R^{5E}, R^{5F}, R^{5AB}, R^{5AE}, R^{5AF}, R^{5BB}, R^{5BE}, R^{5BF}, R^{5CB}, R^{5CE}, R^{5CF}, R^{6B}, R^{6BA}, R^{6BB} and R^{6BC} are independently hydrogen, halogen, —CF₃, —CCl₃, —CBr₃, —CI₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1B} and R^{1C} substituents bonded to the same nitrogen atom may optionally be joined to form a substituted or unsubstituted heterocycloalkyl or substituted or unsubstituted heteroaryl.

The symbol n1 is an integer from 0 to 4. In embodiments, n1 is 0. In embodiments, n1 is 1. In embodiments, n1 is 2. In embodiments, n1 is 3. In embodiments, n1 is 4. The symbol n7 is an integer from 0 to 4. In embodiments, n7 is 0. In embodiments, n7 is 1. In embodiments, n7 is 2. In embodiments, n7 is 3. In embodiments, n1 is 4. The symbol v1 is 1 or 2. The symbols n2, n3, n4, n5 and z3 are independently and integer from 1 to 10. The symbols z1 and z2 are independently an integer from 0 to 10. In embodiments, n2 is 1. In embodiments, n2 is 2. In embodiments, n2 is 3. In embodiments, n2 is 4. In embodiments, n2 is 5. In embodiments, n2 is 6. In embodiments, n2 is 7. In embodiments, n2 is 8. In embodiments, n2 is 9. In embodiments, n2 is 10. In embodiments, n3 is 1. In embodiments, n3 is 2. In embodiments, n3 is 3. In embodiments, n3 is 4. In embodiments, n3 is 5. In embodiments, n3 is 6. In embodiments, n3 is 7. In embodiments, n3 is 8. In embodiments, n3 is 9. In embodiments, n3 is 10. In embodiments, n4 is 1. In embodiments, n4 is 2. In embodiments, n4 is 3. In embodiments, n4 is 4. In embodiments, n4 is 5. In embodiments, n4 is 6. In embodiments, n4 is 7. In embodiments, n4 is 8. In embodiments, n4 is 9. In embodiments, n4 is 10. In embodiments, n5 is 1. In embodiments, n5 is 2. In embodiments, n5 is 3. In embodiments, n5 is 4. In embodiments, n5 is 5. In embodiments, n5 is 6. In embodiments, n5 is 7. In embodiments, n5 is 8. In embodiments, n5 is 9. In embodiments, n5 is 10. In embodiments, z2 is 1. In embodiments, z2 is 2. In embodiments, z2 is 3. In embodiments, z2 is 4. In embodiments, z2 is 5. In embodiments, z2 is 6. In embodiments, z2 is 7. In embodiments, z2 is 8. In embodiments, z2 is 9. In embodiments, z2 is 10. In embodiments, z1 is 1. In embodiments, z1 is 2. In embodiments, z1 is 3. In embodiments, z1 is 4. In embodiments, z1 is 5. In embodiments, z1 is 6. In embodiments, z1 is 7. In embodiments, z1 is 8. In embodiments, z1 is 9. In embodiments, z1 is 10. In embodiments, z3 is 1. In embodiments, z3 is 2. In embodiments, z3 is 3. In embodiments, z3 is 4. In embodiments, z3 is 5. In embodiments, z3 is 6. In embodiments, z3 is 7. In embodiments, z3 is 8. In embodiments, z3 is 9. In embodiments, z3 is 10.

In embodiments, W is covalently attached a cysteine residue within the antibody. In embodiments, the cysteine residue is at Kabat position C214. In embodiments, W is covalently attached to a lysine residue within the antibody.

In embodiments, M is M^{1A} M^{1B} M^{1C}, where M^{1A} is bonded to L³ and M^{1C} is bonded to L⁴.

In embodiments, M^{1A} is a bond, substituted or unsubstituted heteroalkylene or —[NR^{5AB}C(R^{5AE})(R^{5AF})C(O)]ₙ₃. In embodiments, M^{1B} is a bond, substituted or unsubstituted heteroalkylene or —[NR^{5BB}C(R^{5BE})(R^{5BF})C(O)]ₙ₄—. In embodiments, M^{1C} is a bond or substituted or unsubstituted arylene or substituted or unsubstituted heteroarylene. In embodiments, M^{1A} is an amino acid. In embodiments, M^{1B} is an amino acid. In embodiments, at least one of M^{1A} or M^{1B} is valine (val). In embodiments, at least one of M^{1A} or M^{1B} is alanine (ala). In embodiments, at least one of M^{1A} or M^{1B} is citrulline (cit). In embodiments, one of M^{1A}, M^{1B} or M^{1C} is substituted arylene.

In embodiments, at least one of M^{1A}, M^{1B} or M^{1C} has Formula (III):

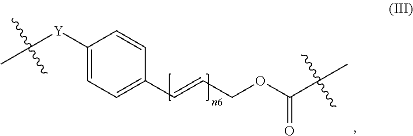

where Y is —NH—, —O—, —C(O)NH— or —C(O)O—; and n6 is an integer from 0 to 3.

In embodiments, W is W^{1A}—W^{1B}—W^{1C}, where W^{1A} is bonded to Ab and W^{1C} is bonded to L³.

In embodiments, P is substituted or unsubstituted alkyl.

In embodiments, z3 is 1 or 2.

In embodiments, L³ is substituted or unsubstituted alkylene or substituted or unsubstituted heteroalkylene.

In embodiments, L⁴ is substituted or unsubstituted alkylene or substituted or unsubstituted heteroalkylene.

In embodiments, W is substituted or unsubstituted heteroalkylene, substituted or unsubstituted cycloalkylene, substituted or unsubstituted heterocycloalkylene, substituted or unsubstituted arylene or substituted or unsubstituted heteroarylene.

In embodiments, W is 5- or 6-membered substituted or unsubstituted heterocycloalkylene.

In embodiments, W has the formula:

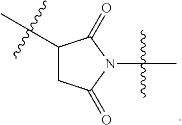 [SMILES: *C1CC(=O)N(*)C1=O]

In embodiments, M comprises a peptide.

In embodiments, —[W-(L³)_{z1}-M-(L⁴)_{z2}-P-D] is:

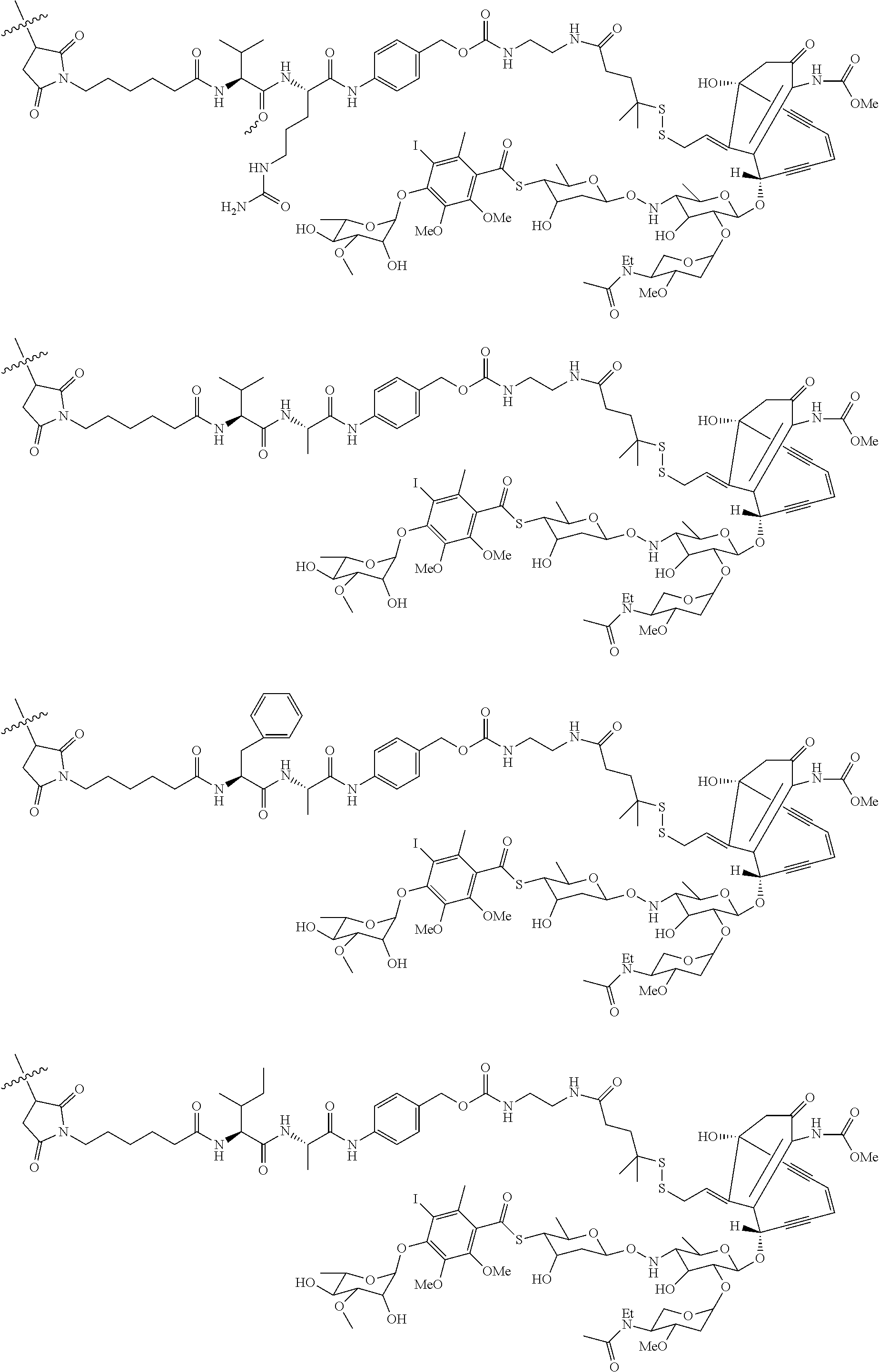

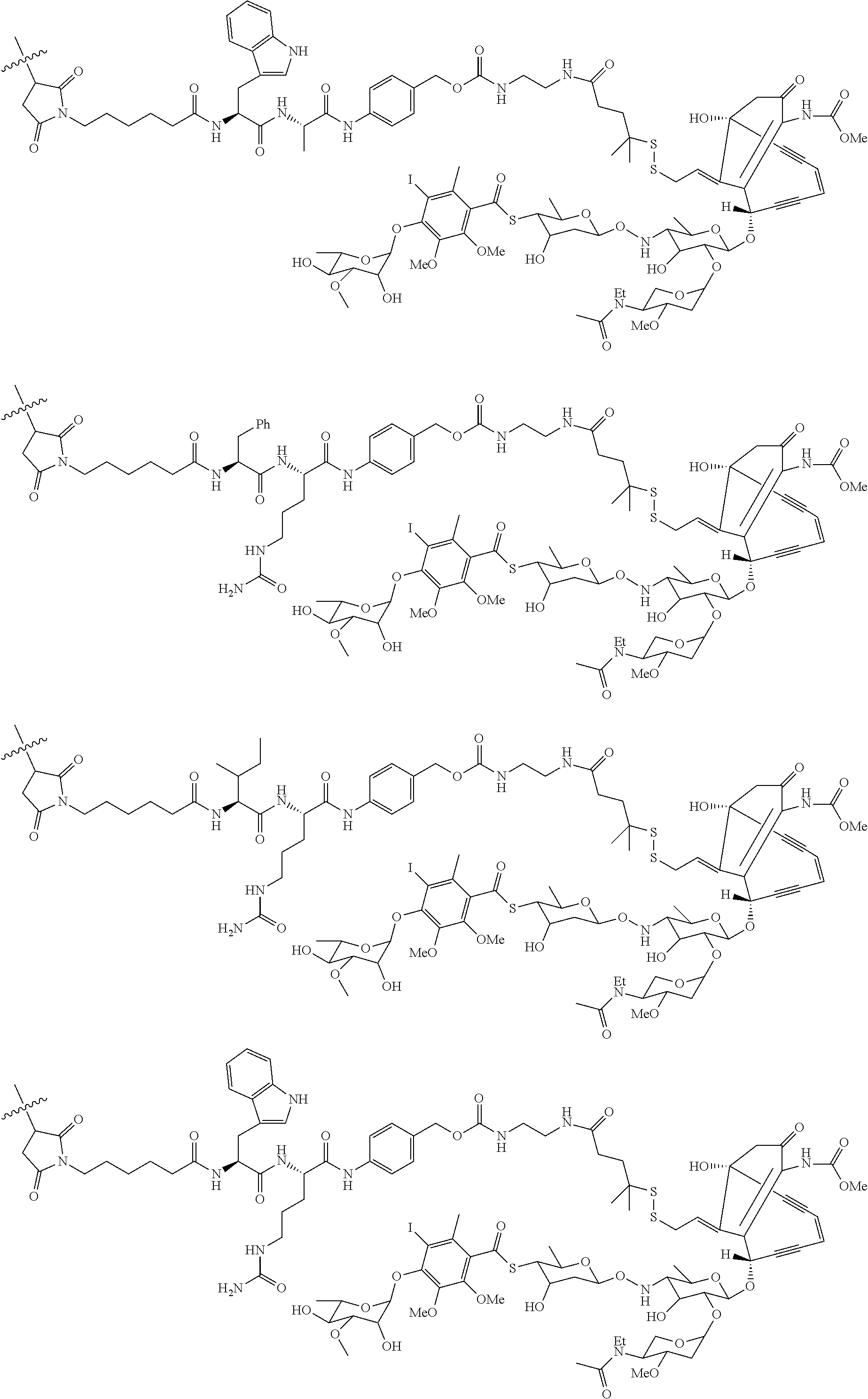

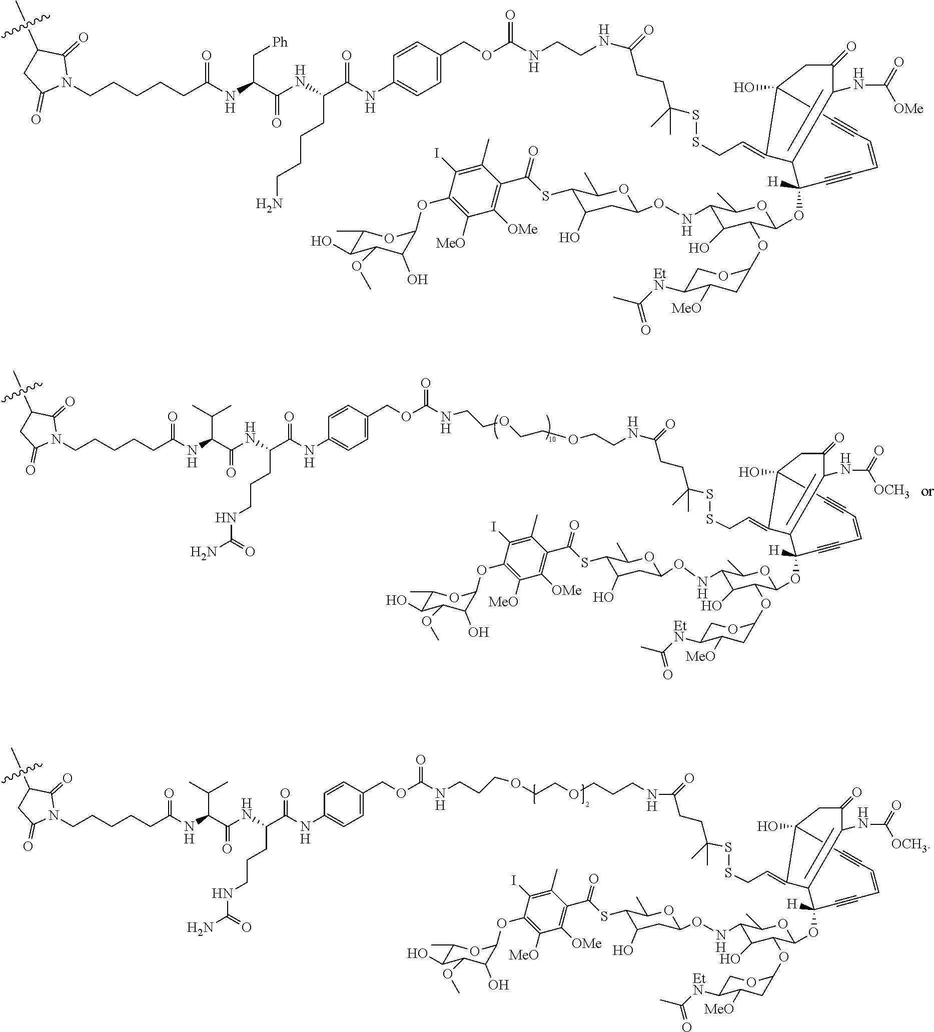

In embodiments, —[W-(L³)_{z1}-M-(L⁴)_{z2}-P-D] is of formula:

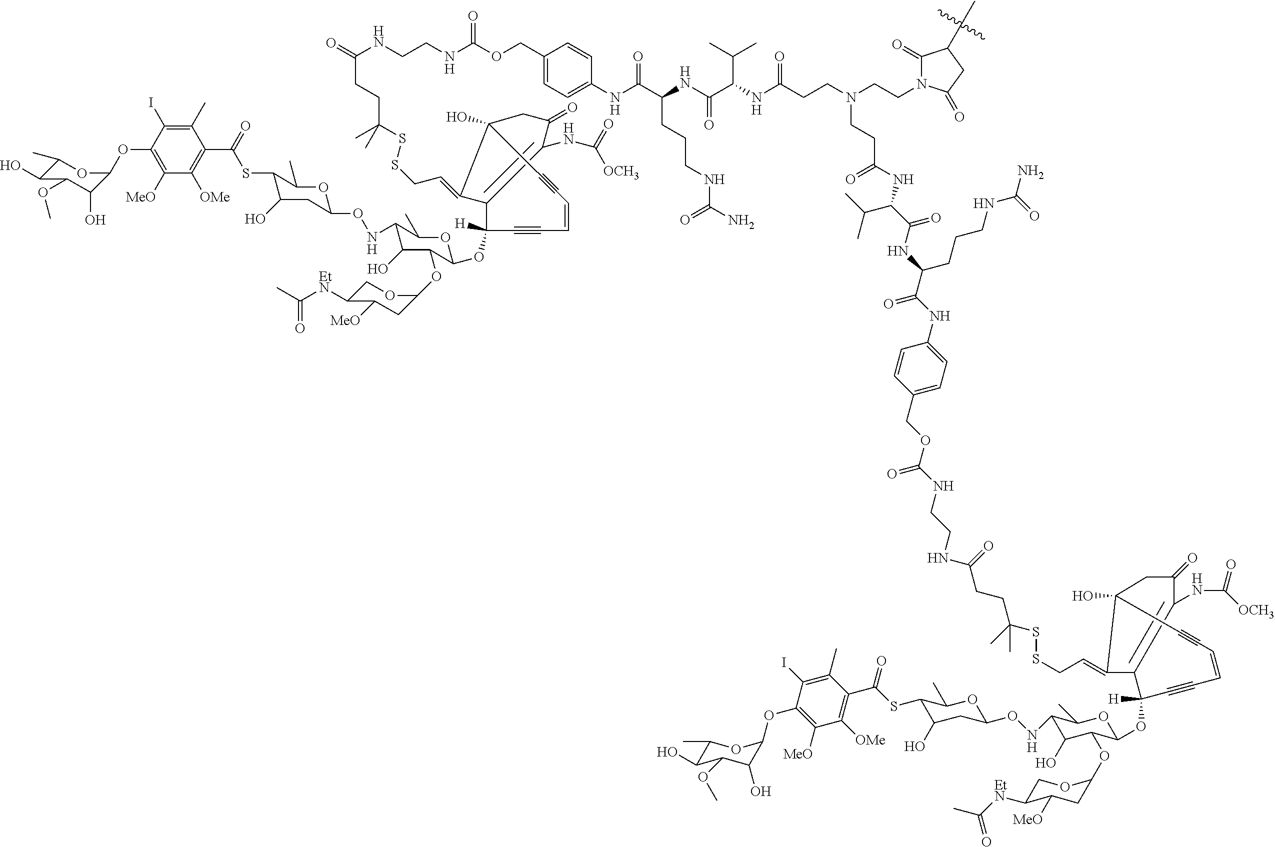

In a further aspect, there is provided a compound of Formula (IV):

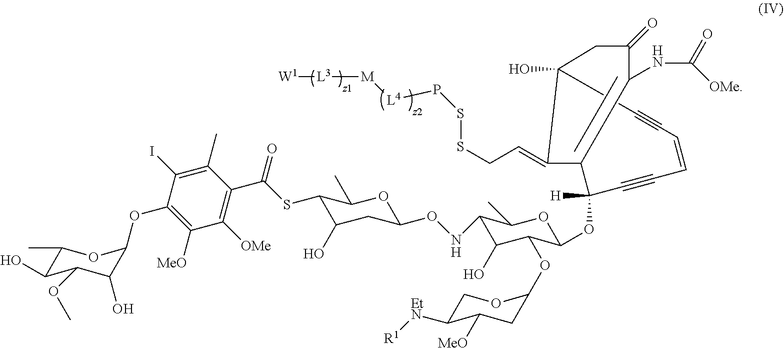

n1, z1, z2, L³, L⁴, R¹, P and M are as described herein.

W¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —N₃, —CF₃, —CCl₃, —CBr₃, CI₃, —CN, —C(O)R^{7E}, —OR^{7A}, —NR^{7B}R^{7C}, —C(O)OR^{7A}, —C(O)NR^{7B}R^{7C}, —NO₂, SR^{7D}, —SOₙ₇R^{7B}, —SOᵥ₇NR^{7B}R^{7C}, —NHNR^{7B}R^{7C}, ONR^{7B}R^{7C}, —NHC(O)NHNR^{7B}R^{7C}.

The symbol n7 is an integer from 0 to 4. The symbol v7 is 1 or 2.

In embodiments, the compound of Formula (IV) has formula:

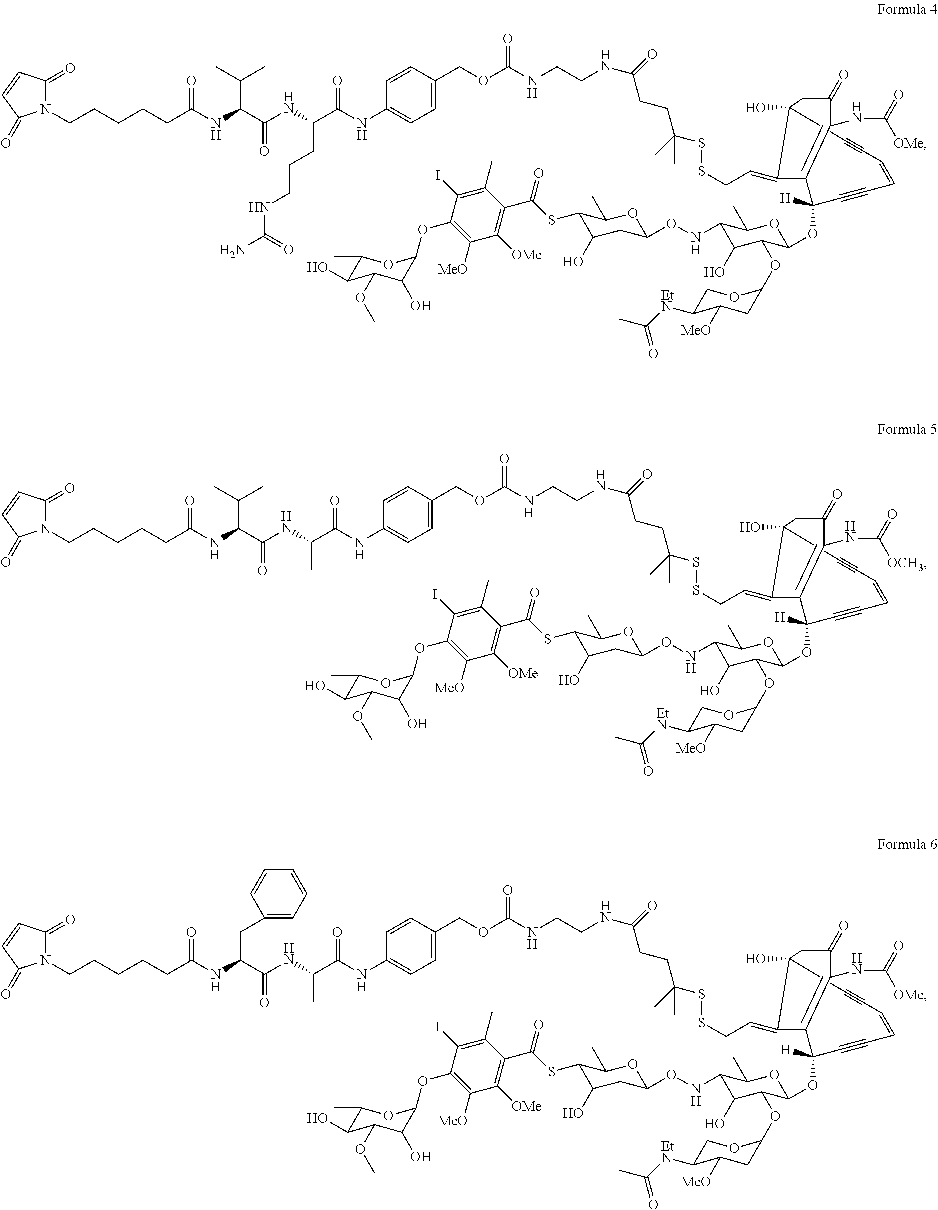

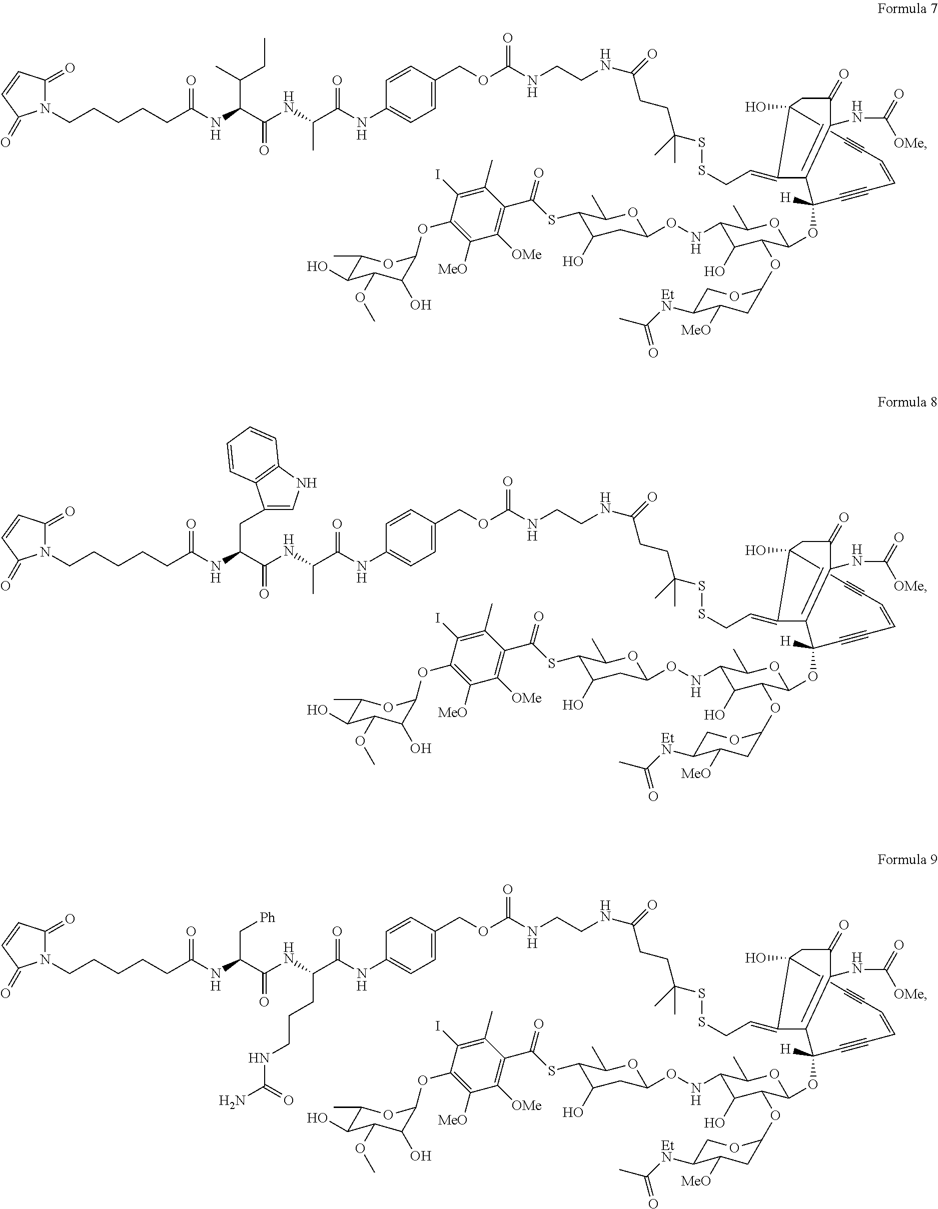

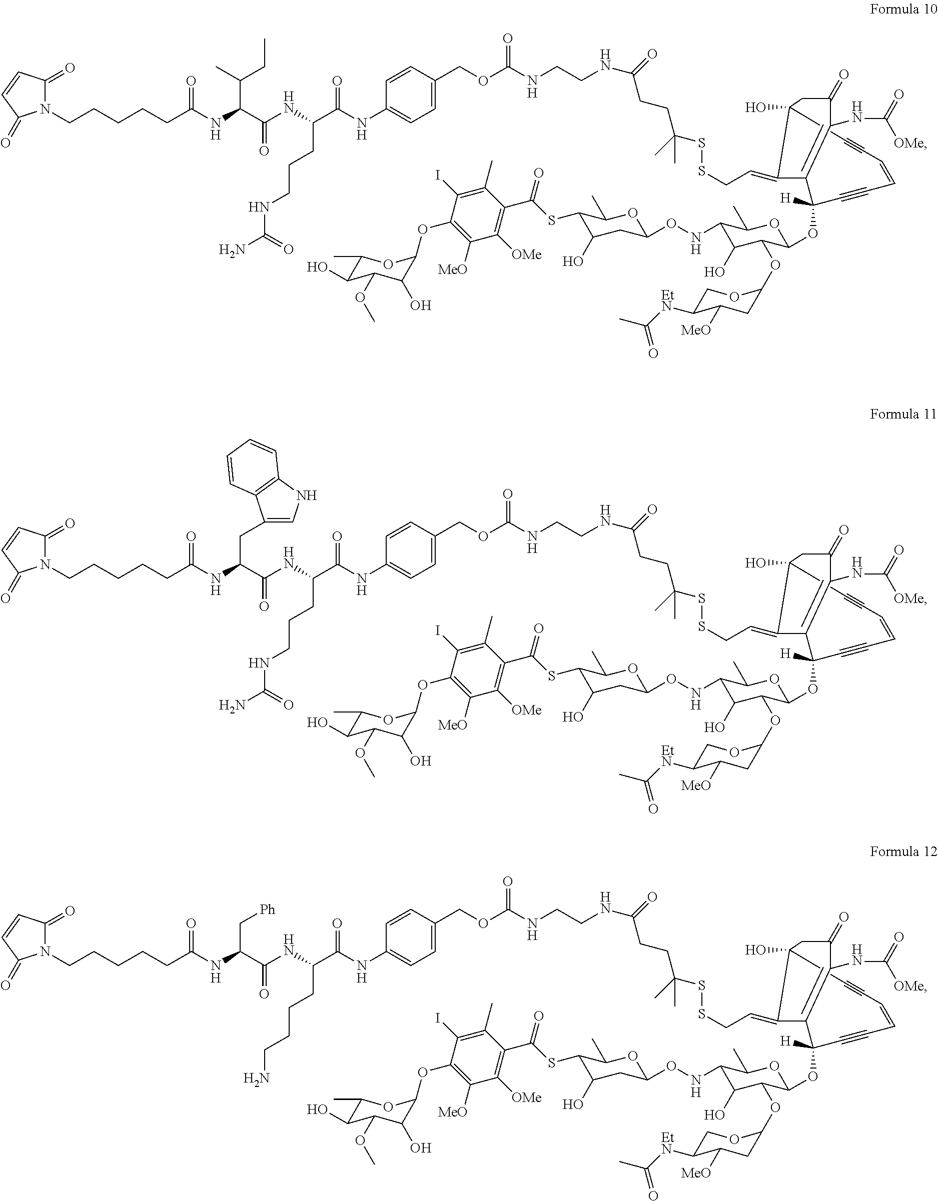

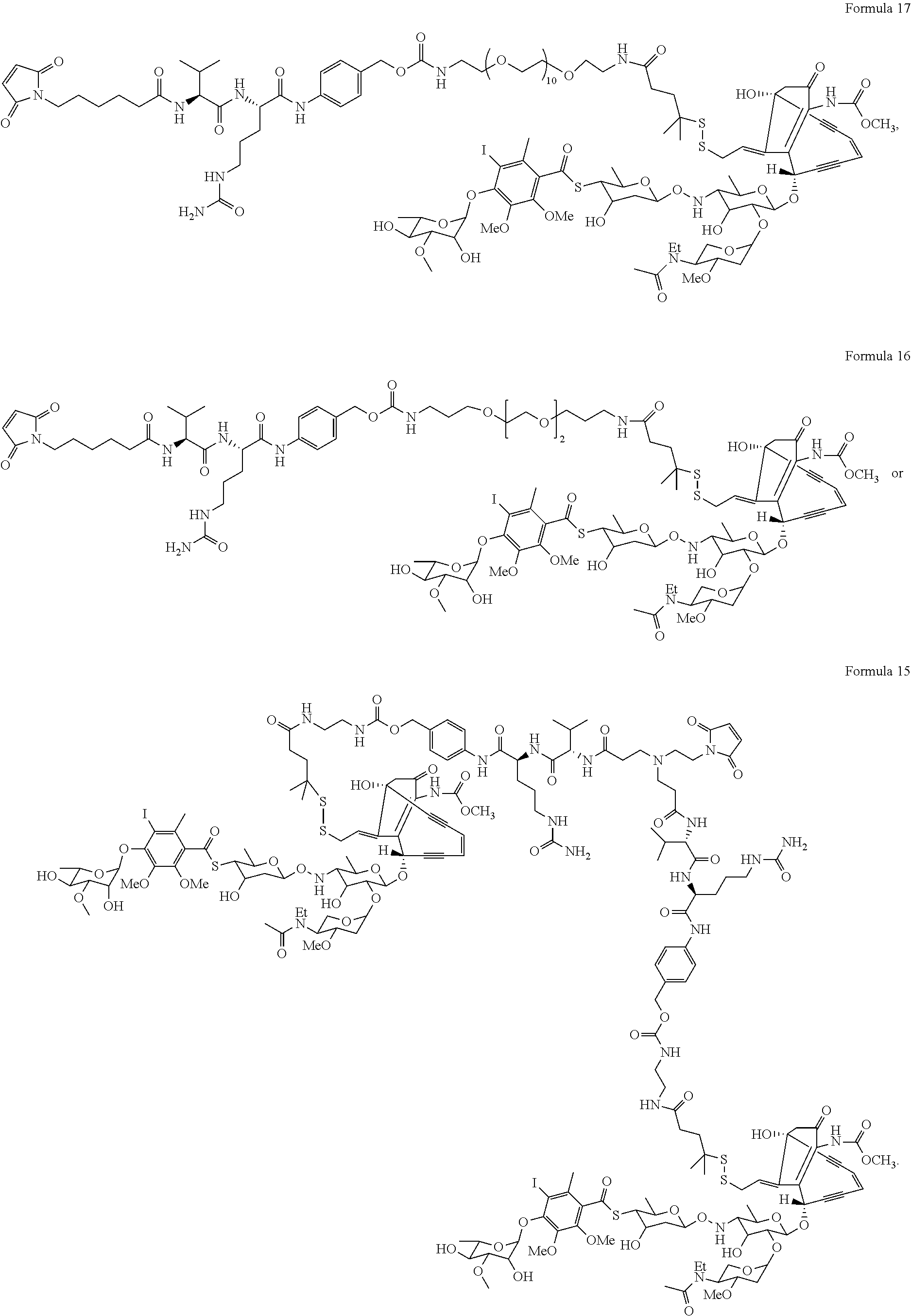

In embodiments, R¹ is hydrogen, substituted or unsubstituted alkyl or —C(O)R^{1E}. In embodiments, R is hydrogen or —C(O)R^{1E}. In embodiments, R is —C(O)R^{1E}. In embodiments, R¹ is —C(O)CH₃, —C(O)CH₂CH₃, —C(O)CH₂CH₂CH₃ or —C(O)CH₂CH₂CH₂CH₃. In embodiments, R¹ is —C(O)CH₃.

In embodiments, L³ is independently bond, —O—, —S—, —NH—, —C(O)—, —C(O)O—, —S(O)—, —S(O)₂, —C(O)NH—, —NHC(O)—, —NHC(O)NH—, R^{3G}-substituted or unsubstituted alkylene or R^{3G-}substituted or unsubstituted heteroalkylene. In embodiments, L³ is independently bond, —O—, —S—, —NH—, —C(O)—, —C(O)O—, —S(O)—, —S(O)₂, —C(O)NH—, —NHC(O)—, —NHC(O)NH—, R^{3G}-substituted or unsubstituted C₁-C₆ alkylene or R^{3G}-substituted or unsubstituted 2 to 6 membered heteroalkylene.

R^{3G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{3H}-substituted or unsubstituted alkyl, R^{3H}-substituted or unsubstituted heteroalkyl, R^{3H}-substituted or unsubstituted cycloalkyl, R^{3H}-substituted or unsubstituted heterocycloalkyl, R^{3H}-substituted or unsubstituted aryl, or R^{3H}-substituted or unsubstituted heteroaryl. In embodiments, R^{3G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{3H}-substituted or unsubstituted C₁-C₆ alkyl, R^{3H}-substituted or unsubstituted 2 to 6 membered heteroalkyl, R^{3H}-substituted or unsubstituted C₃-C₆ cycloalkyl, R^{3H}-substituted or unsubstituted 3 to 6 membered heterocycloalkyl, R^{3H}-substituted or unsubstituted phenyl, or R^{3H}-substituted or unsubstituted 5 to 6 membered heteroaryl.

In embodiments, L⁴ is independently bond, —O—, —S—, —NH—, —C(O)—, —C(O)O—, —S(O)—, —S(O)₂, —C(O)NH—, —NHC(O)—, —NHC(O)NH—, R^{4G}-substituted or unsubstituted alkylene or R^{4G-}substituted or unsubstituted heteroalkylene. In embodiments, L⁴ is independently bond, —O—, —S—, —NH—, —C(O)—, —C(O)O—, —S(O)—, —S(O)₂, —C(O)NH—, —NHC(O)—, —NHC(O)NH—, R^{4G}-substituted or unsubstituted C₁-C₆ alkylene or R^{4G}-substituted or unsubstituted 2 to 6 membered heteroalkylene.

R^{4G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{4H}-substituted or unsubstituted alkyl, R^{4H}-substituted or unsubstituted heteroalkyl, R^{4H}-substituted or unsubstituted cycloalkyl, R^{4H}-substituted or unsubstituted heterocycloalkyl, R^{4H}-substituted or unsubstituted aryl, or R^{4H}-substituted or unsubstituted heteroaryl. In embodiments, R^{4G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{4H}-substituted or unsubstituted C₁-C₆ alkyl, R^{4H}-substituted or unsubstituted 2 to 6 membered heteroalkyl, R^{4H}-substituted or unsubstituted C₃-C₆ cycloalkyl, R^{4H}-substituted or unsubstituted 3 to 6 membered heterocycloalkyl, R^{4H}-substituted or unsubstituted phenyl, or R^{4H}-substituted or unsubstituted 5 to 6 membered heteroaryl.

In embodiments, R¹ is independently hydrogen, halogen, —CF₃, —CCl₃, —CBr₃, —CI₃, —CN, —C(O)H, —OH, —NH₂, —C(O)OH, —C(O)NH₂, —SH, —SO₃H, —SO₄H, —SO₂NH₂, R^{1G}-substituted or unsubstituted alkyl, R^{1G}-substituted or unsubstituted heteroalkyl, R^{1G}-substituted or unsubstituted cycloalkyl, R^{1G}-substituted or unsubstituted heterocycloalkyl, R^{1G}-substituted or unsubstituted aryl, or R^{1G}-substituted or unsubstituted heteroaryl. In embodiments, R¹ is independently hydrogen, halogen, —CF₃, —CCl₃, —CBr₃, —CI₃, —CN, —C(O)H, —OH, —NH₂, —C(O)OH, —C(O)NH₂, —SH, —SO₃H, —SO₄H, —SO₂NH₂, R^{1G}-substituted or unsubstituted C₁-C₆ alkyl, R^{1G}-substituted or unsubstituted 2 to 6 membered heteroalkyl, R^{1G}-substituted or unsubstituted C₃-C₆ cycloalkyl, R^{1G}-substituted or unsubstituted 3 to 6 membered heterocycloalkyl, R^{1G}-substituted or unsubstituted phenyl, or R^{1G} substituted or unsubstituted 5 to 6 membered heteroaryl.

R^{1G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1H}-substituted or unsubstituted alkyl, R^{1H}-substituted or unsubstituted heteroalkyl, R^{1H}-substituted or unsubstituted cycloalkyl, R^{1H}-substituted or unsubstituted heterocycloalkyl, R^{1H}-substituted or unsubstituted aryl, or R^{1H}-substituted or unsubstituted heteroaryl. In embodiments, R^{1G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1H}-substituted or unsubstituted C₁-C₆ alkyl, R^{1H}-substituted or unsubstituted 2 to 6 membered heteroalkyl, R^{1H}-substituted or unsubstituted C₃-C₆ cycloalkyl, R^{1H}-substituted or unsubstituted 3 to 6 membered heterocycloalkyl, R^{1H}-substituted or unsubstituted phenyl, or R^{1H}-substituted or unsubstituted 5 to 6 membered heteroaryl.

In embodiments, P is independently —O—, —S—, —NH—, —C(O)—, —C(O)O—, —S(O)—, —S(O)₂—, —C(O)NH—, —NHC(O)—, —NHC(O)NH—, R^{2G}-substituted or unsubstituted alkyl, R^{2G}-substituted or unsubstituted heteroalkyl, R^{2G}-substituted or unsubstituted cycloalkyl, R^{2G}-substituted or unsubstituted heterocycloalkyl, R^{2G}-substituted or unsubstituted aryl, or R^{2G}-substituted or unsubstituted heteroaryl. In embodiments, P is independently —O—, —S—, —NH—, —C(O)—, —C(O)O—, —S(O)—, —S(O)₂—, —C(O)NH—, —NHC(O)—, —NHC(O)NH—, R^{2G}-substituted or unsubstituted C₁-C₆ alkyl, R^{2G}-substituted or unsubstituted 2 to 6 membered heteroalkyl, R^{2G}-substituted or unsubstituted C₃-C₆ cycloalkyl, R^{2G}-substituted or unsubstituted 3 to 6 membered heterocycloalkyl, R^{2G}-substituted or unsubstituted phenyl, or R^{2G}-substituted or unsubstituted 5 to 6 membered heteroaryl.

R^{2G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{2H}-substituted or unsubstituted alkyl, R^{2H}-substituted or unsubstituted heteroalkyl, R^{2H}-substituted or unsubstituted cycloalkyl, R^{2H}-substituted or unsubstituted heterocycloalkyl, R^{2H}-substituted or unsubstituted aryl, or R^{2H}-substituted or unsubstituted heteroaryl. In embodiments, R^{2G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{2H}-substituted or unsubstituted C₁-C₆ alkyl, R^{2H}-substituted or unsubstituted 2 to 6 membered heteroalkyl, R^{2H}-substituted or unsubstituted C₃-C₆ cycloalkyl, R^{2H}-substituted or unsubstituted 3 to 6 membered heterocycloalkyl, R^{2H}-substituted or unsubstituted phenyl, or R^{2H}-substituted or unsubstituted 5 to 6 membered heteroaryl.

In embodiments, M is independently —O—, —S—, —NH—, —C(O)—, —C(O)O—, —S(O)—, —S(O)₂—, —C(O)NH—, —NHC(O)—, —NHC(O)NH—, —[NHCH₂C(O)]—, —[NHCH₂C(O)]₂—, —[NHCH₂C(O)]₃—, —[NHCH₂C(O)]₄—, —[NHCH₂C(O)]₅—, —[NHCH₂C(O)]₆—, —[NHCH₂C(O)]7-, —[NHCH₂C(O)]₈—, —[NHCH₂C(O)]₉—, —[NHCH₂C(O)]₁₀—, R^{5G}-substituted or unsubstituted alkyl, R G-substituted or unsubstituted heteroalkyl, R^{5G}-substituted or unsubstituted cycloalkyl, R^{5G}-substituted or unsubstituted heterocycloalkyl, R^{5G}-substituted or unsubstituted aryl, R G-substituted or unsubstituted heteroaryl or M^{1A}-M^{1B}-M^{1C}. In embodiments, M is independently —O—, —S—, —NH—, —C(O)—, —C(O)O—, —S(O)—, —S(O)₂—, —C(O)NH—, —NHC(O)—, —NHC(O)NH—, —[NHCH₂C(O)]—, —[NHCH₂C(O)]₂—, —[NHCH₂C(O)]₃—, —[NHCH₂C(O)]₄—, —[NHCH₂C(O)]₅—, —[NHCH₂C(O)]₆—, —[NHCH₂C(O)]₇—, —[NHCH₂C(O)]₈—, —[NHCH₂C(O)]₉—, —[NHCH₂C(O)]₁₀—, R^{5G}-substituted or unsubstituted C₁-C₆ alkyl, R^{5G}-substituted or unsubstituted 2 to 6 membered heteroalkyl, R^{5G}-substituted or unsubstituted C₃-C₆ cycloalkyl, R^{5G}-substituted or unsubstituted 3 to 6 membered heterocycloalkyl, R^{5G}-substituted or unsubstituted phenyl, R^{5G}-substituted or unsubstituted 5 to 6 membered heteroaryl or M^{1A}-M^{1B}-M^{1C}.

R^{5G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5H}-substituted or unsubstituted alkyl, R^{5H}-substituted or unsubstituted heteroalkyl, R^{5H}-substituted or unsubstituted cycloalkyl, R^{5H}-substituted or unsubstituted heterocycloalkyl, R^{5H}-substituted or unsubstituted aryl, or R^{5H}-substituted or unsubstituted heteroaryl. In embodiments, R^{5G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5H}-substituted or unsubstituted C₁-C₆ alkyl, R^{5H}-substituted or unsubstituted 2 to 6 membered heteroalkyl, R^{5H}-substituted or unsubstituted C₃-C₆ cycloalkyl, R^{5H}-substituted or unsubstituted 3 to 6 membered heterocycloalkyl, R^{5H}-substituted or unsubstituted phenyl, or R^{5H}-substituted or unsubstituted 5 to 6 membered heteroaryl.

In embodiments, W is independently —O—, —S—, —NH—, —C(O)—, —C(O)O—, —S(O)—, —S(O)₂—, —C(O)NH—, —NHC(O)—, —NHC(O)NH—, R^{6G}-substituted or unsubstituted alkyl, R^{6G}-substituted or unsubstituted heteroalkyl, R^{6G}-substituted or unsubstituted cycloalkyl, R^{6G}-substituted or unsubstituted heterocycloalkyl, R^{6G}-substituted or unsubstituted aryl, R^{6G}-substituted or unsubstituted heteroaryl or W^{1A}—W^{1B}—W^{1C}. In embodiments, W is independently —O—, —S—, —NH—, —C(O)—, —C(O)O—, —S(O)—, —S(O)₂—, —C(O)NH—, —NHC(O)—, —NHC(O)NH, R^{6G}-substituted or unsubstituted C₁-C₆ alkyl, R^{6G}-substituted or unsubstituted 2 to 6 membered heteroalkyl, R^{6G}-substituted or unsubstituted C₃-C₆ cycloalkyl, R^{6G}-substituted or unsubstituted 3 to 6 membered heterocycloalkyl, R^{6G}-substituted or unsubstituted phenyl, or R^{6G}-substituted or unsubstituted 5 to 6 membered heteroaryl or W^{1A}—W^{1B}—W^{1C}. In embodiments, W is -[(L³)_{z1}-M-(L⁴)_{z2}-P-D] or -[(L³′)_{z1′}, -M′-(L^{4′})_{z2′}-P′-D′], where -[(L³)_{z1}-M-(L⁴)_{z2}-P-D] is the same as -[(L^{3′})_{z1′}-M′-(L⁴′)_{z2′}, —P′-D′] or is optionally different. z1, z2, L³, L⁴, P, M and D are independently the same as z1′, z2′, L^{3′}, L^{4′}, P′, M′ and D′ or are independently optionally different. z1, z2, L³, L⁴, P, M and D are as described herein. z1′, z2′, L³′, L⁴′, P′, M′ and D′ independently correspond to z1, z2, L³, L⁴, P, M and D and as such are as defined herein.

R^{6G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6H}-substituted or unsubstituted alkyl, R^{6H}-substituted or unsubstituted heteroalkyl, R^{6H}-substituted or unsubstituted cycloalkyl, R^{6H}-substituted or unsubstituted heterocycloalkyl, R^{6H}-substituted or unsubstituted aryl, or R^{6H}-substituted or unsubstituted heteroaryl. In embodiments, R^{6G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6H}-substituted or unsubstituted C₁-C₆ alkyl, R^{6H}-substituted or unsubstituted 2 to 6 membered heteroalkyl, R^{6H}-substituted or unsubstituted C₃-C₆ cycloalkyl, R^{6H}-substituted or unsubstituted 3 to 6 membered heterocycloalkyl, R^{6H}-substituted or unsubstituted phenyl, or R^{6H}-substituted or unsubstituted 5 to 6 membered heteroaryl.

In embodiments, W¹ is hydrogen, halogen, —N₃, —CF₃, —CCl₃, —CBr₃, —CI₃, —CN, —C(O)H, —OH, —NH₂, —C(O)OH, —C(O)NH₂, —NO₂, —SH, SO₃H, —SO₄H, —SO₂NH₂, —NHNH₂, —ONH₂, —NHC(O)NHNH₂, R^{7G}-substituted or unsubstituted alkyl, R^{7G}-substituted or unsubstituted heteroalkyl, R^{7G}-substituted or unsubstituted cycloalkyl, R^{7G}-substituted or unsubstituted heterocycloalkyl, R^{7G}-substituted or unsubstituted aryl or R^{7G}-substituted or unsubstituted heteroaryl. In embodiments, W is independently —O—, —S—, —NH—, —C(O)—, —C(O)O—, —S(O)—, —S(O)₂—, —C(O)NH—, —NHC(O)—, —NHC(O)NH, R^{7G}-substituted or unsubstituted C₁-C₆ alkyl, R^{7G-}substituted or unsubstituted 2 to 6 membered heteroalkyl, R^{7G}-substituted or unsubstituted C₃-C₆ cycloalkyl, R^{7G}-substituted or unsubstituted 3 to 6 membered heterocycloalkyl, R^{7G}-substituted or unsubstituted phenyl, or R^{7G}-substituted or unsubstituted 5 to 6 membered heteroaryl.

R^{7G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R⁷H-substituted or unsubstituted alkyl, R⁷H-substituted or unsubstituted heteroalkyl, R^{7H}-substituted or unsubstituted cycloalkyl, R^{7H}-substituted or unsubstituted heterocycloalkyl, R⁷H-substituted or unsubstituted aryl, or R^{7H}-substituted or unsubstituted heteroaryl. In embodiments, R^{7G} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R⁷H-substituted or unsubstituted C₁-C₆ alkyl, R⁷H-substituted or unsubstituted 2 to 6 membered heteroalkyl, R⁷H-substituted or unsubstituted C₃-C₆ cycloalkyl, R^{7H}-substituted or unsubstituted 3 to 6 membered heterocycloalkyl, R^{7H}-substituted or unsubstituted phenyl, or R⁷H-substituted or unsubstituted 5 to 6 membered heteroaryl.

In embodiments, M^{1A} is independently a bond —O—, —S—, —NH—, —C(O)—, —C(O)O—, —S(O)—, —S(O)₂—, —C(O)NH—, —NHC(O)—, —NHC(O)NH—, —[NHCH₂C(O)]—, —[NHCH₂C(O)]₂—, —[NHCH₂C(O)]₃—, —[NHCH₂C(O)]₄—, —[NHCH₂C(O)]₅—, —[NHCH₂C(O)]₆—, —[NHCH₂C(O)]—, —[NHCH₂C(O)]₈—, —[NHCH₂C(O)]₉—, —[NHCH₂C(O)]₁₀—, R^{5AG}-substituted or unsubstituted alkyl, R^{5AG}-substituted or unsubstituted heteroalkyl, R^{5AG}-substituted or unsubstituted cycloalkyl, R^{5AG} substituted or unsubstituted heterocycloalkyl, R^{5AG}-substituted or unsubstituted aryl, R^{5AG}-substituted or unsubstituted heteroaryl. In embodiments, M^{1A} is independently a bond, —O—, —S—, —NH—, —C(O)—, —C(O)O—, —S(O)—, —S(O)₂—, —C(O)NH—, —NHC(O)—, —NHC(O)NH—, —[NHCH₂C(O)]—, —[NHCH₂C(O)]₂—, —[NHCH₂C(O)]₃—, —[NHCH₂C(O)]₄—, —[NHCH₂C(O)]₅—, —[NHCH₂C(O)]₆—, —[NHCH₂C(O)]7-, —[NHCH₂C(O)]₈—, —[NHCH₂C(O)]₉—, —[NHCH₂C(O)]10-, R^{5AG}-substituted or unsubstituted C₁-C₆ alkyl, R^{5AG}-substituted or unsubstituted 2 to 6 membered heteroalkyl, R^{SAG}-substituted or unsubstituted C₃-C₆ cycloalkyl, R^{5AG}-substituted or unsubstituted 3 to 6 membered heterocycloalkyl, R^{5AG}-substituted or unsubstituted phenyl, or R^{5AG}-substituted or unsubstituted 5 to 6 membered heteroaryl.

R^{5AG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5AH}-substituted or unsubstituted alkyl, R^{5AH}-substituted or unsubstituted heteroalkyl, R^{5AH}-substituted or unsubstituted cycloalkyl, R^{5AH}-substituted or unsubstituted heterocycloalkyl, R^{5AH}-substituted or unsubstituted aryl, or R^{5AH}-substituted or unsubstituted heteroaryl. In embodiments, R^{SAG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5AH}-substituted or unsubstituted C₁-C₆ alkyl, R^{5AH}-substituted or unsubstituted 2 to 6 membered heteroalkyl, R^{5AH} substituted or unsubstituted C₃-C₆ cycloalkyl, R^{5AH}-substituted or unsubstituted 3 to 6 membered heterocycloalkyl, R^{5AH}-substituted or unsubstituted phenyl, or R^{5AH}-substituted or unsubstituted 5 to 6 membered heteroaryl.

In embodiments, M^{1B} is independently a bond —O—, —S—, —NH—, —C(O)—, —C(O)O—, —S(O)—, —S(O)₂—, —C(O)NH—, —NHC(O)—, —NHC(O)NH—, —[NHCH₂C(O)]—, —[NHCH₂C(O)]₂—, —[NHCH₂C(O)]₃—, —[NHCH₂C(O)]₄—, —[NHCH₂C(O)]₅—, —[NHCH₂C(O)]₆—, —[NHCH₂C(O)]₇—, —[NHCH₂C(O)]₈—, —[NHCH₂C(O)]₉—, —[NHCH₂C(O)]₁₀—, R^{5BG}-substituted or unsubstituted alkyl, R^{5BG}-substituted or unsubstituted heteroalkyl, R^{5BG}-substituted or unsubstituted cycloalkyl, R^{5BG}-substituted or unsubstituted heterocycloalkyl, R^{5BG}-substituted or unsubstituted aryl, R^{5B}G substituted or unsubstituted heteroaryl. In embodiments, M^{1B} is independently a bond, —O—, —S—, —NH—, —C(O)—, —C(O)O—, —S(O)—, —S(O)₂—, —C(O)NH—, —NHC(O)—, —NHC(O)NH—, —[NHCH₂C(O)]—, —[NHCH₂C(O)]₂—, —[NHCH₂C(O)]₃—, —[NHCH₂C(O)]₄—, —[NHCH₂C(O)]₅—, —[NHCH₂C(O)]₆—, —[NHCH₂C(O)]₇—, —[NHCH₂C(O)]₈—, —[NHCH₂C(O)]₉—, —[NHCH₂C(O)]₁₀—, R^{5BG}-substituted or unsubstituted C₁-C₆ alkyl, R^{5BG}-substituted or unsubstituted 2 to 6 membered heteroalkyl, R^{5B}G substituted or unsubstituted C₃-C₆ cycloalkyl, R^{5BG}-substituted or unsubstituted 3 to 6 membered heterocycloalkyl, R^{5BG}-substituted or unsubstituted phenyl, or R^{5BG}-substituted or unsubstituted 5 to 6 membered heteroaryl.

R^{5BG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5BH}-substituted or unsubstituted alkyl, R^{5BH}-substituted or unsubstituted heteroalkyl, R^{5BH}-substituted or unsubstituted cycloalkyl, R^{5BH}-substituted or unsubstituted heterocycloalkyl, R^{5BH}-substituted or unsubstituted aryl, or R^{5BH}-substituted or unsubstituted heteroaryl. In embodiments, R^{5BG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5BH}-substituted or unsubstituted C₁-C₆ alkyl, R^{5BH}-substituted or unsubstituted 2 to 6 membered heteroalkyl, R^{5BH}-substituted or unsubstituted C₃-C₆ cycloalkyl, R^{5BH}-substituted or unsubstituted 3 to 6 membered heterocycloalkyl, R^{5BH}-substituted or unsubstituted phenyl, or R^{5BH}-substituted or unsubstituted 5 to 6 membered heteroaryl.

In embodiments, M^{1C} is independently a bond —O—, —S—, —NH—, —C(O)—, —C(O)O—, —S(O)—, —S(O)₂—, —C(O)NH—, —NHC(O)—, —NHC(O)NH—, —[NHCH₂C(O)]—, —[NHCH₂C(O)]₂—, —[NHCH₂C(O)]₃—, —[NHCH₂C(O)]₄—, —[NHCH₂C(O)]₅—, —[NHCH₂C(O)]₆—, —[NHCH₂C(O)]₇—, —[NHCH₂C(O)]₈—, —[NHCH₂C(O)]₉—, —[NHCH₂C(O)]₁₀—, R^{5CG}-substituted or unsubstituted alkylene, R^{5CG}-substituted or unsubstituted heteroalkylene, R^{5CG}-substituted or unsubstituted cycloalkylene, R^{5CG}-substituted or unsubstituted heterocycloalkylene, R^{5CG}-substituted or unsubstituted aryl, R^{5CG} substituted or unsubstituted heteroaryl. In embodiments, M^{1C} is independently a bond, —O—, —S—, —NH—, —C(O)—, —C(O)O—, —S(O)—, —S(O)₂—, —C(O)NH—, —NHC(O)—, —NHC(O)NH—, —[NHCH₂C(O)]—, —[NHCH₂C(O)]₂—, —[NHCH₂C(O)]₃—, —[NHCH₂C(O)]₄—, —[NHCH₂C(O)]₅—, —[NHCH₂C(O)]₆—, —[NHCH₂C(O)]₇—, —[NHCH₂C(O)]₈—, —[NHCH₂C(O)]₉—, —[NHCH₂C(O)]₁₀—, R^{5CG}-substituted or unsubstituted C₁-C₆ alkylene, R^{5CG}-substituted or unsubstituted 2 to 6 membered heteroalkylene, R^{5CG}-substituted or unsubstituted C₃-C₆ cycloalkylene, R⁵-substituted or unsubstituted 3 to 6 membered heterocycloalkylene, R^{5CG}-substituted or unsubstituted phenyl, or R^{5CG}-substituted or unsubstituted 5 to 6 membered heteroaryl.

R^{5CG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5CH}-substituted or unsubstituted alkyl, R^{5CH}-substituted or unsubstituted heteroalkyl, R^{5CH}-substituted or unsubstituted cycloalkyl, R^{5CH}-substituted or unsubstituted heterocycloalkyl, R^{5CH}-substituted or unsubstituted aryl, or R^{5CH}-substituted or unsubstituted heteroaryl. In embodiments, R^{5CG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5CH}-substituted or unsubstituted C₁-C₆ alkyl, R^{5CH}-substituted or unsubstituted 2 to 6 membered heteroalkyl, R^{5CH}-substituted or unsubstituted C₃-C₆ cycloalkyl, R^{5CH}-substituted or unsubstituted 3 to 6 membered heterocycloalkyl, R^{5CH}-substituted or unsubstituted phenyl, or R^{5CH}-substituted or unsubstituted 5 to 6 membered heteroaryl.

In embodiments, W^{1A} is independently a bond, —O—, —S—, —NH—, —C(O)—, —C(O)O—, —S(O)—, —S(O)₂—, —C(O)NH—, —NHC(O)—, —NHC(O)NH—, R^{6AG}-substituted or unsubstituted alkylene, R^{6AG}-substituted or unsubstituted heteroalkylene, R^{6AG}-substituted or unsubstituted cycloalkylene, R^{6AG} substituted or unsubstituted heterocycloalkylene, R⁶-substituted or unsubstituted aryl, R^{6AG}-substituted or unsubstituted heteroaryl. In embodiments, W^{1A} is independently a bond, —O—, —S—, —NH—, —C(O)—, —C(O)O—, —S(O)—, —S(O)₂—, —C(O)NH—, —NHC(O)—, —NHC(O)NH, R^{6AG}-substituted or unsubstituted C₁-C₆ alkylene, R^{6AG}-substituted or unsubstituted 2 to 6 membered heteroalkylene, R^{6AG}-substituted or unsubstituted C₃-C₆ cycloalkylene, R^{6AG}-substituted or unsubstituted 3 to 6 membered heterocycloalkylene, R^{6AG}-substituted or unsubstituted phenyl, or R^{6AG}-substituted or unsubstituted 5 to 6 membered heteroaryl.

R^{6AG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6AH}-substituted or unsubstituted alkyl, R^{6AH}-substituted or unsubstituted heteroalkyl, R^{6AH}-substituted or unsubstituted cycloalkyl, R^{6AH}-substituted or unsubstituted heterocycloalkyl, R^{6AH}-substituted or unsubstituted aryl, or R^{6AH}-substituted or unsubstituted heteroaryl. In embodiments, R^{6AG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6AH}-substituted or unsubstituted C₁-C₆ alkyl, R^{6AH}-substituted or unsubstituted 2 to 6 membered heteroalkyl, R^{6AH}-substituted or unsubstituted C₃-C₆ cycloalkyl, R^{6AH}-substituted or unsubstituted 3 to 6 membered heterocycloalkyl, R^{6AH}-substituted or unsubstituted phenyl, or R^{6AH}-substituted or unsubstituted 5 to 6 membered heteroaryl.

In embodiments, W^{1B} is independently a bond, —O—, —S—, —NH—, —C(O)—, —C(O)O—, —S(O)—, —S(O)₂—, —C(O)NH—, —NHC(O)—, —NHC(O)NH—, R^{6BG}-substituted or unsubstituted alkylene, R^{6BG}-substituted or unsubstituted heteroalkylene, R^{6BG}-substituted or unsubstituted cycloalkylene, R^{6BG} substituted or unsubstituted heterocycloalkylene, R^{6BG}-substituted or unsubstituted aryl, R^{6BG}-substituted or unsubstituted heteroaryl. In embodiments, W^{1B} is independently a bond, —O—, —S—, —NH—, —C(O)—, —C(O)O—, —S(O)—, —S(O)₂—, —C(O)NH—, —NHC(O)—, —NHC(O)NH, R^{6BG}-substituted or unsubstituted C₁-C₆ alkylene, R^{6BG}-substituted or unsubstituted 2 to 6 membered heteroalkylene, R^{6BG}-substituted or unsubstituted C₃-C₆ cycloalkylene, R^{6BG}-substituted or unsubstituted 3 to 6 membered heterocycloalkylene, R^{6BG}-substituted or unsubstituted phenyl, or R^{6BG}-substituted or unsubstituted 5 to 6 membered heteroaryl.

R^{6BG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6BH}-substituted or unsubstituted alkyl, R^{6BH}-substituted or unsubstituted heteroalkyl, R^{6BH}-substituted or unsubstituted cycloalkyl, R^{6BH}-substituted or unsubstituted heterocycloalkyl, R^{6BH}-substituted or unsubstituted aryl, or R^{6BH}-substituted or unsubstituted heteroaryl. In embodiments, R^{6BG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6BH}-substituted or unsubstituted C₁-C₆ alkyl, R^{6BH}-substituted or unsubstituted 2 to 6 membered heteroalkyl, R^{6BH}-substituted or unsubstituted C₃-C₆ cycloalkyl, R^{6BH}-substituted or unsubstituted 3 to 6 membered heterocycloalkyl, R^{6BH}-substituted or unsubstituted phenyl, or R^{6BH}-substituted or unsubstituted 5 to 6 membered heteroaryl.

In embodiments, W^{1C} is independently a bond, —O—, —S—, —NH—, —C(O)—, —C(O)O—, —S(O)—, —S(O)₂—, —C(O)NH—, —NHC(O)—, —NHC(O)NH—, R^{6CG}-substituted or unsubstituted alkylene, R^{6CG}-substituted or unsubstituted heteroalkylene, R^{6CG}-substituted or unsubstituted cycloalkylene, R^{6CG}-substituted or unsubstituted heterocycloalkylene, R^{6CG}-substituted or unsubstituted aryl, R^{6CG}-substituted or unsubstituted heteroaryl. In embodiments, W^{1C} is independently a bond, —O—, —S—, —NH—, —C(O)—, —C(O)O—, —S(O)—, —S(O)₂—, —C(O)NH—, —NHC(O)—, —NHC(O)NH, R^{6CG}-substituted or unsubstituted C₁-C₆ alkylene, R^{6CG}-substituted or unsubstituted 2 to 6 membered heteroalkylene, R^{6CG}-substituted or unsubstituted C₃-C₆ cycloalkylene, R^{6CG}-substituted or unsubstituted 3 to 6 membered heterocycloalkylene, R^{6CG}-substituted or unsubstituted phenyl, or R^{6CG}-substituted or unsubstituted 5 to 6 membered heteroaryl.

R^{6CG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6CH}-substituted or unsubstituted alkyl, R^{6CH}-substituted or unsubstituted heteroalkyl, R^{6CH}-substituted or unsubstituted cycloalkyl, R^{6CH}-substituted or unsubstituted heterocycloalkyl, R^{6CH}-substituted or unsubstituted aryl, or R^{6CH}-substituted or unsubstituted heteroaryl. In embodiments, R^{6CG} is independently oxo, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6CH}-substituted or unsubstituted C₁-C₆ alkyl, R^{6CH}-substituted or unsubstituted 2 to 6 membered heteroalkyl, R^{6CH}-substituted or unsubstituted C₃-C₆ cycloalkyl, R^{6CH}-substituted or unsubstituted 3 to 6 membered heterocycloalkyl, R^{6CH}-substituted or unsubstituted phenyl, or R^{6CH}-substituted or unsubstituted 5 to 6 membered heteroaryl.

In embodiments, R^{1A} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1.1}-substituted or unsubstituted alkyl, R^{1.1}-substituted or unsubstituted heteroalkyl, R^{1.1}-substituted or unsubstituted cycloalkyl, R^{1.1}-substituted or unsubstituted heterocycloalkyl, R^{1.1}-substituted or unsubstituted aryl, or R^{1.1}-substituted or unsubstituted heteroaryl. In embodiments, R^{1A} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1.1}-substituted or unsubstituted C₁-C₆ alkyl, R^{1.1}-substituted or unsubstituted 2 to 6 membered heteroalkyl, R^{1.1}-substituted or unsubstituted C₃-C₆ cycloalkyl, R^{1.1}-substituted or unsubstituted 3 to 6 membered heterocycloalkyl, R^{1.1}-substituted or unsubstituted phenyl, or R^{1.1}-substituted or unsubstituted 5 to 6 membered heteroaryl.

In embodiments, R^{1B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1.2}-substituted or unsubstituted alkyl, R^{1.2}-substituted or unsubstituted heteroalkyl, R^{1.2}-substituted or unsubstituted cycloalkyl, R^{1.2} substituted or unsubstituted heterocycloalkyl, R^{1.2}-substituted or unsubstituted aryl, or R^{1.2}-substituted or unsubstituted heteroaryl. In embodiments, R^{1B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1.2} substituted or unsubstituted C₁-C₆ alkyl, R^{1.2}-substituted or unsubstituted 2 to 6 membered heteroalkyl, R^{1.2}-substituted or unsubstituted C₃-C₆ cycloalkyl, R^{1.2}-substituted or unsubstituted 3 to 6 membered heterocycloalkyl, R^{1.2}-substituted or unsubstituted phenyl, or R^{1.2}-substituted or unsubstituted 5 to 6 membered heteroaryl.

In embodiments, R^{1C} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1.3}-substituted or unsubstituted alkyl, R^{1.3}-substituted or unsubstituted heteroalkyl, R^{1.3}-substituted or unsubstituted cycloalkyl, R^{1.3} substituted or unsubstituted heterocycloalkyl, R^{1.3}-substituted or unsubstituted aryl, or R^{1.3}-substituted or unsubstituted heteroaryl. In embodiments, R^{1C} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1.3} substituted or unsubstituted C₁-C₆ alkyl, R^{1.3}-substituted or unsubstituted 2 to 6 membered heteroalkyl, R^{1.3}-substituted or unsubstituted C₃-C₆ cycloalkyl, R^{1.3}-substituted or unsubstituted 3 to 6 membered heterocycloalkyl, R^{1.3}-substituted or unsubstituted phenyl, or R^{1.3}-substituted or unsubstituted 5 to 6 membered heteroaryl. R^{1B} and R^{1C} bonded to the same nitrogen atom may optionally be joined to form a R^{1.3}-substituted or unsubstituted 3 to 6 membered heterocycloalkyl or R^{1.3}-substituted or unsubstituted 5 to 6 membered heteroaryl.

In embodiments, R^{1D} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1.4}-substituted or unsubstituted alkyl, R^{1.4}-substituted or unsubstituted heteroalkyl, R^{1.4}-substituted or unsubstituted cycloalkyl, R^{1.4} substituted or unsubstituted heterocycloalkyl, R^{1.4}-substituted or unsubstituted aryl, or R^{1.4}-substituted or unsubstituted heteroaryl. In embodiments, R^{1D} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1.4} substituted or unsubstituted C₁-C₆ alkyl, R^{1.4}-substituted or unsubstituted 2 to 6 membered heteroalkyl, R^{1.4}-substituted or unsubstituted C₃-C₆ cycloalkyl, R^{1.4}-substituted or unsubstituted 3 to 6 membered heterocycloalkyl, R^{1.4}-substituted or unsubstituted phenyl, or R^{1.4}-substituted or unsubstituted 5 to 6 membered heteroaryl.

In embodiments, R^{1E} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1.5}-substituted or unsubstituted alkyl, R^{1.5}-substituted or unsubstituted heteroalkyl, R^{1.5}-substituted or unsubstituted cycloalkyl, R^{1.5} substituted or unsubstituted heterocycloalkyl, R^{1.5}-substituted or unsubstituted aryl, or R^{1.5}-substituted or unsubstituted heteroaryl. In embodiments, R^{1E} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{1.5}-substituted or unsubstituted C₁-C₆ alkyl, R^{1.5}-substituted or unsubstituted 2 to 6 membered heteroalkyl, R^{1.5}-substituted or unsubstituted C₃-C₆ cycloalkyl, R^{1.5}-substituted or unsubstituted 3 to 6 membered heterocycloalkyl, R^{1.5}-substituted or unsubstituted phenyl, or R¹0.5-substituted or unsubstituted 5 to 6 membered heteroaryl. In embodiments, R^{1E} is unsubstituted alkyl. In embodiments, R^{1E} is unsubstituted C₁-C₆ alkyl. In embodiments, R^{1E} is methyl, ethyl, propyl or butyl. In embodiments, R^{1E} is methyl.

In embodiments, R^{2B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{2.2}-substituted or unsubstituted alkyl, R^{2.2}-substituted or unsubstituted heteroalkyl, R^{2.2}-substituted or unsubstituted cycloalkyl, R^{2.2} substituted or unsubstituted heterocycloalkyl, R^{2.2}-substituted or unsubstituted aryl, or R^{2.2}-substituted or unsubstituted heteroaryl. In embodiments, R^{2B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{2.2} substituted or unsubstituted C₁-C₆ alkyl, R^{2.2}-substituted or unsubstituted 2 to 6 membered heteroalkyl, R^{2.2}-substituted or unsubstituted C₃-C₆ cycloalkyl, R^{2.2}-substituted or unsubstituted 3 to 6 membered heterocycloalkyl, R^{2.2}-substituted or unsubstituted phenyl, or R^{2.2}-substituted or unsubstituted 5 to 6 membered heteroaryl.

In embodiments, R^{3B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{3.2}-substituted or unsubstituted alkyl, R^{3.2}-substituted or unsubstituted heteroalkyl, R^{3.2}-substituted or unsubstituted cycloalkyl, R^{3.2} substituted or unsubstituted heterocycloalkyl, R^{3.2}-substituted or unsubstituted aryl, or R^{3.2}-substituted or unsubstituted heteroaryl. In embodiments, R^{3B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{3.2} substituted or unsubstituted C₁-C₆ alkyl, R^{3.2}-substituted or unsubstituted 2 to 6 membered heteroalkyl, R^{3.2}-substituted or unsubstituted C₃-C₆ cycloalkyl, R^{3.2}-substituted or unsubstituted 3 to 6 membered heterocycloalkyl, R^{3.2}-substituted or unsubstituted phenyl, or R^{3.2}-substituted or unsubstituted 5 to 6 membered heteroaryl.

In embodiments, R^{4B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{4.2}-substituted or unsubstituted alkyl, R^{4.2}-substituted or unsubstituted heteroalkyl, R^{4.2}-substituted or unsubstituted cycloalkyl, R^{4.2} substituted or unsubstituted heterocycloalkyl, R^{4.2}-substituted or unsubstituted aryl, or R^{4.2}-substituted or unsubstituted heteroaryl. In embodiments, R^{4B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{4.2} substituted or unsubstituted C₁-C₆ alkyl, R^{4.2}-substituted or unsubstituted 2 to 6 membered heteroalkyl, R^{4.2}-substituted or unsubstituted C₃-C₆ cycloalkyl, R^{4.2}-substituted or unsubstituted 3 to 6 membered heterocycloalkyl, R^{4.2}-substituted or unsubstituted phenyl, or R^{4.2}-substituted or unsubstituted 5 to 6 membered heteroaryl.

In embodiments, R^{5B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.2}-substituted or unsubstituted alkyl, R^{5.2}-substituted or unsubstituted heteroalkyl, R^{5.2}-substituted or unsubstituted cycloalkyl, R^{5.2} substituted or unsubstituted heterocycloalkyl, R^{5.2}-substituted or unsubstituted aryl, or R^{5.2}-substituted or unsubstituted heteroaryl. In embodiments, R^{5B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.2} substituted or unsubstituted C₁-C₆ alkyl, R^{5.2}-substituted or unsubstituted 2 to 6 membered heteroalkyl, R^{5.2}-substituted or unsubstituted C₃-C₆ cycloalkyl, R^{5.2}-substituted or unsubstituted 3 to 6 membered heterocycloalkyl, R^{5.2}-substituted or unsubstituted phenyl, or R^{5.2}-substituted or unsubstituted 5 to 6 membered heteroaryl.

In embodiments, R^{5E} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.5}-substituted or unsubstituted alkyl, R^{5.5}-substituted or unsubstituted heteroalkyl, R^{5.5}-substituted or unsubstituted cycloalkyl, R^{5.5-}substituted or unsubstituted heterocycloalkyl, R^{5.5}-substituted or unsubstituted aryl, or R^{5.5} substituted or unsubstituted heteroaryl. In embodiments, R^{5E} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.5}-substituted or unsubstituted C₁-C₆ alkyl, R^{5.5}-substituted or unsubstituted 2 to 6 membered heteroalkyl, R^{5.5}-substituted or unsubstituted C₃-C₆ cycloalkyl, R^{5.5}-substituted or unsubstituted 3 to 6 membered heterocycloalkyl, R^{5.5}-substituted or unsubstituted phenyl, or R^{5.5}-substituted or unsubstituted 5 to 6 membered heteroaryl.

In embodiments, R^{5F} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.6}-substituted or unsubstituted alkyl, R^{5.6}-substituted or unsubstituted heteroalkyl, R^{5.6}-substituted or unsubstituted cycloalkyl, R^{5.6} substituted or unsubstituted heterocycloalkyl, R^{5.6}-substituted or unsubstituted aryl, or R^{5.6}-substituted or unsubstituted heteroaryl. In embodiments, R^{5F} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.6} substituted or unsubstituted C₁-C₆ alkyl, R^{5.6}-substituted or unsubstituted 2 to 6 membered heteroalkyl, R^{5.6}-substituted or unsubstituted C₃-C₆ cycloalkyl, R^{5.6}-substituted or unsubstituted 3 to 6 membered heterocycloalkyl, R^{5.6}-substituted or unsubstituted phenyl, or R^{5.6}-substituted or unsubstituted 5 to 6 membered heteroaryl.

In embodiments, R^{5AB} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.7}-substituted or unsubstituted alkyl, R^{5.7}-substituted or unsubstituted heteroalkyl, R^{5.7}-substituted or unsubstituted cycloalkyl, R^{5.7} substituted or unsubstituted heterocycloalkyl, R^{5.7}-substituted or unsubstituted aryl, or R^{5.7} substituted or unsubstituted heteroaryl. In embodiments, R^{5AB} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.7}-substituted or unsubstituted C₁-C₆ alkyl, R^{5.7}-substituted or unsubstituted 2 to 6 membered heteroalkyl, R^{5.7}-substituted or unsubstituted C₃-C₆ cycloalkyl, R^{5.7}-substituted or unsubstituted 3 to 6 membered heterocycloalkyl, R^{5.7}-substituted or unsubstituted phenyl, or R^{5.7}-substituted or unsubstituted 5 to 6 membered heteroaryl.

In embodiments, R^{5AE} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.8}-substituted or unsubstituted alkyl, R^{5.8}-substituted or unsubstituted heteroalkyl, R^{5.8}-substituted or unsubstituted cycloalkyl, R⁵8-substituted or unsubstituted heterocycloalkyl, R^{5.8}-substituted or unsubstituted aryl, or R^{5.8}-substituted or unsubstituted heteroaryl. In embodiments, R^{5AE} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.8}-substituted or unsubstituted C₁-C₆ alkyl, R^{5.8}-substituted or unsubstituted 2 to 6 membered heteroalkyl, R^{5.8}-substituted or unsubstituted C₃-C₆ cycloalkyl, R^{5.8}-substituted or unsubstituted 3 to 6 membered heterocycloalkyl, R^{5.8}-substituted or unsubstituted phenyl, or R^{5.8}-substituted or unsubstituted 5 to 6 membered heteroaryl.

In embodiments, R^{5AE} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.9}-substituted or unsubstituted alkyl, R^{5.9}-substituted or unsubstituted heteroalkyl, R^{5.9}-substituted or unsubstituted cycloalkyl, R^{5.9}-substituted or unsubstituted heterocycloalkyl, R^{5.9}-substituted or unsubstituted aryl, or R^{5.9} substituted or unsubstituted heteroaryl. In embodiments, R^{5AF} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.9}-substituted or unsubstituted C₁-C₆ alkyl, R^{5.9}-substituted or unsubstituted 2 to 6 membered heteroalkyl, R^{5.9}-substituted or unsubstituted C₃-C₆ cycloalkyl, R^{5.9}-substituted or unsubstituted 3 to 6 membered heterocycloalkyl, R^{5.9}-substituted or unsubstituted phenyl, or R^{5.9}-substituted or unsubstituted 5 to 6 membered heteroaryl.

In embodiments, R^{5BB} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.10}-substituted or unsubstituted alkyl, R^{5.10}-substituted or unsubstituted heteroalkyl, R^{5.10}-substituted or unsubstituted cycloalkyl, R^{5.10} substituted or unsubstituted heterocycloalkyl, R^{5.10}-substituted or unsubstituted aryl, or R^{5.10} substituted or unsubstituted heteroaryl. In embodiments, R^{5BB} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.10}-substituted or unsubstituted C₁-C₆ alkyl, R^{5.10}-substituted or unsubstituted 2 to 6 membered heteroalkyl, R^{5.10}-substituted or unsubstituted C₃-C₆ cycloalkyl, R^{5.10}-substituted or unsubstituted 3 to 6 membered heterocycloalkyl, R^{5.10}-substituted or unsubstituted phenyl, or R^{5.10}-substituted or unsubstituted 5 to 6 membered heteroaryl.

In embodiments, R^{5BE} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.11}-substituted or unsubstituted alkyl, R^{5.11}-substituted or unsubstituted heteroalkyl, R^{5.11}-substituted or unsubstituted cycloalkyl, R^{5.11}-substituted or unsubstituted heterocycloalkyl, R^{5.11}-substituted or unsubstituted aryl, or R^{5.11}-substituted or unsubstituted heteroaryl. In embodiments, R^{5BE} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.11}-substituted or unsubstituted C₁-C₆ alkyl, R^{5.11}-substituted or unsubstituted 2 to 6 membered heteroalkyl, R^{5.11}-substituted or unsubstituted C₃-C₆ cycloalkyl, R^{5.11}-substituted or unsubstituted 3 to 6 membered heterocycloalkyl, R^{5.11}-substituted or unsubstituted phenyl, or R^{5.11}-substituted or unsubstituted 5 to 6 membered heteroaryl.

In embodiments, R^{5BF} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.12}-substituted or unsubstituted alkyl, R^{5.12}-substituted or unsubstituted heteroalkyl, R^{5.12}-substituted or unsubstituted cycloalkyl, R^{5.12} substituted or unsubstituted heterocycloalkyl, R^{5.12}-substituted or unsubstituted aryl, or R^{5.12}-substituted or unsubstituted heteroaryl. In embodiments, R^{5BF} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.12}-substituted or unsubstituted C₁-C₆ alkyl, R^{5.12}-substituted or unsubstituted 2 to 6 membered heteroalkyl, R^{5.12}-substituted or unsubstituted C₃-C₆ cycloalkyl, R^{5.12}-substituted or unsubstituted 3 to 6 membered heterocycloalkyl, R^{5.12}-substituted or unsubstituted phenyl, or R^{5.12}-substituted or unsubstituted 5 to 6 membered heteroaryl.

In embodiments, R^{5CB} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.13}-substituted or unsubstituted alkyl, R^{5.13}-substituted or unsubstituted heteroalkyl, R^{5.13}-substituted or unsubstituted cycloalkyl, R^{5.13} substituted or unsubstituted heterocycloalkyl, R^{5.13}-substituted or unsubstituted aryl, or R^{5.13}-substituted or unsubstituted heteroaryl. In embodiments, R^{5CB} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.13}-substituted or unsubstituted C₁-C₆ alkyl, R^{5.13}-substituted or unsubstituted 2 to 6 membered heteroalkyl, R^{5.13}-substituted or unsubstituted C₃-C₆ cycloalkyl, R^{5.13}-substituted or unsubstituted 3 to 6 membered heterocycloalkyl, R^{5.13}-substituted or unsubstituted phenyl, or R^{5.13}-substituted or unsubstituted 5 to 6 membered heteroaryl.

In embodiments, R^{5CE} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.14}-substituted or unsubstituted alkyl, R^{5.14}-substituted or unsubstituted heteroalkyl, R^{5.14}-substituted or unsubstituted cycloalkyl, R^{5.14} substituted or unsubstituted heterocycloalkyl, R^{5.14}-substituted or unsubstituted aryl, or R^{5.14}-substituted or unsubstituted heteroaryl. In embodiments, R^{5CE} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.14}-substituted or unsubstituted C₁-C₆ alkyl, R^{5.14}-substituted or unsubstituted 2 to 6 membered heteroalkyl, R^{5.14}-substituted or unsubstituted C₃-C₆ cycloalkyl, R^{5.14}-substituted or unsubstituted 3 to 6 membered heterocycloalkyl, R^{5.14}-substituted or unsubstituted phenyl, or R^{5.14}-substituted or unsubstituted 5 to 6 membered heteroaryl.

In embodiments, R^{5CF} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.15}-substituted or unsubstituted alkyl, R^{5.15}-substituted or unsubstituted heteroalkyl, R^{5.15}-substituted or unsubstituted cycloalkyl, R^{5.1}-substituted or unsubstituted heterocycloalkyl, R^{5.15}-substituted or unsubstituted aryl, or R^{5.1}-substituted or unsubstituted heteroaryl. In embodiments, R^{5CF} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{5.15}-substituted or unsubstituted C₁-C₆ alkyl, R^{5.15}-substituted or unsubstituted 2 to 6 membered heteroalkyl, R^{5.15}-substituted or unsubstituted C₃-C₆ cycloalkyl, R^{5.15}-substituted or unsubstituted 3 to 6 membered heterocycloalkyl, R^{5.15}-substituted or unsubstituted phenyl, or R^{5.15}-substituted or unsubstituted 5 to 6 membered heteroaryl.

In embodiments, R^{6A} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6.1}-substituted or unsubstituted alkyl, R^{6.1}-substituted or unsubstituted heteroalkyl, R^{6.1}-substituted or unsubstituted cycloalkyl, R^{6.1} substituted or unsubstituted heterocycloalkyl, R^{6.1}-substituted or unsubstituted aryl, or R^{6.1}-substituted or unsubstituted heteroaryl. In embodiments, R^{6A} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6.1} substituted or unsubstituted C₁-C₆ alkyl, R^{6.1}-substituted or unsubstituted 2 to 6 membered heteroalkyl, R^{6.1}-substituted or unsubstituted C₃-C₆ cycloalkyl, R^{6.1}-substituted or unsubstituted 3 to 6 membered heterocycloalkyl, R^{6.1}-substituted or unsubstituted phenyl, or R^{6.1}-substituted or unsubstituted 5 to 6 membered heteroaryl.

In embodiments, R^{6B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6.2}-substituted or unsubstituted alkyl, R^{6.2}-substituted or unsubstituted heteroalkyl, R^{6.2}-substituted or unsubstituted cycloalkyl, R^{6.2} substituted or unsubstituted heterocycloalkyl, R^{6.2}-substituted or unsubstituted aryl, or R^{6.2}-substituted or unsubstituted heteroaryl. In embodiments, R^{6B} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6.2} substituted or unsubstituted C₁-C₆ alkyl, R^{6.2}-substituted or unsubstituted 2 to 6 membered heteroalkyl, R^{6.2}-substituted or unsubstituted C₃-C₆ cycloalkyl, R^{6.2}-substituted or unsubstituted 3 to 6 membered heterocycloalkyl, R^{6.2}-substituted or unsubstituted phenyl, or R^{6.2}-substituted or unsubstituted 5 to 6 membered heteroaryl.

In embodiments, R^{6AB} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6.7}-substituted or unsubstituted alkyl, R^{6.7}-substituted or unsubstituted heteroalkyl, R^{6.7}-substituted or unsubstituted cycloalkyl, R^{6.7} substituted or unsubstituted heterocycloalkyl, R^{6.7}-substituted or unsubstituted aryl, or R^{6.7}-substituted or unsubstituted heteroaryl. In embodiments, R^{6AB} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6.7}-substituted or unsubstituted C₁-C₆ alkyl, R^{6.7}-substituted or unsubstituted 2 to 6 membered heteroalkyl, R^{6.7}-substituted or unsubstituted C₃-C₆ cycloalkyl, R^{6.7}-substituted or unsubstituted 3 to 6 membered heterocycloalkyl, R^{6.7}-substituted or unsubstituted phenyl, or R^{6.7}-substituted or unsubstituted 5 to 6 membered heteroaryl.

In embodiments, R^{6BB} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6.10}-substituted or unsubstituted alkyl, R^{6.10}-substituted or unsubstituted heteroalkyl, R^{6.10}-substituted or unsubstituted cycloalkyl, R^{6.10} substituted or unsubstituted heterocycloalkyl, R^{6.10}-substituted or unsubstituted aryl, or R^{6.10}-substituted or unsubstituted heteroaryl. In embodiments, R^{6BB} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6.10}-substituted or unsubstituted C₁-C₆ alkyl, R^{6.10}-substituted or unsubstituted 2 to 6 membered heteroalkyl, R^{6.10}-substituted or unsubstituted C₃-C₆ cycloalkyl, R^{6.10}-substituted or unsubstituted 3 to 6 membered heterocycloalkyl, R^{6.10}-substituted or unsubstituted phenyl, or R^{6.10}-substituted or unsubstituted 5 to 6 membered heteroaryl.

In embodiments, R^{6BC} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6.13}-substituted or unsubstituted alkyl, R^{6.13}-substituted or unsubstituted heteroalkyl, R^{6.13}-substituted or unsubstituted cycloalkyl, R^{6.13} substituted or unsubstituted heterocycloalkyl, R^{6.13}-substituted or unsubstituted aryl, or R^{6.13}-substituted or unsubstituted heteroaryl. In embodiments, R^{6BC} is independently hydrogen, halogen, —CF₃, —CCl₃, —CBr₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, R^{6.13}-substituted or unsubstituted C₁-C₆ alkyl, R^{6.13}-substituted or unsubstituted 2 to 6 membered heteroalkyl, R^{6.13}-substituted or unsubstituted C₃-C₆ cycloalkyl, R^{6.13}-substituted or unsubstituted 3 to 6 membered heterocycloalkyl, R^{6.13}-substituted or unsubstituted phenyl, or R^{6.13}-substituted or unsubstituted 5 to 6 membered heteroaryl.

R^{1H}, R^{2H}, R^{3H}, R^{4H}, R^{5H}, R^{6H}, R^{7H}, R^{5AH}, R^{5BH}, R^{5CH}, R^{6AH}, R^{6BH}, R^{6CH}, R^{1.1}, R^{1.2}, R^{1.3}, R^{1.4}, R^{1.5}, R^{2.2}, R^{3.2}, R^{4.2}, R^{5.2}, R^{5.5}, R^{5.6}, R^{5.7}, R^{5.8}, R^{5.9}, R^{5.10}, R^{5.11}, R^{5.12}, R^{5.13}, R^{5.14}, R^{5.15}, R^{6.1}, R^{6.2}, R^{6.7}, R^{6.10} and R^{6.13} are independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. In embodiments, R^{1H}, R^{2H}, R^{3H}, R^{4H}, R^{5H}, R^{6H}, R⁷H, R^{5AH}, R^{5BH}, R^{5CH}, R^{6AH}R^{6BH}, R^{6CH}, R^{1.1}, R^{1.2}, R^{1.3}, R^{1.4}, R^{1.5}, R^{2.2}, R^{3.2}, R^{4.2}, R^{5.2}R^{5.5}, R^{5.6}, R^{5.7}, R^{5.8}, R^{5.9}, R^{5.10}, R^{5.11}, R^{5.12}, R^{5.13}, R^{5.14}, R^{5.15}, R^{6.1}, R^{6.2}, R^{6.7}, R^{6.10} and R^{6.13} are independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

### III. Pharmaceutical Compositions

Also provided herein are pharmaceutical formulations. In one aspect, is a pharmaceutical composition that includes a compound or antibody drug conjugate described herein and a pharmaceutically acceptable excipient.

### IV. Methods

Provided herein are methods. In one aspect, there is provided a method of preparing an antibody drug conjugate. The method includes contacting a calicheamicin construct with a cysteine or lysine of an antibody, the calicheamicin construct having the formula W¹-(L³)z-M-(L⁴)_{z2}-P-D, wherein W¹ is a functional group reactive with a lysine side chain or cysteine side chain, M is a cleavable moiety, L³ and L⁴ are independently a linker, P is a disulfide protecting group and D is a calicheamicin or analog thereof.

In embodiments, the calicheamicin construct is contacted with a specific cysteine of the antibody. In embodiments, the specific cysteine is derived from a native disulfide bridge. In embodiments, the antibody is an engineered antibody and the specific cysteine is not derived from a native disulfide bridge. In embodiments, the specific cysteine selectively reduced prior to the contacting. In embodiments, the step of selectively reducing the antibody, comprises the step of contacting the antibody with a stabilizing agent.

In still other preferred embodiments the disclosed calicheamicin-linker constructs are used to fabricate antibody drug conjugates of the formula:

Ab-[W—(X1)ₐ-CM-(X2)_{b}—P-D]ₙ

or a pharmaceutically acceptable salt thereof, wherein:
a) Ab comprises a targeting agent;
b) W comprises a connecting group or linking group;
c) CM comprises a cleavable moiety;
d) P comprises a disulfide protective group;
e) X1 and X2 comprise optional spacer moieties;
f) D comprises calicheamicin;
a and b are independently 0 or 1; and
n is 1, 2, 3, 4, 5, 6, 7, 8, 9 or 10.

In selected embodiments the targeting agent will comprise a site-specific antibody having one or more free cysteines. In selected embodiments the cleavable moiety may comprise peptide bonds, hydrazone moieties, oxime moieties, ester linkages, and disulfide linkages. In yet other preferred embodiments the connecting group will react with a cysteine moiety on the targeting agent to covalently link the calicheamicin-linker construct to the targeting agent.

In addition to the foregoing antibody drug conjugates the invention further provides pharmaceutical compositions generally comprising the disclosed ADCs and methods of using such ADCs to diagnose or treat disorders, including cancer, in a patient. In particularly preferred embodiments the disclosed conjugates will associate with a SEZ6 determinant.

In another embodiment the targeting agent will comprise a site-specific engineered IgG1 isotype antibody comprising at least one unpaired cysteine residue. In some embodiments the unpaired cysteine residue(s) will comprise heavy/light chain interchain residues as opposed to heavy/heavy chain interchain residues. In other embodiments the unpaired cysteine residue will be generated from an intrachain disulfide bridge.

In another embodiment the targeting agent will comprise an engineered antibody wherein the C214 residue (numbered according to the EU index of Kabat) of the light chain comprising said site-specific engineered antibody is substituted with another residue or deleted. In a further embodiment the targeting agent comprises an engineered antibody wherein the C220 residue (numbered according to the EU index of Kabat) of the heavy chain comprising the engineered antibody is substituted with another residue or deleted.

In a related embodiment the invention is directed to a method of killing, reducing the frequency or inhibiting the proliferation of tumor cells or tumorigenic cells comprising treating said tumor cells or tumorigenic cells with a calicheamicin ADC of the instant invention. In a related embodiment the invention provides a method of treating cancer comprising administering to a subject a pharmaceutical composition comprising a calicheamicin conjugate of the instant invention.

In another embodiment the present invention comprises a method of preparing an antibody drug conjugate of the invention comprising the steps of:
a) providing an calicheamicin construct comprising a cleavable linker;
b) reducing the targeting agent to provide an activated residue; and
c) conjugating the selectively reduced targeting agent to the calicheamicin construct.

In selected embodiments the targeting agent will comprise a site-specific antibody having one or more free cysteines. In other embodiments the site-specific antibody will be selectively reduced. In a related preferred embodiment the step of selectively reducing the antibody comprises the step of contacting the antibody with a stabilizing agent. In yet another embodiment the process may further comprise the step of contacting the antibody with a mild reducing agent.

In another aspect, there is provided a method of treating cancer in a subject in need thereof. The method includes administering to the subject a therapeutically effective amount of the pharmaceutical composition of claim or the antibody drug conjugate disclosed herein. In embodiments, the cancer is selected from pancreatic cancer, colorectal cancer, non-small cell lung cancer, small cell lung cancer and gastric cancer. In embodiments, the method further includes administering to the subject an additional chemotherapeutic agent.

In one aspect, there is provided a method of delivering a calicheamicin cytotoxin to a cell. The method includes contacting the cell with an antibody drug conjugate as disclosed herein.

As indicated the disclosed conjugates may be used for the treatment, management, amelioration or prophylaxis of proliferative disorders or recurrence or progression thereof. Selected embodiments of the present invention provide for the use of such calicheamicin conjugates for the immunotherapeutic treatment of malignancies preferably comprising a reduction in tumor initiating cell frequency. The disclosed ADCs may be used alone or in conjunction with a wide variety of anti-cancer compounds such as chemotherapeutic or immunotherapeutic agents (e.g., therapeutic antibodies) or biological response modifiers. In other selected embodiments, two or more discrete calicheamicin conjugates may be used in combination to provide enhanced anti-neoplastic effects.

The present invention also provides kits or devices and associated methods that employ the calicheamicin conjugates disclosed herein, and pharmaceutical compositions of calicheamicin conjugates as disclosed herein, which are useful for the treatment of proliferative disorders such as cancer. To this end the present invention preferably provides an article of manufacture useful for treating such disorders comprising a receptacle containing an antibody drug conjugate of the invention and instructional materials for using the conjugates to treat, ameliorate or prevent a proliferative disorder or progression or recurrence thereof. In selected embodiments the devices and associated methods will comprise the step of contacting at least one cancer stem cell.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### I Calicheamicin

The calicheamicins are a class of enediyne antitumor antibiotics derived from the bacterium Micromonospora echinospora, including calicheamicin γ₁^{I}., calicheamicin β₁^{Br}, calicheamicin γ₁^{Br}, calicheamicin α₂^{I}, calicheamicin α₃^{I}, calicheamicin β₁ⁱ and calicheamicin β₁ⁱ were isolated and characterized. The structures of each of the foregoing calicheamicin analogs are well known in the art (e.g., see Lee et al., Journal of Antibiotics, July 1989 which is incorporated herein by reference in its entirety) and are compatible with the calicheamicin constructs and antibody drug conjugates disclosed herein. In general, calicheamicin γ¹ contains two distinct structural regions, each playing a specific role in the compound's biological activity. The larger of the two consists of an extended sugar residue, comprising four monosaccharide units and one hexasubstituted benzene ring; these are joined together through a highly unusual series of glycosidic, thioester, and hydroxylamine linkages. The second structural region, the aglycon (known as calicheamicinone), contains a compact, highly functionalized bicyclic core, housing a strained enediyne unit within a bridging 10-member ring. This aglycon subunit further comprises an allylic trisulfide which, as described below, functions as an activator to generate the cytotoxic form of the molecule.

By way of example the structure for trisulfide calicheamicin γ₁^{I} is shown immediately below:

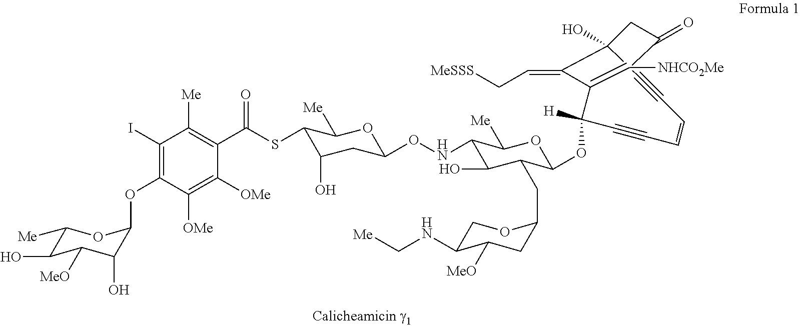

As used herein the term “calicheamicin” shall be held to mean any one of calicheamicin γ₁^{I}, calicheamicin β₁^{Br}, calicheamicin γ₁^{Br}, calicheamicin α₂^{I}, calicheamicin α₃^{I}, calicheamicin β₁ⁱ and calicheamicin δ₁ along with N-acetyl derivatives, sulfide analogs and analogs thereof. As used herein, the term “calicheamicin will be understood to encompass and calicheamicin found in nature as well as calicheamicin moieties with a terminating in a disulfide having a point of attachment to another molecule (e.g., an antibody drug conjugate) and analogs thereof. By way of example, as used herein, calicheamicin γ^{I} is to be understood to be construed as:

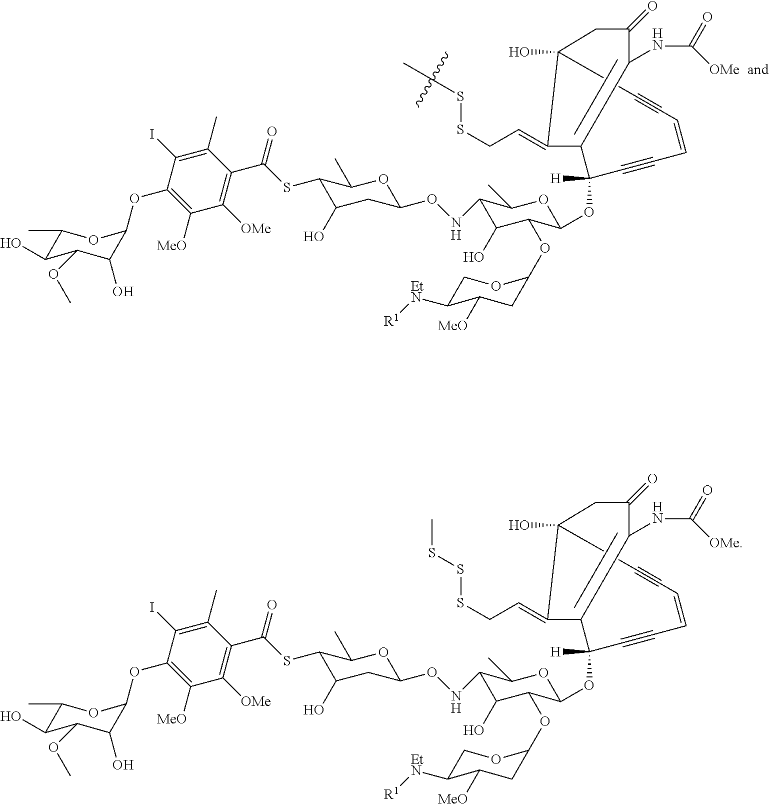

It will be appreciated that any of the aforementioned compounds are compatible with the teachings herein and may be used to fabricate the disclosed calicheamicin constructs and antibody drug conjugates. In certain embodiments the calicheamicin component of the disclosed antibody drug conjugates will comprise N-acetyl Calicheamicin γ₁^{I}.

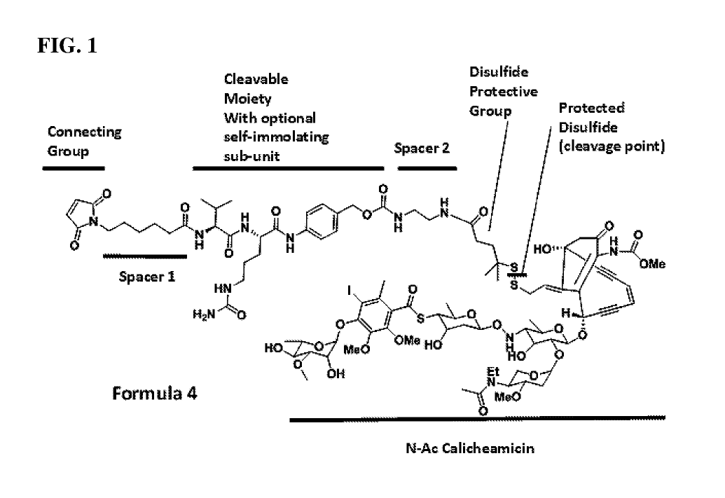

Calicheamicins target nucleic acids and cause strand scission thereby killing the target cell. More specifically, calicheamicins have been found to bind the minor groove of DNA, where they then undergo a reaction analogous to Bergman cyclization to generate a diradical species. In this regard the aryl tetrasaccharide subunit serves to deliver the drug to its target, tightly binding to the minor groove of double helical DNA as demonstrated by Crothers et al. (1999). When a nucleophile (e.g. glutathione) attacks the central sulfur atom of the trisulfide group, it causes a significant change in structural geometry and imposes a great deal of strain on the 10-member enediyne ring. This strain is completely relieved by the enediyne undergoing a cycloaromatization reaction, generating a highly-reactive 1,4-benzenoid diradical and leading, eventually, to DNA cleavage by attracting hydrogen atoms from the deoxyribose DNA backbone which results in strand scission. Note that in the calicheamicin disulfide analog constructs of the instant invention the nucleophile cleaves the protected disulfide bond to produce the desired diradical (see FIG. 1).

In 2000 a CD33 antigen-targeted immunoconjugate comprising N-acetyl dimethyl hydrazide calicheamicin (Mylotarg®) was developed and marketed as a targeted therapy against acute myeloid leukemia (AML). The drug was subsequently withdrawn due to efficacy and toxicity issues. By way of contrast the antibody calicheamicin conjugates of the instant invention exhibit favorable therapeutic profiles that suggest they may be effectively used to treat a number of proliferative disorders.

### II Antibody Conjugates

In preferred embodiments targeting agents compatible with the instant invention are conjugated with the novel calicheamicin constructs to form an “antibody drug conjugate” (ADC) or “antibody conjugate”. The term “conjugate” is used broadly and means the covalent or non-covalent association of any cleavable calicheamicin moiety with a targeting agent (e.g., antibody) compatible with the instant invention regardless of the precise method of association. In certain preferred embodiments the association is effected through a cysteine residue of the targeting agent. In particularly preferred embodiments the calicheamicin may be conjugated to the antibody through a cleavable linker via one or more site-specific free cysteine(s). The disclosed ADCs may be used for therapeutic purposes including the treatment of cancer.

The ADCs of the instant invention may be used to deliver cytotoxins or other payloads to the target location (e.g., tumorigenic cells expressing SEZ6). As used herein the terms “drug” or “warhead” may be used interchangeably and will mean any calicheamicin or calicheamicin analog as described above. In preferred embodiments the disclosed ADCs will direct the bound payload comprising a calicheamicin warhead to the target site in a relatively unreactive, non-toxic state before releasing and activating the warhead. This targeted release of the warhead is preferably assisted through stable conjugation of the payloads (e.g., via one or more cysteines on the antibody) and the relatively homogeneous composition of the ADC preparations which minimize over-conjugated toxic species. Coupled with cleavable drug linkers that are designed to largely release the calicheamicin comprising payload once it has been delivered to the tumor site, the antibody drug conjugates of the instant invention can substantially reduce undesirable non-specific toxicity. This advantageously provides for relatively high levels of the active calicheamicin at the tumor site while minimizing exposure of non-targeted cells and tissue thereby providing an enhanced therapeutic index.

In any event the selected payload comprising calicheamicin may be covalently or non-covalently linked to the antibody and exhibit various stoichiometric molar ratios depending, at least in part, on the method used to effect the conjugation. In preferred embodiments the conjugates of the instant invention may be represented by the formula:

Ab-[W—(X1)ₐ-CM-(X2)_{b}—P-D]ₙ (Formula 2)

or a pharmaceutically acceptable salt thereof, wherein
a) Ab comprises a targeting agent;
b) W comprises a connecting group;
c) CM comprises a cleavable moiety;
d) P comprises a disulfide protective group
e) X1 and X2 comprise optional spacer moieties; and
f) D comprises calicheamicin;
   wherein a and b are independently 0 or 1 and n is 1, 2, 3, 4, 5, 6, 7, 8, 9 or 10.

For the purposes of the instant disclosure the components W—(X1)ₐ-CM-(X2)_{b}—P may be generally referred to as a “linker” or “linker unit” and will be understood to link or connect (e.g., through a series of covalent bonds) the calicheamicin warhead to the targeting agent. Taken together the calicheamicin and linker comprise a payload that is conjugated to the targeting agent as described herein.

In certain embodiments the linker may comprise a branched linker. In other preferred embodiments the targeting agent will comprise an antibody. In particularly preferred embodiments D will comprise N-acetyl calicheamicin as set forth in Formula 3 immediately below:

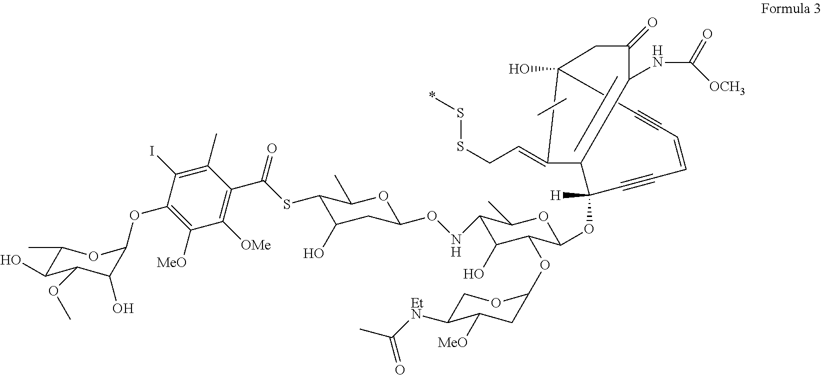

wherein the * symbol represents the disulfide protective group which is covalently bound to the remainder of the linker and ultimately the targeting agent (Ab). Other preferred embodiments and linker components and linker configurations will be discussed in more detail below.

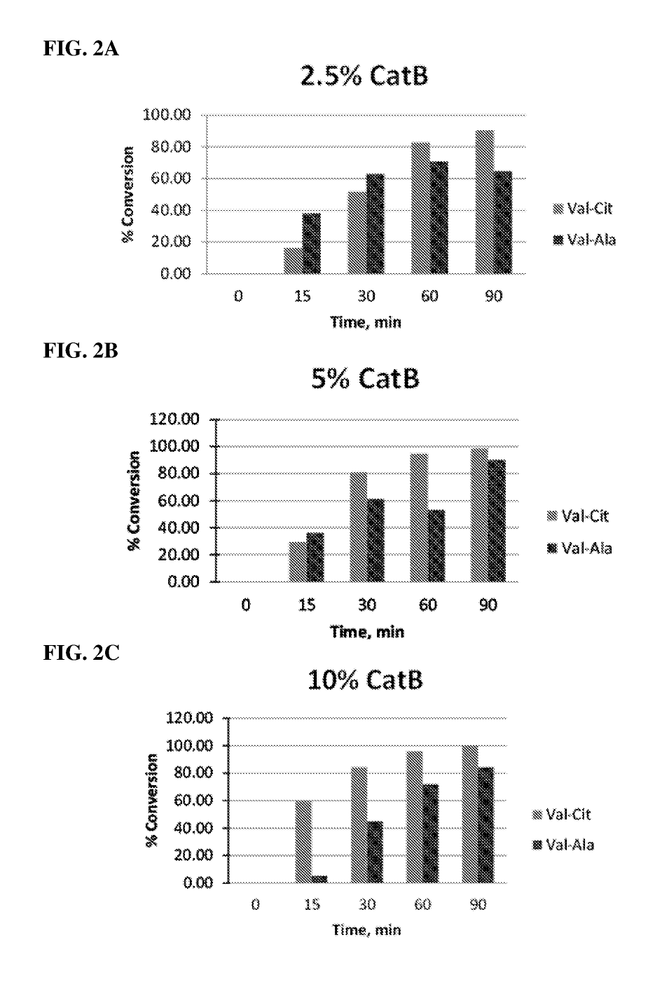

With regard to Formula 3 it will be appreciated that the illustrated compound comprises a disulfide N-acetyl calicheamicin analog bound to a disulfide protective group (represented by *) that is covalently bound to the remainder of the linker. As shown in the Examples below, the disulfide protective group improves stability of the disulfide bond in the bloodstream and allows for effective synthesis of the disclosed calicheamicin-linker constructs. Upon reaching the target (e.g., a cancer cell) the cleavable moiety (CM) will be severed to release the calicheamicin attached to part of the linker (e.g., X2−see FIG. 2) through the disulfide protective group. Once the linker has been initially cleaved at the CM the remainder of the linker attached to the calicheamicin will be degraded under physiological conditions to the point where the disulfide bond is severed (preferably intracellularly) followed by rearrangement and formation of the active biradical calicheamicin species. It is this form of the calicheamicin warhead that binds to the minor groove of the cellular DNA and induces the desired cytotoxic effects (See Walker et al. Biochemistry 89: 4608-4612, 5/92 which is incorporated herein in its entirety by reference). FIG. 1 provides an annotated chemical structure depicting a dipeptide calicheamicin-linker construct of the present invention with individual components delineated for the purposes of explanation.

In any event conjugates according to the aforementioned Formula 2 may be fabricated using a number of different cleavable linkers and that conjugation methodology will vary depending on the selection of components. As such, any cleavable linker compound of Formula 2 that associates with calicheamicin and a reactive residue (e.g., a cysteine) of the disclosed targeting agents are compatible with the teachings herein. Similarly, any reaction conditions that allow for conjugation, including site-specific conjugation, of the selected calicheamicin-linker to an antibody are within the scope of the present invention. Notwithstanding the foregoing, particularly preferred embodiments of the instant invention comprise selective conjugation of the calicheamicin-linker to free cysteines using stabilization agents in combination with mild reducing agents as described herein. Such reaction conditions tend to provide more homogeneous preparations with less non-specific conjugation and contaminants and correspondingly less toxicity.

### III Determinants

Initially it is important to note that the calicheamicin constructs and corresponding antibody drug conjugates of the instant invention are not limited to any particular target or antigen. Rather, as any targeting agent, including any existing antibody or any antibody that may be generated as described herein, may be conjugated to the novel calicheamicin-linker constructs, the advantages conferred by the present invention are broadly applicable and may be used in conjunction with any target antigen (or determinant). More specifically, the beneficial properties imparted by use of the novel calicheamicin-linker constructs (e.g., potential site-specific conjugation, enhanced conjugate stability and reduced non-specific toxicity) are broadly applicable to therapeutic antibodies irrespective of the particular target. Thus, while certain non-limiting targeting agents directed to selected determinants have been used for the purposes of explanation and demonstration of the benefits of the instant invention, they are in no way restrictive as to the scope of the same.

Accordingly, those skilled in the art will appreciate that antibody drug conjugates of the present invention may incorporate any targeting agent (e.g., an antibody) that specifically recognizes or associates with any selected determinant. As used herein “determinant” means any detectable trait, property, marker or factor that is identifiably associated with, or specifically found in or on a particular cell, cell population or tissue. Determinants may be morphological, functional or biochemical in nature and are generally phenotypic. In certain preferred embodiments the determinant is a protein that is differentially modified with regard to its physical structure and/or chemical composition or a protein that is differentially expressed (up- or down-regulated) by specific cell types or by cells under certain conditions (e.g., during specific points of the cell cycle or cells in a particular niche). For the purposes of the instant invention the determinant preferably comprises a cell surface antigen, or a protein(s) which is differentially expressed by aberrant cells as evidenced by chemical modification, form of presentation (e.g., splice variants), timing or amount. In certain embodiments a determinant may comprise a SEZ6 protein, or any of their variants, isoforms or family members, and specific domains, regions or epitopes thereof. An “immunogenic determinant” or “antigenic determinant” or “immunogen” or “antigen” means any fragment, region or domain of a polypeptide that can stimulate an immune response when introduced into an immunocompetent animal and is recognized by the antibodies produced from the immune response. Determinants contemplated herein may identify a cell, cell subpopulation or tissue (e.g., tumors) by their presence (positive determinant) or absence (negative determinant).

In particularly preferred embodiments disclosed antibody drug conjugates will comprise antibodies directed to SEZ6. SEZ6 (also known as seizure related 6 homolog) is a type I transmembrane protein originally cloned from mouse cerebrum cortex-derived cells treated with the convulsant pentylentetrazole (Shimizu-Nishikawa, 1995, PMID: 7723619). SEZ6 has two isoforms, one of approximately 4210 bases (NM_178860) encoding a 994 amino acid protein (NP_849191), and one of approximately 4194 bases (NM_001098635) encoding a 993 amino acid protein (NP_001092105). These differ only in the final ten amino acid residues in their ECDs. SEZ6 has two other family members: SEZ6L and SEZ6L2. The term “SEZ6 family”, refers to SEZ6, SEZ6L, SEZ6L2 and their various isoforms. The mature SEZ6 protein is composed of a series of structural domains: a cytoplasmic domain, a transmembrane domain and an extracellular domain comprising a unique N-terminal domain, followed by two alternating Sushi and CUB-like domains, and three additional tandem Sushi domain repeats. Mutations in the human SEZ6 gene have been linked to febrile seizures, a convulsion associated with a rise in body temperature and the most common type of seizure in childhood (Yu et al., 2007, PMID: 17086543). Review of the structural modules of the SEZ6 protein identified by homology and sequence analysis suggest a possible role in signaling, cell-cell communication, and neural development. Anti-SEZ6 humanized antibodies compatible with the instant invention were generated, as described in WO2015/031541 which is incorporated herein in its entirety, from antibodies isolated from mice immunized with a SEZ6 antigen.

### IV Targeting Agents

### A. Agent Structure

As alluded to above, particularly preferred embodiments of the instant invention comprise the disclosed conjugates with a targeting agent preferably in the form of an antibody, or immunoreactive fragment thereof that preferentially associates with one or more epitopes on a selected determinant. Antibodies and variants and derivatives thereof, including accepted nomenclature and numbering systems, have been extensively described, for example, in Abbas et al. (2010), Cellular and Molecular Immunology (6^{th} Ed.), W.B. Saunders Company; or Murphey et al. (2011), Janeway's Immunobiology (8^{th} Ed.), Garland Science.

As used herein an “antibody” or “intact antibody” typically refers to a Y-shaped tetrameric protein comprising two heavy (H) and two light (L) polypeptide chains held together by covalent disulfide bonds and non-covalent interactions. Each light chain is composed of one variable domain (VL) and one constant domain (CL). Each heavy chain comprises one variable domain (VH) and a constant region, which in the case of IgG, IgA, and IgD antibodies, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (from about 10 to about 60 amino acids in various IgG subclasses). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

As used herein the term “antibody” includes polyclonal antibodies, multiclonal antibodies, monoclonal antibodies, chimeric antibodies, humanized and primatized antibodies, CDR grafted antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. ScFv and ScFvFc); and derivatives thereof including Fc fusions and other modifications, and any other immunoreactive molecule so long as it exhibits preferential association or binding with a determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter α, δ, ε, γ, and a, respectively. Similarly, light chains of the antibodies from any vertebrate species can be assigned to one of two clearly distinct types, called kappa (κ) and lambda (λ), based on the amino acid sequences of their constant domains. Both light chains are compatible with the teachings herein and may be used in the fabrication of the disclosed antibody drug conjugates.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs). The non-covalent association between the V_{H} and the V_{L} region forms the Fv fragment (for “fragment variable”) which contains one of the two antigen-binding sites of the antibody. ScFv fragments (for single chain fragment variable), which can be obtained by genetic engineering, associates in a single polypeptide chain, the V_{H} and the V_{L} region of an antibody, separated by a peptide linker.

As used herein, the assignment of amino acids to each domain, framework region and CDR may be in accordance with one of the numbering schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co or AbM (Oxford Molecular/MSI Pharmacopia) unless otherwise noted. Amino acid residues which comprise CDRs as defined by Kabat, Chothia, MacCallum (also known as Contact) and AbM as obtained from the Abysis website database (infra.) are set out below.

TABLE 1

| | Kabat | Chothia | MacCallum | AbM |
|---|---|---|---|---|
| VH CDR1 | 31-35 | 26-32 | 30-35 | 26-35 |
| VH CDR2 | 50-65 | 52-56 | 47-58 | 50-58 |
| VH CDR3 | 95-102 | 95-102 | 93-101 | 95-102 |
| VL CDR1 | 24-34 | 24-34 | 30-36 | 24-34 |
| VL CDR2 | 50-56 | 50-56 | 46-55 | 50-56 |
| VL CDR3 | 89-97 | 89-97 | 89-96 | 89-97 |

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably the sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, any CDRs set forth herein are derived according to the Abysis database website as per Kabat et al.

For heavy chain constant region amino acid positions discussed in the invention, numbering is according to the Eu index first described in Edelman et al., 1969, Proc. Natl. Acad. Sci. USA 63(1): 78-85 describing the amino acid sequence of myeloma protein Eu, which reportedly was the first human IgG1 sequenced. The EU index of Edelman is also set forth in Kabat et al., 1991 (supra.). Thus, the terms “EU index as set forth in Kabat” or “EU index of Kabat” or “EU index” in the context of the heavy chain refers to the residue numbering system based on the human IgG1 Eu antibody of Edelman et al. as set forth in Kabat et al., 1991 (supra.) The numbering system used for light chain constant region amino acid sequence is similarly set forth in Kabat et al., (supra.) An exemplary kappa light chain constant region amino acid sequence compatible with the present invention is set forth immediately below (the C214 position, which may comprise a free cysteine as discussed below, is underlined).

(SEQ ID NO: 1)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

Similarly, an exemplary IgG1 heavy chain constant region amino acid sequence compatible with the present invention is set forth immediately below (the C220 position, which may comprise a free cysteine as discussed below, is underlined):

(SEQ ID NO: 2)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG.

The disclosed constant region sequences, or variations or derivatives thereof, may be operably associated with the disclosed heavy and light chain variable regions using standard molecular biology techniques to provide full-length antibodies that may be incorporated ADCs of the invention.

Those skilled in the art will appreciate there are two types of disulfide bridges or bonds in immunoglobulin molecules: interchain and intrachain disulfide bonds. As is well known the location and number of interchain disulfide bonds vary according to the immunoglobulin class and species. While the invention is not limited to any particular class or subclass of antibody, the IgG1 immunoglobulin will generally be used throughout the instant disclosure for illustrative purposes. In wild-type IgG1 molecules there are twelve intrachain disulfide bonds (four on each heavy chain and two on each light chain) and four interchain disulfide bonds. Intrachain disulfide bonds are generally somewhat protected and relatively less susceptible to reduction than interchain bonds. Conversely, interchain disulfide bonds are located on the surface of the immunoglobulin, are accessible to solvent and are usually relatively easy to reduce. Two interchain disulfide bonds exist between the heavy chains and one from each heavy chain to its respective light chain. It has been demonstrated that interchain disulfide bonds are not essential for heavy and light chain association. The IgG1 hinge region contain the cysteines in the heavy chain that form the interchain disulfide bonds, which provide structural support along with the flexibility that facilitates Fab movement. The heavy/heavy IgG1 interchain disulfide bonds are located at residues C226 and C229 (Eu numbering) while the IgG1 interchain disulfide bond between the light and heavy chain of IgG1 (heavy/light) are formed between C214 of the kappa or lambda light chain and C220 in the upper hinge region of the heavy chain.

### B. Antibody Generation and Production

Antibodies of the invention can be produced using a variety of methods known in the art.

### 1. Generation of Polyclonal Antibodies in Host Animals

The production of polyclonal antibodies in various host animals is well known in the art (see for example, Harlow and Lane (Eds.) (1988) Antibodies: A Laboratory Manual, CSH Press; and Harlow et al. (1989) Antibodies, NY, Cold Spring Harbor Press). In order to generate polyclonal antibodies, an immunocompetent animal (e.g., mouse, rat, rabbit, goat, non-human primate, etc.) is immunized with an antigenic protein or cells or preparations comprising an antigenic protein. After a period of time, polyclonal antibody-containing serum is obtained by bleeding or sacrificing the animal. The serum may be used in the form obtained from the animal or the antibodies may be partially or fully purified to provide immunoglobulin fractions or isolated antibody preparations.

Any form of antigen, or cells or preparations containing the antigen, can be used to generate an antibody that is specific for a determinant. The term “antigen” is used in a broad sense and may comprise any immunogenic fragment or determinant of the selected target including a single epitope, multiple epitopes, single or multiple domains or the entire extracellular domain (ECD). The antigen may be an isolated full-length protein, a cell surface protein (e.g., immunizing with cells expressing at least a portion of the antigen on their surface), or a soluble protein (e.g., immunizing with only the ECD portion of the protein). The antigen may be produced in a genetically modified cell. Any of the aforementioned antigens may be used alone or in combination with one or more immunogenicity enhancing adjuvants known in the art. The DNA encoding the antigen may be genomic or non-genomic (e.g., cDNA) and may encode at least a portion of the ECD, sufficient to elicit an immunogenic response. Any vectors may be employed to transform the cells in which the antigen is expressed, including but not limited to adenoviral vectors, lentiviral vectors, plasmids, and non-viral vectors, such as cationic lipids.

### 2. Monoclonal Antibodies

In selected embodiments, the invention contemplates use of monoclonal antibodies. The term “monoclonal antibody” or “mAb” refers to an antibody obtained from a population of substantially homogeneous antibodies, i.e., the individual antibodies comprising the population are identical except for possible mutations (e.g., naturally occurring mutations), that may be present in minor amounts.

Monoclonal antibodies can be prepared using a wide variety of techniques including hybridoma techniques, recombinant techniques, phage display technologies, transgenic animals (e.g., a XenoMouse®) or some combination thereof. For example, in preferred embodiments monoclonal antibodies can be produced using hybridoma and biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981). Following generation of a number of monoclonal antibodies that bind specifically to a determinant, particularly suitable antibodies may be selected through various screening processes, based on, for example, affinity for the determinant or rate of internalization. In particularly preferred embodiments monoclonal antibodies produced as described herein may be used as “source” antibodies and further modified to, for example, to improve affinity for the target, improve its production in cell culture, reduce immunogenicity in vivo, create multispecific constructs, etc.

### 3. Human Antibodies

The antibodies may comprise fully human antibodies. The term “human antibody” refers to an antibody (preferably a monoclonal antibody) which possesses an amino acid sequence that corresponds to that of an antibody produced by a human and/or has been made using any of the techniques for making human antibodies described below.

In one embodiment, recombinant human antibodies may be isolated by screening a recombinant combinatorial antibody library prepared using phage display. In one embodiment, the library is a scFv phage or yeast display library, generated using human VL and VH cDNAs prepared from mRNA isolated from B-cells.

Human antibodies can also be made by introducing human immunoglobulin loci into transgenic animals, e.g., mice in which the endogenous immunoglobulin genes have been partially or completely inactivated and human immunoglobulin genes have been introduced. Upon challenge antibody generation is observed which closely resembles that seen in humans in all respects, including gene rearrangement, assembly and fully human antibody repertoire. This approach is described, for example, in U.S. Pat. Nos. 5,545,807; 5,545,806; 5,569,825; 5,625,126; 5,633,425; 5,661,016, and U.S. Pat. Nos. 6,075,181 and 6,150,584 regarding XenoMouse® technology; and Lonberg and Huszar, 1995, PMID: 7494109). Alternatively, a human antibody may be prepared via immortalization of human B lymphocytes producing an antibody directed against a target antigen (such B lymphocytes may be recovered from an individual suffering from a neoplastic disorder or may have been immunized in vitro). See, e.g., Cole et al., Monoclonal Antibodies and Cancer Therapy, Alan R. Liss, p. 77 (1985); Boerner et al., 1991, PMID: 2051030; and U.S. Pat. No. 5,750,373. As with other monoclonal antibodies such human antibodies may be used as source antibodies.

### 4. Derived Antibodies:

Once the source antibodies have been generated, selected and isolated as described above they may be further altered to provide antibodies compatible with the instant invention having improved pharmaceutical characteristics. Preferably the source antibodies are modified or altered using known molecular engineering techniques to provide derived antibodies having the desired therapeutic properties.

### 4.1 Chimeric and Humanized Antibodies

Selected embodiments of the invention comprise murine monoclonal antibodies that immunospecifically bind to a selected determinant (e.g., SEZ6) and, for the purposes of the instant disclosure, may be considered “source” antibodies. In selected embodiments, antibodies compatible with the invention can be derived from such source antibodies through optional modification of the constant region and/or the antigen binding amino acid sequences of the source antibody. In certain embodiments an antibody is derived from a source antibody if selected amino acids in the source antibody are altered through deletion, mutation, substitution, integration or combination. In another embodiment, a “derived” antibody is one in which fragments of the source antibody (e.g., one or more CDRs or the entire heavy and light chain variable regions) are combined with or incorporated into an acceptor antibody sequence to provide the derivative antibody (e.g. chimeric or humanized antibodies). These derived antibodies can be generated using standard molecular biological techniques as described below, such as, for example, to improve affinity for the determinant; to improve antibody stability; to improve production and yield in cell culture; to reduce immunogenicity in vivo; to reduce toxicity; to facilitate conjugation of an active moiety; or to create a multispecific antibody. Such antibodies may also be derived from source antibodies through modification of the mature molecule (e.g., glycosylation patterns or pegylation) by chemical means or post-translational modification.

In one embodiment, the chimeric antibodies of the invention comprise chimeric antibodies that are derived from protein segments from at least two different species or class of antibodies that have been covalently joined. The term “chimeric” antibody is directed to constructs in which a portion of the heavy and/or light chain is identical or homologous to corresponding sequences in antibodies from a particular species or belonging to a particular antibody class or subclass, while the remainder of the chain(s) is identical or homologous to corresponding sequences in antibodies from another species or belonging to another antibody class or subclass, as well as fragments of such antibodies (U.S. Pat. No. 4,816,567; Morrison et al., 1984, PMID: 6436822). In some preferred embodiments chimeric antibodies of the instant invention may comprise all or most of the selected murine heavy and light chain variable regions operably linked to human light and heavy chain constant regions. In other particularly preferred embodiments, antibodies compatible with the invention may be “derived” from the mouse antibodies disclosed herein.

In other embodiments, the chimeric antibodies of the invention are “CDR grafted” antibodies, where the CDRs (as defined using Kabat, Chothia, McCallum, etc.) are derived from a particular species or belonging to a particular antibody class or subclass, while the remainder of the antibody is derived from an antibody from another species or belonging to another antibody class or subclass. For use in humans, one or more selected rodent CDRs (e.g., mouse CDRs) may be grafted into a human acceptor antibody, replacing one or more of the naturally occurring CDRs of the human antibody. These constructs generally have the advantages of providing full strength human antibody functions, e.g., complement dependent cytotoxicity (CDC) and antibody-dependent cell-mediated cytotoxicity (ADCC) while reducing unwanted immune responses to the antibody by the subject. In particularly preferred embodiments the CDR grafted antibodies will comprise one or more CDRs obtained from a mouse incorporated in a human framework sequence.

Similar to the CDR-grafted antibody is a “humanized” antibody. As used herein, a “humanized” antibody is a human antibody (acceptor antibody) comprising one or more amino acid sequences (e.g. CDR sequences) derived from one or more non-human antibodies (a donor or source antibody). In certain embodiments, “back mutations” can be introduced into the humanized antibody, in which residues in one or more FRs of the variable region of the recipient human antibody are replaced by corresponding residues from the non-human species donor antibody. Such back mutations may to help maintain the appropriate three-dimensional configuration of the grafted CDR(s) and thereby improve affinity and antibody stability. Antibodies from various donor species may be used including, without limitation, mouse, rat, rabbit, or non-human primate. Furthermore, humanized antibodies may comprise new residues that are not found in the recipient antibody or in the donor antibody to, for example, further refine antibody performance. CDR grafted and humanized antibodies compatible with the instant invention and comprising the source murine antibodies set forth in the Examples below may therefor readily be provided without undue experimentation using the prior art techniques as set forth herein.

Various art recognized techniques can further be used to determine which human sequences to use as acceptor antibodies to provide humanized constructs in accordance with the instant invention. Compilations of compatible human germline sequences and methods of determining their suitability as acceptor sequences are disclosed, for example, in Tomlinson, I. A. et al. (1992) J. Mol. Biol. 227:776-798; Cook, G. P. et al. (1995) Immunol. Today 16: 237-242; Chothia, D. et al. (1992) J. Mol. Biol. 227:799-817; and Tomlinson et al. (1995) EMBO J 14:4628-4638 each of which is incorporated herein in its entirety. The V-BASE directory (VBASE2−Retter et al., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK) may also be used to identify compatible acceptor sequences. Additionally, consensus human framework sequences described, for example, in U.S. Pat. No. 6,300,064 may also prove to be compatible acceptor sequences are can be used in accordance with the instant teachings. In general, human framework acceptor sequences are selected based on homology with the murine source framework sequences along with an analysis of the CDR canonical structures of the source and acceptor antibodies. The derived sequences of the heavy and light chain variable regions of the derived antibody may then be synthesized using art recognized techniques.

By way of example CDR grafted and humanized antibodies, and associated methods, are described in U.S. Pat. Nos. 6,180,370 and 5,693,762. For further details, see, e.g., Jones et al., 1986, PMID: 3713831); and U.S. Pat. Nos. 6,982,321 and 7,087,409.

The sequence identity or homology of the CDR grafted or humanized antibody variable region to the human acceptor variable region may be determined as discussed herein and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

### 4.2 Site-Specific Antibodies

The antibodies of the instant invention may be engineered to facilitate conjugation to the calicheamicin-linker constructs. It is advantageous for the antibody drug conjugate preparation to comprise a homogenous population of ADC molecules in terms of the position of the cytotoxin on the antibody and the drug to antibody ratio (DAR). Based on the instant disclosure one skilled in the art could readily fabricate site-specific engineered constructs and selectively conjugate them to the calicheamicin-linker constructs as described herein. As used in the instant application a “site-specific antibody” or “site-specific construct” means an antibody, or immunoreactive fragment thereof, wherein at least one amino acid in either the heavy or light chain is deleted, altered or substituted (preferably with another amino acid) to provide at least one free cysteine. Similarly, a “site-specific conjugate” shall be held to mean an ADC comprising a site-specific antibody and at least one calicheamicin compound conjugated to the unpaired cysteine(s). In certain embodiments the unpaired cysteine residue will comprise an unpaired intrachain residue. In other preferred embodiments the free cysteine residue will comprise an unpaired interchain cysteine residue. In still other preferred embodiments, and as will be discussed in more detail below, the unpaired or free cysteines may be engineered into any residue site present in the selected antibody or immunoreactive fragment thereof (i.e., such sites do not require disruption of a naturally occurring native disulfide bond). The engineered antibody can be of various isotypes, for example, IgG, IgE, IgA or IgD; and within those classes the antibody can be of various subclasses, for example, IgG1, IgG2, IgG3 or IgG4. For IgG constructs the light chain of the antibody can comprise either a kappa or lambda isotype each incorporating a C214 that, in preferred embodiments, may be unpaired due to a lack of a C220 residue in the IgG1 heavy chain.

Whether introducing free cysteines at preselected sites or disrupting native disulfide bonds, engineering of the antibodies as described herein provides for regulated stoichiometric conjugation of calicheamicin that allows the drug to antibody ratio (“DAR”) to largely be fixed with precision resulting in the generation of substantially DAR homogeneous preparations. Moreover the disclosed site-specific constructs further provide preparations that are substantially homogeneous with regard to the position of the payload on the antibody. Selective conjugation of the engineered constructs using stabilization agents as described herein increases the desired DAR species percentage and, along with the fabricated unpaired or free cysteine site, imparts conjugate stability and homogeneity that reduces non-specific toxicity caused by the inadvertent leaching of calicheamicin. This reduction in toxicity provided by selective conjugation of free cysteines and the relative homogeneity (both in conjugation positions and DAR) of the preparations also provides for an enhanced therapeutic index that allows for increased calicheamicin payload levels at the tumor site. Additionally, the resulting site-specific conjugates may optionally be purified using various chromatographic methodology to provide highly homogeneous site-specific conjugate preparations comprising desired DAR species (e.g., DAR=2) of greater than 75%, 80%, 85%, 90% or even 95%. Such conjugate homogeneity may further increase the therapeutic index of the disclosed preparations by limiting unwanted higher DAR conjugate impurities (which may be relatively unstable) that could increase toxicity.

It will be appreciated that the favorable properties exhibited by the disclosed engineered conjugate preparations is predicated, at least in part, on the ability to specifically direct the conjugation and largely limit the fabricated conjugates in terms of calicheamicin position and absolute DAR. Unlike most conventional ADC preparations preferred embodiments of the present invention do not rely entirely on partial or total reduction of the antibody to provide random conjugation sites and relatively uncontrolled generation of DAR species. Rather, selected embodiments of the present invention provide one or more predetermined unpaired (or free) cysteine sites by engineering the targeting antibody to disrupt one or more of the naturally occurring (i.e., “native”) interchain or intrachain disulfide bridges or to introduce a cysteine residue at any position. In the latter case it will be appreciated that, in selected embodiments, a cysteine residue may be incorporated anywhere along the antibody (or immunoreactive fragment thereof) heavy or light chain or appended thereto using standard molecular engineering techniques. In yet other preferred embodiments disruption of native disulfide bonds may be effected in combination with the introduction of a non-native cysteine to provide multiple free cysteines that may then be used as conjugation sites.

With regard to the introduction or addition of a cysteine residue or residues to provide a free cysteine (as opposed to disrupting a native disulfide bond) compatible position(s) on the antibody or antibody fragment may readily be discerned by on skilled in the art. Accordingly, in selected embodiments the cysteine(s) may be introduced in the CH1 domain, the CH2 domain or the CH3 domain or any combination thereof depending on the desired DAR, the antibody construct, the selected calicheamicin-linker and the antibody target. In other preferred embodiments the cysteines may be introduced into a kappa or lambda CL domain and, in particularly preferred embodiments, in the c-terminal region of the CL domain. In each case other amino acid residues proximal to the site of cysteine insertion may be altered, removed or substituted to facilitate molecular stability, conjugation efficiency or provide a protective environment for the calicheamicin payload once it is attached. In particular embodiments, the substituted residues occur at any accessible sites of the antibody. By substituting such surface residues with cysteine, reactive thiol groups are thereby positioned at readily accessible sites on the antibody and may be selectively reduced as described further herein.

As used herein, the terms “free cysteine” or “unpaired cysteine” may be used interchangeably unless otherwise dictated by context and shall mean any cysteine constituent of an antibody, whether naturally present or specifically incorporated in a selected residue position using molecular engineering techniques, that does not form a native disulfide bridge with another cysteine on the same antibody. Thus, in certain preferred embodiments the free cysteine may comprise a naturally occurring cysteine whose native interchain or intrachain disulfide bridge partner has been substituted, eliminated or otherwise altered to disrupt the naturally occurring disulfide bridge under physiological conditions thereby rendering the unpaired cysteine suitable for site-specific conjugation. In other preferred embodiments the free or unpaired cysteine will comprise a cysteine residue that is selectively placed at a predetermined site within the antibody heavy or light chain amino acid sequences. It will be appreciated that, prior to conjugation, free or unpaired cysteines may be present as a thiol (reduced cysteine), as a capped cysteine (oxidized) or as a non-natural intramolecular disulfide bond (oxidized) with another free cysteine on the same antibody depending on the oxidation state of the system. As discussed in more detail below, mild reduction of this antibody construct will provide thiols available for site-specific conjugation. In particularly preferred embodiments the free or unpaired cysteines (whether naturally occurring or incorporated) will be subject to selective reduction and subsequent calicheamicin conjugation to provide the disclosed homogenous DAR compositions.

In some embodiments an interchain cysteine residue is deleted. In other embodiments an interchain cysteine is substituted for another amino acid (e.g., a naturally occurring amino acid). For example, the amino acid substitution can result in the replacement of an interchain cysteine with a neutral (e.g. serine, threonine or glycine) or hydrophilic (e.g. methionine, alanine, valine, leucine or isoleucine) residue. In one particularly preferred embodiment an interchain cysteine is replaced with a serine.

In some embodiments contemplated by the invention the deleted or substituted cysteine residue is on the light chain (either kappa or lambda) thereby leaving a free cysteine on the heavy chain. In other embodiments the deleted or substituted cysteine residue is on the heavy chain leaving the free cysteine on the light chain constant region. Upon assembly it will be appreciated that deletion or substitution of a single cysteine in either the light or heavy chain of an intact antibody results in a site-specific antibody having two unpaired cysteine residues.

In one particularly preferred embodiment the cysteine at position 214 (C214) of the IgG light chain (kappa or lambda) is deleted or substituted. In another preferred embodiment the cysteine at position 220 (C220) on the IgG heavy chain is deleted or substituted. In further embodiments the cysteine at position 226 or position 229 on the heavy chain is deleted or substituted. In one embodiment C220 on the heavy chain is substituted with serine (C220S) to provide the desired free cysteine in the light chain. Such engineered constructs are used in the Examples below to provide novel antibody drug conjugates compatible with the teachings herein. In another embodiment C214 in the light chain is substituted with serine (C214S) to provide the desired free cysteine in the heavy chain. A summary of these preferred constructs is shown in Table 2 immediately below where all numbering is according to the EU index as set forth in Kabat and WT stands for “wild-type” or native constant region sequences without alterations and delta (A) designates the deletion of an amino acid residue (e.g., C214Δ indicates that the cysteine at position 214 has been deleted).

TABLE 2

| | Antibody | |
|---|---|---|
| Designation | Component | Alteration |
| ss1 | Heavy Chain | C220S |
| | Light Chain | WT |
| ss2 | Heavy Chain | C220Δ |
| | Light Chain | WT |
| ss3 | Heavy Chain | WT |
| | Light Chain | C214Δ |
| ss4 | Heavy Chain | WT |
| | Light Chain | C214S |

With regard to the introduction or addition of a cysteine residue or residues to provide a free cysteine (as opposed to disrupting a native disulfide bond) compatible position(s) on the antibody or antibody fragment may readily be discerned by one skilled in the art. Accordingly, in selected embodiments the cysteine(s) may be introduced in the CH1 domain, the CH2 domain or the CH3 domain or any combination thereof depending on the desired DAR, the antibody construct, the selected payload and the antibody target. In other preferred embodiments the cysteines may be introduced into a kappa or lambda CL domain and, in particularly preferred embodiments, in the c-terminal region of the CL domain. In each case other amino acid residues proximal to the site of cysteine insertion may be altered, removed or substituted to facilitate molecular stability, conjugation efficiency or provide a protective environment for the payload once it is attached. In particular embodiments, the substituted residues occur at any accessible sites of the antibody. By substituting such surface residues with cysteine, reactive thiol groups are thereby positioned at readily accessible sites on the antibody and may be selectively reduced as described further herein. In particular embodiments, the substituted residues occur at accessible sites of the antibody. By substituting those residues with cysteine, reactive thiol groups are thereby positioned at accessible sites of the antibody and may be used to selectively conjugate the antibody. In certain embodiments, any one or more of the following residues may be substituted with cysteine: V205 (Kabat numbering) of the light chain; A118 (Eu numbering) of the heavy chain; and S400 (Eu numbering) of the heavy chain Fc region. Additional substitution positions and methods of fabricating compatible site-specific antibodies are set forth in U.S. Pat. No. 7,521,541 which is incorporated herein in its entirety.

Once the site-specific construct is provided the resulting free cysteines may be selectively reduced using the novel techniques disclosed herein without substantially disrupting intact native disulfide bridges, to provide reactive thiols predominantly at the selected cysteine sites. These manufactured thiols are then subject to directed conjugation with the disclosed calicheamicin-linker constructs without substantial non-specific conjugation. That is, the engineered constructs and, optionally, the selective reduction techniques disclosed herein may be used to largely eliminate non-specific, random conjugation of the calicheamicin payloads. Significantly this provides preparations that are substantially homogeneous in both DAR species distribution and calicheamicin position on the targeting antibody. As discussed below the elimination of relatively high DAR contaminants can, in and of itself, reduce non-specific toxicity and expand the therapeutic index of the preparation. Moreover, such selectivity allows the calicheamicin payloads to largely be placed in particularly advantageous predetermined positions (such as the terminal region of the light chain constant region) where the calicheamicin-linker construct is somewhat protected until it reaches the tumor but is suitably presented and processed upon delivery. Thus, design of the engineered antibody to facilitate specific calicheamicin payload positioning may also be used to reduce the non-specific toxicity of the disclosed preparations. Finally, the ability to selectively and reproducibly direct conjugation of the antibody greatly simplifies characterization of the resulting composition thereby facilitating drug development.

It will be appreciated that creation of these predetermined free cysteine sites may be achieved using art-recognized molecular engineering techniques to introduce a cysteine at a preselected site on the antibody or to remove, alter or replace one of the constituent cysteine residues of the disulfide bond. Using these techniques one skilled in the art will appreciate that any antibody class or isotype may be engineered to exhibit one or more free cysteine(s) capable of being selectively conjugated in accordance with the instant invention. Moreover, the selected antibody maybe engineered to specifically exhibit 1, 2, 3, 4, 5, 6, 7 or even 8 free cysteines depending on the desired DAR. More preferably the selected antibody will be engineered to contain 2 or 4 free cysteines and even more preferably to contain 2 free cysteines. It will also be appreciated that the free cysteines may be positioned in engineered antibody to facilitate delivery of the conjugated calicheamicin to the target while reducing non-specific toxicity. In this respect selected embodiments of the invention comprising IgG1 antibodies will position the calicheamicin payload on the CH1 domain and more preferably on the C-terminal end of the domain. In other preferred embodiments the antibody constructs will be engineered to position the calicheamicin on the light chain constant region and more preferably at the C-terminal end of the constant region.

Significantly, limiting payload conjugation to the engineered free cysteines may also be facilitated by selective reduction of the construct using novel stabilization agents and the calicheamicin-linker constructs set forth below. “Selective reduction” as used herein will mean exposure of the engineered constructs to reducing conditions that reduce the free cysteines (thereby providing reactive thiols) without substantially disrupting intact native disulfide bonds. In general selective reduction may be effected using any reducing agents, or combinations thereof that provide the desired thiols without disrupting the intact disulfide bonds. In certain preferred embodiments, and as set forth in the Examples below, selective reduction may be effected using a stabilizing agent and mild reducing conditions to prepare the engineered construct for conjugation. As discussed in more detail herein compatible stabilizing agents will generally facilitate reduction of the free cysteines and allow the desired conjugation to proceed under less stringent reducing conditions. This allows a substantial majority of the native disulfide bonds to remain intact and markedly reduces the amount of non-specific conjugation thereby limiting unwanted contaminants and potential toxicity. The relatively mild reducing conditions may be attained through the use of a number of systems but preferably comprises the use of thiol containing compounds. It will be appreciated that one skilled in the art could readily derive compatible reducing systems in view of the instant disclosure.

### 4.3 Constant Region Modifications and Altered Glycosylation

Selected embodiments of the present invention may also comprise substitutions or modifications of the constant region (i.e. the Fc region), including without limitation, amino acid residue substitutions, mutations and/or modifications, which result in a compound with preferred characteristics including, but not limited to: altered pharmacokinetics, increased serum half-life, increase binding affinity, reduced immunogenicity, increased production, altered Fc ligand binding to an Fc receptor (FcR), enhanced or reduced ADCC or CDC, altered glycosylation or modified constant region binding specificity.

Compounds with improved Fc effector functions can be generated, for example, through changes in amino acid residues involved in the interaction between the Fc domain and an Fc receptor (e.g., FcγRI, FcγRIIA and B, FcγRIII and FcRn), which may lead to increased cytotoxicity and/or altered pharmacokinetics, such as increased serum half-life (see, for example, Ravetch and Kinet, Annu. Rev. Immunol 9:457-92 (1991); Capel et al., Immunomethods 4:25-34 (1994); and de Haas et al., J. Lab. Clin. Med. 126:330-41 (1995).

In selected embodiments, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S.P.N. 2003/0190311). With regard to such embodiments, Fc variants may provide half-lives in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-life results in a higher serum titer which thus reduces the frequency of the administration of the antibody drug conjugates or reduces the concentration of the antibodies to be administered. Binding to human FcRn in vivo and serum half-life of human FcRn high affinity binding polypeptides can be assayed, e.g., in transgenic mice or transfected human cell lines expressing human FcRn, or in primates to which the polypeptides with a variant Fc region are administered. WO 2000/42072 describes antibody variants with improved or diminished binding to FcRns. See also, e.g., Shields et al. J. Biol. Chem. 9(2):6591-6604 (2001).

In other embodiments, Fc alterations may lead to enhanced or reduced ADCC or CDC activity. As in known in the art, CDC refers to the lysing of a target cell in the presence of complement, and ADCC refers to a form of cytotoxicity in which secreted Ig bound onto FcRs present on certain cytotoxic cells (e.g., Natural Killer cells, neutrophils, and macrophages) enables these cytotoxic effector cells to bind specifically to an antigen-bearing target cell and subsequently kill the target cell with cytotoxins. In the context of the instant invention antibody variants are provided with “altered” FcR binding affinity, which is either enhanced or diminished binding as compared to a parent or unmodified antibody or to an antibody comprising a native sequence FcR. Such variants which display decreased binding may possess little or no appreciable binding, e.g., 0-20% binding to the FcR compared to a native sequence, e.g. as determined by techniques well known in the art. In other embodiments the variant will exhibit enhanced binding as compared to the native immunoglobulin Fc domain. It will be appreciated that these types of Fc variants may advantageously be used to enhance the effective anti-neoplastic properties of the disclosed antibodies. In yet other embodiments, such alterations lead to increased binding affinity, reduced immunogenicity, increased production, altered glycosylation and/or disulfide bonds (e.g., for conjugation sites), modified binding specificity, increased phagocytosis; and/or down regulation of cell surface receptors (e.g. B cell receptor; BCR), etc.

Still other embodiments comprise one or more engineered glycoforms, e.g., a site-specific antibody comprising an altered glycosylation pattern or altered carbohydrate composition that is covalently attached to the protein (e.g., in the Fc domain). See, for example, Shields, R. L. et al. (2002) J. Biol. Chem. 277:26733-26740. Engineered glycoforms may be useful for a variety of purposes, including but not limited to enhancing or reducing effector function, increasing the affinity of the antibody for a target or facilitating production of the antibody. In certain embodiments where reduced effector function is desired, the molecule may be engineered to express an aglycosylated form. Substitutions that may result in elimination of one or more variable region framework glycosylation sites to thereby eliminate glycosylation at that site are well known (see e.g. U.S. Pat. Nos. 5,714,350 and 6,350,861). Conversely, enhanced effector functions or improved binding may be imparted to the Fc containing molecule by engineering in one or more additional glycosylation sites.

Other embodiments include an Fc variant that has an altered glycosylation composition, such as a hypofucosylated antibody having reduced amounts of fucosyl residues or an antibody having increased bisecting GlcNAc structures. Such altered glycosylation patterns have been demonstrated to increase the ADCC ability of antibodies. Engineered glycoforms may be generated by any method known to one skilled in the art, for example by using engineered or variant expression strains, by co-expression with one or more enzymes (for example N-acetylglucosaminyltransferase III (GnTIII)), by expressing a molecule comprising an Fc region in various organisms or cell lines from various organisms or by modifying carbohydrate(s) after the molecule comprising Fc region has been expressed (see, for example, WO 2012/117002).

### 4.4 Fragments

Regardless of which form of antibody (e.g. chimeric, humanized, etc.) is selected to practice the invention it will be appreciated that immunoreactive fragments, as the targeting agent of an antibody drug conjugate, of the same may be used in accordance with the teachings herein. An “antibody fragment” comprises at least a portion of an intact antibody. As used herein, the term “fragment” of an antibody molecule includes antigen-binding fragments of antibodies, and the term “antigen-binding fragment” or “immunoreactive fragment” refers to a polypeptide fragment of an immunoglobulin or antibody that immunospecifically binds or reacts with a selected antigen or immunogenic determinant thereof or competes with the intact antibody from which the fragments were derived for specific antigen binding.

Exemplary site-specific fragments include: variable light chain fragments (VL), an variable heavy chain fragments (VH), scFv, F(ab′)2 fragment, Fab fragment, Fd fragment, Fv fragment, single domain antibody fragments, diabodies, linear antibodies, single-chain antibody molecules and multispecific antibodies formed from antibody fragments. In addition, an active site-specific fragment comprises a portion of the antibody that retains its ability to interact with the antigen/substrates or receptors and modify them in a manner similar to that of an intact antibody (though maybe with somewhat less efficiency). Such antibody fragments may further be engineered to comprise one or more free cysteines.

In other embodiments, an antibody fragment is one that comprises the Fc region and that retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fc sequence comprising at least one free cysteine capable of conferring in vivo stability to the fragment.

As would be well recognized by those skilled in the art, fragments can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragments.

### 4.5 Multivalent Constructs

In other embodiments, the antibody drug conjugates of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105.

In one embodiment, the antibodies are bispecific antibodies in which the two chains have different specificities, as described in Millstein et al., 1983, Nature, 305:537-539. Other embodiments include antibodies with additional specificities such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

Multivalent antibodies may immunospecifically bind to different epitopes of the desired target molecule or may immunospecifically bind to both the target molecule as well as a heterologous epitope, such as a heterologous polypeptide or solid support material. While preferred embodiments only bind two antigens (i.e. bispecific antibodies), antibodies with additional specificities such as trispecific antibodies are also encompassed by the instant invention. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

### 5. Recombinant Production of Antibodies

Antibodies and fragments thereof may be produced or modified using genetic material obtained from antibody producing cells and recombinant technology (see, for example, Berger and Kimmel, Guide to Molecular Cloning Techniques, Methods in Enzymology vol. 152 Academic Press, Inc., San Diego, Calif.; Sambrook and Russell (Eds.) (2000) Molecular Cloning: A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc.; and U.S. Pat. No. 7,709,611). The nucleic acids may be present in whole cells, in a cell lysate, or in a partially purified or substantially pure form. A nucleic acid is “isolated” or rendered substantially pure when separated from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the invention can be, for example, DNA (e.g. genomic DNA, cDNA), RNA and artificial variants thereof (e.g., peptide nucleic acids), whether single-stranded or double-stranded or RNA, RNA and may or may not contain introns. In a preferred embodiment, the nucleic acid is a cDNA molecule.

Nucleic acids can be obtained using standard molecular biology techniques. For antibodies expressed by hybridomas cDNAs encoding the light and heavy chains of the antibody can be obtained by standard PCR amplification or cDNA cloning techniques. For antibodies obtained from an immunoglobulin gene library (e.g., such as when using phage display techniques), nucleic acids encoding the immunoreactive fragment of the antibody can be recovered from the library using standard art-recognized techniques.

DNA fragments encoding VH and VL segments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, means that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, et al. (1991) (supra)) and DNA fragments encompassing these regions can be obtained by standard PCR amplification. The heavy chain constant region can be an IgG, IgG2, IgG3, IgG4, IgA, IgE, IgM or IgD constant region, but most preferably is an IgG1 or IgG4 constant region. An exemplary IgG1 constant region is set forth in SEQ ID NO: 2. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, et al. (1991) (supra)) and DNA fragments encompassing these regions can be obtained by standard PCR amplification. The light chain constant region can be a kappa or lambda constant region, but most preferably is a kappa constant region. In this respect an exemplary compatible kappa light chain constant region is set forth in SEQ ID NO: 1.

Antibodies compatible with the instant invention may be produced using vectors comprising such nucleic acids as described above, which may be operably linked to a promoter (see, e.g., WO 86/05807; WO 89/01036; and U.S. Pat. No. 5,122,464); and other transcriptional regulatory and processing control elements of the eukaryotic secretory pathway. Host cells harboring such vectors and host-expression systems are then cultured using art-recognized techniques to provide the desired antibodies.

As used herein, the term “host-expression system” includes any kind of cellular system that can be engineered to generate either nucleic acids or the polypeptides and antibodies compatible with the invention. Such host-expression systems include, but are not limited to microorganisms (e.g., E. coli or B. subtilis) transformed or transfected with recombinant bacteriophage DNA or plasmid DNA; yeast (e.g., Saccharomyces) transfected with recombinant yeast expression vectors; or mammalian cells (e.g., COS, CHO-S, HEK-293T, 3T3 cells) harboring recombinant expression constructs containing promoters derived from the genome of mammalian cells or viruses (e.g., the adenovirus late promoter). The host cell may be co-transfected with two expression vectors, for example, the first vector encoding a heavy chain derived polypeptide and the second vector encoding a light chain derived polypeptide.

Methods of transforming mammalian cells are well known in the art. See, for example, U.S. Pat. Nos. 4,399,216, 4,912,040, 4,740,461, and 4,959,455. The host cell may also be engineered to allow the production of an antigen binding molecule with various characteristics (e.g. modified glycoforms or proteins having GnTIII activity).

For long-term, high-yield production of recombinant proteins stable expression is preferred. Accordingly, cell lines that stably express the selected antibody may be engineered using standard art recognized techniques and form part of the invention. Rather than using expression vectors that contain viral origins of replication, host cells can be transformed with DNA controlled by appropriate expression control elements (e.g., promoter or enhancer sequences, transcription terminators, polyadenylation sites, etc.), and a selectable marker. Any of the selection systems well known in the art may be used, including the glutamine synthetase gene expression system (the GS system) which provides an efficient approach for enhancing expression under certain conditions. The GS system is discussed in whole or part in connection with EP 0 216 846, EP 0 256 055, EP 0 323 997 and EP 0 338 841 and U.S. Pat. Nos. 5,591,639 and 5,879,936. Another preferred expression system for the development of stable cell lines is the Freedom™ CHO-S Kit (Life Technologies).

Once an antibody compatible with the invention has been produced by recombinant expression or any other of the disclosed techniques, it may be purified or isolated by methods known in the art, meaning that it is identified, characterized, separated and/or recovered from its natural environment and from contaminants that would interfere with therapeutic uses for the antibody including ADCs. Isolated antibodies include antibodies in situ within recombinant cells.

These isolated preparations may be purified using various art recognized techniques, such as, for example, ion exchange and size exclusion chromatography, dialysis, diafiltration, and affinity chromatography, particularly Protein A or Protein G affinity chromatography.

### 6. Post-Production Selection

No matter how obtained, antibody-producing cells (e.g., hybridomas, yeast colonies, etc.) may be selected, cloned and further screened for desirable characteristics including, for example, robust growth, high antibody production and desirable antibody characteristics such as high affinity for the antigen of interest. Hybridomas can be expanded in vitro in cell culture or in vivo in syngeneic immunocompromised animals. Methods of selecting, cloning and expanding hybridomas and/or colonies are well known to those of ordinary skill in the art. Once the desired antibodies are identified the relevant genetic material may be isolated, manipulated and expressed using common, art-recognized molecular biology and biochemical techniques.

The antibodies produced by naïve libraries (either natural or synthetic) may be of moderate affinity (K_{A} of about 10⁶ to 10⁷ M⁻¹). To enhance affinity, affinity maturation may be mimicked in vitro by constructing antibody libraries (e.g., by introducing random mutations in vitro by using error-prone polymerase) and reselecting antibodies with high affinity for the antigen from those secondary libraries (e.g. by using phage or yeast display). WO 9607754 describes a method for inducing mutagenesis in a CDR of an immunoglobulin light chain to create a library of light chain genes.

Various techniques can be used to select antibodies, including but not limited to, phage or yeast display in which a library of human combinatorial antibodies or scFv fragments is synthesized on phages or yeast, the library is screened with the antigen of interest or an antibody-binding portion thereof, and the phage or yeast that binds the antigen is isolated, from which one may obtain the antibodies or immunoreactive fragments (Vaughan et al., 1996, PMID: 9630891; Sheets et al., 1998, PMID: 9600934; Boder et al., 1997, PMID: 9181578; Pepper et al., 2008, PMID: 18336206). Kits for generating phage or yeast display libraries are commercially available. There also are other methods and reagents that can be used in generating and screening antibody display libraries (see U.S. Pat. No. 5,223,409; WO 92/18619, WO 91/17271, WO 92/20791, WO 92/15679, WO 93/01288, WO 92/01047, WO 92/09690; and Barbas et al., 1991, PMID: 1896445). Such techniques advantageously allow for the screening of large numbers of candidate antibodies and provide for relatively easy manipulation of sequences (e.g., by recombinant shuffling).

### V Characteristics of Antibodies

In selected embodiments, antibody-producing cells (e.g., hybridomas or yeast colonies) may be selected, cloned and further screened for favorable properties including, for example, robust growth, high antibody production and, as discussed in more detail below, desirable antibody drgu conjugate characteristics. In other cases characteristics of the antibody may be imparted by selecting a particular antigen (e.g., a specific protein domain) or immunoreactive fragment of the target antigen for inoculation of the animal. In still other embodiments the selected antibodies may be engineered as described above to enhance or refine immunochemical characteristics such as affinity or pharmacokinetics.

### A. Neutralizing Antibodies

In selected embodiments antibodies compatible with the invention may be “antagonists” or “neutralizing” antibodies, meaning that the antibody may associate with a determinant and block or inhibit the activities of said determinant either directly or by preventing association of the determinant with a binding partner such as a ligand or a receptor, thereby interrupting the biological response that otherwise would result from the interaction of the molecules. A neutralizing or antagonist antibody will substantially inhibit binding of the determinant to its ligand or substrate when an excess of antibody reduces the quantity of binding partner bound to the determinant by at least about 20%, 30%, 40%, 50%, 60%, 70%, 80%, 85%, 90%, 95%, 97%, 99% or more as measured, for example, by target molecule activity or in an in vitro competitive binding assay. It will be appreciated that the modified activity may be measured directly using art recognized techniques or may be measured by the impact the altered activity has downstream (e.g., oncogenesis or cell survival).

### B. Internalizing Antibodies

In many cases selected determinants remain associated with tumorigenic cell surfaces, thereby allowing for localization and internalization of the disclosed ADCs. In preferred embodiments such antibodies will be associated with, or conjugated to, one or more calicheamicin payload(s) that kill the cell upon internalization. In particularly preferred embodiments the ADCs of the instant invention will comprise an internalizing site-specific ADC with calicheamicin payload(s).

As used herein, an antibody that “internalizes” is one that is taken up (along with any cytotoxin) by the cell upon binding to an associated antigen or receptor. For therapeutic applications, internalization will preferably occur in vivo in a subject in need thereof. The number of ADCs internalized may be sufficient to kill an antigen-expressing cell, especially an antigen-expressing cancer stem cell. Depending on the potency of calicheamicin or the ADC as a whole (e.g., based on DAR), the uptake of a single antibody molecule into the cell may be sufficient to kill the target cell to which the antibody binds. For example, with higher DAR and efficient delivery of the attached calicheamicin some ADCs may be so highly potent that the internalization of a few molecules is sufficient to kill the tumor cell. Whether an antibody internalizes upon binding to a mammalian cell can be determined by various art-recognized assays (e.g., saporin assays such as Mab-Zap and Fab-Zap; Advanced Targeting Systems). Methods of detecting whether an antibody internalizes into a cell are also described in U.S. Pat. No. 7,619,068.

### C. Depleting Antibodies

In other embodiments the antibodies of the invention are depleting antibodies. The term “depleting” antibody refers to an antibody that preferably binds to an antigen on or near the cell surface and induces, promotes or causes the death of the cell (e.g., by CDC, ADCC or introduction of a cytotoxic agent). In preferred embodiments, the selected depleting antibodies will be conjugated to a cytotoxin. Preferably a depleting antibody will be able to kill at least 20%, 30%, 40%, 50%, 60%, 70%, 80%, 85%, 90%, 95%, 97%, or 99% of SEZ6-expressing cells in a defined cell population. In some embodiments the cell population may comprise enriched, sectioned, purified or isolated tumorigenic cells, including cancer stem cells. In other embodiments the cell population may comprise whole tumor samples or heterogeneous tumor extracts that comprise cancer stem cells. Standard biochemical techniques may be used to monitor and quantify the depletion of tumorigenic cells in accordance with the teachings herein.

### D. Binding Affinity

Antibodies compatible with the instant invention preferably have a high binding affinity for the selected determinant (e.g. SEZ6). The term “K_{D}” refers to the equilibrium dissociation constant or apparent affinity of a particular antibody-antigen interaction. An antibody compatible with the invention can immunospecifically bind its target antigen when the dissociation constant K_{D}(k_{off}/kₒₙ) is ≤10⁻⁶ M. The antibody specifically binds antigen with high affinity when the K_{D} is ≤5×10⁻⁹ M, and with very high affinity when the K_{D} is ≤5×10⁻¹⁰ M. In one embodiment of the invention, the antibody has a K_{D} of ≤10⁻⁹ M and an off-rate of about 1×10⁻⁴/sec. In one embodiment of the invention, the off-rate is ≤1×10⁻⁵/sec. In other embodiments of the invention, the antibodies will bind to a determinant with a K_{D} of between about 10⁻⁷ M and 10⁻¹⁰ M, and in yet another embodiment it will bind with a K_{D}≤2×10⁻¹⁰ M. Still other selected embodiments of the invention comprise antibodies that have a K_{D} (k_{off}/kₒₙ) of less than 10⁻⁶ M, less than 5×10⁻⁶ M, less than 10⁻⁷ M, less than 5×10⁻⁷ M, less than 10⁻⁸ M, less than 5×10⁹ M, less than 10⁻⁹ M, less than 5×10⁻⁹ M, less than 10⁻¹⁰ M, less than 5×10⁻¹ M, less than 10⁻¹¹ M, less than 5×10⁻¹¹ M, less than 10-12 M, less than 5×10⁻¹² M, less than 10⁻¹³ M, less than 5×10⁻¹³ M, less than 10⁻¹⁴ M, less than 5×10⁻¹⁴M, less than 10⁻¹⁵ M or less than 5×10⁻¹⁵ M.

In certain embodiments, an antibody compatible with the invention immunospecifically binds to a determinant with an association rate constant or kₒₘ (or kₐ) rate (antibody+antigen (Ag)^{k}ₒₙ←antibody-Ag) of at least 10⁵ M⁻¹s⁻¹, at least 2×10⁵ M⁻¹s⁻¹, at least 5×10⁵ M⁻¹s⁻¹, at least 10⁶ M⁻¹ s⁻¹, at least 5×10⁶ M⁻¹ s⁻¹, at least 10⁷ M⁻¹s⁻¹, at least 5×10⁷ M⁻¹s⁻¹, or at least 108 M⁻¹s⁻¹.

In another embodiment, an antibody compatible with the invention immunospecifically binds to a determinant with a disassociation rate constant or k_{off} (or k_{d}) rate (antibody+antigen (Ag)^{k}_{off}←antibody-Ag) of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻³ s⁻¹, less than 10⁴s⁻¹, less than 5×10⁴ s⁻¹, less than 10⁻⁵ s⁻¹, less than 5×10⁻⁵ s⁻¹, less than 10⁻⁶ s⁻¹, less than 5×10⁻⁶ s⁻¹ less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹ or less than 10⁻¹⁰ s⁻¹.

Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry.

### E. Binning and Epitope Mapping

As used herein, the term “binning” refers to methods used to group antibodies into “bins” based on their antigen binding characteristics and whether they compete with each other. The initial determination of bins may be further refined and confirmed by epitope mapping and other techniques as described herein. However it will be appreciated that empirical assignment of antibodies to individual bins provides information that may be indicative of the therapeutic potential of the disclosed antibody drug conjugates.

More specifically, one can determine whether a selected reference antibody (or fragment thereof) competes for binding with a second test antibody (i.e., is in the same bin) by using methods known in the art. In one embodiment, a reference antibody is associated with a selected antigen under saturating conditions and then the ability of a secondary or test antibody to bind to the same antigen is determined using standard immunochemical techniques. If the test antibody is able to substantially bind to the antigen at the same time as the reference antibody, then the secondary or test antibody binds to a different epitope than the primary or reference antibody. However, if the test antibody is not able to substantially bind to the antigen at the same time, then the test antibody binds to the same epitope, an overlapping epitope, or an epitope that is in close proximity (at least sterically) to the epitope bound by the reference antibody. That is, the test antibody competes for antigen binding and is in the same bin as the reference antibody.

The term “compete” or “competing antibody” when used in the context of the disclosed antibodies means competition between antibodies as determined by an assay in which a test antibody or immunologically functional fragment being tested inhibits specific binding of a reference antibody to a common antigen. Typically, such an assay involves the use of purified antigen (or a domain or fragment thereof) bound to a solid surface or expressed cells, an unlabeled test antibody and a labeled reference antibody. Competitive inhibition is measured by determining the amount of label bound to the solid surface or cells in the presence of the test antibody. Usually the test antibody is present in excess and/or allowed to bind first. Additional details regarding methods for determining competitive binding are provided in the Examples herein. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more.

Conversely, when the reference antibody is bound it will preferably inhibit binding of a subsequently added test antibody by at least 30%, 40%, 45%, 50%, 55%, 60%, 65%, 70% or 75%. In some instance, binding of the test antibody is inhibited by at least 80%, 85%, 90%, 95%, or 97% or more.

Generally binning or competitive binding may be determined using various art-recognized techniques, such as, for example, immunoassays such as western blots, radioimmunoassays, enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such immunoassays are routine and well known in the art (see, Ausubel et al, eds, (1994) Current Protocols in Molecular Biology, Vol. 1, John Wiley & Sons, Inc., New York). Additionally, cross-blocking assays may be used (see, for example, WO 2003/48731; and Harlow et al. (1988) Antibodies, A Laboratory Manual, Cold Spring Harbor Laboratory, Ed Harlow and David Lane).

Other technologies used to determine competitive inhibition (and hence “bins”), include: surface plasmon resonance using, for example, the BIAcore™ 2000 system (GE Healthcare); bio-layer interferometry using, for example, a ForteBio® Octet RED (ForteBio); or flow cytometry bead arrays using, for example, a FACSCanto II (BD Biosciences) or a multiplex LUMINEX™ detection assay (Luminex).

Luminex is a bead-based immunoassay platform that enables large scale multiplexed antibody pairing. The assay compares the simultaneous binding patterns of antibody pairs to the target antigen. One antibody of the pair (capture mAb) is bound to Luminex beads, wherein each capture mAb is bound to a bead of a different color. The other antibody (detector mAb) is bound to a fluorescent signal (e.g. phycoerythrin (PE)). The assay analyzes the simultaneous binding (pairing) of antibodies to an antigen and groups together antibodies with similar pairing profiles. Similar profiles of a detector mAb and a capture mAb indicates that the two antibodies bind to the same or closely related epitopes. In one embodiment, pairing profiles can be determined using Pearson correlation coefficients to identify the antibodies which most closely correlate to any particular antibody on the panel of antibodies that are tested. In preferred embodiments a test/detector mAb will be determined to be in the same bin as a reference/capture mAb if the Pearson's correlation coefficient of the antibody pair is at least 0.9. In other embodiments the Pearson's correlation coefficient is at least 0.8, 0.85, 0.87 or 0.89. In further embodiments, the Pearson's correlation coefficient is at least 0.91, 0.92, 0.93, 0.94, 0.95, 0.96, 0.97, 0.98, 0.99 or 1. Other methods of analyzing the data obtained from the Luminex assay are described in U.S. Pat. No. 8,568,992. The ability of Luminex to analyze 100 different types of beads (or more) simultaneously provides almost unlimited antigen and/or antibody surfaces, resulting in improved throughput and resolution in antibody epitope profiling over a biosensor assay (Miller, et al., 2011, PMID: 21223970).

“Surface plasmon resonance,” refers to an optical phenomenon that allows for the analysis of real-time specific interactions by detection of alterations in protein concentrations within a biosensor matrix.

In other embodiments, a technique that can be used to determine whether a test antibody “competes” for binding with a reference antibody is “bio-layer interferometry”, an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. Such biolayer interferometry assays may be conducted using a ForteBio® Octet RED machine as follows. A reference antibody (Ab1) is captured onto an anti-mouse capture chip, a high concentration of non-binding antibody is then used to block the chip and a baseline is collected. Monomeric, recombinant target protein is then captured by the specific antibody (Ab1) and the tip is dipped into a well with either the same antibody (Ab1) as a control or into a well with a different test antibody (Ab2). If no further binding occurs, as determined by comparing binding levels with the control Ab1, then Ab1 and Ab2 are determined to be “competing” antibodies. If additional binding is observed with Ab2, then Ab1 and Ab2 are determined not to compete with each other. This process can be expanded to screen large libraries of unique antibodies using a full row of antibodies in a 96-well plate representing unique bins. In preferred embodiments a test antibody will compete with a reference antibody if the reference antibody inhibits specific binding of the test antibody to a common antigen by at least 40%, 45%, 50%, 55%, 60%, 65%, 70% or 75%. In other embodiments, binding is inhibited by at least 80%, 85%, 90%, 95%, or 97% or more.

Once a bin, encompassing a group of competing antibodies, has been defined further characterization can be carried out to determine the specific domain or epitope on the antigen to which the antibodies in a bin bind. Domain-level epitope mapping may be performed using a modification of the protocol described by Cochran et al., 2004, PMID: 15099763. Fine epitope mapping is the process of determining the specific amino acids on the antigen that comprise the epitope of a determinant to which the antibody binds. The term “epitope” is used in its common biochemical sense and refers to that portion of the target antigen capable of being recognized and specifically bound by a particular antibody. In certain embodiments, epitopes or immunogenic determinants include chemically active surface groupings of molecules such as amino acids, sugar side chains, phosphoryl groups, or sulfonyl groups, and, in certain embodiments, may have specific three-dimensional structural characteristics, and/or specific charge characteristics. In certain embodiments, an antibody is said to specifically bind an antigen when it preferentially recognizes its target antigen in a complex mixture of proteins and/or macromolecules.

When the antigen is a polypeptide such as SEZ6, epitopes may generally be formed from both contiguous amino acids and noncontiguous amino acids juxtaposed by tertiary folding of a protein (“conformational epitopes”). In such conformational epitopes the points of interaction occur across amino acid residues on the protein that are linearly separated from one another. Epitopes formed from contiguous amino acids (sometimes referred to as “linear” or “continuous” epitopes) are typically retained upon protein denaturing, whereas epitopes formed by tertiary folding are typically lost upon protein denaturing. An antibody epitope typically includes at least 3, and more usually, at least 5 or 8-10 amino acids in a unique spatial conformation. Methods of epitope determination or “epitope mapping” are well known in the art and may be used in conjunction with the instant disclosure to identify epitopes on SEZ6 bound by the disclosed antibody drug conjugates.

Compatible epitope mapping techniques include alanine scanning mutants, peptide blots (Reineke (2004) Methods Mol Biol 248:443-63), or peptide cleavage analysis. In addition, methods such as epitope excision, epitope extraction and chemical modification of antigens can be employed (Tomer (2000) Protein Science 9: 487-496). In other embodiments Modification-Assisted Profiling (MAP), also known as Antigen Structure-based Antibody Profiling (ASAP) provides a method that categorizes large numbers of monoclonal antibodies directed against the same antigen according to the similarities of the binding profile of each antibody to chemically or enzymatically modified antigen surfaces (U.S.P.N. 2004/0101920). This technology allows rapid filtering of genetically identical antibodies, such that characterization can be focused on genetically distinct antibodies. It will be appreciated that MAP may be used to sort the antibodies compatible with the invention into groups of antibodies binding different epitopes.

Once a desired epitope on an antigen is determined, it is possible to generate antibodies to that epitope, e.g., by immunizing with a peptide comprising the epitope using techniques described in the present invention. Alternatively, during the discovery process, the generation and characterization of antibodies may elucidate information about desirable epitopes located in specific domains or motifs. From this information, it is then possible to competitively screen antibodies for binding to the same epitope. An approach to achieve this is to conduct competition studies to find antibodies that compete for binding to the antigen. A high throughput process for binning antibodies based upon their cross-competition is described in WO 03/48731. Other methods of binning or domain level or epitope mapping comprising antibody competition or antigen fragment expression on yeast are well known in the art.

### VI Linker Components

Numerous linker compounds of the general formula [—W—(X1)ₐ-CM-(X2)_{b}—P—] can be used to conjugate targeting agents of the invention to the selected calicheamicin warhead. The linkers merely need to covalently bind with the reactive residue on the targeting agent (preferably a cysteine or lysine) and the selected calicheamicin or calicheamicin analog. Accordingly, any disclosed calicheamicin-linker construct that reacts with the selected residue of the targeting agent may be used to provide the relatively stable conjugates (site-specific or otherwise) of the instant invention and is compatible with the teachings herein.

In preferred embodiments compatible linkers will confer stability on the ADCs in the extracellular environment, prevent aggregation of the ADC molecules and keep the ADC freely soluble in aqueous media and in a monomeric state. Before transport or delivery into a cell, the ADC is preferably stable and remains intact, i.e. the targeting agent remains linked to the calicheamicin. While the linkers are stable outside the target cell they are specifically designed to be cleaved and/or degraded at some efficacious rate at the target or, more preferably, inside the cell. Accordingly an effective linker will: (i) maintain the specific binding properties of the targeting agent; (ii) facilitate intracellular delivery of the payload or calicheamicin warhead; (iii) remain stable and intact, i.e. not cleaved or degraded, until the warhead has been delivered or transported to its targeted site; and (iv) maintain a cytotoxic, cell-killing effect or a cytostatic effect of the selected calicheamicin (including, in some cases, any bystander effects). As shown in the appended Examples fabrication and stability of the ADC may be measured by standard analytical techniques such as HPLC/UPLC, mass spectroscopy, HPLC, and the separation/analysis techniques LC/MS and LC/MS/MS.

### A. Cleavable Moiety—(CM)

Linkers compatible with the present invention may broadly be classified as cleavable and comprise at least one cleavable moiety as defined herein. Cleavable linkers, which may include acid-labile linkers, protease cleavable linkers and disulfide linkers, are preferably internalized into the target cell and are cleaved in the endosomal-lysosomal pathway inside the cell. In such cases release and activation of the calicheamicin warhead may rely on endosome/lysosome acidic compartments that facilitate cleavage of acid-labile chemical linkages such as hydrazone or oxime. Lysosomal-specific protease cleavage site(s) may also be engineered into the linker to preferably release the disclosed calicheamicin warheads in proximity to their intracellular target. Alternatively, linkers containing a cleavable disulfide moiety (in addition to the one proximal to the calicheamicin warhead) provide an approach by which the calicheamicin is released intracellularly as the disulfide bonds are selectively cleaved in the reducing environment of the cell, but not in the oxygen-rich environment in the bloodstream.

Accordingly, certain preferred embodiments of the invention comprise a linker that is cleavable by a cleaving agent that is present in the intracellular environment (e.g., within a lysosome or endosome or caveolae). The linker can be, for example, a peptidyl linker that is cleaved by an intracellular peptidase or protease enzyme including, but not limited to, a lysosomal or endosomal protease. In some embodiments, the peptidyl linker is at least two amino acids long or at least three amino acids long. Cleaving agents can include cathepsins B and D and plasmin, each of which is known to hydrolyze dipeptide drug derivatives resulting in the release of active calicheamicin inside target cells. Exemplary peptidyl linkers that are cleavable by the thiol-dependent protease Cathepsin-B are peptides comprising Phe-Leu since cathepsin-B has been found to be highly expressed in cancerous tissue. Other examples of such linkers are described, for example, in U.S. Pat. No. 6,214,345. In a specific preferred embodiment, the peptidyl linker cleavable by an intracellular protease is a Val-Cit linker, a Val-Ala linker or a Phe-Lys linker such as is described in U.S. Pat. No. 6,214,345. One advantage of using intracellular proteolytic release of the therapeutic agent is that the agent is typically attenuated when conjugated and the serum stabilities of the conjugates are typically high.

Thus, in particularly preferred embodiments the cleavable moiety comprises a peptide bond that is cleaved, preferentially by a protease at the intended site of action, as opposed to by a protease in the serum. Typically, the peptide component of the cleavable moiety comprises from 1 to 20 amino acids, preferably from 1 to 6 amino acids, more preferably from 1 to 3 amino acids. The amino acid(s) can be natural and/or unnatural α-amino acids. Natural amino acids are those encoded by the genetic code, as well as amino acids derived therefrom, e.g., hydroxyproline, γ-carboxyglutamate, citrulline, and 0-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. An amino acid mimetic is a compound that has a structure different from the general chemical structure of an α-amino acid but functions in a manner similar to one. The term “unnatural amino acid” is intended to represent the “D” stereochemical form, the natural amino acids being of the “L” form.

In particularly preferred embodiments compatible peptidyl linkers will comprise:

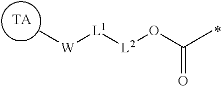

where the asterisk indicates the point of attachment to an optional spacer (or linker) X2 or the disulfide protective group, TA is a targeting agent such as disclosed herein, L¹ comprises a peptidyl cleavable moiety, W is a connecting group (optionally comprising a spacer (or linker) X1) connecting L¹ to a reactive residue on the targeting agent, L² is a covalent bond or together with —OC(═O)— forms a self-immolative linker. L¹-L²-OC(O)— corresponds to —(X1)ₐ-CM- in Formula 2 and -(L³)Z₁-M- in Formula (I/Ia).

As a peptidyl cleavable linker L¹ is preferably the trigger that initiates linker degradation resulting in cleavage of the disulfide bond and generation of the active biradical calicheamicin species at the target site.

It will be appreciated that the nature of L¹ and L² can vary widely. These groups are chosen on the basis of their cleavage characteristics, which may be dictated by the conditions at the site to which the conjugate is delivered. While those moieties that are cleaved by the action of enzymes are preferred in some instances it must be emphasized that moieties that are cleavable by changes in pH (e.g. acid or base labile), temperature or upon irradiation (e.g. photolabile) are compatible with the instant invention and may be employed as the CM. Moieties that are cleavable under reducing or oxidizing conditions are also compatible and may be used as cleavable moieties.

In particularly preferred embodiments L¹ may comprise a contiguous sequence of amino acids. The amino acid sequence, or cleavable peptide, may be the target substrate for enzymatic cleavage, thereby allowing release of the drug. The term “cleavable peptide” refers to peptides containing a cleavage recognition sequence of a protease. A cleavage recognition sequence for a protease is an amino acid sequence recognized by the protease during proteolytic cleavage. Many protease cleavage sites are known in the art, and these and other cleavage sites can be included a linker, a spacer, or a linker moiety. See, e.g., Matayoshi et al., Science 247:954 (1990); Dunn et al., Meth. Enzymol. 241:254 (1994); Seidah et al, Meth. Enzymol. 244: 175 (1994); Thornberry, Meth. Enzymol. 244:615 (1994); Weber et al., Meth. Enzymol. 244:595 (1994); Smith et al., Meth. Enzymol. 244:412 (1994); Bouvier et al., Meth. Enzymol. 248: 614(1995), Hardy et al, in AMYLOID PROTEIN PRECURSOR IN DEVELOPMENT, AGING, AND ALZHEIMER'S DISEASE, Ed. Masters et al., pp. 190-198 (1994).

Thus, in selected embodiments L¹ is cleavable by the action of an enzyme. In other selected embodiments the enzyme may comprise an esterase or a peptidase. In still other embodiments the peptide sequence is chosen based on its ability to be cleaved by a tumor-associated protease, e.g., a protease that is found on the surface of a cancerous cell or extracellularly in the vicinity of tumor cells. The examples of such proteases include thimet oligopeptidase (TOP), CDIO (neprilysin), a matrix metalloprotease (such as MMP2 or MMP9), a type II transmembrane serine protease (such as Hepsin, testisin, TMPRSS4 or matriptase/MT-SPl) and legumain. The ability of a peptide to be cleaved by tumor-associated protease can be tested using in vitro protease cleavage assays known in the art.

For conjugates that are designed to be internalized by a cell, the cleavable moiety preferably comprises an amino acid sequence selected for cleavage by an endosomal or lysosomal protease. Non-limiting examples of such proteases include cathepsins B, C, D, H, L and S, especially cathepsin B. Cathepsin B preferentially cleaves peptides at a sequence -AA²-AA¹- where AA¹ is a basic or strongly hydrogen bonding amino acid (such as lysine, arginine, or citrulline) and AA² is a hydrophobic amino acid (such as phenylalanine, valine, alanine, leucine, or isoleucine), for example Val-Cit (where Cit denotes citrulline) or Val-Lys. (Herein, amino acid sequences are written in the N-to-C direction, as in H₂N-AA²-AA¹-CO₂H, unless the context clearly indicates otherwise). For additional information regarding cathepsin-cleavable groups, see Dubowchik et al., Biorg. Med. Chem. Lett. 8, 3341-3346 (1998); Dubowchik et al., Bioorg. Med. Chem. Lett., 8 3347-3352 (1998); and Dubowchik et al., Bioconjugate Chem. 13, 855-869 (2002); the disclosures of which are incorporated by reference. Another enzyme that can be utilized for cleaving peptidyl linkers is legumain, a lysosomal cysteine protease that preferentially cleaves at Ala-Ala-Asn.

Accordingly, in preferred embodiments L¹ comprises a peptide. In certain selected embodiments can be a dipeptide that is represented as —NH-AA²-AA¹-CO—, where —NH— and —CO— represent the N- and C-terminals of the amino acid groups respectively. In other embodiments cleavable peptide can be a tripeptide, a quatrapeptide or a pentapeptide where each amino acid is independently an L or D isomer.

In certain embodiments, the peptide is selected from the group consisting of Val-Ala, Val-Cit, Val-Lys, Phe-Lys, Lys-Lys, Ala-Lys, Phe-Cit, Leu-Cit, Lle-Cit, Trp-Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO:3), β-Ala-Leu-Ala-Leu (SEQ ID NO:4), Gly-Phe-Leu-Gly (SEQ ID NO:5), Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, and D-Ala-D-Ala, Gly-Gly-Gly, Ala-Ala-Ala, D-Ala-Ala-Ala, Ala-D-Ala-Ala, Ala-Ala-D-Ala, Ala-Val-Cit, and Ala-Val-Ala. In another alternative, the peptide is selected from the group consisting of Gly-Gly-Gly, Ala-Ala-Ala, D-Ala-Ala-Ala, Ala-D-Ala-Ala, and Ala-Val-Ala. Alternatively, the peptide is Gly-Gly-Ala, Val-Ala, Glu-Ala, or Glu(OMe)-Ala. In a related embodiment, any of the peptide sequences herein above may be in either direction, as defined above.

Additionally, for those amino acids groups having carboxyl or amino side chain functionality, for example Glu and Lys respectively, CO and NH may represent that side chain functionality.

In one embodiment, the group -AA²-AA¹- in dipeptide, —NH-AA²-AA¹-CO—, is selected from: -Phe-Lys-, -Val-Ala-, -Val-Lys-, -Ala-Lys-, -Val-Cit-, -Phe-Cit-, -Leu-Cit-, -Ile-Cit-, -Phe-Arg- and -Trp-Cit-where Cit is citrulline.

Preferably, the group -AA²-AA¹- in dipeptide, —NH-AA²-AA¹-CO—, is selected from: -Phe-Lys-, -Val-Ala-, -Val-Lys-, -Ala-Lys-, and -Val-Cit-.

Most preferably, the group -AA²-AA¹- in dipeptide, —NH-AA²-AA¹-CO—, is -Val-Cit-, -Phe-Lys- or -Val-Ala-.

In certain preferred embodiments, L² is present and together with —C(═O)O— forms a self-immolative linker. In other embodiments, L² is a substrate for enzymatic activity, thereby further modulating release of the drug.

In one embodiment, where L¹ is cleavable by the action of an enzyme and L² is present, the enzyme cleaves the bond between L¹ and L².

In certain embodiments L¹ and L², where present, may be connected by a bond selected from: —C(═O)NH—, —C(═O)O—, —NHC(═O)—, —NH(Ar), —OC(═O)—, —OC(═O)O—, —NHC(═O)O—, —OC(═O)NH—, and —NHC(═O)NH—.

An amino group of L¹ that connects to L² may be the N-terminus of an amino acid or may be derived from an amino group of an amino acid side chain, for example a lysine amino acid side chain.

Particularly preferred embodiments of compatible calicheamicin-linker constructs comprising peptidyl cleavable moieties are set forth immediately below as Formulas 4-12. It will be appreciated that the constructs of Formulas 6-12 may be fabricated substantially as set forth in Examples 3 (Formula 4, Val-Cit) and 4 (Formula 5, Val-Ala) by merely substituting in the desired dipeptide moiety. Moreover, in view of the instant disclosure the skilled artisan could readily fabricate additional peptidyl linker calicheamicin constructs using similar synthetic schemes.

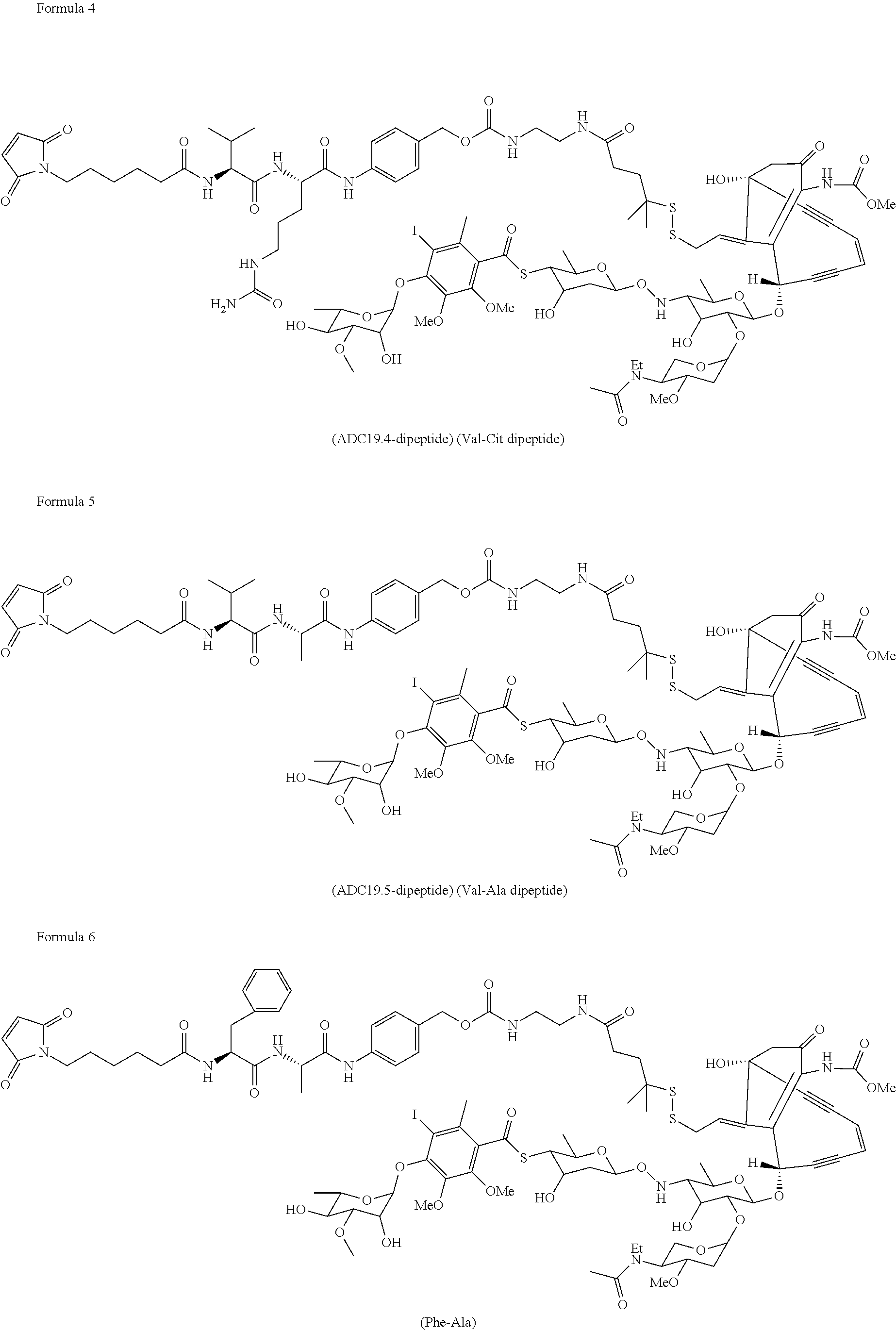

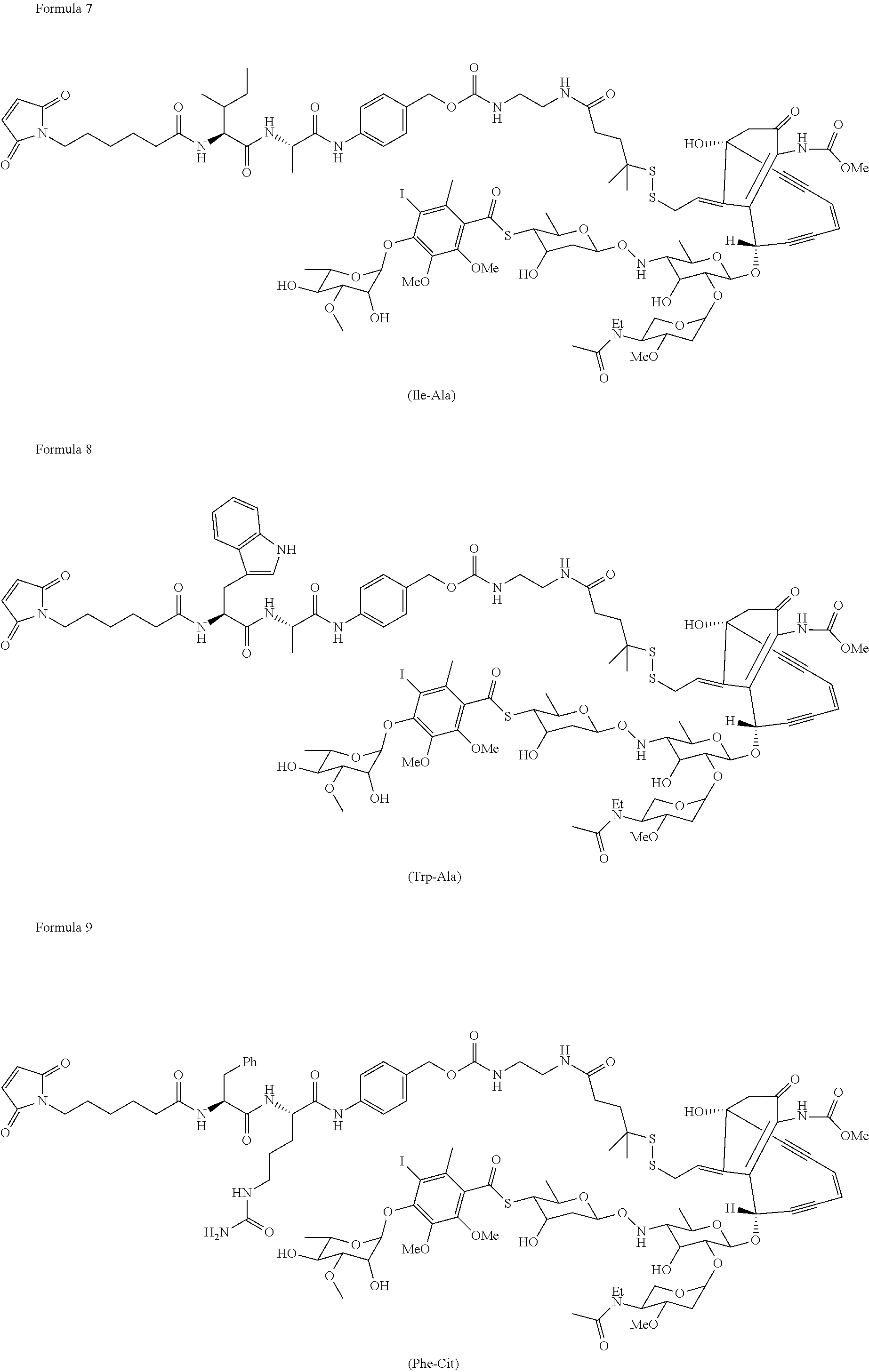

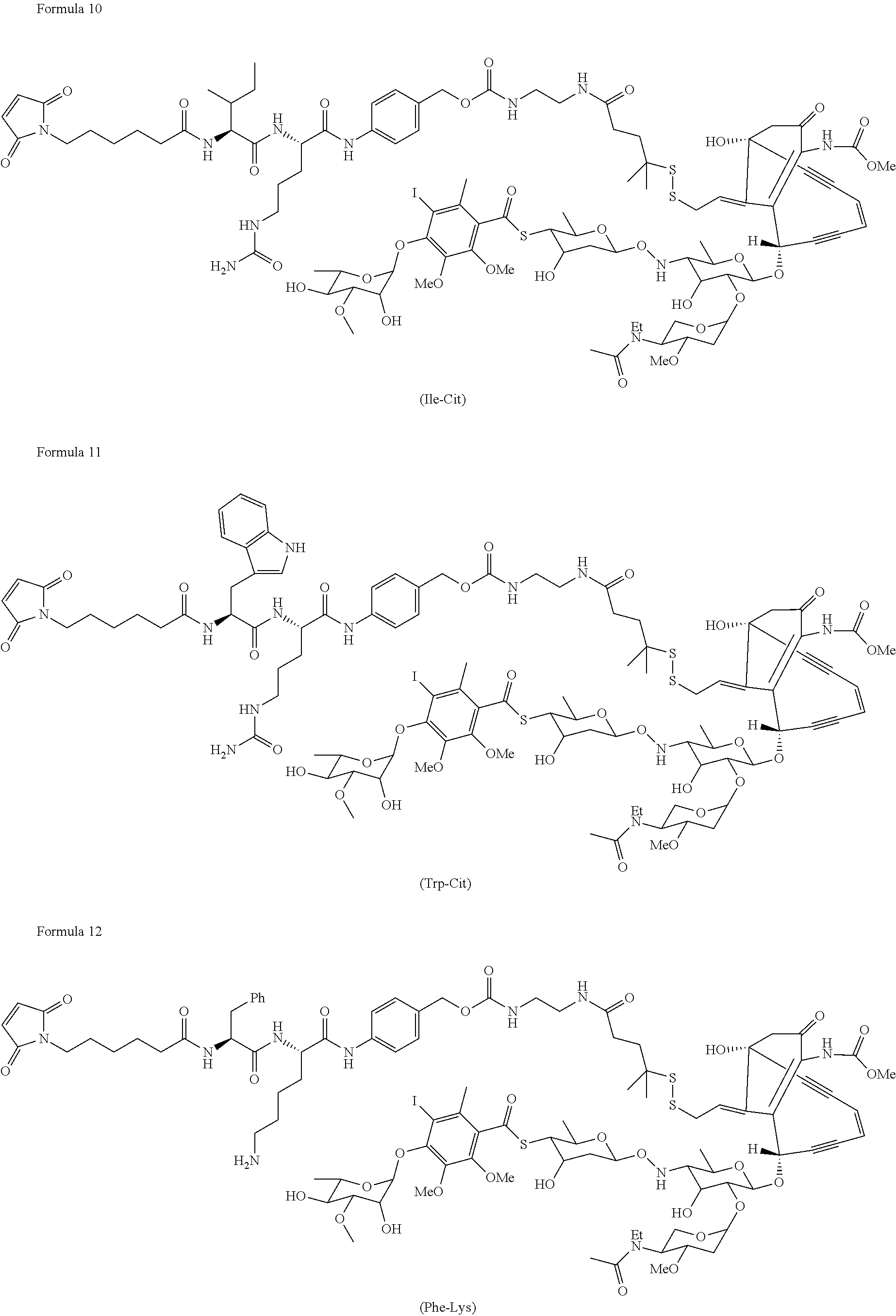

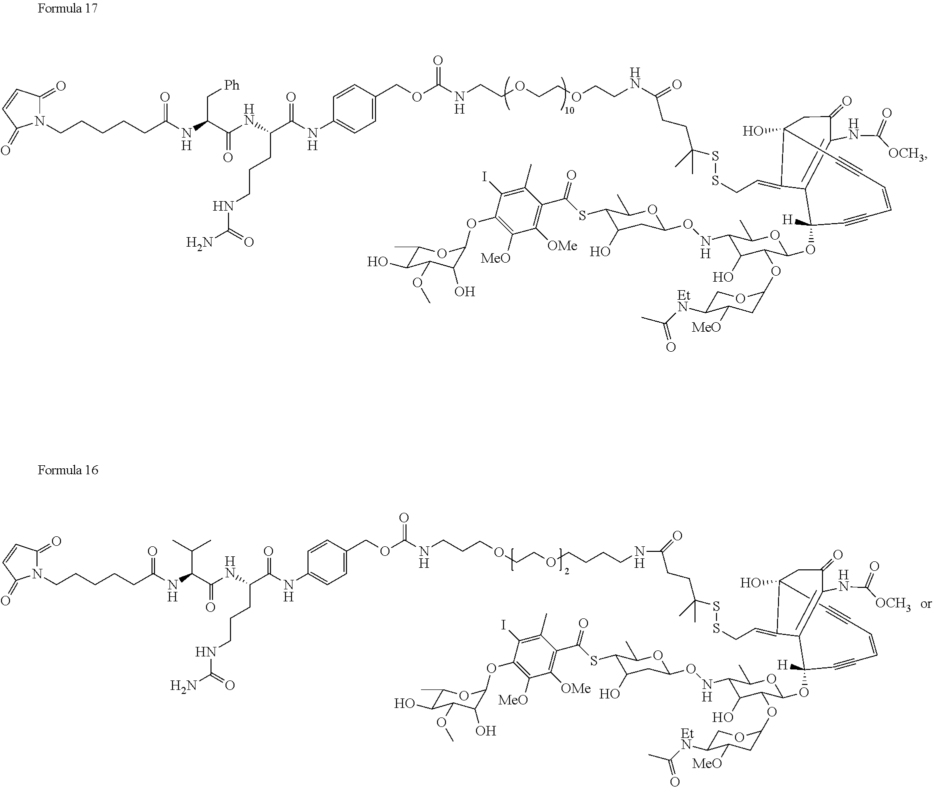

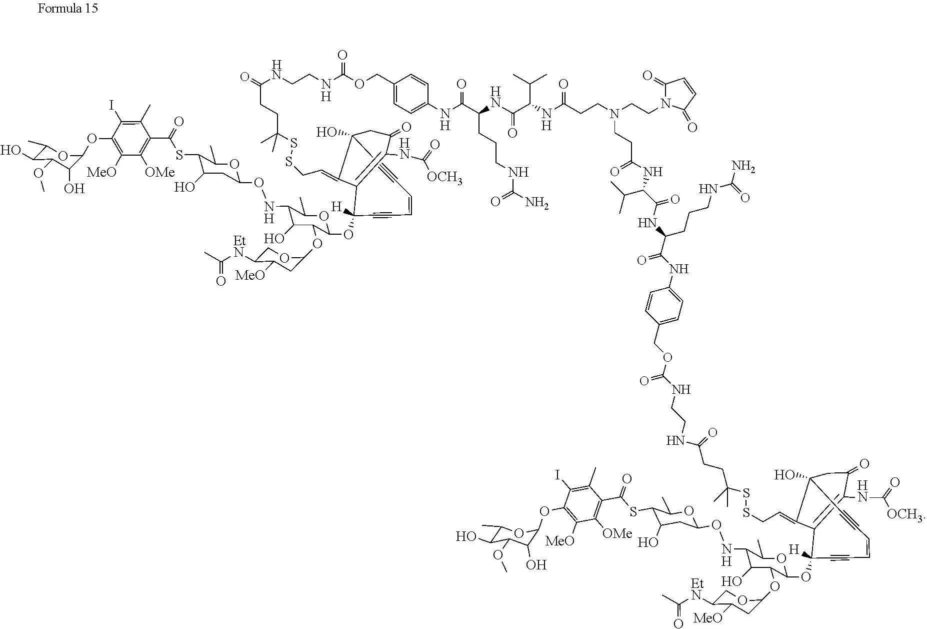

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴C, ¹⁵N), protected forms, and racemic mixtures thereof.

In one embodiment, —C(═O)O— and L² together form the group:

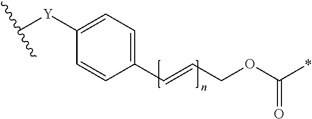

where the asterisk indicates the point of attachment to the optional spacer X2 or the disulfide protective group, the wavy line indicates the point of attachment to the cleavable moiety, Y is —N(H)—, —O—, —C(═O)N(H)— or —C(═O)O—, and n is 0 to 3. The phenylene ring is optionally substituted with one, two or three substituents as described herein. In one embodiment, the phenylene group is optionally substituted with halo, NO₂, R or OR (where R is as defined above).

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

In particularly preferred embodiments the linker may include a self-immolative linker and the dipeptide together form the group —NH-Val-Ala-CO—NH-PABC- (see Formula 5), which is illustrated below:

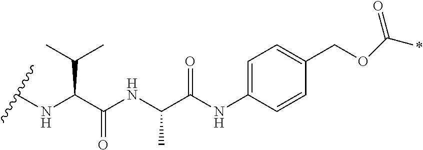 [SMILES: *N[C@H](C(=O)N[C@@H](C)C(=O)Nc1ccc(COC(*)=O)cc1)C(C)C]

where the asterisk indicates the point of attachment to an optional spacer or the disulfide protective group proximal to the calicheamicin warhead, and the wavy line indicates the point of attachment to the remaining portion of the linker (e.g., the optional spacer-connecting group segments) which may be conjugated to the antibody. Upon enzymatic cleavage of the dipeptide the self-immolative linker will allow for clean release of the protected compound (i.e., the calicheamicin disulfide analog) when a remote site is activated, proceeding along the lines shown below:

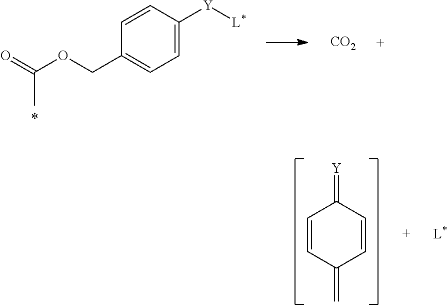

where L* is the form of the remaining portion of the linker comprising the now cleaved peptidyl unit and the targeting antigen. The clean release of the calicheamicin analog along with the disulfide protective group facilitates degradation of the remaining linker fragment and generation of the desired diradical species. In other particularly preferred embodiments the selected linker will comprise —NH-Val-Cit-CO—NH-PABC- (see Formula 4).

For additional disclosures regarding self-immolating moieties, see Carl et al., J. Med. Chem., 24 (3), 479-480 (1981); Carl et al., WO 81/01145 (1981); Dubowchik et al., Pharmacology &Therapeutics, 83, 67-123 (1999); Firestone et al., U.S. Pat. No. 6,214,345 B1 (2001); Toki et al., J. Org. Chem. 67, 1866-1872 (2002); Doronina et al., Nature Biotechnology 21 (7), 778-784 (2003) (erratum, p. 941); Boyd et al., U.S. Pat. No. 7,691,962 B2; Boyd et al., US 2008/0279868 A1; Sufi et al., WO 2008/083312 A2; Feng, U.S. Pat. No. 7,375,078 B2; and Senter et al., US 2003/0096743 A1; the disclosures of which are incorporated by reference.

In other embodiments, the cleavable linker is pH-sensitive (e.g., see Formula 13 and Formula 14). Typically, the pH-sensitive linker will be hydrolyzable under acidic conditions. For example, an acid-labile linker that is hydrolyzable in the lysosome (e.g., a hydrazone, oxime, semicarbazone, thiosemicarbazone, cis-aconitic amide, orthoester, acetal, ketal, or the like) can be used (See, e.g., U.S. Pat. Nos. 5,122,368; 5,824,805; 5,622,929). Such linkers are relatively stable under neutral pH conditions, such as those in the blood, but are unstable at below pH 5.5 or 5.0, the approximate pH of the lysosome. Thus, a cleavable moiety whose cleavage is acid catalyzed will cleave at a rate several orders of magnitude faster inside a lysosome than in the blood plasma rate. Examples of suitable acid-sensitive groups include cis-aconityl amides and hydrazones, as described in Shen et al., U.S. Pat. No. 4,631,190 (1986); Shen et al., U.S. Pat. No. 5,144,011 (1992); Shen et al., Biochem. Biophys. Res. Commun. 102, 1048-1054 (1981) and Yang et al., Proc. Natl Acad. Sci (USA), 85, 1189-1193 (1988); the disclosures of which are incorporated herein by reference.

In yet other embodiments, the linker is cleavable under reducing conditions (e.g., a disulfide linker). Disulfides can be cleaved by a thiol-disulfide exchange mechanism, at a rate dependent on the ambient thiol concentration. As the intracellular concentration of glutathione and other thiols is higher than their serum concentrations, the cleavage rate of a disulfide will be higher intracellularly. Further, the rate of thiol-disulfide exchange can be modulated by adjustment of the steric and electronic characteristics of the disulfide (e.g., an alkyl-aryl disulfide versus an alkyl-alkyl disulfide; substitution on the aryl ring, etc.), enabling the design of disulfide linkages that have enhanced serum stability or a particular cleavage rate. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio) butyrate) and SMPT (N-succinimidyl-oxycarbonyl-alpha-methyl-alpha-(2-pyridyl-dithio)toluene). For additional disclosures relating to disulfide cleavable groups in conjugates, see, e.g., Thorpe et al., Cancer Res. 48, 6396-6403 (1988); Santi et al., U.S. Pat. No. 7,541,530 B2 (2009); Ng et al., U.S. Pat. No. 6,989,452 B2 (2006); Ng et al., WO 2002/096910 A1; Boyd et al., U.S. Pat. No. 7,691,962 B2; and Sufi et al., US 2010/0145036 A1; the disclosures of which are incorporated herein by reference.

### B. Optional Spacers—(X1 and X2)

As previously alluded to the disclosed cleavable moieties can be flanked by one or more optional spacers (X1 and X2) or may be directly associated with either the targeting agent or the disulfide protective group; that is, spacers X1 and X2 may be absent or independently present. For example, if the cleavable moiety comprises a disulfide, one of the two sulfurs can be a cysteine residue or its surrogate the targeting agent. In other embodiments the cleavable moiety may be a hydrazone bonded to an aldehyde on a carbohydrate side chain of an antibody. In other preferred embodiments the cleavable moiety (potentially along with an optional self-immolative group) may be bound to two spacers of selected configurations.

The term “spacer” as used herein includes a chemical moiety interposed between any two chemical groups. For example, in some embodiments one end of a spacer (e.g., X1) is linked directly to the targeting agent or, in other embodiments, a reactive functional group (i.e., a connecting group) that can form a covalent bond with a cell-binding agent. In still other embodiments, one end of a spacer (e.g., X2) is linked to the disulfide protective group or a reactive functional group that can form a covalent bond with a disulfide protective group. In some embodiments, one end of the spacer is linked to a branched scaffold. In some embodiments, the spacer is interposed between (1) a targeting agent, or a reactive functional group that can form a covalent bond with a targeting agent; and (2) a branched scaffold. In some embodiments, the spacer is interposed between (1) the disulfide protective group, or a reactive functional group that can form a covalent bond with the disulfide protective group; and (2) a branched scaffold. In certain preferred embodiments a spacer may be attached to a reactive functional group at one end to form a linker moiety that can further react with a targeting agent or the protective disulfide group.

The term “branched scaffold” as used herein includes a chemical moiety (i.e., a “branching unit”) linked to two or more spacers. A branched scaffold allows two or more calicheamicin moieties to be attached to the targeting agent (Formula 15). Exemplary branched scaffolds may be derived from an amino acid with a side chain comprising an amino group (such as Lys) or a carboxyl group (such as a Glu or an Asp), or a peptide comprising two or more such amino acids (e.g., Lys-Lys dimer etc.). In other embodiments the branching unit may be derived from or comprise a reactive moiety such as tertiary amine.

In certain embodiments, the spacer creates a desired distance between the two chemical groups to, for example, avoid steric hindrance or to promote molecular flexibility. In certain embodiments, the presence of the spacer does not hinder, inhibit, or otherwise negatively affect the function of the flanking chemical groups (e.g., the ability of the cell-binding agent to bind a target molecule on a cell, or the cytotoxicity of the cytotoxic drug). In certain embodiments, the spacer confers additional beneficial characteristics, such as enhanced potency, solubility, serum stability, and/or efficacy, to the immunoconjugate or linker compound comprising the spacer. In certain embodiments, the spacer may comprise one or more amino acid residues (e.g., 1, 2, 3, 4, 5, 6, 7, 8, 9, 10, or more residues), which may or may not be resistant to protease or peptidases (such as intracellular/lysosomal peptidase) cleavage. In certain embodiments, the spacer may comprise one or more repeats of polyethylene glycol (PEG) units —(CH₂—CH₂—O)—, such as 1-1000 PEG units, 1-500 PEG units, 1-24 PEG units, or 2-8 PEG units (2, 4, 6, or 8 PEG units). In other embodiments preferred spacers will comprise a straight or branched, substituted or unsubstituted alkyl or aryl moieties. In still other embodiments either optional spacer X1 or X2 may comprise a self-immolating moiety.

### C. Disulfide Protective Group—(P)

As indicated previously the calicheamicin disulfide group is preferably protected by a short chain substituted or unsubstituted bifunctional aliphatic or aryl group (“disulfide protective group”) that provides stability (e.g., plasma stability) until the ADC reaches the target cell. In this respect the disulfide protective group covalently links the calicheamicin disulfide group with the optional spacer X2 or, in the event no spacer is present, directly with the cleavable moiety or optional self immolative group. In doing so the disulfide protective group provides a degree of steric hindrance for the disulfide bond thereby reducing its susceptibility to cleavage via thiol-disulfide exchange reactions. In view of the instant disclosure those of skill in the art could readily select compatible disulfide protective groups that provide the desired stability and optimize the therapeutic index of the calicheamicin ADC (See Kellogg et al., Bioconj. Chem, 2011, 22, 717-727). Additional methods of providing stabilized disulfide bonds may be found in USPN 20010036926 which is incorporated herein by reference.

In particularly preferred embodiments the disulfide protective group will comprise a cyclic or acyclic straight or branched chain C₁-C₁₂ saturated or unsaturated aliphatic moiety. In certain preferred embodiments the aliphatic moiety may be substituted. In other preferred embodiments the aliphatic moiety may be unsubstituted. Still other disulfide protective group embodiments comprise an aliphatic moiety having one or two methyl groups bound to the carbon proximal to the disulfide moiety. In yet other embodiments the aliphatic moiety will comprise a single methyl group bound to the carbon proximal to the disulfide moiety. Other preferred embodiments will comprise aliphatic moieties having one or more methyl groups one, two or three carbons away from the proximal carbon. The stability imparted by each such construct may be readily measured using art-recognized techniques. In each instance the selected disulfide protective group will act to increase the stability of the disulfide bond and prolong the half-life of the calicheamicin ADC in vivo.

### D. Connecting Group—(W)

Connecting groups are used to associate the disclosed calicheamicin constructs to targeting agents to provide the antibody drug conjugates of the instant invention. In preferred embodiments such connecting agents may comprise moieties known to participate in chemoselective modification of selected natural amino acids on the surface of a protein targeting agent (cysteine, lysine, tyrosine, tryptophan); reactive functionalities known to participate in glucoconjugation; reactive moieties suitable for chemoselective reactions with unnatural amino acids; reactive groups suitable for bioconjugation through enzymatic reactions with specific peptide tags (for general descriptions of these methods refer to Bioconj. Chemistry 2015, 26, 176-192). As discussed in detail herein, thiol based connecting groups suitable for the generation of site-specific antibody drug conjugates are particularly preferred.

Numerous compatible linkers can advantageously bind to reduced cysteines and lysines, which are nucleophilic. Conjugation reactions involving reduced cysteines and lysines include, but are not limited to, thiol-maleimide, thiol-dibromomaleimide, thiol-halogeno (acyl halide), thiol-ene, thiol-yne, thiol-vinylsulfone, thiol-bisulfone, thiol-thiosulfonate, thiol-pyridyl disulfide and thiol-parafluoro reactions. As further discussed herein, thiol-maleimide bioconjugation is one of the most widely used approaches due to its fast reaction rates and mild conjugation conditions. One issue with this approach is the possibility of the retro-Michael reaction and loss or transfer of the maleimido-linked payload from the antibody to other proteins in the plasma, such as, for example, human serum albumin. However, in preferred embodiments the use of selective reduction and site-specific antibodies as set forth herein in Examples 8 and 9 may be used to stabilize the conjugate and reduce this undesired transfer. Thiol-acyl halide reactions provide bioconjugates that cannot undergo retro-Michael reaction and therefore are more stable. Unfortunately, the thiol-halide reactions in general have slower reaction rates compared to maleimide-based conjugations and are thus not as efficient in providing undesired drug to antibody ratios. Thiol-pyridyl disulfide reaction is another popular bioconjugation route. The pyridyl disulfide undergoes fast exchange with free thiol resulting in the mixed disulfide and release of pyridine-2-thione. Mixed disulfides can be cleaved in the reductive cell environment releasing the payload. Other approaches gaining more attention in bioconjugation are thiol-vinylsulfone and thiol-bisulfone reactions, each of which are compatible with the teachings herein and expressly included within the scope of the invention. Those skilled in the art will appreciate that each of the aforementioned conjugation techniques and reagents are compatible with the instant invention and may be employed to provide the disclosed antibody drug conjugates.

Notwithstanding the aforementioned procedures the calicheamicin-linkers of the instant invention will preferably be linked to reactive thiol nucleophiles on cysteines, including those provided by free cysteines. To this end the cysteines of the targeting agents may be made reactive for conjugation with linker reagents by treatment with various reducing agent such as DTT or TCEP or mild reducing agents as set forth herein.

In this regard preferable connecting groups contain an electrophilic functional group for reaction with a nucleophilic functional group on the protein target agent. Nucleophilic groups on proteins include, but are not limited to: (i)N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) maleimide groups (ii) activated disulfides, (iii) active esters such as NHS (N-hydroxysuccinimide) esters, HOBt (N-hydroxybenzotriazole) esters, haloformates, and acid halides; (iv) alkyl and benzyl halides such as haloacetamides; and (v) aldehydes, ketones, and carboxyl groups.

Preferred connecting groups comprise the following:

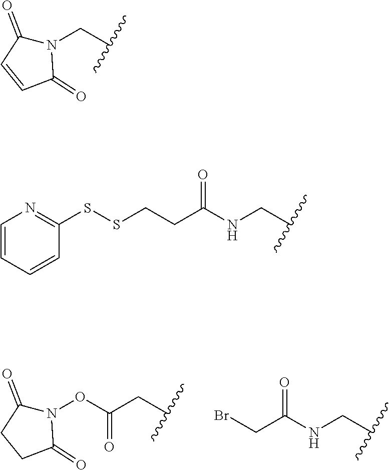 [SMILES: *CN1C(=O)C=CC1=O ; *CNC(=O)CCSSc1ccccn1 ; *CC(=O)ON1C(=O)CCC1=O ; *CNC(=O)CBr]

In selected embodiments the connection between a targeting agent and the calicheamicin-linker moiety is through a thiol residue of a cysteine (e.g., a free cysteine) on the targeting agent and a terminal maleimide group (i.e., a connecting group) present on the linker. In such embodiments, the connection between the protein targeting agent and the calicheamicin-linker is:

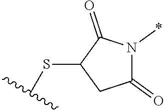 [SMILES: *SC1CC(=O)N(*)C1=O]

where the asterisk indicates the point of attachment to the remaining portion of calicheamicin-linker and the wavy line indicates the point of attachment to the remaining portion of the targeting agent. In selected embodiments the sulfur atom may preferably be derived from a site-specific free cysteine. With regard to other compatible linkers the connecting group comprises a terminal iodoacetamide that may be reacted with activated residues to provide the desired conjugate. In any event one skilled in the art could readily conjugate each of the disclosed calicheamicin-linker constructs with a compatible targeting agent (e.g., a site-specific antibody) in view of the instant disclosure.

In addition to activated thiol groups lysine conjugations may be effected through a variety of activated esters including but not limited to N-hydroxy succinaimide (NHS) ester, pentafluorophenol ester, tetrafluoropheno ester, para-nitrophenol ester, hydroxyl-benzotriazol (HOBt) ester and others. In certain cases of lysine with perturbed pKa, site specific lysine conjugates, may be generated by reaction with azatedinone moieties and beta-diketones.

In other embodiments tyrosine and tryptophan antibody constituents may be conjugated using diazonium salts, oxadiazole 3,5-dione derivatives, cyclic imines and other functionalities.

Other embodiments comprise conjugating the disclosed calicheamicin constructs to N-glycans present on certain targeting agents (e.g., antibodies). One commonly employed method comprises oxidation of the glycans with vicinal diols by treatment with periodate to generate aldehydes. The connecting group on the linker is then selected from aldehyde reactive functionalities, such as hydrazines, aminooxy compounds or amines suitable for reductive amination. In other preferred embodiments the connecting group on the linker is selected from a variety of strained cyclooctynes. Other compatible approaches, involve the methabolic expression of thiol-functionalized glycans on the surface of the targeting agent. Conjugation of the thiol will then be possible through the cysteine active connecting groups set forth above.

In still other compatible embodiments the incorporation of unnatural amino acids in the targeting agent allows for efficient conjugation of biorthogonal chemical functionalities to a preselected site. Connecting groups are then selected from complementary biorthogonal reactive functional groups. For example, an incorporated p-acetylphenylalanine residue may be conjugated using ketone-reactive connecting groups such as hydrazines, aminooxy compounds and amines suitable for reductive amination. Alternatively azide-functionalized unnatural amino acids may be incorporated and conjugated using no-copper click chemistry reagents such as strained cyclooctynes.

Yet other compatible embodiments comprise enzymatically mediated conjugation of the calicheamicin constructs with the disclosed targeting agents. To this end biotin ligase, translutaminase and lipoic acid ligase may be used to ligate small molecules to proteins site specifically. For example, transglutaminase catalyzes amide bond formation between a glutamine side chain and small molecules containing primary amine connecting groups. One particularly preferred embodiment involves modification of the specific peptide tag (LLQGA) by transglutaminase Streptovertticillium mobaranese and subsequent conjugation. This peptide tag has been shown to be conjugated most efficiently when a single tag is incorporated in the heavy chain and in a light chain of the antibody. Such configurations can reproducibly provide drug antibody ratio levels on the order of 1.8-1.9 in reaction with MMAD-amine. As an alternative strategy, formylgllycine generating enzyme has been employed. The enzyme transforms a cysteine residue within the peptide tag CXPXR into formylglycine. Formylglycine, although compatible with oxime and hydrazine formation with appropriate connecting groups, is preferably conjugated through Pictet-Spengler ligation with aminooxy- or hydrazine-functionalized tryptamine connecting groups. Products of such reactions have been shown to be very stable under physiological conditions and can readily provide calicheamicin ADCs in accordance with the instant invention.

Examples of calicheamicin-linker constructs connected to a generic antibody are set forth immediately below as Formulas 4′-12′ and 14′-17′. The symbol [symbol] represents the point of attachment to Ab in Formula I.

In view of the instant disclosure the skilled artisan could readily fabricate additional peptidyl linker calicheamicin constructs using similar synthetic schemes

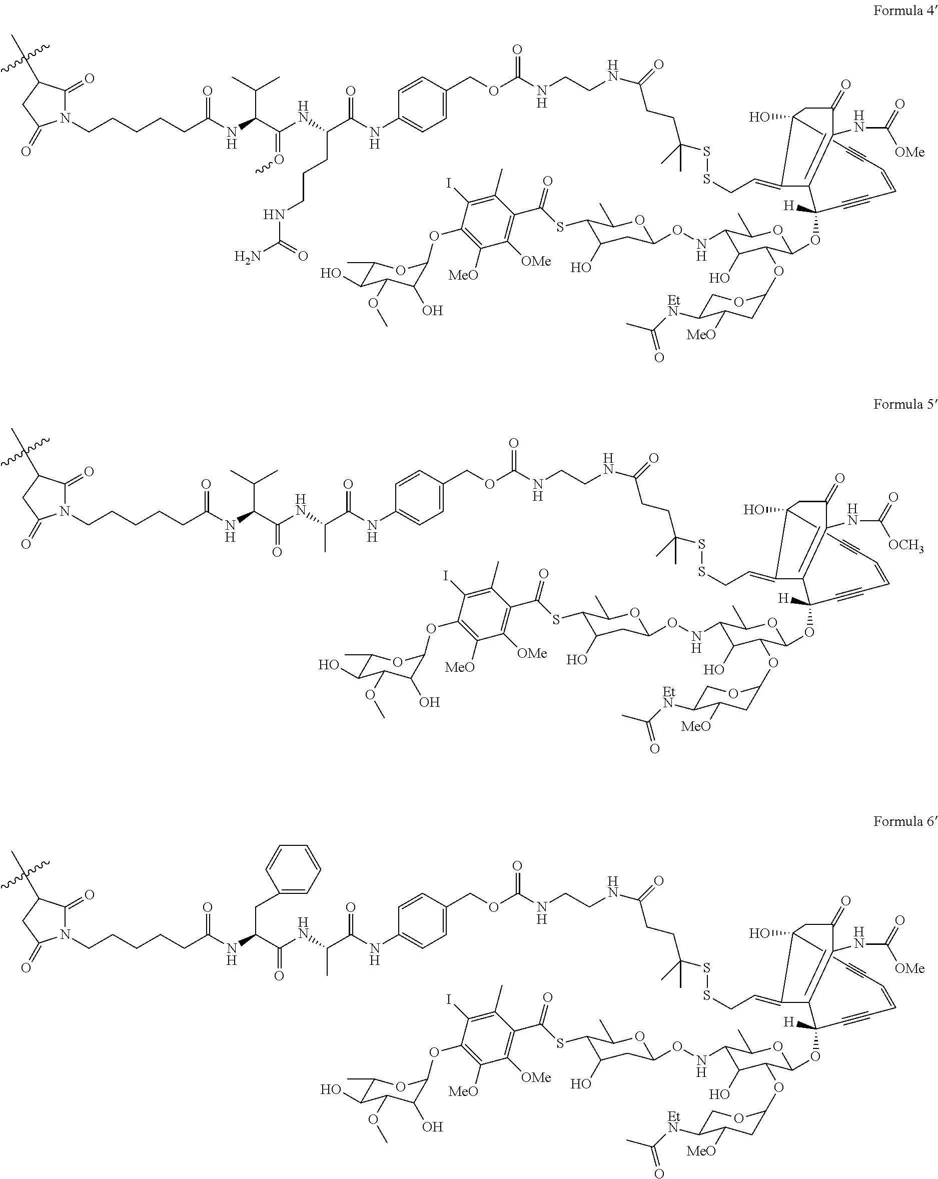

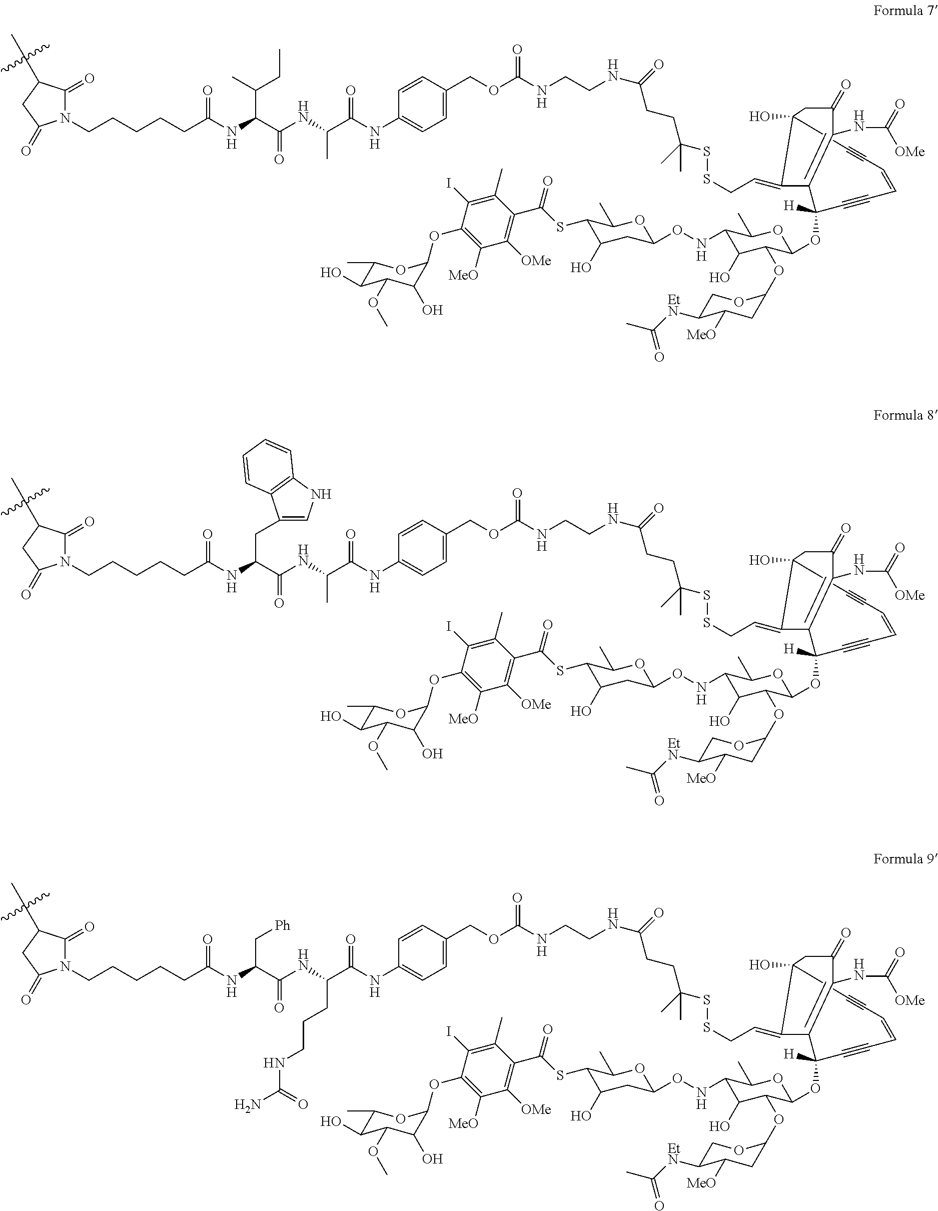

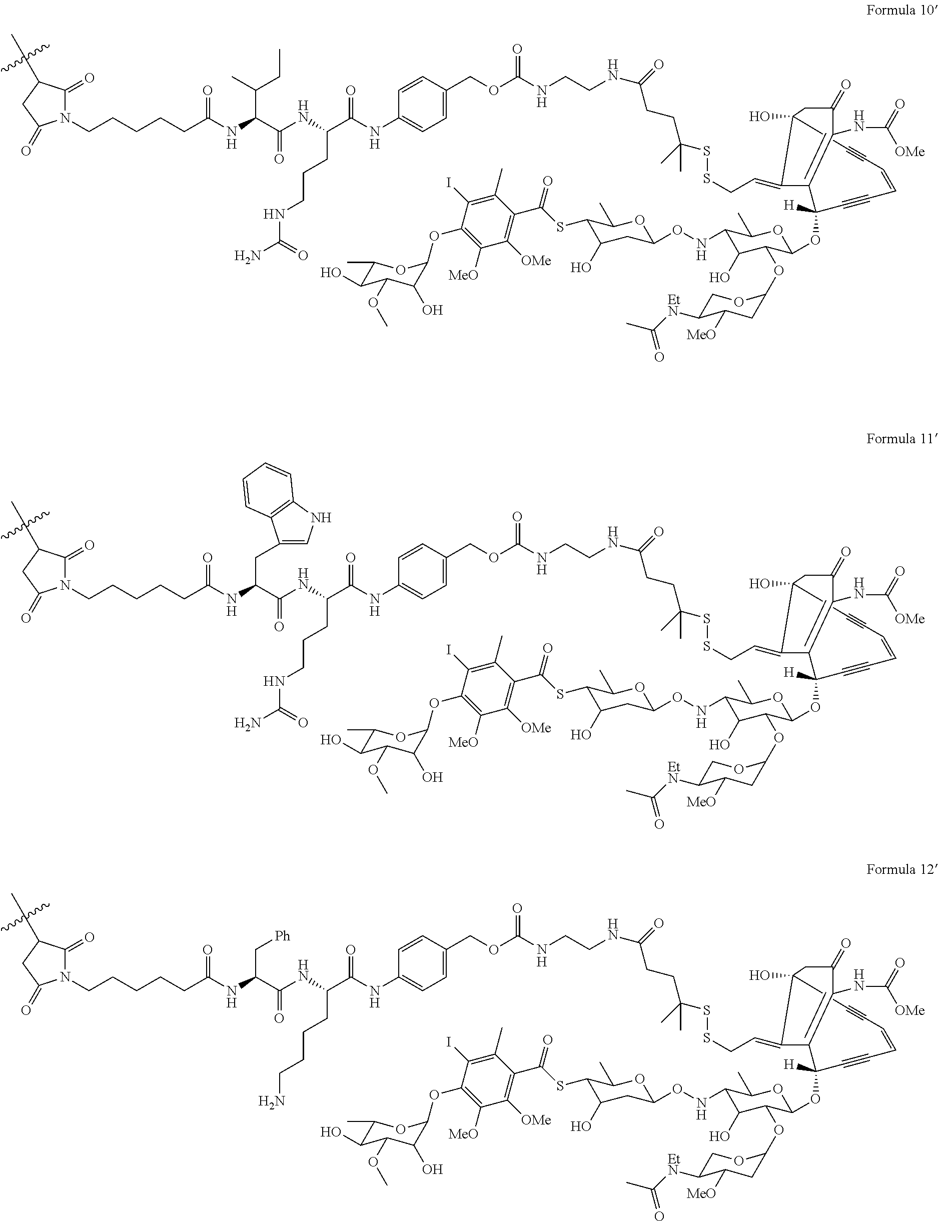

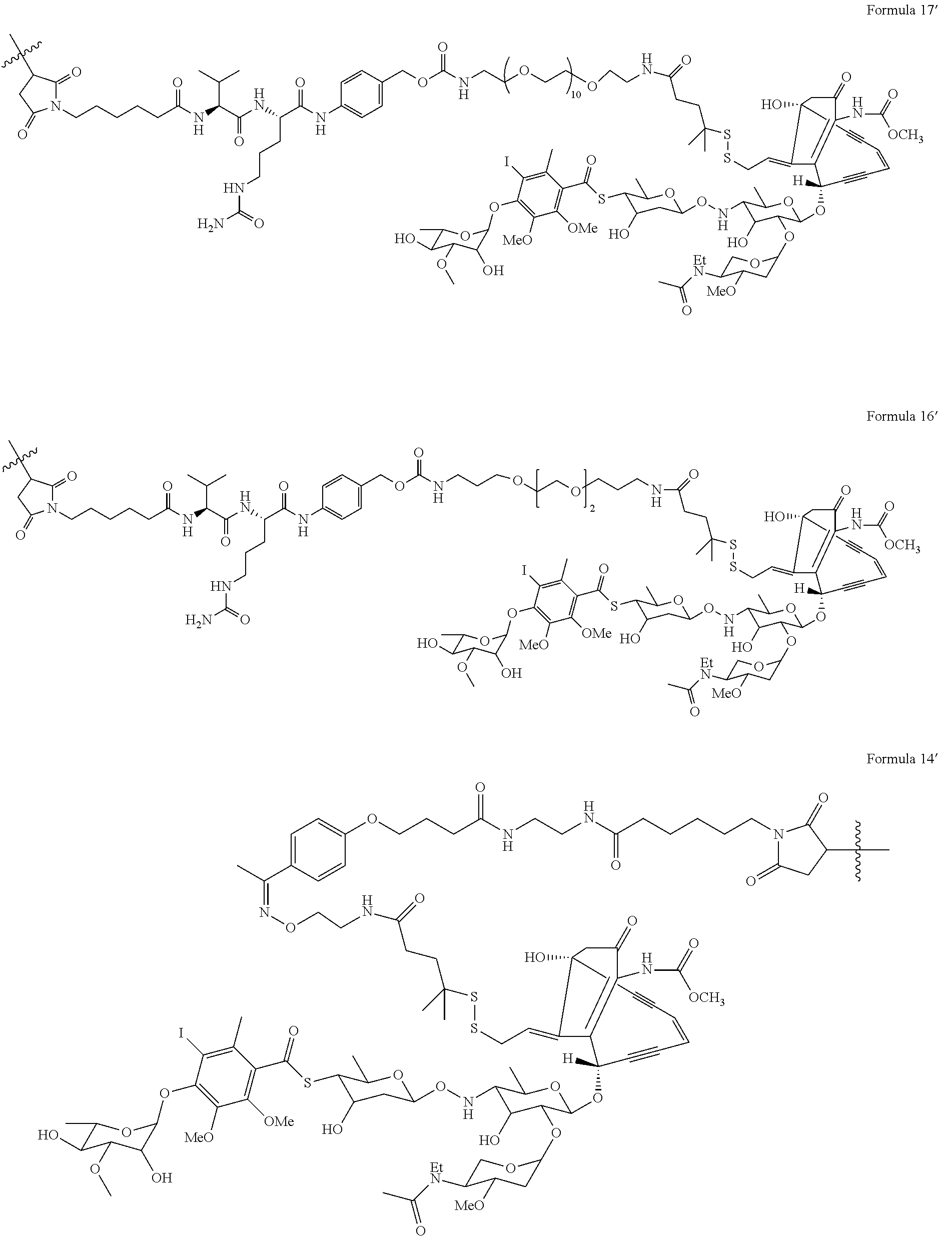

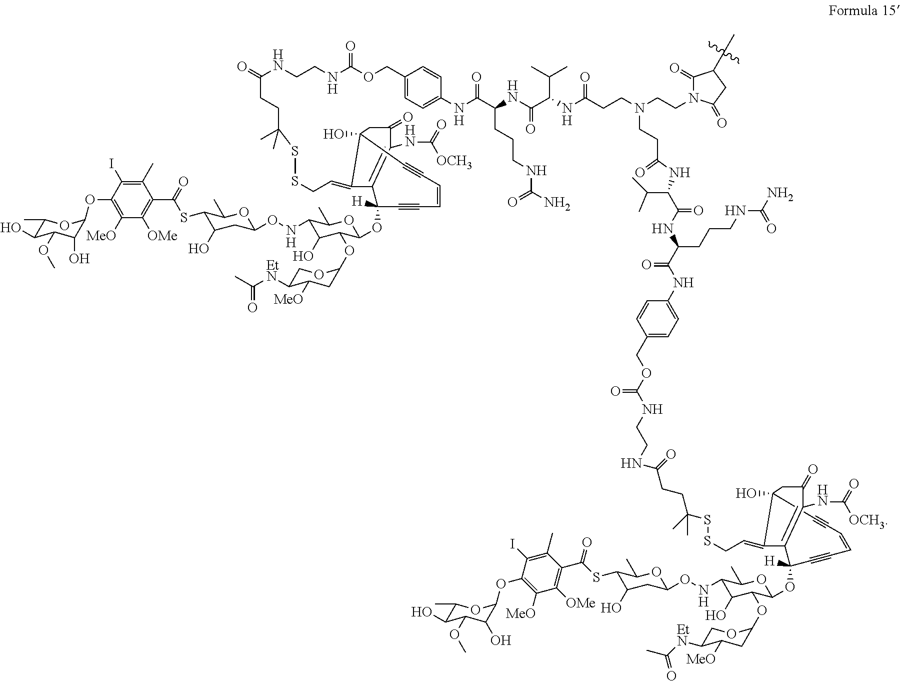

### VII Conjugation Preparation

### A. Conjugation Procedures

As alluded to above a number of well-known different reactions may be used to attach the disclosed calicheamicin-linker constructs to the selected targeting agent. For example, various reactions exploiting sulfhydryl groups of cysteines may be employed to conjugate the desired payload. Particularly preferred embodiments will comprise conjugation of antibodies comprising one or more free cysteines as discussed in detail below. In other embodiments ADCs of the instant invention may be generated through conjugation of calicheamicin to solvent-exposed amino groups of lysine residues present in the selected antibody. Still other embodiments comprise activation of the N-terminal threonine and serine residues which may then be used to attach the disclosed payloads to the antibody. The selected conjugation methodology will preferably be tailored to optimize the number of drugs attached to the antibody and provide a relatively high therapeutic index.

Various methods are known in the art for conjugating a therapeutic compound to a cysteine residue and will be apparent to the skilled artisan. Under basic conditions the cysteine residues will be deprotonated to generate a thiolate nucleophile which may be reacted with soft electrophiles, such as maleimides and iodoacetamides. Generally reagents for such conjugations may react directly with a cysteine thiol of a cysteine to form the conjugated protein or with a linker-drug to form a linker-drug intermediate. In the case of a linker, several routes, employing organic chemistry reactions, conditions, and reagents are known to those skilled in the art, including: (1) reaction of a cysteine group of the protein of the invention with a linker reagent, to form a protein-linker intermediate, via a covalent bond, followed by reaction with an activated compound; and (2) reaction of a nucleophilic group of a compound with a linker reagent, to form a drug-linker intermediate, via a covalent bond, followed by reaction with a cysteine group of a protein of the invention. In preferred embodiments the disclosed bifunctional linkers may comprise a thiol modification group for covalent linkage to the cysteine residue(s) and at least one attachment moiety (e.g., a second thiol modification moiety) for covalent or non-covalent linkage to the calicheamicin.

Prior to conjugation, antibodies may be made reactive for conjugation with linker reagents by treatment with a reducing agent such as dithiothreitol (DTT) or (tris(2-carboxyethyl)phosphine (TCEP). In other embodiments additional nucleophilic groups can be introduced into antibodies through the reaction of lysines with reagents, including but not limited to, 2-iminothiolane (Traut′s reagent), SATA, SATP or SAT(PEG)4, resulting in conversion of an amine into a thiol.

With regard to such conjugations cysteine thiol or lysine amino groups are nucleophilic and capable of reacting to form covalent bonds with electrophilic groups on linker reagents or compound-linker intermediates or drugs including: (i) active esters such as NHS esters, HOBt esters, haloformates, and acid halides; (ii) alkyl and benzyl halides, such as haloacetamides; (iii) aldehydes, ketones, carboxyl, and maleimide groups; and (iv) disulfides, including pyridyl disulfides, via sulfide exchange. Nucleophilic groups on a compound or linker include, but are not limited to amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents.

Preferred labeling reagents include maleimide, haloacetyl, iodoacetamide succinimidyl ester, isothiocyanate, sulfonyl chloride, 2,6-dichlorotriazinyl, pentafluorophenyl ester, and phosphoramidite, although other functional groups can also be used. In certain embodiments methods include, for example, the use of maleimides, iodoacetimides or haloacetyl/alkyl halides, aziridne, acryloyl derivatives to react with the thiol of a cysteine to produce a thioether that is reactive with a compound. Disulphide exchange of a free thiol with an activated piridyldisulphide is also useful for producing a conjugate (e.g., use of 5-thio-2-nitrobenzoic (TNB) acid). Preferably, a maleimide is used.

As indicated above, lysine may also be used as a reactive residue to effect conjugation as set forth herein. The nucleophilic lysine residue is commonly targeted through amine-reactive succinimidylesters. To obtain an optimal number of deprotonated lysine residues, the pH of the aqueous solution must be below the pKa of the lysine ammonium group, which is around 10.5, so the typical pH of the reaction is about 8 and 9. The common reagent for the coupling reaction is NHS-ester which reacts with nucleophilic lysine through a lysine acylation mechanism. Other compatible reagents that undergo similar reactions comprise isocyanates and isothiocyanates which also may be used in conjunction with the teachings herein to provide ADCs. Once the lysines have been activated, many of the aforementioned linking groups may be used to covalently bind the warhead to the antibody.

Methods are also known in the art for conjugating a compound to a threonine or serine residue (preferably a N-terminal residue). For example methods have been described in which carbonyl precursors are derived from the 1,2-aminoalcohols of serine or threonine, which can be selectively and rapidly converted to aldehyde form by periodate oxidation. Reaction of the aldehyde with a 1,2-aminothiol of cysteine in a compound to be attached to a protein of the invention forms a stable thiazolidine product. This method is particularly useful for labeling proteins at N-terminal serine or threonine residues.

In particularly preferred embodiments reactive thiol groups may be introduced into the selected antibody (or fragment thereof) by introducing one, two, three, four, or more free cysteine residues (e.g., preparing antibodies comprising one or more free non-native cysteine amino acid residues). As set forth above such site-specific or engineered antibodies allow for conjugate preparations that exhibit enhanced stability and substantial homogeneity due, at least in part, to the provision of engineered free cysteine site(s) and/or the novel conjugation procedures set forth herein. Unlike conventional conjugation methodology that fully or partially reduces each of the intrachain or interchain antibody disulfide bonds to provide conjugation sites (and is fully compatible with the instant invention), the present invention additionally provides for the selective reduction of certain prepared free cysteine sites and direction of the calicheamicin-linker to the same. The conjugation specificity promoted by the engineered sites and the selective reduction allows for a high percentage of site directed conjugation at the desired positions. Significantly some of these conjugation sites, such as those present in the terminal region of the light chain constant region, are typically difficult to conjugate effectively as they tend to cross-react with other free cysteines. However, through molecular engineering and selective reduction of the resulting free cysteines, efficient conjugation rates may be obtained which considerably reduces unwanted high-DAR contaminants and non-specific toxicity. More generally the engineered constructs and disclosed novel conjugation methods comprising selective reduction provide ADC preparations having improved pharmacokinetics and/or pharmacodynamics and, potentially, an improved therapeutic index.

As discussed above site-specific constructs present free cysteine(s) which, when reduced, comprise thiol groups that are nucleophilic and capable of reacting to form covalent bonds with electrophilic groups on linker moieties such as those disclosed above. Preferred antibodies of the instant invention will have reducible unpaired interchain or intrachain cysteines, i.e. cysteines providing such nucleophilic groups. Thus, in certain embodiments the reaction of free sulfhydryl groups of the reduced unpaired cysteines and the terminal maleimido or haloacetamide groups of the disclosed drug-linkers will provide the desired conjugation. In such cases the free cysteines of the antibodies may be made reactive for conjugation with linker reagents by treatment with a reducing agent such as dithiothreitol (DTT) or (tris (2-carboxyethyl)phosphine (TCEP). Each free cysteine will thus present, theoretically, a reactive thiol nucleophile. While such reagents are compatible it will be appreciated that conjugation of the site-specific antibodies may be effected using various reactions, conditions and reagents known to those skilled in the art.

In addition it has been found that the free cysteines of engineered antibodies may be selectively reduced to provide enhanced site-directed conjugation and a reduction in unwanted, potentially toxic contaminants. More specifically “stabilizing agents” such as arginine have been found to modulate intra- and inter-molecular interactions in proteins and may be used, in conjunction with selected reducing agents (preferably relatively mild), to selectively reduce the free cysteines and to facilitate site-specific conjugation as set forth herein.

As used herein the terms “selective reduction” or “selectively reducing” may be used interchangeably and shall mean the reduction of free cysteine(s) without substantially disrupting native disulfide bonds present in the engineered antibody. In selected embodiments this may be affected by certain reducing agents. In other preferred embodiments selective reduction of an engineered construct will comprise the use of stabilization agents in combination with reducing agents (including mild reducing agents). It will be appreciated that the term “selective conjugation” shall mean the conjugation of an engineered antibody that has been selectively reduced with a calicheamicin as described herein. In this respect the use of such stabilizing agents in combination with selected reducing agents can markedly improve the efficiency of site-specific conjugation as determined by extent of conjugation on the heavy and light antibody chains and DAR distribution of the preparation.

While not wishing to be bound by any particular theory, such stabilizing agents may act to modulate the electrostatic microenvironment and/or modulate conformational changes at the desired conjugation site, thereby allowing relatively mild reducing agents (which do not materially reduce intact native disulfide bonds) to facilitate conjugation at the desired free cysteine site. Such agents (e.g., certain amino acids) are known to form salt bridges (via hydrogen bonding and electrostatic interactions) and may modulate protein-protein interactions in such a way as to impart a stabilizing effect that may cause favorable conformation changes and/or may reduce unfavorable protein-protein interactions. Moreover, such agents may act to inhibit the formation of undesired intramolecular (and intermolecular) cysteine-cysteine bonds after reduction thus facilitating the desired conjugation reaction wherein the engineered site-specific cysteine is bound to the drug (preferably via a linker). Since selective reduction conditions do not provide for the significant reduction of intact native disulfide bonds, the subsequent conjugation reaction is naturally driven to the relatively few reactive thiols on the free cysteines (e.g., preferably 2 free thiols per antibody). As previously alluded to this considerably reduces the levels of non-specific conjugation and corresponding impurities in conjugate preparations fabricated as set forth herein.

In selected embodiments stabilizing agents compatible with the present invention will generally comprise compounds with at least one moiety having a basic pKa. In certain embodiments the moiety will comprise a primary amine while in other preferred embodiments the amine moiety will comprise a secondary amine. In still other preferred embodiments the amine moiety will comprise a tertiary amine or a guanidinium group. In other selected embodiments the amine moiety will comprise an amino acid while in other compatible embodiments the amine moiety will comprise an amino acid side chain. In yet other embodiments the amine moiety will comprise a proteinogenic amino acid. In still other embodiments the amine moiety comprises a non-proteinogenic amino acid. In particularly preferred embodiments, compatible stabilizing agents may comprise arginine, lysine, proline and cysteine. In addition compatible stabilizing agents may include guanidine and nitrogen containing heterocycles with basic pKa.

In certain embodiments compatible stabilizing agents comprise compounds with at least one amine moiety having a pKa of greater than about 7.5, in other embodiments the subject amine moiety will have a pKa of greater than about 8.0, in yet other embodiments the amine moiety will have a pKa greater than about 8.5 and in still other embodiments the stabilizing agent will comprise an amine moiety having a pKa of greater than about 9.0. Other preferred embodiments will comprise stabilizing agents where the amine moiety will have a pKa of greater than about 9.5 while certain other embodiments will comprise stabilizing agents exhibiting at least one amine moiety having a pKa of greater than about 10.0. In still other preferred embodiments the stabilizing agent will comprise a compound having the amine moiety with a pKa of greater than about 10.5, in other embodiments the stabilizing agent will comprise a compound having a amine moiety with a pKa greater than about 11.0, while in still other embodiments the stabilizing agent will comprise a amine moiety with a pKa greater than about 11.5. In yet other embodiments the stabilizing agent will comprise a compound having an amine moiety with a pKa greater than about 12.0, while in still other embodiments the stabilizing agent will comprise an amine moiety with a pKa greater than about 12.5. Those of skill in the art will understand that relevant pKa's may readily be calculated or determined using standard techniques and used to determine the applicability of using a selected compound as a stabilizing agent.

The disclosed stabilizing agents are shown to be particularly effective at targeting conjugation to free site-specific cysteines when combined with certain reducing agents. For the purposes of the instant invention, compatible reducing agents may include any compound that produces a reduced free site-specific cysteine for conjugation without significantly disrupting the engineered antibody native disulfide bonds. Under such conditions, provided by the combination of selected stabilizing and reducing agents, the activated calicheamicin-linker is largely limited to binding to the desired free site-specific cysteine site. Relatively mild reducing agents or reducing agents used at relatively low concentrations to provide mild conditions are particularly preferred. As used herein the terms “mild reducing agent” or “mild reducing conditions” shall be held to mean any agent or state brought about by a reducing agent (optionally in the presence of stabilizing agents) that provides thiols at the free cysteine site(s) without substantially disrupting native disulfide bonds present in the engineered antibody. That is, mild reducing agents or conditions are able to effectively reduce free cysteine(s) (provide a thiol) without significantly disrupting the protein's native disulfide bonds. The desired reducing conditions may be provided by a number of sulfhydryl-based compounds that establish the appropriate environment for selective conjugation. In preferred embodiments mild reducing agents may comprise compounds having one or more free thiols while in particularly preferred embodiments mild reducing agents will comprise compounds having a single free thiol. Non-limiting examples of reducing agents compatible with the instant invention comprise glutathione, n-acetyl cysteine, cysteine, 2-aminoethane-1-thiol and 2-hydroxyethane-1-thiol.

It will be appreciated that selective reduction process set forth above is particularly effective at targeted conjugation to the free cysteine. In this respect the extent of conjugation to the desired target site (defined here as “conjugation efficiency”) in site-specific antibodies may be determined by various art-accepted techniques. The efficiency of the site-specific conjugation of a drug to an antibody may be determined by assessing the percentage of conjugation on the target conjugation site (in this invention the free cysteine on the c-terminus of the light chain) relative to all other conjugated sites. In certain embodiments, the method herein provides for efficiently conjugating a drug to an antibody comprising free cysteines. In some embodiments, the conjugation efficiency is at least 5%, at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 98% or more as measured by the percentage of target conjugation relative to all other conjugation sites.

It will further be appreciated that engineered antibodies capable of conjugation may contain free cysteine residues that comprise sulfhydryl groups that are blocked or capped as the antibody is produced or stored. Such caps include small molecules, proteins, peptides, ions and other materials that interact with the sulfhydryl group and prevent or inhibit conjugate formation. In some cases the unconjugated engineered antibody may comprise free cysteines that bind other free cysteines on the same or different antibodies. As discussed herein such cross-reactivity may lead to various contaminants during the fabrication procedure. In some embodiments, the engineered antibodies may require uncapping prior to a conjugation reaction. In specific embodiments, antibodies herein are uncapped and display a free sulfhydryl group capable of conjugation. In specific embodiments, antibodies herein are subjected to an uncapping reaction that does not disturb or rearrange the naturally occurring disulfide bonds. It will be appreciated that in most cases the uncapping reactions will occur during the normal reduction reactions (reduction or selective reduction).

### B. DAR Distribution and Purification

One of the advantages of conjugation with site specific antibodies of the present invention is the ability to generate relatively homogeneous ADC preparations comprising a narrow DAR distribution. In this regard the disclosed constructs and/or selective conjugation provides for homogeneity of the ADC species within a sample in terms of the stoichiometric ratio between the drug and the engineered antibody. As briefly discussed above the term “drug to antibody ratio” or “DAR” refers to the molar ratio of drug to antibody. In some embodiments a conjugate preparation may be substantially homogeneous with respect to its DAR distribution, meaning that within the preparation is a predominant species of site-specific ADC with a particular DAR (e.g., a DAR of 2 or 4) that is also uniform with respect to the site of loading (i.e., on the free cysteines). In certain embodiments of the invention it is possible to achieve the desired homogeneity through the use of site-specific antibodies and/or selective reduction and conjugation. In other preferred embodiments the desired homogeneity may be achieved through the use of site-specific constructs in combination with selective reduction. In yet other particularly preferred embodiments the preparations may be further purified using analytical or preparative chromatography techniques. In each of these embodiments the homogeneity of the ADC sample can be analyzed using various techniques known in the art including but not limited to mass spectrometry, HPLC (e.g. size exclusion HPLC, RP-HPLC, HIC-HPLC etc.) or capillary electrophoresis.

With regard to the purification of ADC preparations it will be appreciated that standard pharmaceutical preparative methods may be employed to obtain the desired purity. As discussed herein liquid chromatography methods such as reverse phase (RP) and hydrophobic interaction chromatography (HIC) may separate compounds in the mixture by drug loading value. In some cases, ion-exchange (IEC) or mixed-mode chromatography (MMC) may also be used to isolate species with a specific drug load.

The disclosed ADCs and preparations thereof may comprise calicheamicin and antibody moieties in various stoichiometric molar ratios depending on the configuration of the antibody and, at least in part, on the method used to effect conjugation. In certain embodiments the calicheamicin loading per ADC may comprise from 1-20 warheads (i.e., n is 1-20). Other selected embodiments may comprise ADCs with a drug loading of from 1 to 15 warheads. In still other embodiments the ADCs may comprise from 1-12 warheads or, more preferably, from 1-10 warheads. In certain preferred embodiments the ADCs will comprise from 1 to 8 warheads.

While theoretical drug loading may be relatively high, practical limitations such as free cysteine cross reactivity and warhead hydrophobicity tend to limit the generation of homogeneous preparations comprising such DAR due to aggregates and other contaminants. That is, higher drug loading, e.g. >10, may cause aggregation, insolubility, toxicity, or loss of cellular permeability of certain antibody-drug conjugates. In view of such concerns practical drug loading provided by the instant invention preferably ranges from 1 to 10 drugs per conjugate, i.e. where 1, 2, 3, 4, 5, 6, 7, 8, 9 or 10 drugs are covalently attached to each antibody (e.g., for IgG, other antibodies may have different loading capacity depending the number of disulfide bonds). Preferably the DAR of compositions of the instant invention will be approximately 2, 4 or 6 and in particularly preferred embodiments the DAR will comprise approximately 2 or 4.

Despite the relatively high level of homogeneity provided by the instant invention the disclosed compositions actually comprise a mixture of conjugates with a range of calicheamicin compounds, from 1 to 10 (in the case of an IgG1). As such, the disclosed ADC compositions include mixtures of conjugates where most of the constituent antibodies are covalently linked to one or more calicheamicin moieties and (despite the conjugate specificity of selective reduction) where the calicheamicin may be attached to the antibody by various thiol groups. That is, following conjugation ADC compositions of the invention will comprise a mixture of conjugates with different calicheamicin loads (e.g., from 1 to 10 drugs per IgG1 antibody) at various concentrations (along with certain reaction contaminants primarily caused by free cysteine cross reactivity). Using selective reduction and post-fabrication purification the conjugate compositions may be driven to the point where they largely contain a single predominant desired ADC species (e.g., with a drug loading of 2 or 4) with relatively low levels of other ADC species (e.g., with a drug loading of 1, 3, 5, etc.). The average DAR value represents the weighted average of calicheamicin loading for the composition as a whole (i.e., all the ADC species taken together). Due to inherent uncertainty in the quantification methodology employed and the difficulty in completely removing the non-predominant ADC species in a commercial setting, acceptable DAR values or specifications are often presented as an average, a range or distribution (i.e., an average DAR of 2+/−0.5). Preferably compositions comprising a measured average DAR within the range (i.e., 1.5 to 2.5) would be used in a pharmaceutical setting.

Thus, in certain preferred embodiments the present invention will comprise compositions having an average DAR of 1, 2, 3, 4, 5, 6, 7 or 8 each +/−0.5. In other preferred embodiments the present invention will comprise an average DAR of 2, 4, 6 or 8+/−0.5. Finally, in selected preferred embodiments the present invention will comprise an average DAR of 2+/−0.5. It will be appreciated that the range or deviation may be less than 0.4 in certain preferred embodiments. Thus, in other embodiments the compositions will comprise an average DAR of 1, 2, 3, 4, 5, 6, 7 or 8 each +/−0.3, an average DAR of 2, 4, 6 or 8+/−0.3, even more preferably an average DAR of 2 or 4+/−0.3 or even an average DAR of 2+/−0.3. In other embodiments IgG1 conjugate compositions will preferably comprise a composition with an average DAR of 1, 2, 3, 4, 5, 6, 7 or 8 each +/−0.4 and relatively low levels (i.e., less than 30%) of non-predominant ADC species. In other preferred embodiments the ADC composition will comprise an average DAR of 2, 4, 6 or 8 each +/−0.4 with relatively low levels (<30%) of non-predominant ADC species. In particularly preferred embodiments the ADC composition will comprise an average DAR of 2+/−0.4 with relatively low levels (<30%) of non-predominant ADC species. In yet other embodiments the predominant ADC species (e.g., DAR of 2 or 4) will be present at a concentration of greater than 65%, at a concentration of greater than 70%, at a concentration of greater than 75%, at a concentration of greater that 80%, at a concentration of greater than 85%, at a concentration of greater than 90%, at a concentration of greater than 93%, at a concentration of greater than 95% or even at a concentration of greater than 97% when measured against other DAR species.

As detailed in the Examples below the distribution of calicheamicin per antibody in preparations of ADC from conjugation reactions may be characterized by conventional means such as UV-Vis spectrophotometry, reverse phase HPLC, HIC, mass spectroscopy, ELISA, and electrophoresis. The quantitative distribution of ADC in terms of drugs per antibody may also be determined. By ELISA, the averaged value of the drugs per antibody in a particular preparation of ADC may be determined. However, the distribution of drug per antibody values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues.

### VIII Pharmaceutical Preparations and Therapeutic Uses

### A. Formulations and Routes of Administration

The calicheamicin ADCs of the invention can be formulated in various ways using art recognized techniques. In some embodiments, the therapeutic ADC compositions of the invention can be administered neat or with a minimum of additional components while others may optionally be formulated to contain suitable pharmaceutically acceptable carriers. As used herein, “pharmaceutically acceptable carriers” comprise excipients, vehicles, adjuvants and diluents that are well known in the art and can be available from commercial sources for use in pharmaceutical preparation (see, e.g., Gennaro (2003) Remington: The Science and Practice of Pharmacy with Facts and Comparisons: Drugfacts Plus, 20th ed., Mack Publishing; Ansel et al. (2004) Pharmaceutical Dosage Forms and Drug Delivery Systems, 7^{th} ed., Lippencott Williams and Wilkins; Kibbe et al. (2000) Handbook of Pharmaceutical Excipients, 3^{rd} ed., Pharmaceutical Press.)

Suitable pharmaceutically acceptable carriers comprise substances that are relatively inert and can facilitate administration of the ADC or can aid processing of the active compounds into preparations that are pharmaceutically optimized for delivery to the site of action.

Such pharmaceutically acceptable carriers include agents that can alter the form, consistency, viscosity, pH, tonicity, stability, osmolarity, pharmacokinetics, protein aggregation or solubility of the formulation and include buffering agents, wetting agents, emulsifying agents, diluents, encapsulating agents and skin penetration enhancers. Certain non-limiting examples of carriers include saline, buffered saline, dextrose, arginine, sucrose, water, glycerol, ethanol, sorbitol, dextran, sodium carboxymethyl cellulose and combinations thereof. ADCs for systemic administration may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington: The Science and Practice of Pharmacy (2000) 20th Ed. Mack Publishing.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additionally contain other pharmaceutically acceptable carriers, such as anti-oxidants, buffers, preservatives, stabilizers, bacteriostats, suspending agents, thickening agents, and solutes that render the formulation isotonic with the blood (or other relevant bodily fluid) of the intended recipient. Examples of excipients include, for example, water, alcohols, polyols, glycerol, vegetable oils, and the like. Examples of suitable isotonic pharmaceutically acceptable carriers for use in such formulations include Sodium Chloride Injection, Ringer's Solution, or Lactated Ringer's Injection.

In particularly preferred embodiments formulated compositions of the present invention may be lyophilized to provide a powdered form of the antibody or ADC which may then be reconstituted prior to administration. Sterile powders for the preparation of injectable solutions may be generated by lyophilizing a solution comprising the disclosed antibodies or ADCs to yield a powder comprising the active ingredient along with any optional co-solubilized biocompatible ingredients. Generally, dispersions or solutions are prepared by incorporating the active compound into a sterile vehicle that contains a basic dispersion medium or solvent (e.g., a diluent) and, optionally, other biocompatible ingredients. A compatible diluent is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH1 buffered solution (e.g. phosphate-buffered saline), sterile saline solution, Ringer's solution or dextrose solution. In an alternative embodiment, diluents can include aqueous solutions of salts and/or buffers.

In certain preferred embodiments the antibodies or ADCs will be lyophilized in combination with a pharmaceutically acceptable sugar. A “pharmaceutically acceptable sugar” is a molecule which, when combined with a protein of interest, significantly prevents or reduces chemical and/or physical instability of the protein upon storage. When the formulation is intended to be lyophilized and then reconstituted. As used herein pharmaceutically acceptable sugars may also be referred to as a “lyoprotectant”. Exemplary sugars and their corresponding sugar alcohols include: an amino acid such as monosodium glutamate or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; PLURONICS®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred pharmaceutically-acceptable sugars are the non-reducing sugars trehalose or sucrose. Pharmaceutically acceptable sugars are added to the formulation in a “protecting amount” (e.g. pre-lyophilization) which means that the protein essentially retains its physical and chemical stability and integrity during storage (e.g., after reconstitution and storage).

Those skilled in the art will appreciate that compatible lyprotecatants may be added to the liquid or lyophilized formulation at concentrations ranging from about 1 mM to about 1000 mM, from about 25 mM to about 750 mM, from about 50 mM to about 500 mM, from about 100 mM to about 300 mM, from about 125 mM to about 250 mM, from about 150 mM to about 200 mM or from about 165 mM to about 185 mM. In certain embodiments the lyoprotectant(s) may be added to provide a concentration of about 10 mM, about 25 mM, about 50 mM, about 75 mM, about 100 mM, about 125 mM, about 130 mM, about 140 mM, about 150 mM, about 160 mM, about 165 mM, about 170 mM, about 175 mM, about 180 mM, about 185 mM about 190 mM, about 200 mM, about 225 mM, about 250 mM, about 300 mM, about 400 mM, about 500 mM, about 600 mM, about 700 mM, about 800 mM about 900 mM, or about 1000 mM. In certain preferred embodiments the lyoprotectant(s) may comprise pharmaceutically acceptable sugars. In particularly preferred aspects the pharmaceutically acceptable sugars will comprise trehalose or sucrose.

In other selected embodiments liquid and lyophilized formulations of the instant invention may comprise certain compounds, including amino acids or pharmaceutically acceptable salts thereof, to act as stabilizing or buffering agents. Such compounds may be added at concentrations ranging from about 1 mM to about 100 mM, from about 5 mM to about 75 mM, from about 5 mM to about 50 mM, from about 10 mM to about 30 mM or from about 15 mM to about 25 mM. In certain embodiments the buffering agent(s) may be added to provide a concentration of about 1 mM, about 5 mM, about 10 mM, about 15 mM, about 20 mM, about 25 mM, about 30 mM, about 35 mM, about 40 mM, about 50 mM, about 60 mM, about 70 mM, about 80 mM, about 90 mM or about 100 mM. In other selected embodiments the buffering agent may be added to provide a concentration of about 5 mM, about 10 mM, about 15 mM, about 20 mM, about 25 mM, about 30 mM, about 35 mM, about 40 mM, about 50 mM, about 60 mM, about 70 mM, about 80 mM, about 90 mM or about 100 mM. In certain preferred embodiments the buffering agent will comprise histidine hydrochloride.

In yet other selected embodiments liquid and lyophilized formulations of the instant invention may comprise nonionic surfactants such as polysorbate 20, polysorbate 40, polysorbate 60 or polysorbate 80 as stabilizing agents. Such compounds may be added at concentrations ranging from about 0.1 mg/ml to about 2.0 mg/ml, from about 0.1 mg/ml to about 1.0 mg/ml, from about 0.2 mg/ml to about 0.8 mg/ml, from about 0.2 mg/ml to about 0.6 mg/ml or from about 0.3 mg/ml to about 0.5 mg/ml. In certain embodiments the surfactant may be added to provide a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml or about 1.0 mg/ml. In other selected embodiments the surfactant may be added to provide a concentration of about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml or about 2.0 mg/ml. In certain preferred embodiments the surfactant will comprise polysorbate 20 or polysorbate 40.

Whether reconstituted from a lyophilized powder or a native solution, compatible formulations of the disclosed antibodies or ADCs for parenteral administration (e.g., intravenous injection) may comprise ADC or antibody concentrations of from about 10 μg/mL to about 100 mg/mL. In certain selected embodiments antibody or ADC concentrations will comprise 20 μg/mL, 40 μg/mL, 60 μg/mL, 80 μg/mL, 100 μg/mL, 200 μg/mL, 300, μg/mL, 400 μg/mL, 500 μg/mL, 600 μg/mL, 700 μg/mL, 800 μg/mL, 900 μg/mL or 1 mg/mL. In other embodiments ADC concentrations will comprise 2 mg/mL, 3 mg/mL, 4 mg/mL, 5 mg/mL, 6 mg/mL, 8 mg/mL, 10 mg/mL, 12 mg/mL, 14 mg/mL, 16 mg/mL, 18 mg/mL, 20 mg/mL, 25 mg/mL, 30 mg/mL, 35 mg/mL, 40 mg/mL, 45 mg/mL, 50 mg/mL, 60 mg/mL, 70 mg/mL, 80 mg/mL, 90 mg/mL or 100 mg/mL.

In any event it will be appreciated that the compounds and compositions of the invention may be administered in vivo, to a subject in need thereof, by various routes, including, but not limited to, oral, intravenous, intra-arterial, subcutaneous, parenteral, intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

### B. Dosages

The particular dosage regimen, i.e., dose, timing and repetition, will depend on the particular individual, as well as empirical considerations such as pharmacokinetics (e.g., half-life, clearance rate, etc.). Determination of the frequency of administration may be made by persons skilled in the art, such as an attending physician based on considerations of the condition and severity of the condition being treated, age and general state of health of the subject being treated and the like. Frequency of administration may be adjusted over the course of therapy based on assessment of the efficacy of the selected composition and the dosing regimen. Such assessment can be made on the basis of markers of the specific disease, disorder or condition. In embodiments where the individual has cancer, these include direct measurements of tumor size via palpation or visual observation; indirect measurement of tumor size by x-ray or other imaging techniques; an improvement as assessed by direct tumor biopsy and microscopic examination of a tumor sample; the measurement of an indirect tumor marker (e.g., PSA for prostate cancer) or an antigen identified according to the methods described herein; reduction in the number of proliferative or tumorigenic cells, maintenance of the reduction of such neoplastic cells; reduction of the proliferation of neoplastic cells; or delay in the development of metastasis.

The calicheamicin ADCs of the invention may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In selected embodiments the ADCs will be administered (preferably intravenously) at approximately 10, 20, 30, 40, 50, 60, 70, 80, 90 or 100 μg/kg body weight per dose. Other embodiments may comprise the administration of ADCs at about 200, 300, 400, 500, 600, 700, 800, 900, 1000, 1100, 1200, 1300, 1400, 1500, 1600, 1700, 1800, 1900 or 2000 μg/kg body weight per dose. In other preferred embodiments the disclosed conjugates will be administered at 2.5, 3, 3.5, 4, 4.5, 5, 5.5, 6, 6.5, 7, 7.5, 8, 9 or 10 mg/kg. In still other embodiments the conjugates may be administered at 12, 14, 16, 18 or 20 mg/kg body weight per dose. In yet other embodiments the conjugates may be administered at 25, 30, 35, 40, 45, 50, 55, 60, 65, 70, 75, 80, 90 or 100 mg/kg body weight per dose. With the teachings herein one of skill in the art could readily determine appropriate dosages for the ADCs based on the particular target, preclinical animal studies, clinical observations and standard medical and biochemical techniques and measurements.

Other dosing regimens may be predicated on Body Surface Area (BSA) calculations as disclosed in U.S. Pat. No. 7,744,877. As is well known, the BSA is calculated using the patient's height and weight and provides a measure of a subject's size as represented by the surface area of his or her body. In certain embodiments, the conjugates may be administered in dosages from 1 mg/m² to 800 mg/m², from 50 mg/m² to 500 mg/m² and at dosages of 100 mg/m², 150 mg/m², 200 mg/m², 250 mg/m², 300 mg/m², 350 mg/m², 400 mg/m² or 450 mg/m². It will also be appreciated that art recognized and empirical techniques may be used to determine appropriate dosage.

The disclosed ADCs may be administered on a specific schedule. Generally, an effective dose of the calicheamicin conjugate is administered to a subject one or more times. More particularly, an effective dose of the disclosed ADCs are administered once a week, once every two weeks, once every three weeks, once a month or less than once a month. In certain embodiments, the effective dose of the selected ADC may be administered multiple times, including for periods of at least a month, at least six months, at least a year, at least two years or a period of several years. In yet other embodiments, several days (2, 3, 4, 5, 6 or 7), several weeks (1, 2, 3, 4, 5, 6, 7 or 8) or several months (1, 2, 3, 4, 5, 6, 7 or 8) or even a year or several years may lapse between administration of the disclosed antibodies or ADCs.

In certain preferred embodiments the course of treatment involving conjugated antibodies will comprise multiple doses of the selected drug product over a period of weeks or months. More specifically, antibodies or ADCs of the instant invention may administered once every day, every two days, every four days, every week, every ten days, every two weeks, every three weeks, every month, every six weeks, every two months, every ten weeks or every three months. In this regard it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

### C. Combination Therapies

Combination therapies may be particularly useful in decreasing or inhibiting unwanted neoplastic cell proliferation, decreasing the occurrence of cancer, decreasing or preventing the recurrence of cancer, or decreasing or preventing the spread or metastasis of cancer. In such cases the antibodies or ADCs of the instant invention may function as sensitizing or chemosensitizing agents by removing CSCs that would otherwise prop up and perpetuate the tumor mass and thereby allow for more effective use of current standard of care debulking or anti-cancer agents. That is, the disclosed antibodies or ADCs may, in certain embodiments, provide an enhanced effect (e.g., additive or synergistic in nature) that potentiates the mode of action of another administered therapeutic agent. In the context of the instant invention “combination therapy” shall be interpreted broadly and merely refers to the administration of a calicheamicin antibody or ADC and one or more anti-cancer agents that include, but are not limited to, cytotoxic agents, cytostatic agents, anti-angiogenic agents, debulking agents, chemotherapeutic agents, radiotherapy and radiotherapeutic agents, targeted anti-cancer agents (including both monoclonal antibodies and small molecule entities), BRMs, therapeutic antibodies, cancer vaccines, cytokines, hormone therapies, radiation therapy and anti-metastatic agents and immunotherapeutic agents, including both specific and non-specific approaches.

There is no requirement for the combined results to be additive of the effects observed when each treatment (e.g., calicheamicin ADC and an anti-cancer agent) is conducted separately. Although at least additive effects are generally desirable, any increased anti-tumor effect above one of the single therapies is beneficial. Furthermore, the invention does not require the combined treatment to exhibit synergistic effects. However, those skilled in the art will appreciate that with certain selected combinations that comprise preferred embodiments, synergism may be observed.

As such, in certain aspects the combination therapy has therapeutic synergy or improves the measurable therapeutic effects in the treatment of cancer over (i) the ADC used alone, or (ii) the therapeutic moiety used alone, or (iii) the use of the therapeutic moiety in combination with another therapeutic moiety without the addition of the ADC. The term “therapeutic synergy”, as used herein, means the combination of the ADC and one or more therapeutic moiety(ies) having a therapeutic effect greater than the additive effect of the combination of the ADC and the one or more therapeutic moiety(ies).

Desired outcomes of the disclosed combinations are quantified by comparison to a control or baseline measurement. As used herein, relative terms such as “improve,” “increase,” or “reduce” indicate values relative to a control, such as a measurement in the same individual prior to initiation of treatment described herein, or a measurement in a control individual (or multiple control individuals) in the absence of the disclosed ADCs described herein but in the presence of other therapeutic moiety(ies) such as standard of care treatment. A representative control individual is an individual afflicted with the same form of cancer as the individual being treated, who is about the same age as the individual being treated (to ensure that the stages of the disease in the treated individual and the control individual are comparable).

Changes or improvements in response to therapy are generally statistically significant. As used herein, the term “significance” or “significant” relates to a statistical analysis of the probability that there is a non-random association between two or more entities. To determine whether or not a relationship is “significant” or has “significance,” a “p-value” can be calculated. P-values that fall below a user-defined cut-off point are regarded as significant. A p-value less than or equal to 0.1, less than 0.05, less than 0.01, less than 0.005, or less than 0.001 may be regarded as significant.

A synergistic therapeutic effect may be an effect of at least about two-fold greater than the therapeutic effect elicited by a single therapeutic moiety or calicheamicin ADC, or the sum of the therapeutic effects elicited by the ADC or the single therapeutic moiety(ies) of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single therapeutic moiety or ADC, or the sum of the therapeutic effects elicited by the ADC or the single therapeutic moiety(ies) of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

In practicing combination therapy, the calicheamicin ADC and therapeutic moiety(ies) may be administered to the subject simultaneously, either in a single composition, or as two or more distinct compositions using the same or different administration routes. Alternatively, treatment with the ADC may precede or follow the therapeutic moiety treatment by, e.g., intervals ranging from minutes to weeks. In one embodiment, both the therapeutic moiety and the ADC are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the ADC and the therapeutic moiety.

The combination therapy can be administered until the condition is treated, palliated or cured on various schedules such as once, twice or three times daily, once every two days, once every three days, once weekly, once every two weeks, once every month, once every two months, once every three months, once every six months, or may be administered continuously. The selected ADC and therapeutic moiety(ies) may be administered on alternate days or weeks; or a sequence of ADC treatments may be given, followed by one or more treatments with the additional therapeutic moiety. In one embodiment the ADC is administered in combination with one or more therapeutic moiety(ies) for short treatment cycles. In other embodiments the combination treatment is administered for long treatment cycles. The combination therapy can be administered via any route.

In some embodiments the calicheamicin ADCs may be used in combination with various first line cancer treatments. In one embodiment the combination therapy comprises the use of an ADC and a cytotoxic agent such as ifosfamide, mytomycin C, vindesine, vinblastine, etoposide, ironitecan, gemcitabine, taxanes, vinorelbine, methotrexate, and pemetrexed) and optionally one or more other therapeutic moiety(ies).

In another embodiment the combination therapy comprises the use of the ADC and a platinum-based drug (e.g. carboplatin or cisplatin) and optionally one or more other therapeutic moiety(ies) (e.g. vinorelbine; gemcitabine; a taxane such as, for example, docetaxel or paclitaxel; irinotican; or pemetrexed).

In selected embodiments the compounds and compositions of the present invention may be used in conjunction with checkpoint inhibitors such as PD-1 inhibitors or PD-L1 inhibitors. PD-1, together with its ligand PD-L1, are negative regulators of the antitumor T lymphocyte response. In one embodiment the combination therapy may comprise the administration of calicheamicin ADCs together with an anti-PD-1 antibody (e.g. pembrolizumab, nivolumab, pidilizumab) and optionally one or more other therapeutic moiety(ies). In another embodiment the combination therapy may comprise the administration of calicheamicin ADCs together with an anti-PD-L1 antibody (e.g. avelumab, atezolizumab, durvalumab) and optionally one or more other therapeutic moiety(ies). In yet another embodiment, the combination therapy may comprise the administration of calicheamicin ADCs together with an anti PD-1 antibody or anti-PD-L1 administered to patients who continue progress following treatments with checkpoint inhibitors and/or targeted BRAF combination therapies (e.g. vemurafenib or dabrafinib).

In one embodiment, for example, in the treatment of BR-ERPR, BR-ER or BR-PR cancer, the combination therapy comprises the use of the ADC and one or more therapeutic moieties described as “hormone therapy”. “Hormone therapy” as used herein, refers to, e.g., tamoxifen; gonadotropin or luteinizing releasing hormone (GnRH or LHRH); everolimus and exemestane; toremifene; or aromatase inhibitors (e.g. anastrozole, letrozole, exemestane or fulvestrant).

In another embodiment, for example, in the treatment of BR-HER2, the combination therapy comprises the use of the ADC and trastuzumab or ado-trastuzumab emtansine and optionally one or more other therapeutic moiety(ies) (e.g. pertuzumab and/or docetaxel).

In some embodiments, for example, in the treatment of metastatic breast cancer, the combination therapy comprises the use of a disclosed ADC and a taxane (e.g. docetaxel or paclitaxel) and optionally an additional therapeutic moiety(ies), for example, an anthracycline (e.g. doxorubicin or epirubicin) and/or eribulin.

In another embodiment, for example, in the treatment of metastatic or recurrent breast cancer or BRCA-mutant breast cancer, the combination therapy comprises the use of a disclosed ADC and megestrol and optionally an additional therapeutic moiety(ies).

In further embodiments, for example, in the treatment of BR-TNBC, the combination therapy comprises the use of a calicheamicin ADC and a poly ADP ribose polymerase (PARP) inhibitor (e.g. BMN-673, olaparib, rucaparib and veliparib) and optionally an additional therapeutic moiety(ies).

In another embodiment, for example, in the treatment of breast cancer, the combination therapy comprises the use of an disclosed ADC and cyclophosphamide and optionally an additional therapeutic moiety(ies) (e.g. doxorubicin, a taxane, epirubicin, 5-FU and/or methotrexate.

In another embodiment combination therapy for the treatment of EGFR-positive NSCLC comprises the use of a disclosed ADC and afatinib and optionally one or more other therapeutic moiety(ies) (e.g. erlotinib and/or bevacizumab).

In another embodiment combination therapy for the treatment of EGFR-positive NSCLC comprises the use of an ADC and erlotinib and optionally one or more other therapeutic moiety(ies) (e.g. bevacizumab).

In another embodiment combination therapy for the treatment of ALK-positive NSCLC comprises the use of an ADC and ceritinib and optionally one or more other therapeutic moiety(ies).

In another embodiment combination therapy for the treatment of ALK-positive NSCLC comprises the use of an ADC and crizotinib and optionally one or more other therapeutic moiety(ies).

In another embodiment the combination therapy comprises the use of an ADC and bevacizumab and optionally one or more other therapeutic moiety(ies) (e.g. a taxane such as, for example, docetaxel or paclitaxel; and/or a platinum analog).

In another embodiment the combination therapy comprises the use of an ADC and bevacizumab and optionally one or more other therapeutic moiety(ies) (e.g. gemcitabine and/or a platinum analog).

In a particular embodiment the combination therapy for the treatment of platinum-resistant tumors comprises the use of an ADC and doxorubicin and/or etoposide and/or gemcitabine and/or vinorelbine and/or ifosfamide and/or leucovorin-modulated 5-fluoroucil and/or bevacizumab and/or tamoxifen; and optionally one or more other therapeutic moiety(ies).

In another embodiment the combination therapy comprises the use of an ADC and a PARP inhibitor and optionally one or more other therapeutic moiety(ies).

In another embodiment the combination therapy comprises the use of an ADC and bevacizumab and optionally cyclophosphamide.

The combination therapy may comprise an ADC and a chemotherapeutic moiety that is effective on a tumor comprising a mutated or aberrantly expressed gene or protein (e.g. BRCA1).

T lymphocytes (e.g., cytotoxic lymphocytes (CTL)) play an important role in host defense against malignant tumors. CTL are activated by the presentation of tumor associated antigens on antigen presenting cells. Active specific immunotherapy is a method that can be used to augment the T lymphocyte response to cancer by vaccinating a patient with peptides derived from known cancer associated antigens. In one embodiment the combination therapy may comprise an ADC and a vaccine to a cancer associated antigen (e.g. melanocyte-lineage specific antigen tyrosinase, gp100, Melan-A/MART-1 or gp75.) In other embodiments the combination therapy may comprise administration of an ADC together with in vitro expansion, activation, and adoptive reintroduction of autologous CTLs or natural killer cells. CTL activation may also be promoted by strategies that enhance tumor antigen presentation by antigen presenting cells. Granulocyte macrophage colony stimulating factor (GM-CSF) promotes the recruitment of dendritic cells and activation of dendritic cell cross-priming. In one embodiment the combination therapy may comprise the isolation of antigen presenting cells, activation of such cells with stimulatory cytokines (e.g. GM-CSF), priming with tumor-associated antigens, and then adoptive reintroduction of the antigen presenting cells into patients in combination with the use of disclosed ADCs and optionally one or more different therapeutic moiety(ies).

In other embodiments an ADC of the invention may be used in combination with one or more of the anti-cancer agents described below.

The term “anti-cancer agent” or “chemotherapeutic agent” as used herein is one subset of “therapeutic moieties”, which in turn is a subset of the agents described as “pharmaceutically active moieties”. More particularly “anti-cancer agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, biological response modifiers, therapeutic antibodies, cancer vaccines, cytokines, hormone therapy, anti-metastatic agents and immunotherapeutic agents. It will be appreciated that in selected embodiments as discussed above, such anti-cancer agents may comprise antibody drug conjugates and may be associated with antibodies prior to administration. In certain embodiments the resulting anti-cancer agent ADC may be used in combination with the ADCs of the present invention as disclosed herein.

The term “cytotoxic agent”, which may be an anti-cancer agent, means a substance that is toxic to the cells and decreases or inhibits the function of cells and/or causes destruction of cells. Typically, the substance is a naturally occurring molecule derived from a living organism (or a synthetically prepared natural product). Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, saponaria officinalis inhibitor, mitegellin, restrictocin, phenomycin, neomycin, and the tricothecenes) or animals, (e.g., cytotoxic RNases, such as extracellular pancreatic RNases; DNase I, including fragments and/or variants thereof).

An anti-cancer agent can include any chemical agent that inhibits, or is designed to inhibit, a cancerous cell or a cell likely to become cancerous or generate tumorigenic progeny (e.g., tumorigenic cells). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. Such agents are often administered, and are often most effective, in combination, e.g., in the formulation CHOP. Again, in selected embodiments such anti-cancer agents may be conjugated to the disclosed antibodies.

Examples of anti-cancer agents that may be used in combination with the calicheamicin ADCs of the invention include, but are not limited to, alkylating agents, alkyl sulfonates, anastrozole, amanitins, aziridines, ethylenimines and methylamelamines, acetogenins, a camptothecin, BEZ-235, bortezomib, bryostatin, callystatin, CC-1065, ceritinib, crizotinib, cryptophycins, dolastatin, duocarmycin, eleutherobin, erlotinib, pancratistatin, a sarcodictyin, spongistatin, nitrogen mustards, antibiotics, enediyne dynemicin, bisphosphonates, esperamicin, chromoprotein enediyne antiobiotic chromophores, aclacinomysins, actinomycin, authramycin, azaserine, bleomycins, cactinomycin, canfosfamide, carabicin, carminomycin, carzinophilin, chromomycinis, cyclosphosphamide, dactinomycin, daunorubicin, detorubicin, 6-diazo-5-oxo-L-norleucine, doxorubicin, epirubicin, esorubicin, exemestane, fluorouracil, fulvestrant, gefitinib, idarubicin, lapatinib, letrozole, lonafarnib, marcellomycin, megestrol acetate, mitomycins, mycophenolic acid, nogalamycin, olivomycins, pazopanib, peplomycin, potfiromycin, puromycin, quelamycin, rapamycin, rodorubicin, sorafenib, streptonigrin, streptozocin, tamoxifen, tamoxifen citrate, temozolomide, tepodina, tipifarnib, tubercidin, ubenimex, vandetanib, vorozole, XL-147, zinostatin, zorubicin; anti-metabolites, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecocline, diaziquone, elfornithine, elliptinium acetate, epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, polysaccharide complex, razoxane; rhizoxin; SF-1126, sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, chloranbucil; gemcitabine; 6-thioguanine; mercaptopurine; methotrexate; platinum analogs, vinblastine; platinum; etoposide; ifosfamide; mitoxantrone; vincristine; vinorelbine; novantrone; teniposide; edatrexate; daunomycin; aminopterin; xeloda; ibandronate; irinotecan, topoisomerase inhibitor RFS 2000; difluorometlhylornithine; retinoids; capecitabine; combretastatin; leucovorin; oxaliplatin; XL518, inhibitors of PKC-alpha, Raf, H-Ras, EGFR and VEGF-A that reduce cell proliferation and pharmaceutically acceptable salts or solvates, acids or derivatives of any of the above. Also included in this definition are anti-hormonal agents that act to regulate or inhibit hormone action on tumors such as anti-estrogens and selective estrogen receptor antibodies, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts or solvates, acids or derivatives of any of the above.

Particularly preferred anti-cancer agents comprise commercially or clinically available compounds such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®). Additional commercially or clinically available anti-cancer agents comprise oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, I1), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca).

In other embodiments the ADCs of the instant invention may be used in combination with any one of a number of antibodies (or immunotherapeutic agents) presently in clinical trials or commercially available. The disclosed antibodies may be used in combination with an antibody selected from the group consisting of abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lambrolizumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nivolumab, nofetumomabn, obinutuzumab, ocaratuzumab, ofatumumab, olaratumab, olaparib, onartuzumab, oportuzumab, oregovomab, panitumumab, parsatuzumab, patritumab, pemtumomab, pertuzumab, pidilizumab, pintumomab, pritumumab, racotumomab, radretumab, ramucirumab, rilotumumab, rituximab, robatumumab, satumomab, selumetinib, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8, MDX-1105 and MEDI4736 and combinations thereof.

Other particularly preferred embodiments comprise the use of antibodies approved for cancer therapy including, but not limited to, rituximab, gemtuzumab ozogamcin, alemtuzumab, ibritumomab tiuxetan, tositumomab, bevacizumab, cetuximab, patitumumab, ofatumumab, ipilimumab and brentuximab vedotin. Those skilled in the art will be able to readily identify additional anti-cancer agents that are compatible with the teachings herein.

### D. Radiotherapy

The present invention also provides for the combination of ADCs with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed ADCs may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### IX Indications

The invention provides for the use of ADCs of the invention for the diagnosis, theragnosis, treatment and/or prophylaxis of various disorders including neoplastic, inflammatory, angiogenic and immunologic disorders and disorders caused by pathogens. Particularly, key targets for treatment are neoplastic conditions comprising solid tumors, although hematologic malignancies are within the scope of the invention. In certain embodiments the ADCs of the invention will be used to treat tumors or tumorigenic cells expressing a particular determinant (e.g. SEZ6). Preferably the “subject” or “patient” to be treated will be human although, as used herein, the terms are expressly held to comprise any mammalian species.

It will be appreciated that the compounds and compositions of the instant invention may be used to treat subjects at various stages of disease and at different points in their treatment cycle. Accordingly, in certain embodiments the antibodies and ADCs of the instant invention will be used as a front line therapy and administered to subjects who have not previously been treated for the cancerous condition. In other embodiments the antibodies and ADCs of the invention will be used to treat second and third line patients (i.e., those subjects that have previously been treated for the same condition one or two times respectively). Still other embodiments will comprise the treatment of fourth line or higher patients (e.g., SCLC patients) that have been treated for the same or related condition three or more times with the disclosed ADCs or with different therapeutic agents. In other embodiments the compounds and compositions of the present invention will be used to treat subjects that have previously been treated (with antibodies or ADCs of the present invention or with other anti-cancer agents) and have relapsed or are determined to be refractory to the previous treatment. In selected embodiments the compounds and compositions of the instant invention may be used to treat subjects that have recurrent tumors.

In certain aspects the proliferative disorder will comprise a solid tumor including, but not limited to, adrenal, liver, kidney, bladder, breast, gastric, ovarian, cervical, uterine, esophageal, colorectal, prostate, pancreatic, lung (both small cell and non-small cell), thyroid, carcinomas, sarcomas, glioblastomas and various head and neck tumors. In other preferred embodiments, and as shown in the Examples below, the disclosed ADCs are particularly effective at treating small cell lung cancer (SCLC) and non-small cell lung cancer (NSCLC) (e.g., squamous cell non-small cell lung cancer or squamous cell small cell lung cancer). In certain embodiments the lung cancer is refractory, relapsed or resistant to a platinum based agent (e.g., carboplatin, cisplatin, oxaliplatin, topotecan) and/or a taxane (e.g., docetaxel, paclitaxel, larotaxel or cabazitaxel). In another embodiment the subject to be treated is suffering from large cell neuroendocrine carcinoma (LCNEC). In still other aspects of the invention the disclosed antibodies and ADCs may be used for the treatment of medullary thyroid cancer, glioblastoma, neuroendocrine prostate cancer, (NEPC), high-grade gastroenteropancreatic cancer (GEP) and malignant melanoma.

More generally exemplary neoplastic conditions subject to treatment in accordance with the instant invention may be benign or malignant; solid tumors or other blood neoplasia; and may be selected from the group including, but not limited to: adrenal gland tumors, AIDS-associated cancers, alveolar soft part sarcoma, astrocytic tumors, autonomic ganglia tumors, bladder cancer (squamous cell carcinoma and transitional cell carcinoma), blastocoelic disorders, bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, epithelial disorders, Ewing's tumors, extraskeletal myxoid chondrosarcoma, fibrogenesis imperfecta ossium, fibrous dysplasia of the bone, gallbladder and bile duct cancers, gastric cancer, gastrointestinal, gestational trophoblastic disease, germ cell tumors, glandular disorders, head and neck cancers, hypothalamic, intestinal cancer, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), macrophagal disorders, medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, stromal disorders, synovial sarcoma, testicular cancer, thymic carcinoma, thymoma, thyroid metastatic cancer, and uterine cancers (carcinoma of the cervix, endometrial carcinoma, and leiomyoma).

In particularly preferred embodiments the subject will be suffering from pancreatic cancer, colorectal cancer, non-small cell lung cancer, and gastric cancer. In the preferred embodiments the subject will be refractory as to pancreatic cancer, colorectal cancer, non-small cell lung cancer, and gastric cancer.

In other preferred embodiments, the ADCs are especially effective at treating lung cancer, including the following subtypes: small cell lung cancer and non-small cell lung cancer (e.g. squamous cell non-small cell lung cancer or squamous cell small cell lung cancer). In selected embodiments the antibodies and ADCs can be administered to patients exhibiting limited stage disease or extensive stage disease. In other preferred embodiments the disclosed conjugated antibodies will be administered to refractory patients (i.e., those whose disease recurs during or shortly after completing a course of initial therapy); sensitive patients (i.e., those whose relapse is longer than 2-3 months after primary therapy); or patients exhibiting resistance to a platinum based agent (e.g. carboplatin, cisplatin, oxaliplatin) and/or a taxane (e.g. docetaxel, paclitaxel, larotaxel or cabazitaxel).

In another particularly preferred embodiment the disclosed ADCs are effective at treating ovarian cancer, including ovarian-serous carcinoma and ovarian-papillary serous carcinoma.

The invention also provides for a preventative or prophylactic treatment of subjects who present with benign or precancerous tumors. No particular type of tumor or proliferative disorder is excluded from treatment using the antibodies of the invention.

### X Articles of Manufacture

The invention includes pharmaceutical packs and kits comprising one or more containers, wherein a container can comprise one or more doses of an ADC of the invention. In certain embodiments, the pack or kit contains a unit dosage, meaning a predetermined amount of a composition comprising, for example, an ADC of the invention, with or without one or more additional agents and optionally, one or more anti-cancer agents.

The kit of the invention will generally contain in a suitable container a pharmaceutically acceptable formulation of the ADC of the invention and, optionally, one or more anti-cancer agents in the same or different containers. The kits may also contain other pharmaceutically acceptable formulations or devices, either for diagnosis or combination therapy. Examples of devices or instruments include those that can be used to detect, monitor, quantify or profile cells or markers associated with proliferative disorders. The kits contemplated by the invention can also contain appropriate reagents to combine the ADC of the invention with an anti-cancer agent or diagnostic agent (e.g., see U.S. Pat. No. 7,422,739).

When the components of the kit are provided in one or more liquid solutions, the liquid solution can be non-aqueous, however, an aqueous solution is preferred, with a sterile aqueous solution being particularly preferred. The formulation in the kit can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, phosphate-buffered saline, Ringer's solution or dextrose solution. Where the kit comprises the ADC of the invention in combination with additional therapeutics or agents, the solution may be pre-mixed, either in a molar equivalent combination, or with one component in excess of the other. Alternatively, the ADC of the invention and any optional anti-cancer agent or other agent can be maintained separately within distinct containers prior to administration to a patient.

The kit can comprise one or multiple containers and a label or package insert in, on or associated with the container(s), indicating that the enclosed composition is used for diagnosing or treating the disease condition of choice. Suitable containers include, for example, bottles, vials, syringes, etc. The containers can be formed from a variety of materials such as glass or plastic. The container(s) can comprise a sterile access port, for example, the container may be an intravenous solution bag or a vial having a stopper that can be pierced by a hypodermic injection needle.

In some embodiments the kit can contain a means by which to administer the ADC and any optional components to a patient, e.g., one or more needles or syringes (pre-filled or empty), an eye dropper, pipette, or other such like apparatus, from which the formulation may be injected or introduced into the subject or applied to a diseased area of the body. The kits of the invention will also typically include a means for containing the vials, or such like, and other components in close confinement for commercial sale, such as, e.g., blow-molded plastic containers into which the desired vials and other apparatus are placed and retained.

### XI Miscellaneous

Unless otherwise defined herein, scientific and technical terms used in connection with the invention shall have the meanings that are commonly understood by those of ordinary skill in the art. Further, unless otherwise required by context, singular terms shall include pluralities and plural terms shall include the singular. In addition, ranges provided in the specification and appended claims include both end points and all points between the end points. Therefore, a range of 2.0 to 3.0 includes 2.0, 3.0, and all points between 2.0 and 3.0.

Generally, techniques of cell and tissue culture, molecular biology, immunology, microbiology, genetics and chemistry described herein are those well-known and commonly used in the art. The nomenclature used herein, in association with such techniques, is also commonly used in the art. The methods and techniques of the invention are generally performed according to conventional methods well known in the art and as described in various references that are cited throughout the present specification unless otherwise indicated.

### XII References

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described.

### XIII Sequence Listing Summary

Appended to the instant application are figures comprising a number of nucleic acid and amino acid sequences. The following Table 3 provides a summary of the included sequences.

TABLE 3

| SEQ ID NO | Description |
|---|---|
| 1 | Kappa light chain constant region protein |
| 2 | IgG1 heavy chain constant region protein |
| 3 | Cleavable peptide |
| 4 | Cleavable peptide |
| 5 | Cleavable peptide |

### Embodiments

Embodiments disclosed herein include embodiments P1 to P27 following.

### Embodiment P1

An antibody drug conjugate of the formula Ab-[W—(X1)ₐ-CM-(X2)_{b}—P-D]ₙ or a pharmaceutically acceptable salt thereof wherein a) Ab comprises a targeting agent; b) W comprises a connecting group; CM comprises a cleavable moiety; d) P comprises a disulfide protective group; e) X1 and X2 comprise optional spacer moieties; and f) D comprises calicheamicin; wherein a and b are independently 0 or 1 and n is 1, 2, 3, 4, 5, 6, 7, 8, 9 or 10.

### Embodiment P2

The antibody drug conjugate of embodiment P1 wherein the targeting agent comprises an antibody.

### Embodiment P3

The antibody drug conjugate of embodiment P2 where the antibody comprises a chimeric, CDR grafted, humanized or human antibody or an immunoreactive fragment thereof.

### Embodiment P4

The antibody drug conjugate of embodiments P2 or P3 where the antibody comprises an anti-SEZ6 antibody.

### Embodiment P5

The antibody drug conjugate of any one of embodiments P2 to P4 where the antibody comprises a site-specific antibody.

### Embodiment P6

An antibody drug conjugate of any of embodiments P2 to P5 wherein the antibody comprises two unpaired cysteines.

### Embodiment P7

An antibody drug conjugate according to embodiment P6 wherein each antibody light chain comprises an unpaired cysteine residue.

### Embodiment P8

An antibody drug conjugate according to embodiment P7 wherein each unpaired cysteine residue is at position C214.

### Embodiment P9

The antibody drug conjugate of any one of embodiments P1 to P8 where n comprises an integer of from 2 to 8.

### Embodiment P10

The antibody drug conjugate of any one of embodiments P1 to P9 where n comprises an integer of 2.

### Embodiment P11

The antibody drug conjugate of any one of embodiments P1 to P10 wherein D comprises an analog of calicheamicin γ₁^{I}.

### Embodiment P12

The antibody/drug conjugate of any of embodiments P1 to P11, wherein the calicheamicin is an N-acetyl derivative or disulfide analog of calicheamicin.

### Embodiment P13

The antibody/drug conjugate of any of embodiments P1 to P12, wherein the calicheamicin is N-acetyl-γ-calicheamicin.

### Embodiment P14

The antibody drug conjugate of any of embodiments P1 to P13 wherein the cleavable moiety comprises a peptide bond, a hydrazone moiety, an oxime moiety, an ester linkage, or a disulfide linkage.

### Embodiment P15

The antibody drug conjugate of any of embodiments P1 to P14 wherein the cleavable moiety comprises and peptide bond.

### Embodiment P16

A pharmaceutical composition comprising an antibody drug conjugate of any one of embodiments P1 to P15.

### Embodiment P17

A method of treating cancer comprising administering a pharmaceutical composition of embodiment 16 to a subject in need thereof.

### Embodiment P18

The method of embodiment P17, wherein the cancer is selected from pancreatic cancer, colorectal cancer, non-small cell lung cancer, small cell lung cancer and gastric cancer.

### Embodiment P19

The method of embodiments P17 or P18, further comprising administering to the subject at least one additional therapeutic moiety.

### Embodiment P20

A method of delivering a calicheamicin cytotoxin to a cell comprising contacting the cell with an antibody drug conjugate of any one of embodiments P1 to P15.

### Embodiment P21

A method of preparing an antibody drug conjugate comprising the steps of: a) providing an calicheamicin construct comprising a cleavable linker; b) reducing the targeting agent to provide an activated residue; and c) conjugating the reduced targeting agent to the calicheamnicin construct.

### Embodiment P22

The method of embodiment P21 where the targeting agent comprises a site-specific antibody

### Embodiment P23

The method of embodiment P22 wherein the site-specific antibody comprises a free cysteine derived from a native disulfide bridge.

### Embodiment P24

The method of embodiment P22 wherein the engineered antibody comprises a free cysteine that is not derived from a native disulfide bridge.

### Embodiment P25

The method of embodiment P22 wherein the free cysteine comprises an introduced cysteine residue or a substituted cysteine residue.

### Embodiment P26

The method of any of embodiments P21 to P25 wherein the step of reducing the targeting agent comprises selectively reducing the target agent.

### Embodiment 27

The method of embodiment P26 wherein the step of selectively reducing the antibody comprises the step of contacting the antibody with a stabilizing agent.

Further embodiments include embodiments 1 to 44 following.

### Embodiment 1

A compound, or a pharmaceutically acceptable salt thereof, having the Formula (I): Ab-[W-(L³)_{z1}-M-(L⁴)_{z2}-P-D]_{z3} (I), wherein: Ab is a targeting agent; W is a connecting group; M is a cleavable moiety; L³ and L⁴ are independently a linker; P is a disulfide protecting group; D is a calicheamicin or analog thereof; z1 and z2 are independently an integer from 0 to 10; and z3 is an integer from 1 to 10.

### Embodiment 2

The compound of embodiment 1, wherein D comprises Formula (Ia):

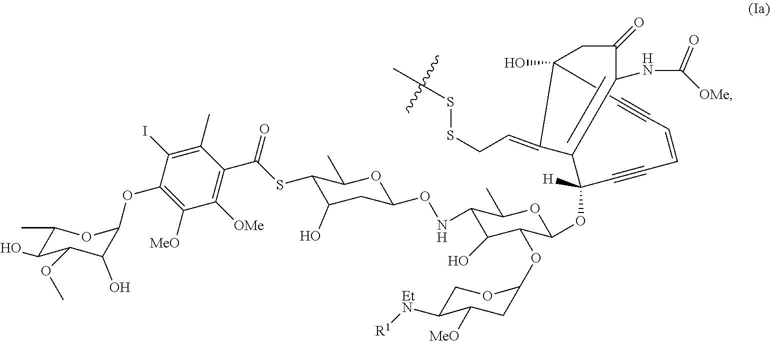

wherein: R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —CI₃, —CN, —C(O)R^{1E}, —OR^{1A}, —NR^{1B}R^{1C}, —C(O)OR^{1A}, —C(O)NR^{1B}R^{1C}, —SR^{1D}, —SO₁R^{1B} or —SOₙᵥ₁NR^{1B}R^{1C}; R^{1A}, R^{1B}, R^{1C}, R^{1D} and R^{1E} are independently hydrogen, halogen, —CF₃, —CCl₃, —CBr₃, —CI₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{1B} and R^{1C} substituents bonded to the same nitrogen atom may optionally be joined to form a substituted or unsubstituted heterocycloalkyl or substituted or unsubstituted heteroaryl; n1 is an integer from 0 to 4; and v1 is 1 or 2.

### Embodiment 3

The compound of embodiment 2, wherein R¹ is hydrogen, substituted or unsubstituted alkyl or —C(O)R^{1E}

### Embodiment 4

The compound of embodiment 2, wherein the targeting agent is an antibody.

### Embodiment 5

The compound of embodiment 4, wherein the antibody is a chimeric antibody, a CDR grafted antibody, a humanized antibody or a human antibody or an immunoreactive fragment thereof.

### Embodiment 6

The compound of embodiment 4, wherein the antibody is an anti-SEZ6 antibody.

### Embodiment 7

The compound of embodiment 4, wherein W is covalently attached a cysteine residue within the antibody.

### Embodiment 8

The compound of embodiment 7, wherein the cysteine residue is at Kabat position C214.

### Embodiment 9

The compound of embodiment 4, wherein W is covalently attached to a lysine residue within the antibody.

### Embodiment 10

The compound of embodiment 1, or a pharmaceutically acceptable salt thereof, having the Formula (II):

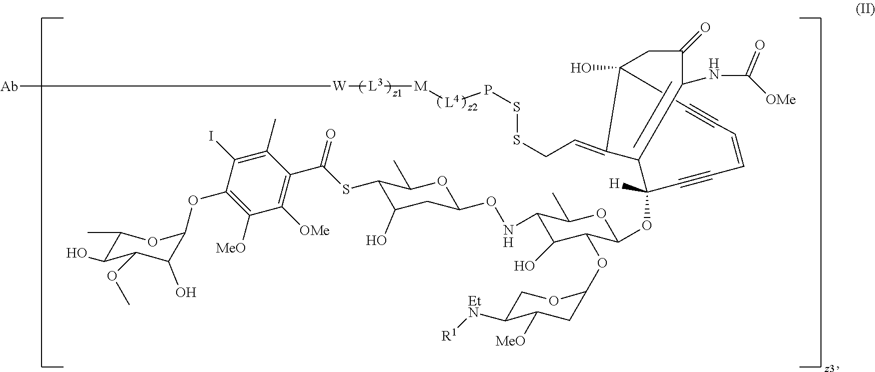

wherein: Ab is an antibody; L³ is a bond, —O—, —S—, —NR^{3B}—, —C(O)—, —C(O)O—, —S(O)—, —S(O)₂—, —C(O)NR^{3B}—, —NR^{3B}C(O)—, —NR^{3B}C(O)NH—, —NHC(O)NR^{3B}—, substituted or unsubstituted alkylene or substituted or unsubstituted heteroalkylene; L⁴ is a bond, —O—, —S—, —NR^{4B}—, —C(O)—, —C(O)O—, —S(O)—, —S(O)₂—, —C(O)NR^{4B}—, —NR^{4B}C(O)—, —NR^{4B}C(O)NH—, —NHC(O)NR^{4B}—, substituted or unsubstituted alkylene or substituted or unsubstituted heteroalkylene; R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —CI₃, —CN, —C(O)R^{1E}, —OR^{1A}, —NR^{1B}R^{1C}, —C(O)OR^{1A}, —C(O)NR^{1B}R^{1C}, —SR^{1D}, —SOₙ₁R^{1B} or —SOᵥ₁NR^{1B}R^{1C}; P is —O—, —S—, —NR^{2B}—, —C(O)—, —C(O)O—, —S(O)—, —S(O)₂—, —C(O)NR^{2B}—, —NR^{2B}C(O)—, —NR^{2B}C(O)NH—, —NHC(O)NR^{2B}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; M is —O—, —S—, —NR^{5B}—, —C(O)—, —C(O)O—, —S(O), —S(O)₂—, —C(O)NR^{5B}, —NR^{5B}C(O)—, —NR^{5B}C(O)NH—, —NHC(O)NR^{5B}—, —[NR^{5B}C(R^{5E})(R^{5F})C(O)]ₙ₂—, substituted or unsubstituted alkylene, substituted or unsubstituted heteroalkylene, substituted or unsubstituted cycloalkylene, substituted or unsubstituted heterocycloalkylene, substituted or unsubstituted arylene substituted or unsubstituted heteroarylene or M^{1A}-M^{1B}-M^{1C}; W is —O—, —S—, —NR^{6B}—, —C(O)—, —C(O)O—, —S(O)—, —S(O)₂—, —C(O)NR^{6B}—, —NR^{6B}C(O)—, —NR^{6B}C(O)NH—, —NHC(O)NR^{6B}—, substituted or unsubstituted alkylene, substituted or unsubstituted heteroalkylene, substituted or unsubstituted cycloalkylene, substituted or unsubstituted heterocycloalkylene, substituted or unsubstituted arylene substituted or unsubstituted heteroarylene or W^{1A}—W^{1B}—W^{1C}; M^{1A} is bonded to L³ and M^{1C} is bonded to L⁴; M^{1A} is a bond, —O—, —S—, —NR^{5AB}—, —C(O)—, —C(O)O—, —S(O)—, —S(O)₂—, —C(O)NR^{5AB}—, —NR^{5AB}C(O)—, —NR^{5AB}C(O)NH—, —NHC(O)NR^{5AB}—, —[NR^{5AB}CR^{5AE}R^{5AF}C(O)]ₙ₃—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; M^{1B} is a bond, —O—, —S—, —NR^{5BB}—, —C(O)—, —C(O)O—, —S(O)—, —S(O)₂—, —C(O)NR^{5BB}—, —NR^{5BB}C(O)—, —NR^{5BB}C(O)NH—, —NHC(O)NR^{5BB}—, —[NR^{5BB}C(R^{5BE})(R^{5BF})C(O)]ₙ₄—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; M^{1C} is a bond, —O—, —S—, —NR^{5CB}—, —C(O)—, —C(O)O—, —S(O)—, —S(O)₂—, —C(O)NR^{5CB}—, —NR^{5CB}C(O)—, —NR^{5CB}C(O)NH—, —NHC(O)NR^{5CB}, —[NR^{5CB}CR^{5CE}R^{5CF}C(O)]ₙ₅—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; W^{1A} is bonded to Ab and W^{1C} is bonded to L³; W^{1A} is a bond, —O—, —S—, —NR^{6AB}, —C(O)—, C(O)O—, —S(O)—, —S(O)₂—, —C(O)NR^{6AB}—, —NR^{6AB}C(O)—, —NR^{6AB}C(O)NH—, —NHC(O)NR^{6AB}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; W^{1B} is a bond, —O—, —S—, —NR^{6BB}—, —C(O)—, —C(O)O—, —S(O)—, —S(O)₂—, —C(O)NR^{6BB}—, —NR^{6BB}C(O)—, —NR^{6BB}C(O)NH—, —NHC(O)NR^{6BB}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; W^{1C} is a bond, —O—, —S—, —NR^{6CB}—, —C(O)—, —C(O)O—, —S(O)—, —S(O)₂—, —C(O)NR^{6CB}—, —NR^{6CB}C(O)—, —NR^{6CB}C(O)NH—, —NHC(O)NR^{6CB}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; R^{1A}, R^{1B}, R^{1C}, R^{1D}, R^{1E}, R^{2B}, R^{3B}, R^{4B}, R^{5B}, R^{5E}, R^{5F}R^{5A}, R^{5AE}, R^{5AF}, R^{5BB}, R^{5BE}, R^{5BF}, R^{5CB}, R^{5CE}, R^{5CF}, R^{6B}, R^{6AB}, R^{6BB} and R^{6CB} are independently hydrogen, halogen, —CF₃, —CCl₃, —CBr₃, —CI₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{1B} and R^{1C} substituents bonded to the same nitrogen atom may optionally be joined to form a substituted or unsubstituted heterocycloalkyl or substituted or unsubstituted heteroaryl; n1 is an integer from 0 to 4; v1 is 1 or 2; n2, n3, n4 and n5 are independently and integer from 1 to 10; z1 and z2 are independently an integer from 0 to 10; and z3 is an integer from 1 to 10.

### Embodiment 11

The compound of embodiment 10, wherein M is M^{1A}-M^{1B}-M^{1C}, wherein: M^{1A} is bonded to L³ and M^{1C} is bonded to L⁴.

### Embodiment 12

The compound of embodiment 10, wherein W is W^{1A}—W^{1B}—W^{1C}, wherein W^{1A} is bonded to Ab and W^{1C} is bonded to L³.

### Embodiment 13

The compound of embodiment 10, wherein P is substituted or unsubstituted alkyl.

### Embodiment 14

The compound of embodiment 10, wherein z3 is 1 or 2.

### Embodiment 15

The compound of embodiment 10, wherein L³ is substituted or unsubstituted alkylene or substituted or unsubstituted heteroalkylene.

### Embodiment 16

The compound of embodiment 10, wherein L⁴ is substituted or unsubstituted alkylene or substituted or unsubstituted heteroalkylene.

### Embodiment 17

The compound of embodiment 10, wherein R¹ is hydrogen or —C(O)R^{1E}

### Embodiment 18

The compound of embodiment 10, wherein W is substituted or unsubstituted heteroalkylene, substituted or unsubstituted cycloalkylene, substituted or unsubstituted heterocycloalkylene, substituted or unsubstituted arylene or substituted or unsubstituted heteroarylene.

### Embodiment 19

The compound of embodiment 18, wherein W is 5- or 6-membered substituted or unsubstituted heterocycloalkylene.

### Embodiment 20

The compound of embodiment 19, wherein W has the formula:

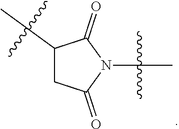 [SMILES: *C1CC(=O)N(*)C1=O]

### Embodiment 21

The compound of embodiment 10, wherein M comprises a peptide.

### Embodiment 22

The compound of embodiment 10, wherein: M^{1A} is a bond, substituted or unsubstituted heteroalkylene or —[NR^{5AB}C(R^{5AE})(R^{5AF})C(O)]ₙ₃; M^{1B} is a bond, substituted or unsubstituted heteroalkylene or —[NR^{5BB}C(R^{5BE})(R^{5BF})C(O)]ₙ₄—; and M^{1C} is a bond or substituted or unsubstituted arylene or substituted or unsubstituted heteroarylene.

### Embodiment 23

The compound of embodiment 10, wherein M^{1A} and M^{1B} are independently amino acids.

### Embodiment 24

The compound of embodiment 10, wherein at least one of M^{1A} or M^{1B} is valine (val).

### Embodiment 25

The compound of embodiment 10, wherein at least one of M^{1A} or M^{1B} is alanine (ala).

### Embodiment 26

The compound of embodiment 10, wherein at least one of M^{1A} or M^{1B} is citrulline (cit).

### Embodiment 27

The compound of embodiment 10, wherein at least one of M^{1A}, M^{1B} or MiC is substituted arylene.

### Embodiment 28

The compound of embodiment 10, wherein at least one of M^{1A}, M^{1B} or M^{1C} has Formula (III):

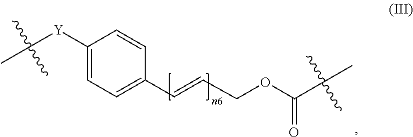

wherein: Y is —NH—, —O—, —C(O)NH— or —C(O)O—; and n6 is an integer from 0 to 3.

### Embodiment 29

The compound of embodiment 10, wherein —[W-(L³)_{z1}-M-(L⁴)_{z2}-P-D] is:

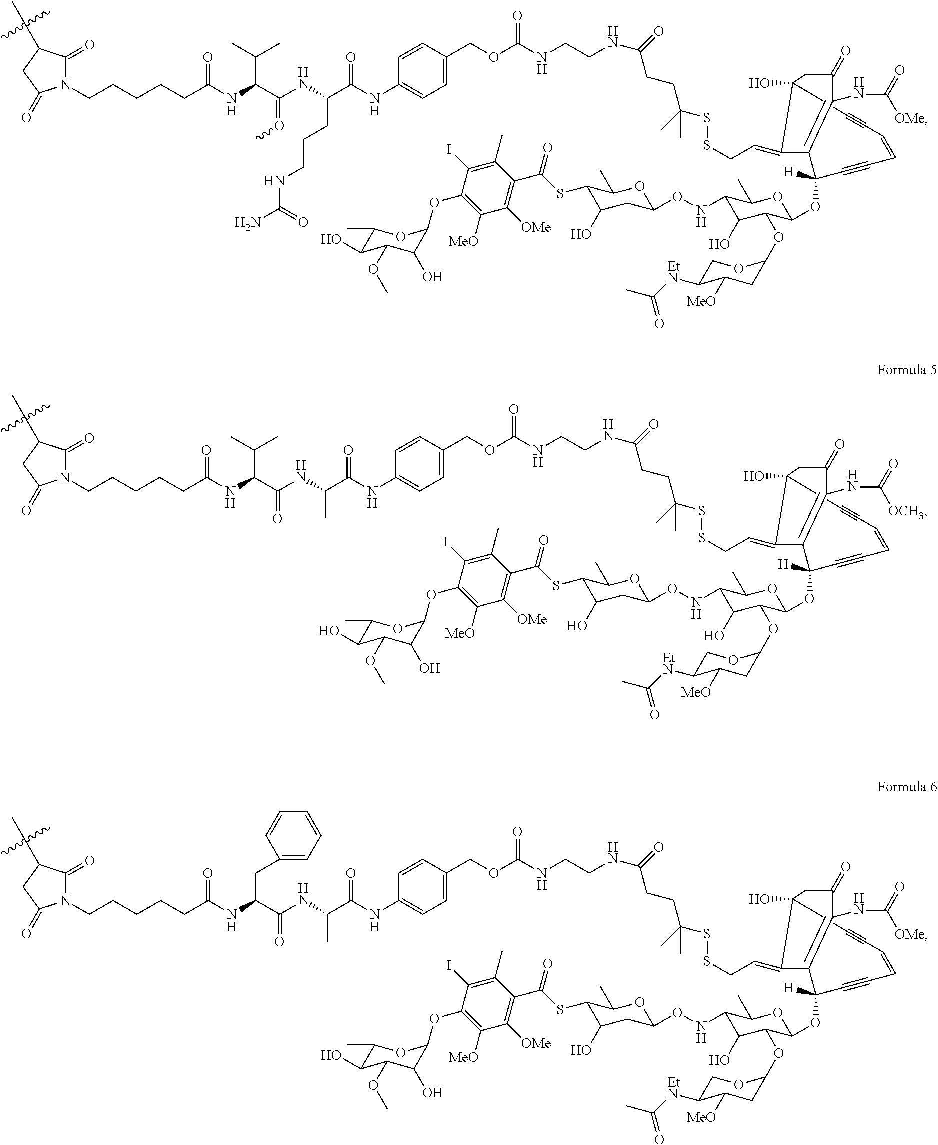

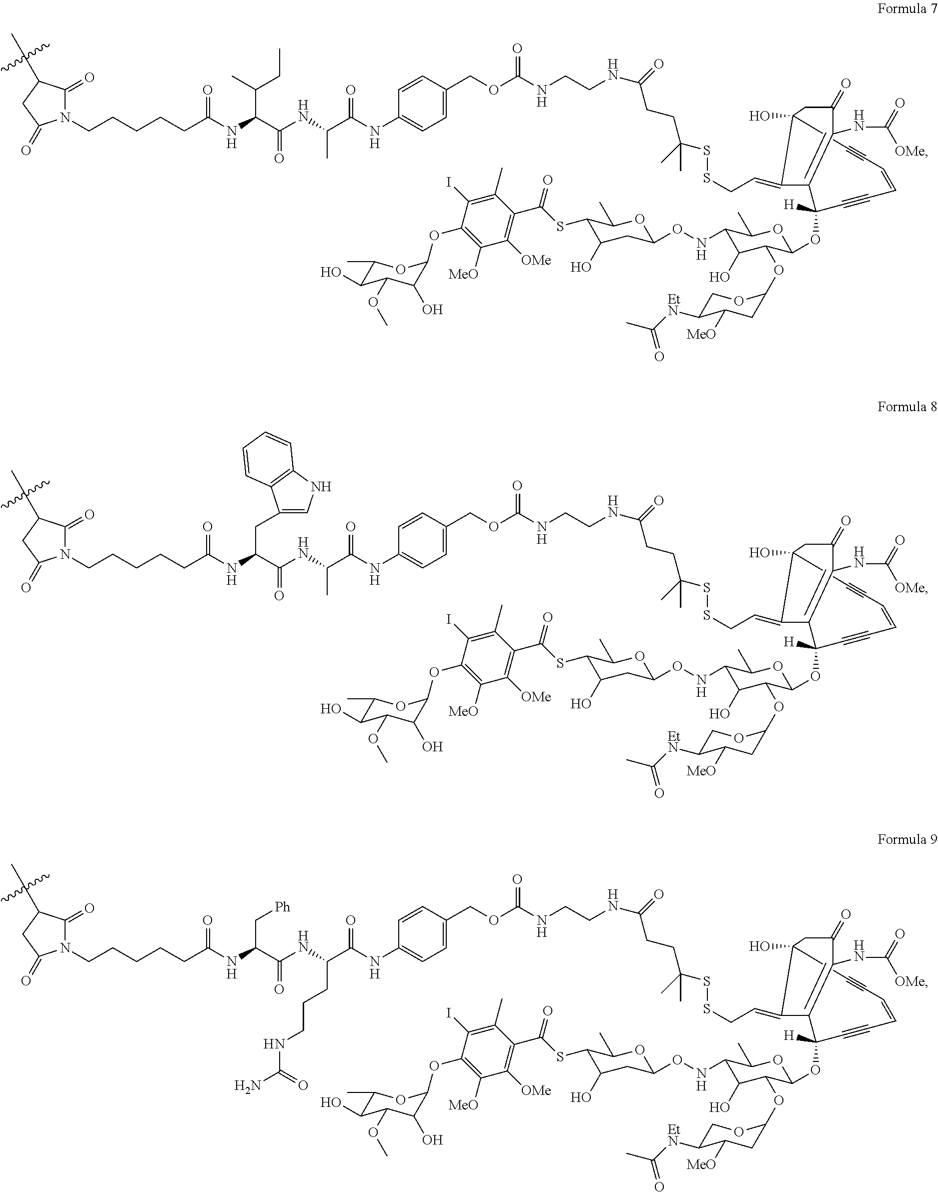

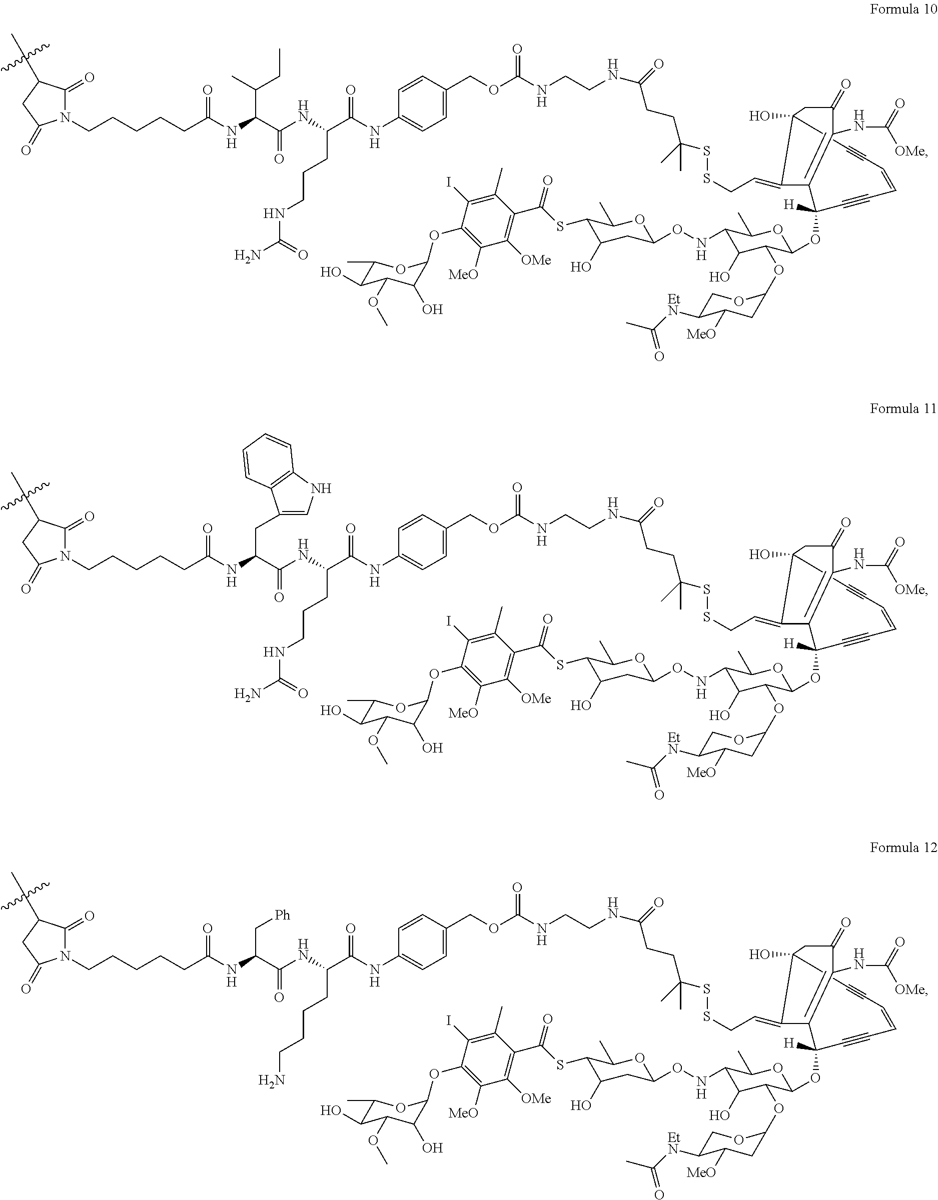

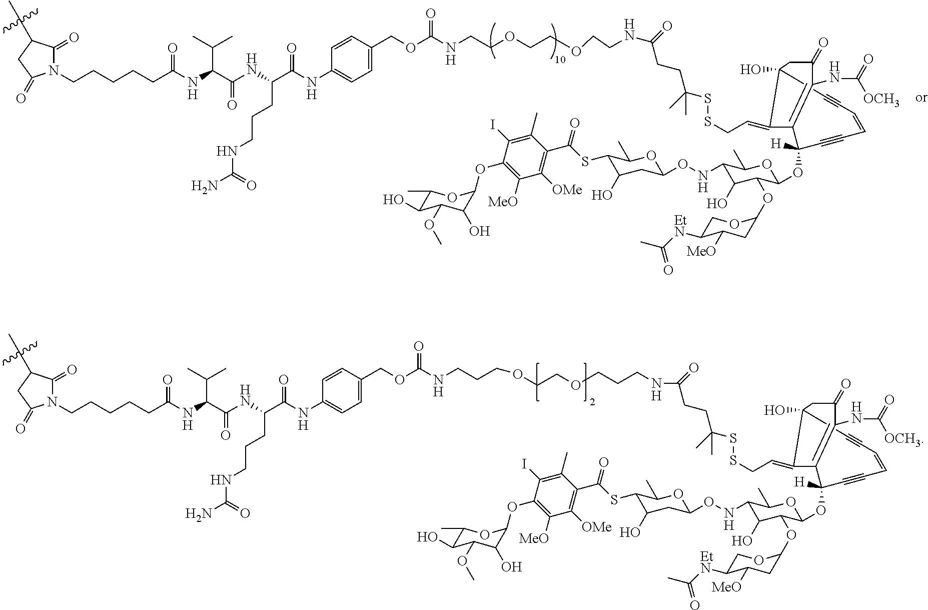

### Embodiment 30

The compound of embodiment 10, wherein —[W-(L³)_{z1}-M-(L⁴)_{z2}-P-D] is of formula:

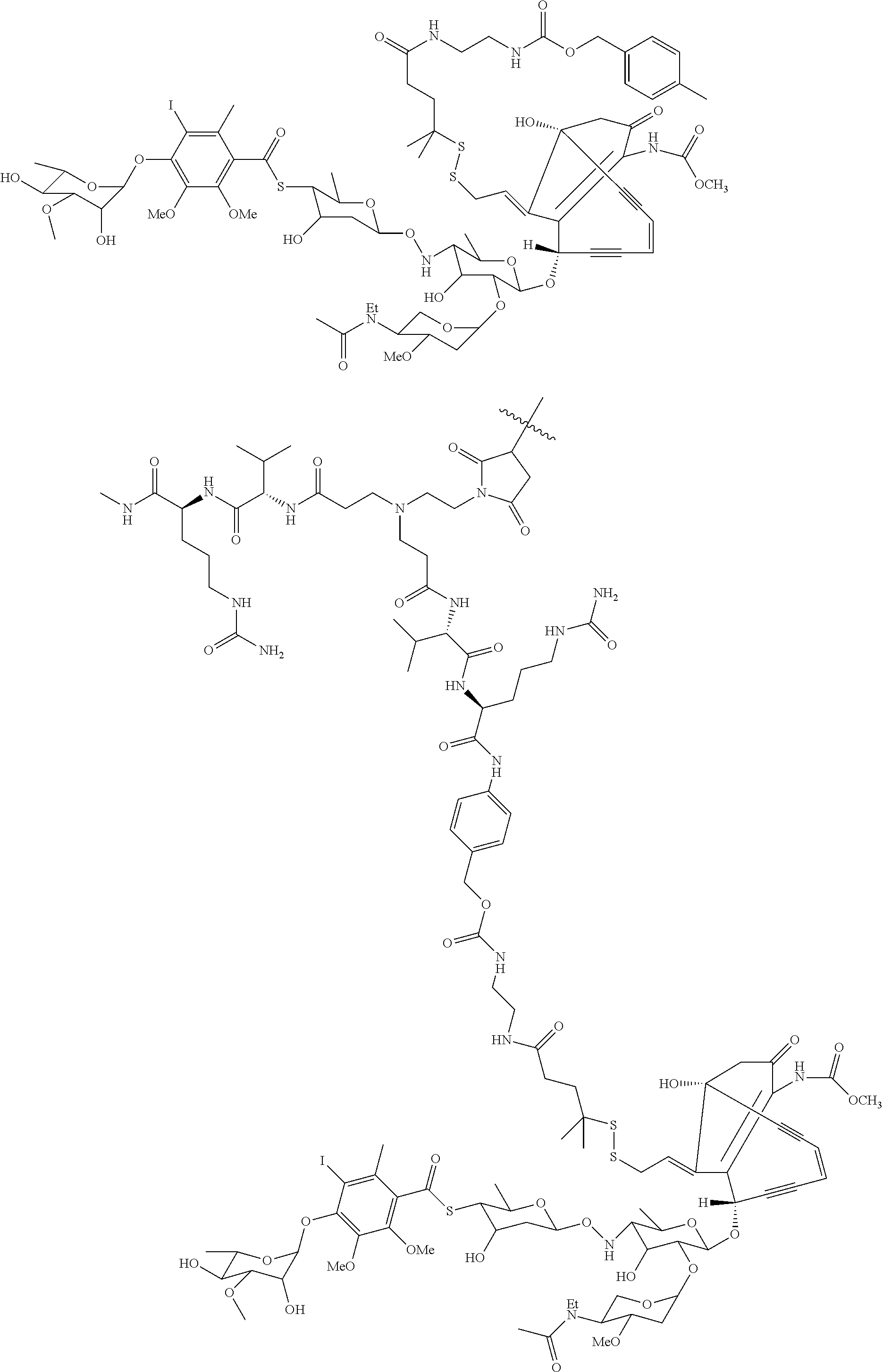

### Embodiment 31

A pharmaceutical composition comprising a compound of any one of embodiments 1 to 30.

### Embodiment 32

A method of treating cancer in a subject in need thereof comprising administering a therapeutically effective amount of the pharmaceutical composition of embodiment 31 or the compound of one of embodiments 1 to 30 to the subject.

### Embodiment 33

The method of embodiment 32, wherein the cancer is selected from pancreatic cancer, colorectal cancer, non-small cell lung cancer, small cell lung cancer and gastric cancer.

### Embodiment 34

The method of embodiment 32, further comprising administering to the subject an additional chemotherapeutic agent.

### Embodiment 35

A method of delivering a calicheamicin cytotoxin to a cell comprising contacting the cell with a compound of any one of embodiments 1 to 30.

### Embodiment 36

A method of preparing an antibody drug conjugate comprising contacting a calicheamicin construct with a cysteine or lysine of an antibody, the calichearnicin construct having the formula W¹-(L³)_{z1}-M-(L⁴)_{z2}-P-D, wherein W¹ is a functional group reactive with a lysine side chain or cysteine side chain, M is a cleavable moiety, L³ and L⁴ are independently a linker, P is a disulfide protecting group and D is a calicheamicin or analog thereof.

### Embodiment 37

The method of embodiment 36, wherein the calicheamicin construct is contacted with a specific cysteine of the antibody.

### Embodiment 38

The method of embodiment 37, wherein the specific cysteine is derived from a native disulfide bridge.

### Embodiment 39

The method of embodiment 37, wherein the antibody is an engineered antibody and the specific cysteine is not derived from a native disulfide bridge.

### Embodiment 40

The method of any of embodiments 36 to 39, wherein the specific cysteine is selectively reduced prior to the contacting.

### Embodiment 41

The method of embodiment 40, wherein the step of selectively reducing the antibody, comprises the step of contacting the antibody with a stabilizing agent.

### Embodiment 42

A compound having the Formula (IV):

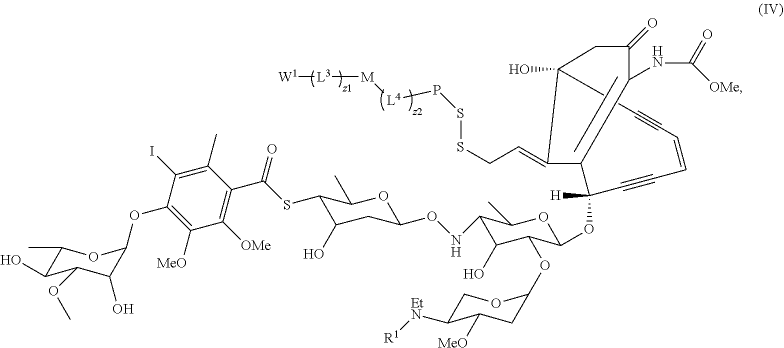

wherein L³ is a bond, —O—, —S—, —NR^{3B}—, —C(O)—, —C(O)O—, —S(O)—, —S(O)₂—, —C(O)NR^{3B}—, —NR^{3B}C(O)—, —NR^{3B}C(O)NH—, —NHC(O)NR^{3B}—, substituted or unsubstituted alkylene or substituted or unsubstituted heteroalkylene; L⁴ is a bond, —O—, —S—, —NR^{4B}—, —C(O)—, —C(O)O—, —S(O)—, —S(O)₂—, —C(O)NR^{4B}—, —NR^{4B}C(O)—, —NR^{4B}C(O)NH—, —NHC(O)NR^{4B}—, substituted or unsubstituted alkylene or substituted or unsubstituted heteroalkylene; R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —CI₃, —CN, —C(O)R^{1E}, —OR^{1A}, —NR^{1B}R^{1C}, —C(O)OR^{1A}, —C(O)NR^{1B}R^{1C}, —SR^{1D}, —SOₙ₁R^{1B} or —SOᵥ₁NR^{1B}R^{1C}; P is —O—, —S—, —NR^{2B}—, —C(O)—, —C(O)O—, —S(O)—, —S(O)₂—, —C(O)NR^{2B}—, —NR^{2B}C(O)—, —NR^{2B}C(O)NH—, —NHC(O)NR^{2B}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; M is —O—, —S—, —NR^{5B}—, —C(O)—, —C(O)O—, —S(O)—, —S(O)₂—, —C(O)NR^{5B}—, —NR^{5B}C(O)—, —NR^{5B}C(O)NH—, —NHC(O)NR^{5B}—, —[NR^{5B}C(R^{5E})(R^{5F})C(O)]ₙ₂—, substituted or unsubstituted alkylene, substituted or unsubstituted heteroalkylene, substituted or unsubstituted cycloalkylene, substituted or unsubstituted heterocycloalkylene, substituted or unsubstituted arylene substituted or unsubstituted heteroarylene or M^{1A}-M^{1B}-M^{1C}; W¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —N₃, —CF₃, —CCl₃, —CBr₃, —CI₃, —CN, —C(O)R^{7E}, —OR^{7A}, —NR^{7B}R^{7C}, —C(O)OR^{7A}, —C(O)NR^{7B}R^{7C}, —NO₂, —SR^{7D}, —SOᵥ₇R⁷, —SOᵥ₇NR^{7B}R^{7C}, —NHNR^{7B}R^{7C}, —ONR^{7B}R^{7C}, —NHC(O)NHNR^{7B}R^{7C}; M^{1A} is bonded to L³ and M^{1C} is bonded to L4; M^{1A} is a bond, —O—, —S—, —NR^{AB}—, —C(O)—, —C(O)O—, —S(O)—, —S(O)₂—, —C(O)NR^{5AB}—, —NR^{5AB}C(O)—, —NR^{5AB}C(O)NH—, —NHC(O)NR^{5AB}—, —[NR^{5AB}CR^{5AE}R^{5AF}C(O)]ₙ₃—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; M^{1B} is a bond, —O—, —S—, —NR^{5BB}—, —C(O)—, —C(O)O—, —S(O)—, —S(O)₂—, —C(O)NR^{5BB}—, —NR^{5BB}C(O)—, —NR^{5BB}C(O)NH—, —NHC(O)NR^{5BB}—, —[NR^{5BB}C(R^{5BE})(R^{5BF})C(O)]ₙ₄—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; M^{1C} is a bond, —O—, —S—, —NR^{5CB}—, —C(O)—, —C(O)O—, —S(O)—, —S(O)₂—, —C(O)NR^{5CB}—, —NR^{5CB}C(O)—, —NR^{5CB}C(O)NH—, —NHC(O)NR^{5CB}, —[NR^{5CB}CR^{5CE}R^{5CF}C(O)]ₙ₅—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; R^{1A}, R^{1B}, R^{1C}, R^{1D}, R^{1E}, R^{2B}, R^{3B}, R^{4B}, R^{5B}, R^{5E}, R^{5F}, R^{5AB}, R^{5AE}, R^{5AF}, R^{5BB}, R^{5BE}, R^{5BF}, R^{5CB}CB, R^{5CE}, R^{5CF}, R^{6B}, R^{7A}, R^{7B}, R^{7C}, R^{7D}, R^{7E}, are independently hydrogen, halogen, —CF₃, —CCl₃, —CBr₃, —CI₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{1B} and R^{1C} substituents bonded to the same nitrogen atom may optionally be joined to form a substituted or unsubstituted heterocycloalkyl or substituted or unsubstituted heteroaryl; n1 and n7 are independently an integer from 0 to 4; v1 and v7 are independently 1 or 2; and n2, n3, n4 and n5 are independently and integer from 1 to 10.

### Embodiment 43

The compound of embodiment 42, wherein the compound is:

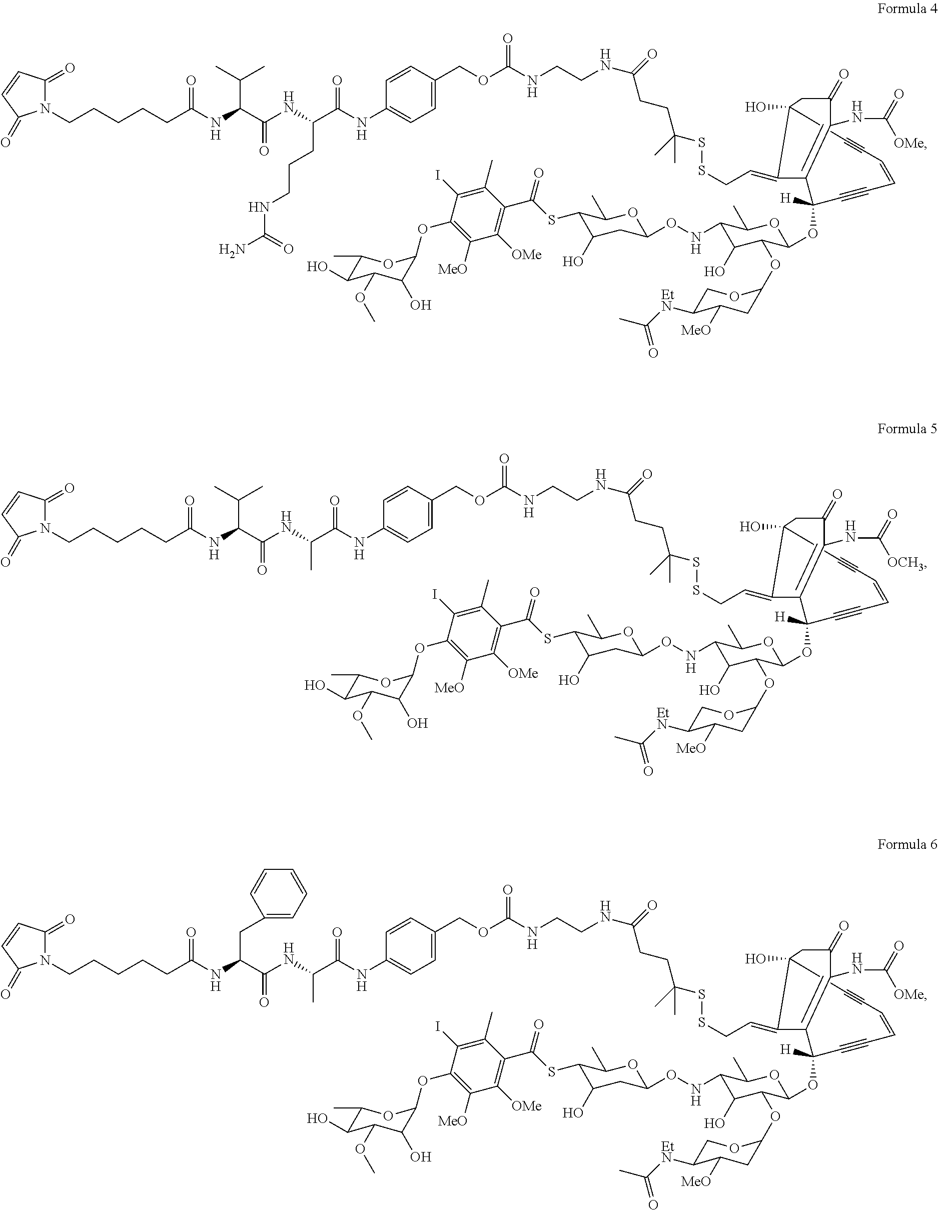

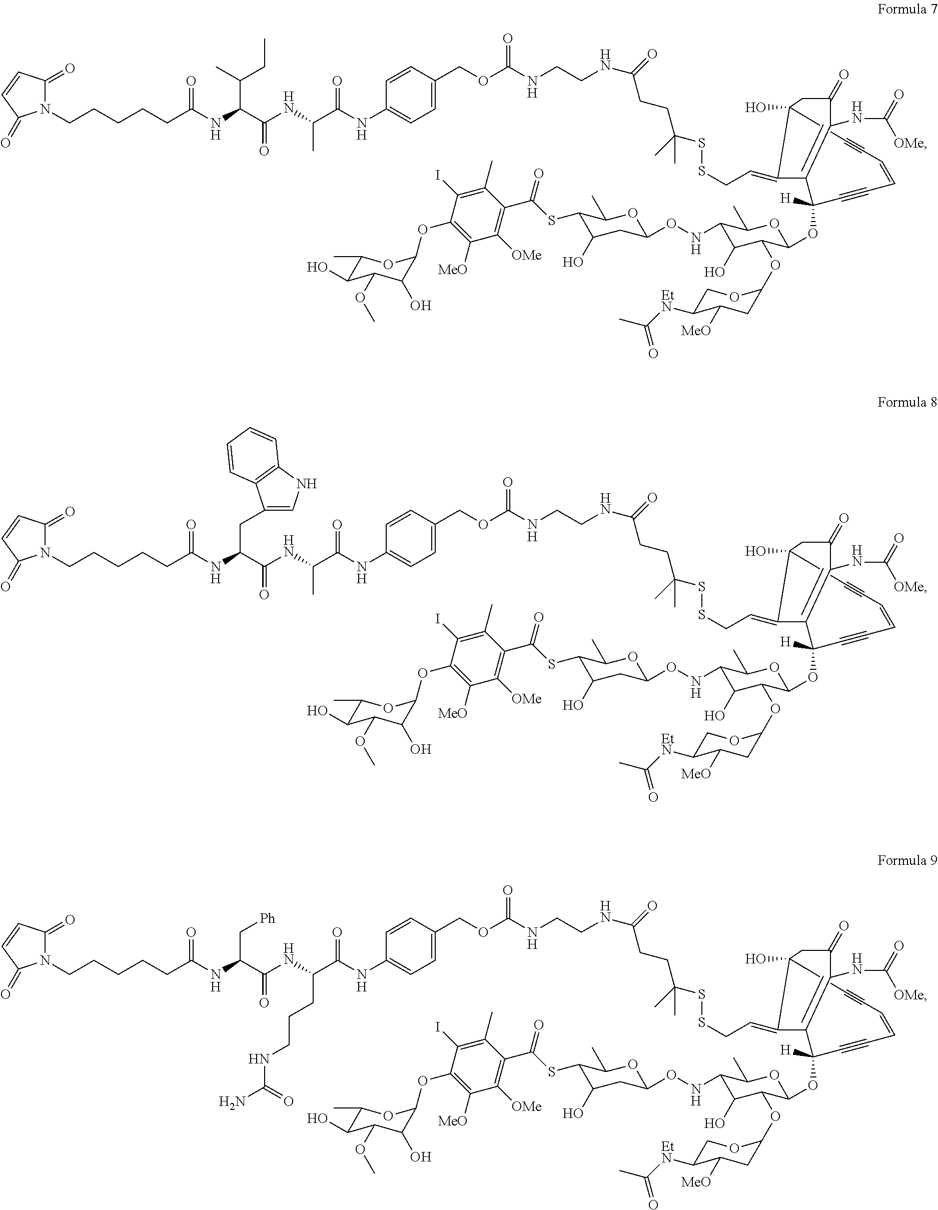

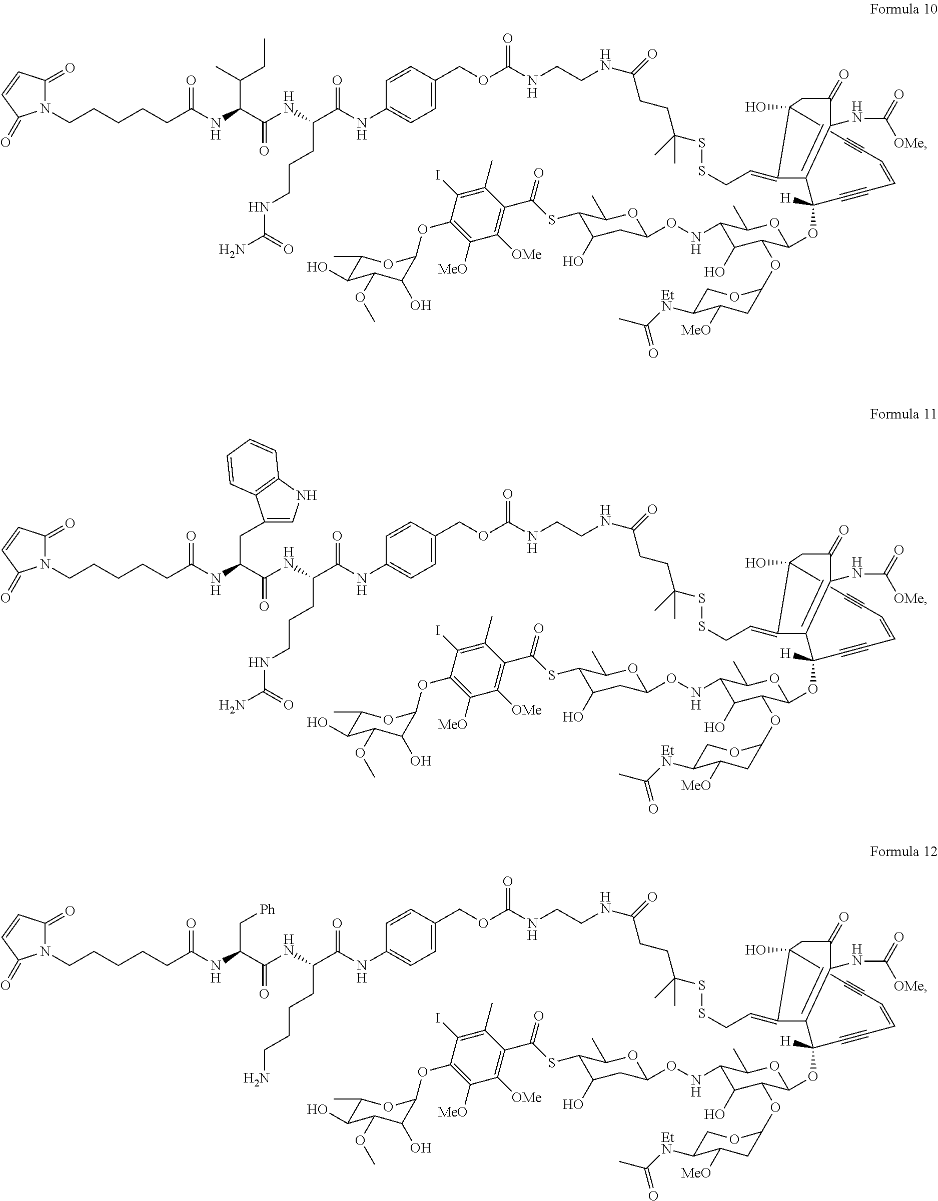

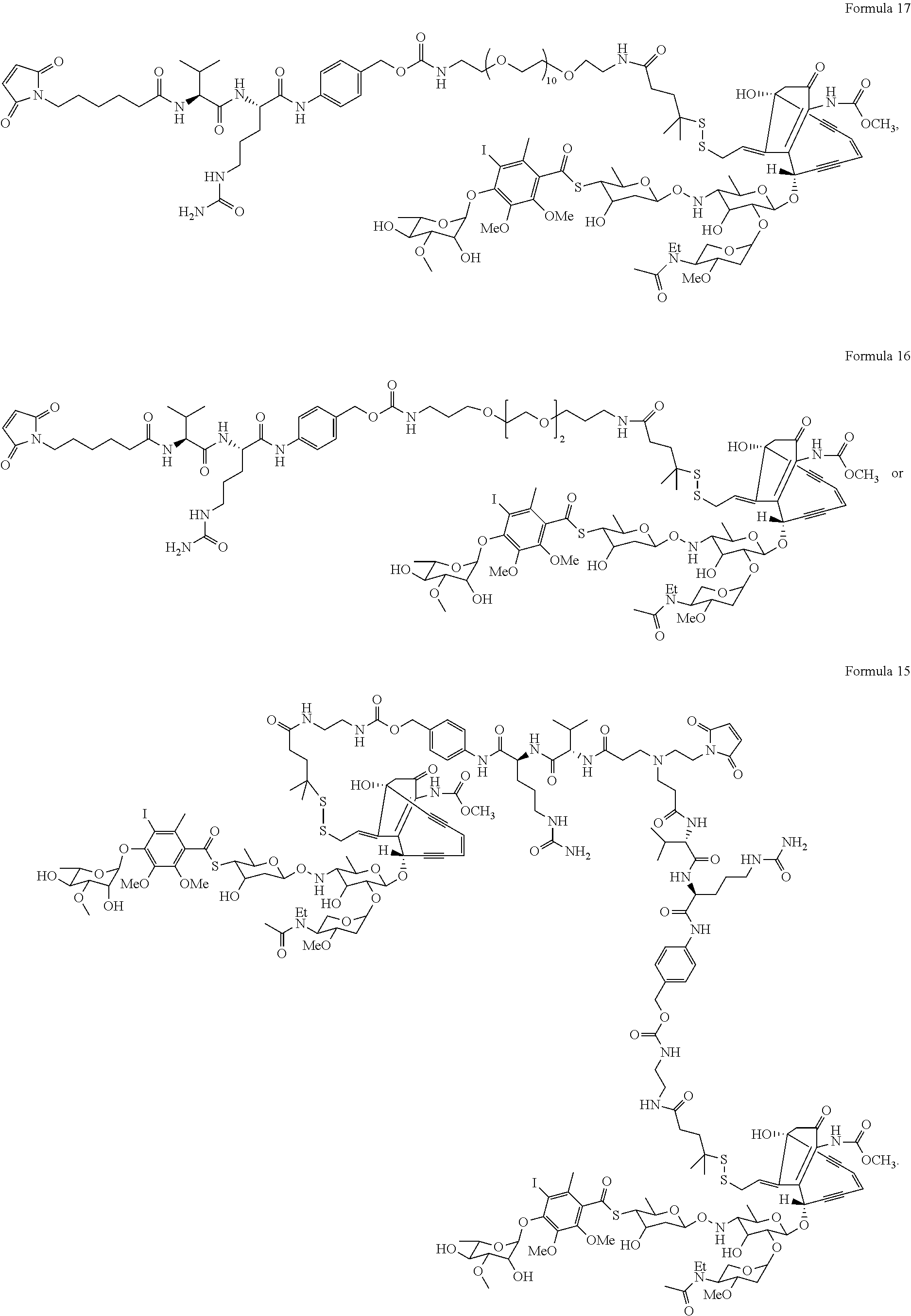

### XIV Examples

The invention, thus generally described above, will be understood more readily by reference to the following examples, which are provided by way of illustration and are not intended to be limiting of the instant invention. The examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

PDX tumor cell types are denoted by an abbreviation followed by a number, which indicates the particular tumor cell line. The passage number of the tested sample is indicated by p0-p# appended to the sample designation where p0 is indicative of an unpassaged sample obtained directly from a patient tumor and p# is indicative of the number of times the tumor has been passaged through a mouse prior to testing. As used herein, the abbreviations of the tumor types and subtypes are shown in Table 4 as follows:

TABLE 4

| | Abbre- | | Abbre- |
|---|---|---|---|
| Tumor Type | viation | Tumor subtype | viation |
| Breast | BR | | |
| | | estrogen receptor positive | BR-ERPR |
| | | and/or progesterone receptor | |
| | | positive | |
| | | ERBB2/Neu positive | BR- |
| | | | ERBB2/Neu |
| | | HER2 positive | BR-HER2 |
| | | triple-negative | TNBC |
| | | claudin subtype of triple- | TNBC-CLDN |
| | | negative | |
| colorectal | CR | | |
| endometrial | EN | | |
| gastric | GA | | |
| | | diffuse adenocarcinoma | GA-Ad-Dif/ |
| | | | Muc |
| | | intestinal adenocarcinoma | GA-Ad-Int |
| | | stromal tumors | GA-GIST |
| glioblastoma | GB | | |
| head and | HN | | |
| neck | | | |
| kidney | KDY | | |
| | | clear renal cell carcinoma | KDY-CC |
| | | papillary renal cell carcinoma | KDY-PAP |
| | | transitional cell or urothelial | KDY-URO |
| | | carcinoma | |
| | | unknown | KDY-UNK |
| liver | LIV | | |
| | | hepatocellular carcinoma | LIV-HCC |
| | | cholangiocarcinoma | LIV-CHOL |
| lymphoma | LN | | |
| lung | LU | | |
| | | adenocarcinoma | LU-Ad |
| | | carcinoid | LU-CAR |
| | | large cell neuroendocrine | LU-LCC |
| | | non-small cell | NSCLC |
| | | squamous cell | LU-SCC |
| | | small cell | SCLC |
| | | spindle cell | LU-SPC |
| melanoma | MEL | | |
| ovarian | OV | | |
| | | clear cell | OV-CC |
| | | endometroid | OV-END |
| | | mixed subtype | OV-MIX |
| | | malignant mixed mesodermal | OV-MMMT |
| | | mucinous | OV-MUC |
| | | neuroendocrine | OV-NET |
| | | papillary serous | OV-PS |
| | | serous | OV-S |
| | | small cell | OV-SC |
| | | transitional cell carcinoma | OV-TCC |
| pancreatic | PA | | |
| | | acinar cell carcinoma | PA-ACC |
| | | duodenal carcinoma | PA-DC |
| | | mucinous adenocarcinoma | PA-MAD |
| | | Neuroendocrine | PA-NET |
| | | adenocarcinoma | PA-PAC |
| | | adenocarcinoma exocrine type | PA-PACe |
| | | ductal adenocarcinoma | PA-PDAC |
| | | ampullary adenocarcinoma | PA-AAC |
| prostate | PR | | |
| skin | SK | | |
| | | melanoma | MEL |
| | | squamous cell carcinomas | SK-SCC |

General Information on analytical and preparative HPLC methods.

Analytical Method A:

MS: Acuity Ultra SQ Detector ESI, Scan range 120-2040 Da.

### Column: Waters Acuity UPLC BEH C18, 1.7 μm, 2.1×50 mm

Column temperature: 50° C.
Flow rate: 0.6 ml/min
Mobile phase A: 0.1% formic acid in water.
Mobile phase B: 0.1% formic acid in acetonitrile.

### Gradient:

| Time, min | % A | % B |
|---|---|---|
| 0 | 95 | 5 |
| 0.25 | 95 | 5 |
| 2 | 0 | 100 |
| 2.5 | 0 | 100 |
| 3 | 95 | 5 |
| 4 | 95 | 5 |

Analytical Method B:

MS: Acuity Ultra SQ Detector ESI, Scan range 120-2040 Da,

### Column: Waters Acuity UPLC BEH C18, 1.7 am, 2.1×50 mm

Column temperature: 60° C.
Flow rate: 0.4 ml/min
Mobile phase A: 0.1% formic acid in water.
Mobile phase B: 0.1% formic acid in acetonitrile.

### Gradient:

| Time, min | % A | % B |
|---|---|---|
| 0 | 95 | 5 |
| 2 | 95 | 5 |
| 3 | 80 | 20 |
| 13 | 20 | 80 |
| 14 | 20 | 80 |
| 14.10 | 5 | 95 |
| 15 | 5 | 95 |
| 15.10 | 95 | 5 |
| 20 | 95 | 5 |

Analytical Method C:

HRMS: ABSciex 5600 Plus Triple Time-of-Flight (TOF), scan range 250-2500 Da

### Column: Waters Acuity UPLC BEH C18, 1.7 μm, 2.1×50 mm

Column temperature: 60° C.
Flow rate: 0.4 ml/min
Mobile phase A: 0.1% formic acid in water.
Mobile phase B: 0.1% formic acid in acetonitrile.

### Gradient:

| Time, min | % A | % B |
|---|---|---|
| 0 | 95 | 5 |
| 2 | 95 | 5 |
| 3 | 80 | 20 |
| 13 | 20 | 80 |
| 14 | 20 | 80 |
| 14.10 | 5 | 95 |
| 15 | 5 | 95 |
| 15.10 | 95 | 5 |
| 20 | 95 | 5 |

Preparative HPLC Method A:

Column: Waters XBridge prep C18 5 μm OBD, 19×100 mm
Column temperature: ambient
Flow rate: 15 ml/min
Mobile phase A: 0.1% formic acid in water.
Mobile phase B: 0.1% formic acid in acetonitrile.

### Gradient:

| Time, min | % A | % B |
|---|---|---|
| 0 | 95 | 5 |
| 5 | 95 | 5 |
| 8 | 80 | 20 |
| 50 | 20 | 80 |
| 52.59 | 20 | 80 |
| 52.92 | 5 | 95 |
| 55.87 | 5 | 95 |
| 56.20 | 95 | 5 |
| 60 | 95 | 5 |

Preparative HPLC Method B:

Column: Waters XBridge prep C18 5 μm OBD, 19×100 mm
Column temperature: ambient
Flow rate: 15 ml/min
Mobile phase A: water.
Mobile phase B: acetonitrile.

### Gradient:

| Time, min | % A | % B |
|---|---|---|
| 0 | 95 | 5 |
| 5 | 95 | 5 |
| 8 | 80 | 20 |
| 50 | 20 | 80 |
| 52.59 | 20 | 80 |
| 52.92 | 5 | 95 |
| 55.87 | 5 | 95 |
| 56.20 | 95 | 5 |
| 60 | 95 | 5 |

### Example 1

### Synthesis of a Calicheamicin Construct Comprising a Hydrazone Linker

A drug-linker compound according to Formula 13

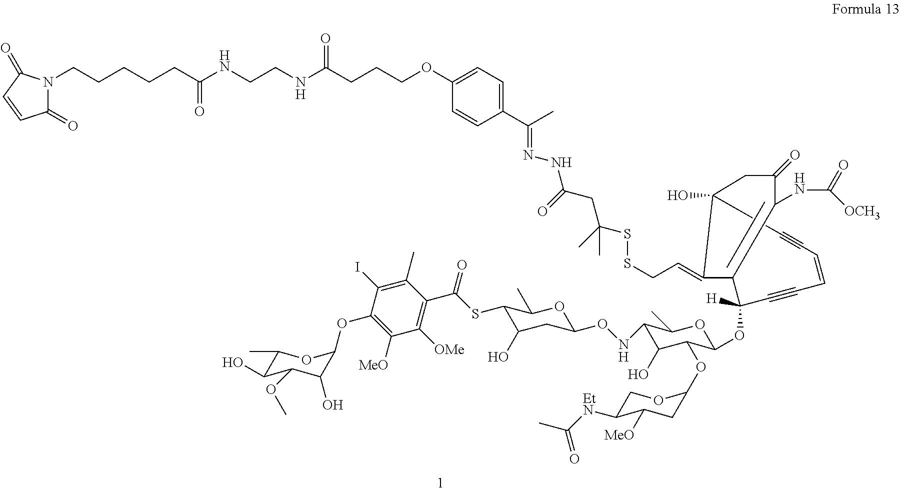

was synthesized using three different methods as set forth immediately below.

Synthesis Route 1:

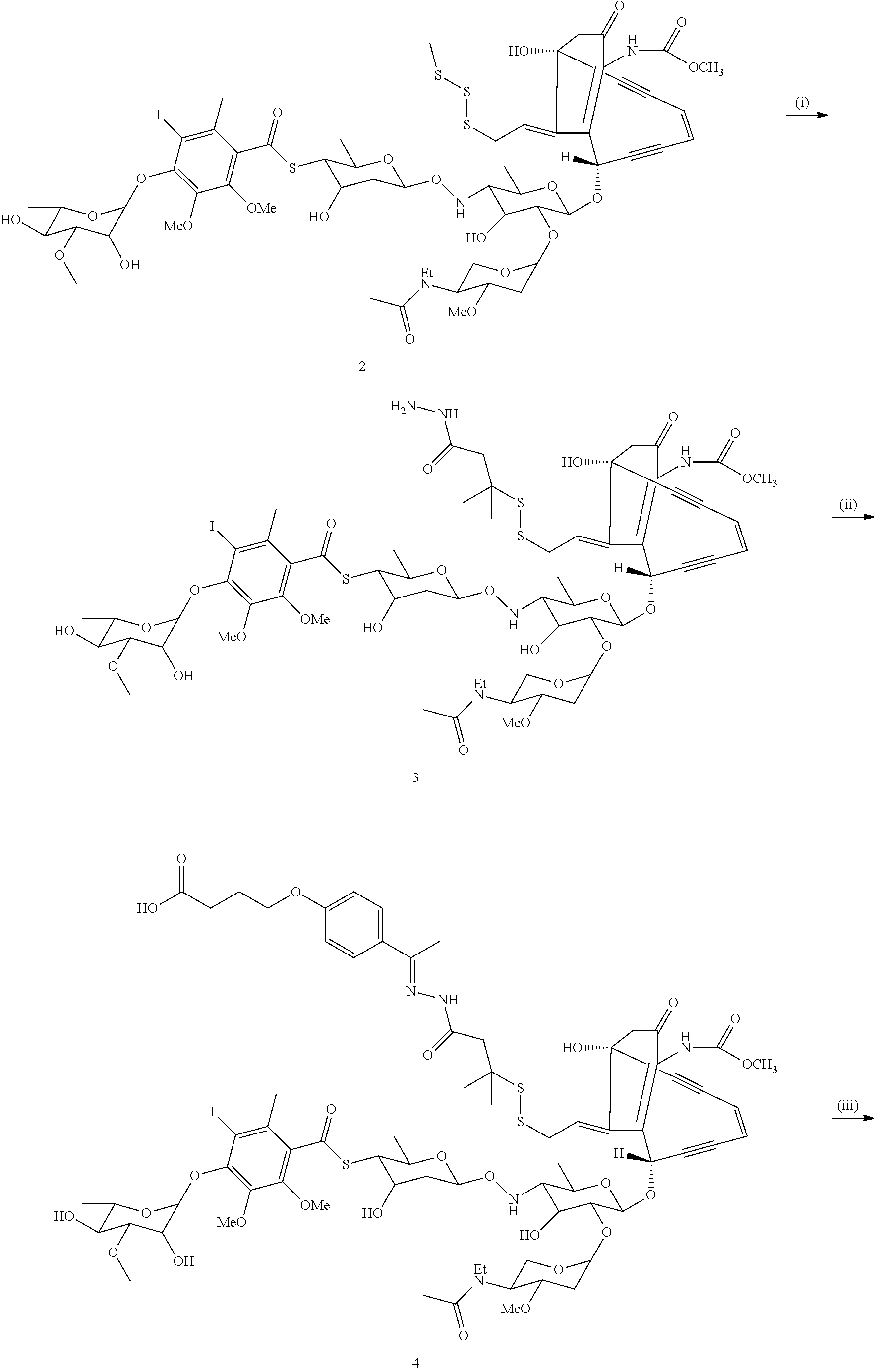

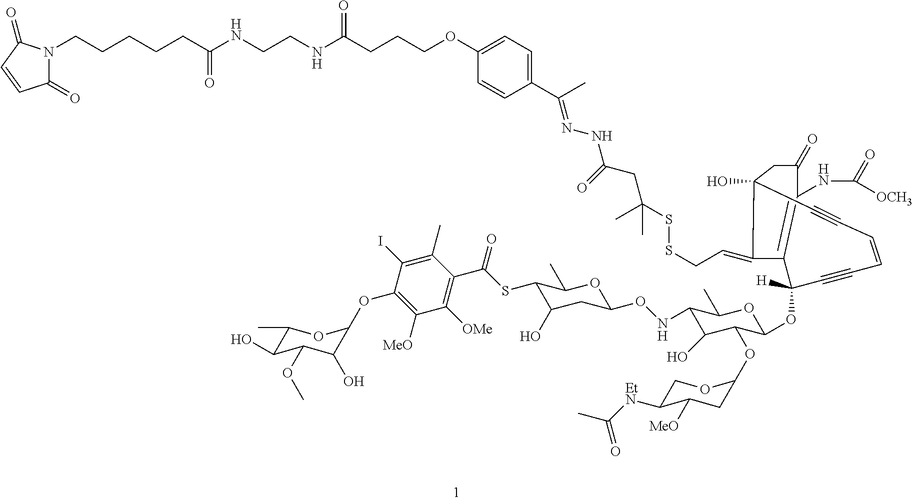

### (i)S-((2R,3S,4S,6S)-6-((((2R,3S,4R,5R,6R)-5-(((2S,4S,5S)-5-(N-ethylacetamido)-4-methoxytetrahydro-2H-pyran-2-yl)oxy)-6-(((2S,5Z,9R,13E)-13-(2-((4-hydrazinyl-2-methyl-4-oxobutan-2-yl)disulfanyl)ethylidene)-9-hydroxy-12-((methoxycarbonyl)amino)-11-oxobicyclo[7.3.1]trideca-1 (12),5-dien-3, 7-diyn-2-yl)oxy)-4-hydroxy-2-methyltetrahydro-2H-pyran-3-yl)amino)oxy)-4-hydroxy-2-methyltetrahydro-2H-pyran-3-yl) 4-(((2S,3R,4R,5S,6S)-3,5-dihydroxy-4-methoxy-6-methyltetrahydro-2H-pyran-2-yl)oxy)-3-iodo-5, 6-dimethoxy-2-methylbenzothioate (3)

N-acetyl calicheamicin (2, 20 mg, 14 μmol) was dissolved in 2 ml of acetonitrile and chilled to −15 C. 3-Mercapto-3-methylbutanehydrazide (21 mg, 0.14 mmol, 10 eq) was dissolved in 0.5 ml of acetonitrile and added slowly to the chilled solution of calicheamicin followed by addition of triethyl amine (18.8 μL, 014 mmol, 10 eq). The reaction was allowed to warm up until completion. After 3 hours, the reaction was concentrated and purified by column chromatography (MeOH/DCM 1 to 20%) on silica gel column to afford 3 (18.7 mg, 89%) as a white solid. LCMS (analytical method A): Rt=1.80 min, [M+H]⁺=1478.57.

### (ii) 4-(4-((E)-1-(2-(3-(((E)-2-((1R,8S,Z)-8-(((2R,3R,4R,5S,6R)-5-((((2S,4S,5S,6R)-5-((4-(((2S,3R,4R,5S,6S)-3,5-dihydroxy-4-methoxy-6-methyltetrahydro-2H-pyran-2-yl)oxy)-3-iodo-5,6-dimethoxy-2-methylbenzoyl)thio)-4-hydroxy-6-methyltetrahydro-2H-pyran-2-yl)oxy)amino)-3-(((2S,4S,5S)-5-(N-ethylacetamido)-4-methoxytetrahydro-2H-pyran-2-yl)oxy)-4-hydroxy-6-methyltetrahydro-2H-pyran-2-yl)oxy)-1-hydroxy-10-((methoxycarbonyl)amino)-11-oxobicyclo[7.3.1]trideca-4,9-dien-2,6-diyn-13-ylidene)ethyl)disulfanyl)-3-methylbutanoyl)hydrazono)ethyl)phenoxy)butanoic acid (4)

4-(4-acetylphenoxy)butanoic acid (3.8 mg, 17 μmol, 5 eq) was added to compound 3 (5 mg, 3.4 μmol) in alcohol (100 μL) in the presence of molecular sieves. Acetic acid (15 μL, 80 eq) was added and the reaction was stirred at 37° C. for 3 days. After that time, 80% conversion was observed and the reaction was concentrated and purified by column chromatography (MeOH/DCM 1 to 20%) on silica gel column to afford 4 (1.1 mg, 20%). LCMS (analytical method A): Rt=1.96 min, [M+H]⁺=1682.53

### (iii)S-((2R,3S,4S,6S)-6-((((2R,3S,4R,5R,6R)-6-(((2S,5Z,9R,13E)-13-(2-((4-(2-((E)-1-(4-(4-((2-(6-(2, 5-dioxo-2, 5-dihydro-1H-pyrrol-1-yl)hexanamido)ethyl)amino)-4-oxobutoxy)phenyl)ethylidene)hydrazinyl)-2-methyl-4-oxobutan-2-yl)disulfanyl)ethylidene)-9-hydroxy-12-((methoxycarbonyl)amino)-11-oxobicyclo[7.3.1]trideca-1(12),5-dien-3, 7-diyn-2-yl)oxy)-5-(((2S,4S,5S)-5-(N-ethylacetamido)-4-methoxytetrahydro-2H-pyran-2-yl)oxy)-4-hydroxy-2-methyltetrahydro-2H-pyran-3-yl)amino)oxy)-4-hydroxy-2-methyltetrahydro-2H-pyran-3-yl) 4-(((2S,3R,4R,5S,6S)-3,5-dihydroxy-4-methoxy-6-methyltetrahydro-2H-pyran-2-yl)oxy)-3-iodo-5,6-dimethoxy-2-methylbenzothioate (1)

5 μL of DIPEA (10 eq) was added to 500 μL of solution of N-(2-aminoethyl)-6-(2,5-dioxo-2,5-dihydro-1H-pyrrol-1-yl)hexanamide (7 mg/mL, 14 μmol, 5 eq) in DMF. This solution was added to a solution of 4 (480 μL of 10 mg/mL of DCM) with 5 μL of DIPEA (10 eq). Finally 11 mg EDCI (11 mg, 28 μmol, 10 eq) was added and the mixture was stirred at room temperature for 15 hours. The starting material was consumed and the desired product was observed by LCMS. LCMS (analytical method A): Rt=1.98 min, [M+H]⁺=1918.29

Synthesis Route 2:

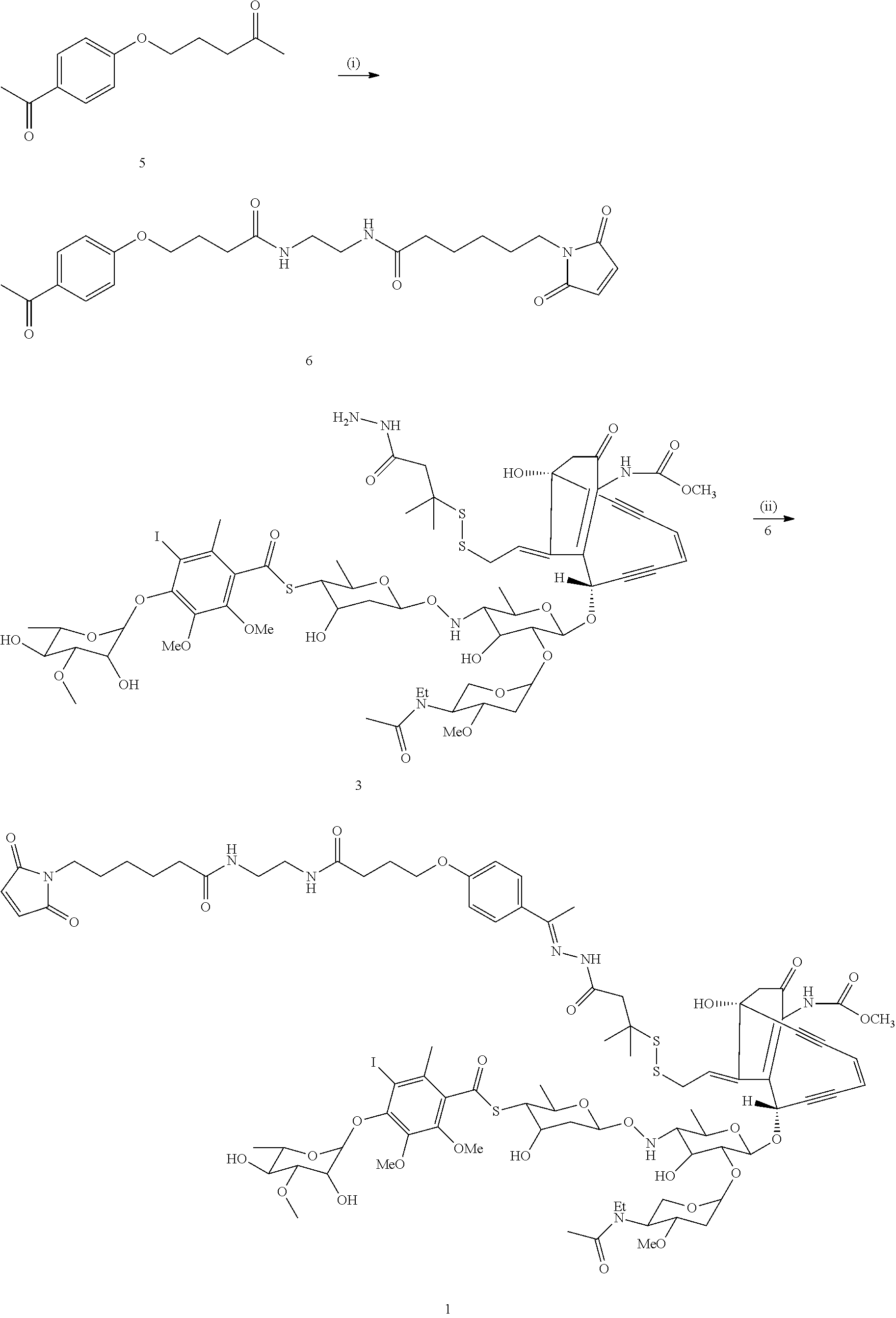

### (i)N-(2-(4-(4-acetylphenoxy)butanamido)ethyl)-6-(2,5-dioxo-2,5-dihydro-1H-pyrrol-1-yl)hexanamide (6)

N-(2-aminoethyl)-6-(2,5-dioxo-2,5-dihydro-1H-pyrrol-1-yl)hexanamide (137 mg, 0.54 mmol, 1.2 eq) was added to a solution of 5 (100 mg, 0.45 mmol) in THF (2 mL) followed by HATU (205.3 mg, 0.54 mmol, 1.2 eq) and HOBt hydrate (82.6 mg, 0.54 mmol, 1.2 eq). DIPEA (1.57 mL, 9.00 mmol, 20 eq) was then added and the reaction was stirred at room temperature for 15 hours. Solvent was evaporated and the crude product was purified by column chromatography to afford the desired product 6 (200 mg, 97%) as a white solid. LCMS (analytical method A): Rt=1.60 min, [M+H]⁺=458.37.

### (ii)S-((2R,3S,4S,6S)-6-((((2R,3S,4R,5R,6R)-6-(((2S,5Z,9R,13E)-13-(2-((4-(2-((E)-1-(4-(4-((2-(6-(2,5-dioxo-2,5-dihydro-1H-pyrrol-1-yl)hexanamido)ethyl)amino)-4-oxobutoxy)phenyl)ethylidene)hydrazinyl)-2-methyl-4-oxobutan-2-yl)disulfanyl)ethylidene)-9-hydroxy-12-((methoxycarbonyl)amino)-11-oxobicyclo [7.3.1] trideca-1(12),5-dien-3,7-diyn-2-yl)oxy)-5-(((2S,4S,5S)-5-(N-ethylacetamido)-4-methoxytetrahydro-2H-pyran-2-yl)oxy)-4-hydroxy-2-methyltetrahydro-2H-pyran-3-yl)amino)oxy)-4-hydroxy-2-methyltetrahydro-2H-pyran-3-yl) 4-(((2S,3R,4R,5S,6S)-3,5-dihydroxy-4-methoxy-6-methyltetrahydro-2H-pyran-2-yl)oxy)-3-iodo-5,6-dimethoxy-2-methylbenzothioate (1)

Solution of N-(2-(4-(4-acetylphenoxy)butanamido)ethyl)-6-(2,5-dioxo-2,5-dihydro-1H-pyrrol-1-yl)hexanamide 6 (0.3 mg, 0.6 μmol, 5 eq) in 20 μL of alcohol was added to compound 3 (0.2 mg, 0.14 μmol) in DMF (20 μL). Acetic acid (1 μL, 100 eq) was added and the reaction was stirred at 37° C. for 24 hours. After that time, desired product was observed. LCMS (analytical method A): Rt=1.98 min, [M+H]⁺=1918.69.

Synthesis Route 3:

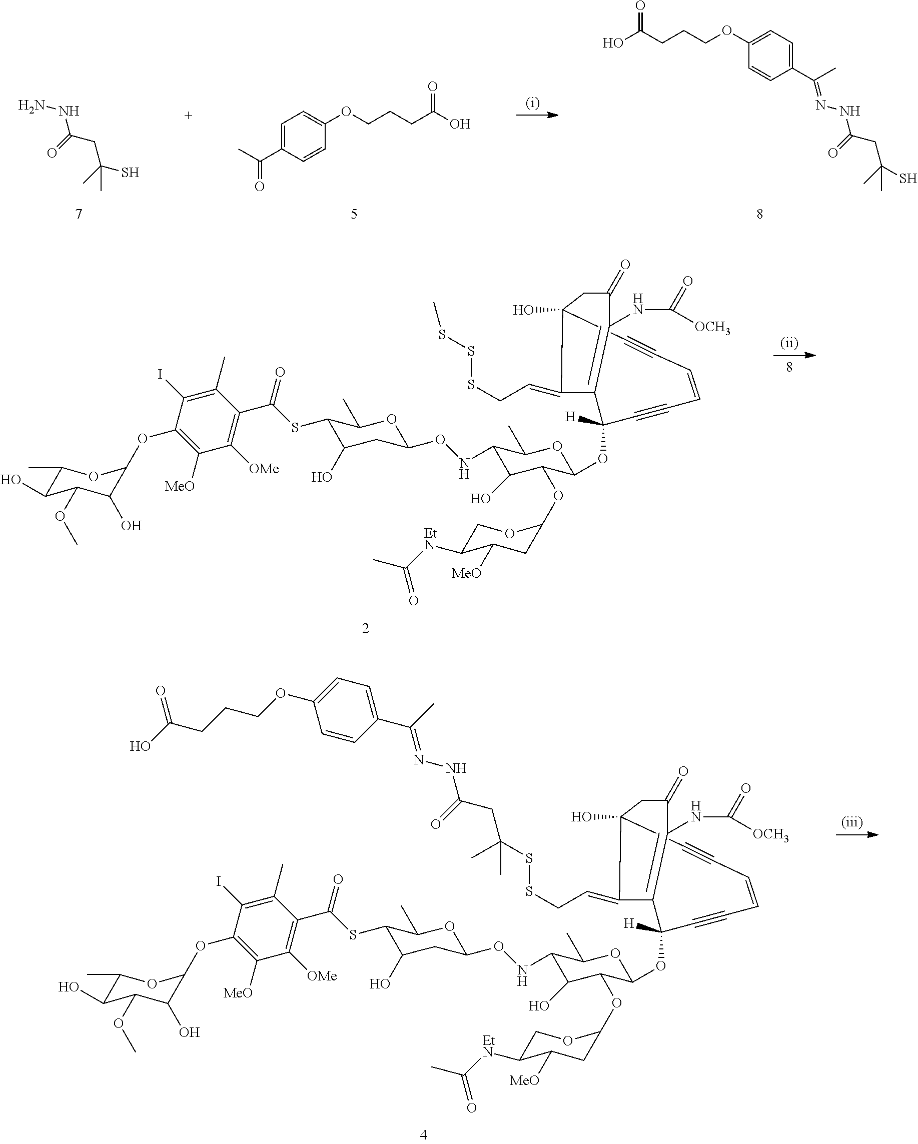

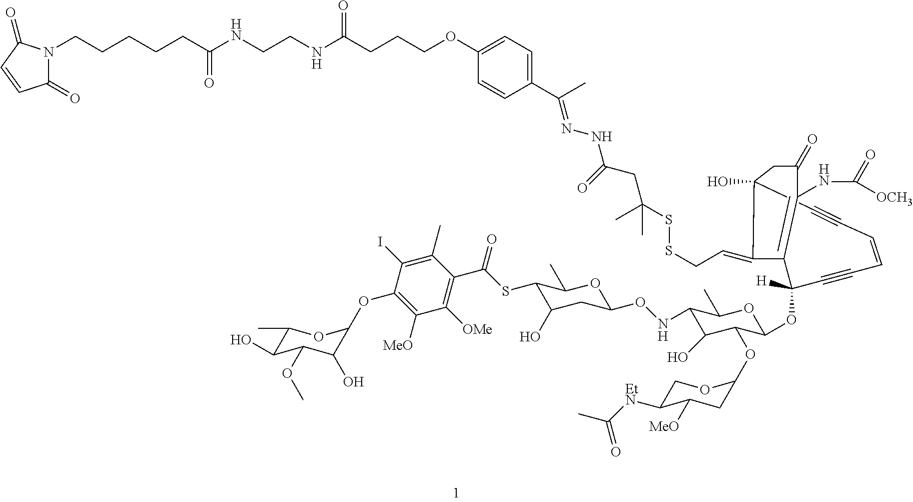

### (i) (E)-4-(4-(1-(2-(3-mercapto-3-methylbutanoyl)hydrazono)ethyl)phenoxy)butanoic acid (8)

4-(4-acetylphenoxy)butanoic acid (5, 750 mg, 3.37 mmol, 5 eq) was added to 3-mercapto-3-methylbutanehydrazide (7, 100 mg, 0.67 mmol) in DMF (5 mL). Acetic acid (3.0 mL, 80 eq) was added and the reaction was stirred at 37° C. for 3 days. After that time, 80% conversion was observed and the reaction was concentrated and purified by column chromatography (MeOH/DCM 1 to 20%) on silica gel column to afford 8 (7.0 mg, 3%). LCMS (analytical method A): Rt=1.74 min, [M+H]⁺=353.28.

### (ii) 4-(4-((E)-1-(2-(3-(((E)-2-((1R,8S,Z)-8-(((2R,3R,4R,5S,6R)-5-((((2S,4S,5S,6R)-5-((4-(((2S,3R,4R,5S,6S)-3,5-dihydroxy-4-methoxy-6-methyltetrahydro-2H-pyran-2-yl)oxy)-3-iodo-5,6-dimethoxy-2-methylbenzoyl)thio)-4-hydroxy-6-methyltetrahydro-2H-pyran-2-yl)oxy)amino)-3-(((2S,4S,5S)-5-(N-ethylacetamido)-4-methoxytetrahydro-2H-pyran-2-yl)oxy)-4-hydroxy-6-methyltetrahydro-2H-pyran-2-yl)oxy)-1-hydroxy-10-((methoxycarbonyl)amino)-11-oxobicyclo[7.3.1]trideca-4,9-dien-2,6-diyn-13-ylidene)ethyl)disulfanyl)-3-methylbutanoyl)hydrazono)ethyl)phenoxy)butanoic acid (4)

N-acetyl calicheamicin (2, 5 mg, 3.5 μmol) was dissolved in 50 μL of acetonitrile and chilled to −15 C. Compound 8 (6.2 mg, 17.7 μmol, 5 eq) was dissolved in 50 μL of acetonitrile and added slowly to the chilled solution of calicheamicin followed by addition of triethyl amine (2.3 μL, 17.7 μmol, 5 eq). The reaction was allowed to warm up until completion. After 3 hours, the reaction was concentrated and purified by column chromatography (MeOH/DCM 1 to 20%) on silica gel column to afford 4. LCMS (analytical method A) Rt=1.96 min, [M+H]⁺=1682.80.

### (iii)S-((2R,3S,4S,6S)-6-((((2R,3S,4R,5R,6R)-6-(((2S,5Z,9R,13E)-13-(2-((4-(2-((E)-1-(4-(4-((2-(6-(2,5-dioxo-2,5-dihydro-1H-pyrrol-1-yl)hexanamido)ethyl)amino)-4-oxobutoxy)phenyl)ethylidene)hydrazinyl)-2-methyl-4-oxobutan-2-yl)disulfanyl)ethylidene)-9-hydroxy-12-((methoxycarbonyl)amino)-11-oxobicyclo [7.3.1] trideca-1(12),5-dien-3,7-diyn-2-yl)oxy)-5-(((2S,4S,5S)-5-(N-ethylacetamido)-4-methoxytetrahydro-2H-pyran-2-yl)oxy)-4-hydroxy-2-methyltetrahydro-2H-pyran-3-yl)amino)oxy)-4-hydroxy-2-methyltetrahydro-2H-pyran-3-yl) 4-(((2S,3R,4R,5S,6S)-3,5-dihydroxy-4-methoxy-6-methyltetrahydro-2H-pyran-2-yl)oxy)-3-iodo-5,6-dimethoxy-2-methylbenzothioate (1)

See Synthesis route 1.

### Example 2

### Synthesis of a Calicheamicin Construct Comprising an Oxime Linker

A drug-linker compound according to Formula 14

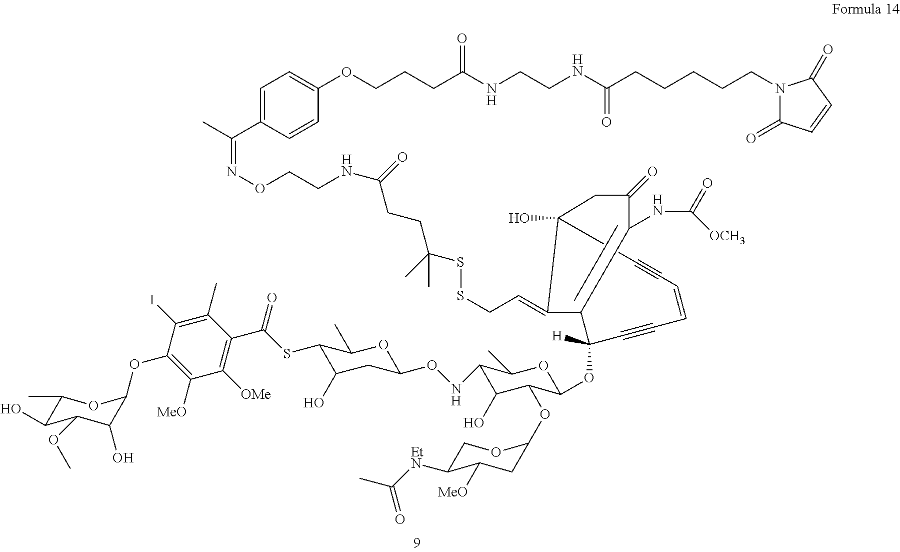

was synthesized as set forth immediately below.

Synthesis Part 1: Linker Formation

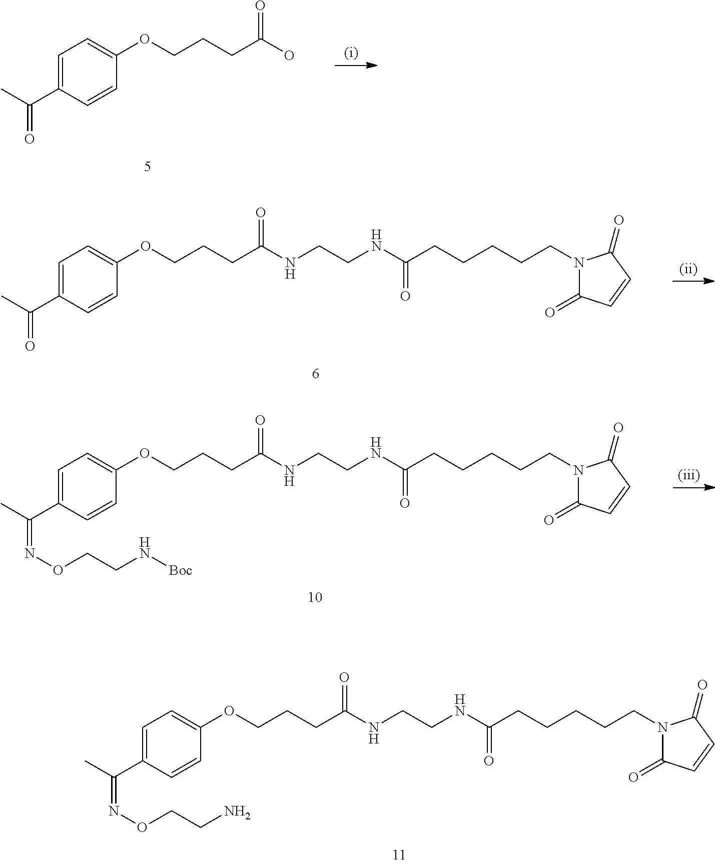 [SMILES: CC(=O)c1ccc(OCCCC(=O)O)cc1 ; CC(=O)c1ccc(OCCCC(=O)NCCNC(=O)CCCCCN2C(=O)C=CC2=O)cc1 ; C/C(=N/OCCNC(=O)OC(C)(C)C)c1ccc(OCCCC(=O)NCCNC(=O)CCCCCN2C(=O)C=CC2=O)cc1 ; C/C(=N/OCCN)c1ccc(OCCCC(=O)NCCNC(=O)CCCCCN2C(=O)C=CC2=O)cc1]

### (i) N-(2-(4-(4-acetylphenoxy)butanamido)ethyl)-6-(2,5-dihydro-1H-pyrrol-1-yl)hexanamide (6)

Same procedure as Example 1/Synthesis route 2

### (ii) tert-butyl (Z)-(2-(((1-(4-(4-((2-(6-(2,5-dioxo-2,5-dihydro-1H-pyrrol-1-yl)hexanamido)ethyl)amino)-4-oxobutoxy)phenyl)ethylidene)amino)oxy)ethyl)carbamate (10)

Tert-butyl (2-(aminooxy)ethyl)carbamate (46.2 mg, 0.26 mmol, 1.2 eq) was added to a solution of N-(2-(4-(4-acetylphenoxy)butanamido)ethyl)-6-(2,5-dioxo-2,5-dihydro-1H-pyrrol-1-yl)hexanamide 6 (100 mg, 0.22 mmol) in dimethylformamide (200 μL). The reaction was stirred at 40° C. for 15 hours. Reaction was concentrated and purification by column chromatography afforded the desired product 10 (65.5 mg, 50%) as a white solid. LCMS (analytical method A): Rt=1.92 min, [M+H]⁺=616.44.

### (iii) (Z)—N-(2-(4-(4-(1-((2-aminoethoxy)imino)ethyl)phenoxy)butanamido)ethyl)-6-(2,5-dioxo-2,5-dihydro-1H-pyrrol-1-yl)hexanamide (11)

tert-butyl (Z)-(2-(((1-(4-(4-((2-(6-(2,5-dioxo-2,5-dihydro-1H-pyrrol-1-yl)hexanamido)ethyl)amino)-4-oxobutoxy)phenyl)ethylidene)amino)oxy)ethyl)carbamate 10 was dissolved in a solution of 10% TFA in dichloromethane. The reaction mixture was stirred at room temperature for 1 hour before evaporation of the solvent. The resulting crude mixture was used in the next step.

Synthesis Part 2: Linker-Drug Fabrication

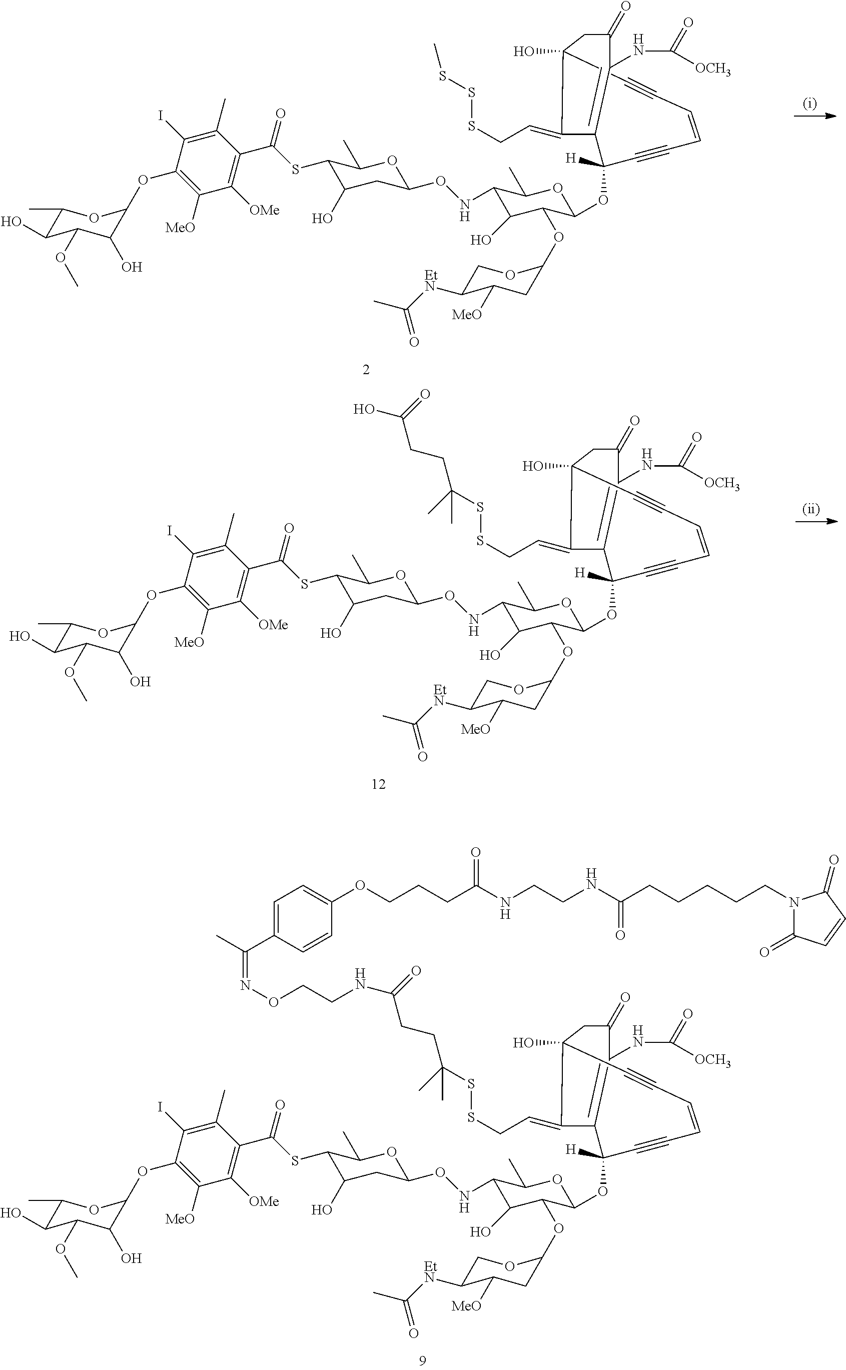

### (i) 4-(((E)-2-((1R,8S,Z)-8-(((2R,3R,4R,5S,6R)-5-((((2S,4S,5S,6R)-5-((4-(((2S,3R,4R,5S,6S)-3,5-dihydroxy-4-methoxy-6-methyltetrahydro-2H-pyran-2-yl)oxy)-3-iodo-5,6-dimethoxy-2-methylbenzoyl)thio)-4-hydroxy-6-methyltetrahydro-2H-pyran-2-yl)oxy)amino)-3-(((2S,4S,5S)-5-(N-ethylacetamido)-4-methoxytetrahydro-2H-pyran-2-yl)oxy)-4-hydroxy-6-methyltetrahydro-2H-pyran-2-yl)oxy)-1-hydroxy-10-((methoxycarbonyl)amino)-11-oxobicyclo[7.3.1]trideca-4,9-dien-2,6-diyn-13-ylidene)ethyl)disulfanyl)-4-methylpentanoic acid (12)

N-acetyl calicheamicin γ 1 (0.2 g, 0.142 mmol, 1 eq) was dissolved in 30 ml of acetonitrile and solution was chilled to −15° C. 4-mercapto-4-methylpentanoic acid (0.420 ml, 2.837 mmol, 20 eq) was dissolved in 10 ml of acetonitrile and added slowly to the cooled solution of N-acetyl calicheamicn. Triethylamine (0.377 ml, 2.837 mmol, 20 eq) was added to the reaction mixture and reaction was allowed to warm up to room temperature over 3-18h. Upon completion of the reaction, the mixture was concentrated and dry loaded onto silica gel for flash chromatography purification. Flash chromatography purification with 2-20% MeOH in DCM resulted in the isolation of desired product as glassy solid (0.19 g, 90.5% yield), that can be precipitated out of cold diethyl ether as white powder. LCMS (analytical method A): Rt=1.92 min, [M+H]⁺=1478.64.

### (ii)S-((2R,3S,4S,6S)-6-((((2R,3S,4R,5R,6R)-6-(((2S,5Z,9R,13E)-13-((Z)-2-(4-(4-((2-(6-(2,5-dioxo-2,5-dihydro-1H-pyrrol-1-yl)hexanamido)ethyl)amino)-4-oxobutoxy)phenyl)-11,11-dimethyl-8-oxo-4-oxa-12,13-dithia-3,7-diazapentadec-2-en-15-ylidene)-9-hydroxy-12-((methoxycarbonyl)amino)-11-oxobicyclo [7.3.1]trideca-1 (12),5-dien-3,7-diyn-2-yl)oxy)-5-(((2S,4S,5S)-5-(N-ethylacetamido)-4-methoxytetrahydro-2H-pyran-2-yl)oxy)-4-hydroxy-2-methyltetrahydro-2H-pyran-3-yl)amino)oxy)-4-hydroxy-2-methyltetrahydro-2H-pyran-3-yl) 4-(((2S,3R,4R,5S,6S)-3,5-dihydroxy-4-methoxy-6-methyltetrahydro-2H-pyran-2-yl)oxy)-3-iodo-5,6-dimethoxy-2-methylbenzothioate (9)

(Z)—N-(2-(4-(4-(1-((2-aminoethoxy)imino)ethyl)phenoxy)butanamido)ethyl)-6-(2,5-dioxo-2,5-dihydro-1H-pyrrol-1-yl)hexanamide 11 (2.1 mg, 4 μmol, 1.5 eq) was dissolved in 100 μL of dimethylformamide and 5 μL of DIPEA (10 eq) was added. This solution was then added to a solution of 12 (4.0 mg, 2.7 μmol) in 100 μL of dimethylformamide with 5 μL of DIPEA (10 eq). EDCI (2.6 mg, 13.5 μmol, 5 eq) and HOBt hydrate (4.1 mg, 27 μL, 10 eq) were added and the reaction was stirred at room temperature for 20 hours. Full conversion was observed and the reaction was concentrated before purification on preparative HPLC (preparative HPLC Method B) to give the desired product 9 (0.4 mg, 7.5%). LC/HRMS (analytical method C): Rt=9.06 min, M/Z observed for [M+2H]⁺=988.3195. ¹H NMR (500 MHz, Chloroform-d) δ 7.56 (d, J=8.8 Hz, 2H), 6.90 (d, J=8.8 Hz, 1H), 6.68 (s, 2H), 6.46 (s, 2H), 6.23 (d, J=65.9 Hz, 3H), 5.73 (s, 1H), 4.68 (d, J=11.6 Hz, 1H), 4.48 (s, 1H), 4.32 (s, 1H), 4.24 (s, 3H), 4.04 (q, J=6.3 Hz, 4H), 3.89 (s, 3H), 3.84 (s, 4H), 3.82-3.54 (m, 13H), 3.49 (t, J=7.1 Hz, 3H), 3.42-3.25 (m, 10H), 2.61 (d, J=17.7 Hz, 1H), 2.39 (d, J=21.7 Hz, 8H), 2.29 (d, J=7.6 Hz, 2H), 2.21 (s, 5H), 2.11 (d, J=8.0 Hz, 7H), 2.02 (s, 2H), 1.93 (s, 2H), 1.67-1.49 (m, 42H), 1.41 (d, J=6.3 Hz, 4H), 1.31 (d, J=6.2 Hz, 5H), 1.28-1.15 (m, 12H).

### Example 3

### Synthesis of a Calicheamicin Construct Comprising a Val-Cit Dipeptide Linker

A drug-linker compound according to Formula 4 (FIG. 1)

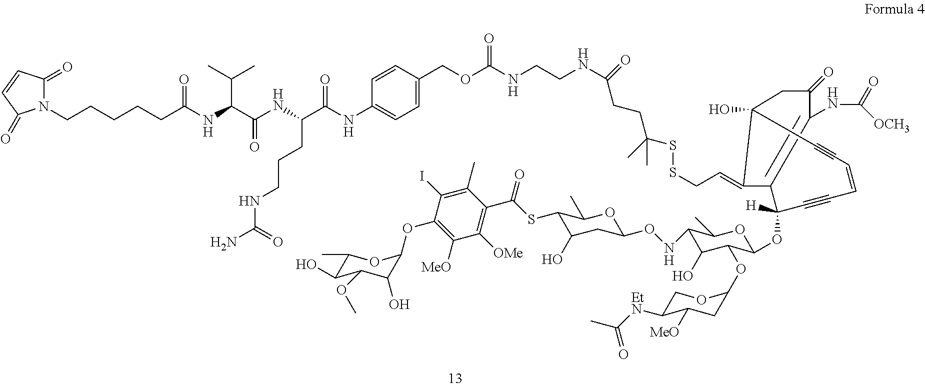

was synthesized as set forth immediately below.

Synthesis Part 1: Linker Formation

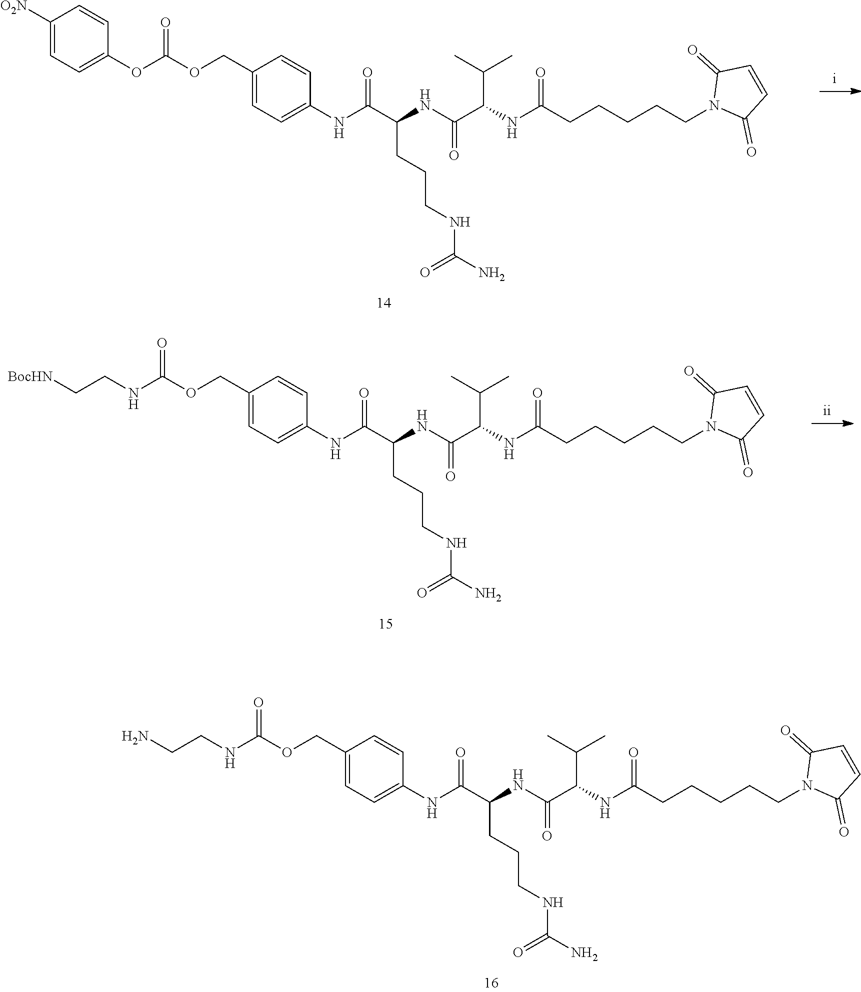 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CC(C)[C@H](NC(=O)CCCCCN1C(=O)C=CC1=O)C(=O)N[C@@H](CCCNC(N)=O)C(=O)Nc1ccc(COC(=O)NCCNC(=O)OC(C)(C)C)cc1 ; CC(C)[C@H](NC(=O)CCCCCN1C(=O)C=CC1=O)C(=O)N[C@@H](CCCNC(N)=O)C(=O)Nc1ccc(COC(=O)NCCN)cc1]

### 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate 14

Synthesis of 14 has been previously described (U.S. Pat. No. 6,214,345 B 1).

### tert-butyl (4-((S)-2-((S)-2-(6-(2,5-dioxo-2,5-dihydro-H-pyrrol-1-yl)hexanamido)-3-methylbutanamido)-5-ureidopentanamido)benzyl) ethane-1,2-diyldicarbamate 15

4-Nitrophenyl carbonate 14 (100 mg, 0.136 mmol, 1 eq) was dissolved in 5 ml anhydrous DMF, cooled to 0° C. and treated with tert-butyl (2-aminoethyl)carbamate (21.4 uL, 0.136 mmol, 1 eq). Reaction mixture was stirred for 2h, concentrated and purified by column chromatography (gradient 2-50% MeOH/DCM) to result in off-white solid (55 mg, 53%). LCMS (analytical method A): Rt=1.73 min, [M+H]⁺=759.38.

### 4-((S)-2-((S)-2-(6-(2,5-dioxo-2,5-dihydro-1H-pyrrol-1-yl)hexanamido)-3-methylbutanamido)-5-ureidopentanamido)benzyl (2-aminoethyl)carbamate 16

Boc-amine linker 15 (50 mg, 0.066 mmol, 1 eq) was dissolved in 10% TFA/DCM solution (5 ml) and stirred at room temperature for 30 min. Reaction completion was confirmed by LCMS and the solvent was removed in vacuo. Resulting TFA salt of free amine was used for the next step immediately. LCMS (analytical method A): Rt=1.36 min, [M+H]⁺=659.52.

Synthesis Part 2: Drug-Linker Fabrication

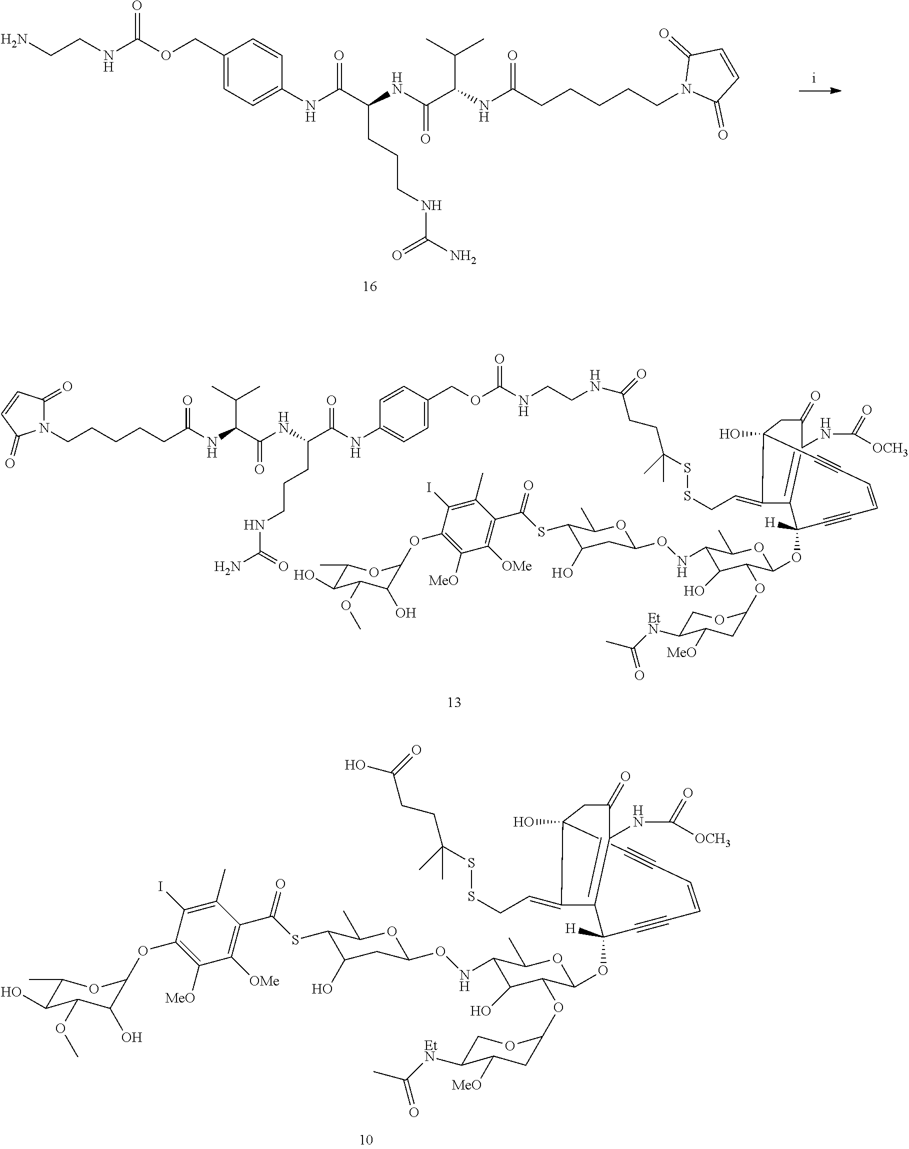

### S-((2R,3S,4S,6S)-6-((((2R,3S,4R,5R,6R)-6-(((2S,5Z,9R,13E)-13-(1-(4-((S)-2-((S)-2-(6-(2,5-dioxo-2,5-dihydro-1H-pyrrol-1-yl)hexanamido)-3-methylbutanamido)-5-ureidopentanamido)phenyl)-11,11-dimethyl-3,8-dioxo-2-oxa-12,13-dithia-4,7-diazapentadecan-15-ylidene)-9-hydroxy-12-((methoxycarbonyl)amino)-11-oxobicyclo[7.3.1]trideca-1(12),5-dien-3,7-diyn-2-yl)oxy)-5-(((2S,4S,5S)-5-(N-ethylacetamido)-4-methoxytetrahydro-2H-pyran-2-yl)oxy)-4-hydroxy-2-methyltetrahydro-2H-pyran-3-yl)amino)oxy)-4-hydroxy-2-methyltetrahydro-2H-pyran-3-yl) 4-(((2S,3R,4R,5S,6S)-3,5-dihydroxy-4-methoxy-6-methytetrahydro-2H-pyran-2-yl)oxy)-3-iodo-5,6-dimethoxy-2-methylbenzothioate 13

Calicheamicin-acid derivative 10 (108 mg, 0.073 mmol 1 eq) was dissolved in 20 ml of dry DMF followed by addition of EDCI (140.1 mg, 0.731 mmol, 10 eq), HOBt (111.8 mg, 0.731 mmol, 10 eq) and dry DIPEA (0.253 ml, 1.46 mmol, 20 eq). Reaction was stirred at room temperature for 10 min. Linker amine 16 (144.2 mg, 0.219 mmol, 3 eq) was dissolved in 3 ml of dry DMF and dry DIPEA(0.253 ml, 1.46 mmol, 20 eq), was added to the linker solution. Linker-amine solution was then added to the activated acid solution. Reaction was stirred at 37° C. overnight and monitored by LCMS. Upon reaction completion, DMF was removed in vacuo and obtained residue was dissolved in 1:1 acetonitrile:water for preparative HPLC purification (Method A). Desired product was isolated by preparative HPLC method A as white powder (20 mg, 12.9%). LCMS: Rt (analytical method A or C)=8.52 min, M/Z observed for [M+H]⁺=2118.7134. ¹H NMR (500 MHz, Chloroform-d) δ 7.52 (d, J=8.1 Hz, 2H), 7.26 (d, J=8.1 Hz, 2H), 6.95-6.86 (m, 2H), 6.68 (s, 2H), 6.44-6.36 (m, 1H), 6.23 (s, 1H), 5.91 (d, J=9.4 Hz, 1H), 5.82-5.73 (m, 2H), 5.67 (d, J=1.7 Hz, 2H), 5.03 (dd, J=16.4, 7.7 Hz, 4H), 4.73-4.49 (m, 5H), 4.46 (d, J=2.9 Hz, 1H), 4.27 (s, 2H), 4.24-4.14 (m, 3H), 3.88 (s, 4H), 3.83 (d, J=2.5 Hz, 4H), 3.81 (d, J=3.2 Hz, 1H), 3.77-3.59 (m, 9H), 3.57 (s, 4H), 3.49 (q, J=8.2, 7.4 Hz, 3H), 3.42-3.20 (m, 13H), 3.18-3.04 (m, 3H), 2.44-2.33 (m, 6H), 2.29 (t, J=9.8 Hz, 2H), 2.23 (t, J=7.2 Hz, 3H), 2.20-1.96 (m, 31H), 1.87 (d, J=7.2 Hz, 4H), 1.80-1.47 (m, 11H), 1.46-1.35 (m, 5H), 1.35-1.14 (m, 18H), 0.92 (dd, J=6.7, 3.2 Hz, 6H).

### Example 4

### Synthesis of a Calicheamicin Construct Comprising a Val-Ala Dipeptide Linker

A drug-linker compound according to Formula 5

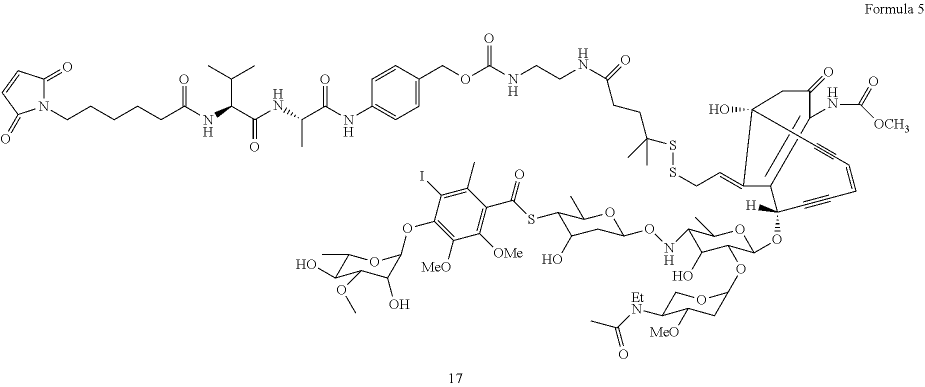

was synthesized as set forth immediately below.

Synthesis Part 1: Linker Formation

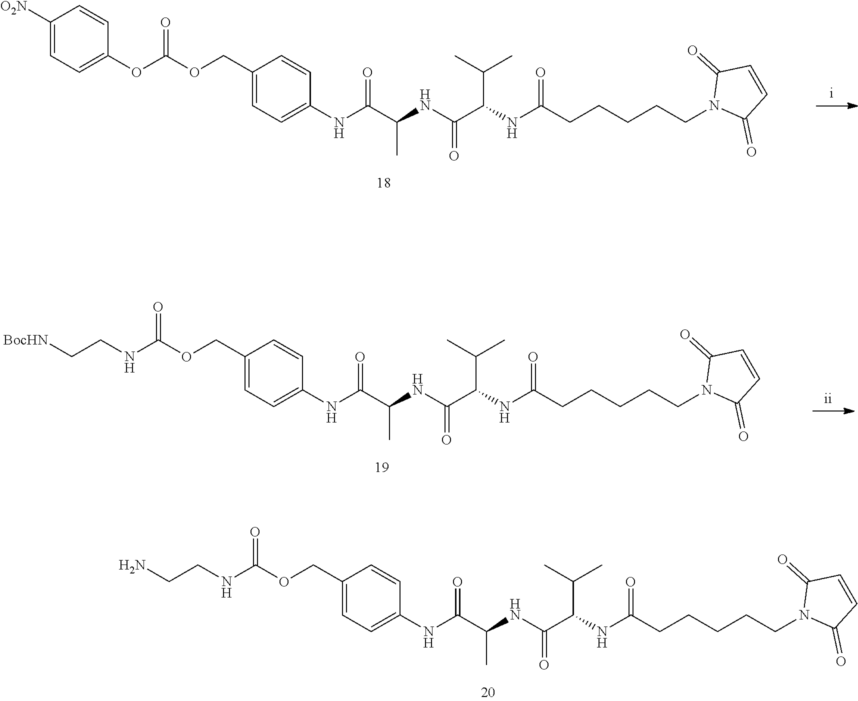 [SMILES: CC(C)[C@H](NC(=O)CCCCCN1C(=O)C=CC1=O)C(=O)N[C@@H](C)C(=O)Nc1ccc(COC(=O)Oc2ccc([N+](=O)[O-])cc2)cc1 ; CC(C)[C@H](NC(=O)CCCCCN1C(=O)C=CC1=O)C(=O)N[C@@H](C)C(=O)Nc1ccc(COC(=O)NCCNC(=O)OC(C)(C)C)cc1 ; CC(C)[C@H](NC(=O)CCCCCN1C(=O)C=CC1=O)C(=O)N[C@@H](C)C(=O)Nc1ccc(COC(=O)NCCN)cc1]

### 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate 18

Synthesis of 4-nitrophenyl carbonate 18 has been previously described (U.S. Pat. No. 6,214,345 B 1).

### tert-butyl (4-((S)-2-((S)-2-(6-(2,5-dioxo-2,5-dihydro-1H-pyrrol-1-yl)hexanamido)-3-methylbutanamido)propanamido)benzyl) ethane-1,2-diyldicarbamate 19

Synthesis was performed using the same synthetic procedure as for the preparation of linker 15. Isolated yield 68 mg (63%), LCMS (analytical method A): Rt=1.85 min, [M+H]⁺=673.39.

### 4-((S)-2-((S)-2-(6-(2,5-dioxo-2,5-dihydro-1H-pyrrol-1-yl)hexanamido)-3-methylbutanamido)propanamido)benzyl (2-aminoethyl)carbamate 20

Synthetic procedure same as for the preparation of linker 16. LCMS (analytical method A): Rt=1.38 min, [M+H]⁺=573.44.

Synthesis Part 2. Drug-Linker Fabrication

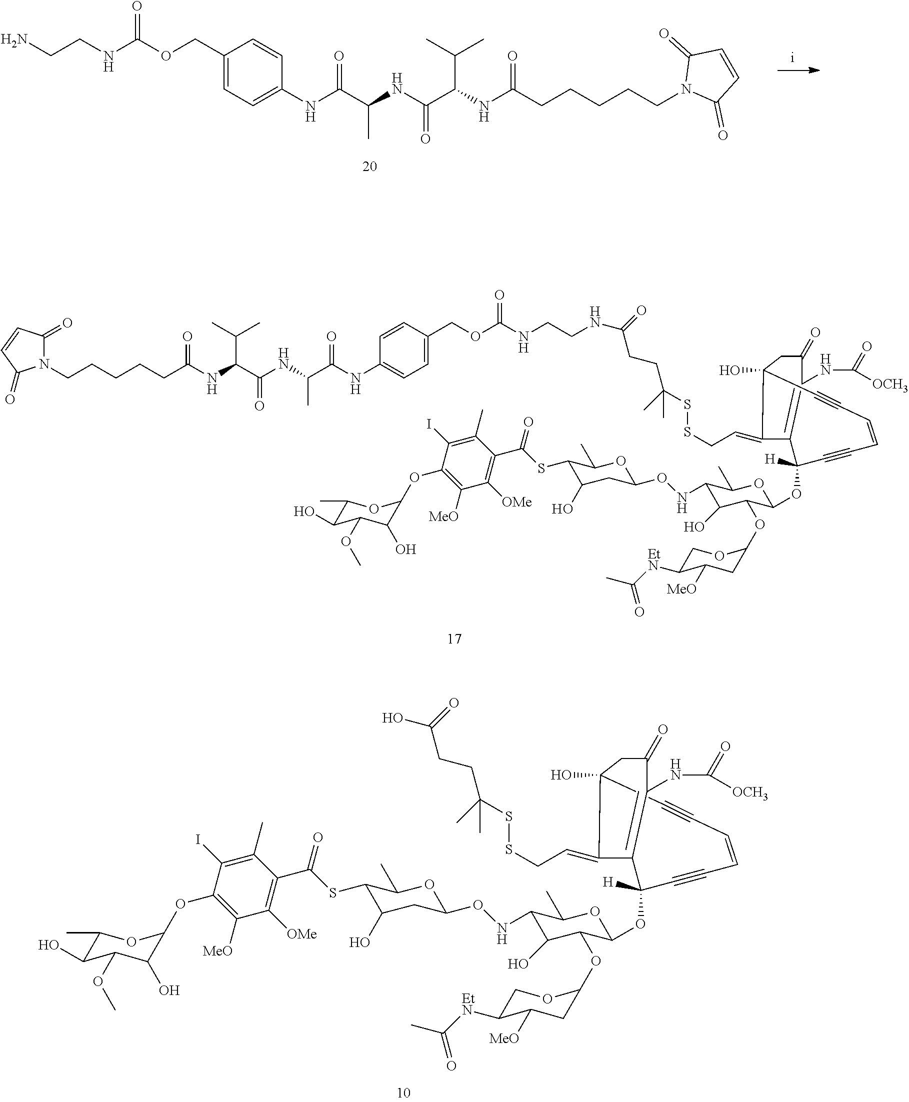

### S-((2R,3S,4S,6S)-6-((((2R,3S,4R,5R,6R)-6-(((2S,5Z,9R,13E)-13-(1-(4-((S)-2-((S)-2-(6-(2,5-dioxo-2,5-dihydro-1H-pyrrol-1-yl)hexanamido)-3-methylbutanamido)propanamido)phenyl)-11,11-dimethyl-3,8-dioxo-2-oxa-12,13-dithia-4,7-diazapentadecan-15-ylidene)-9-hydroxy-12-((methoxycarbonyl)amino)-11-oxobicyclo [7.3.1]trideca-1 (12),5-dien-3,7-diyn-2-yl)oxy)-5-(((2S,4S,5S)-5-(N-ethylacetamido)-4-methoxytetrahydro-2H-pyran-2-yl)oxy)-4-hydroxy-2-methyltetrahydro-2H-pyran-3-yl)amino)oxy)-4-hydroxy-2-methyltetrahydro-2H-pyran-3-yl) 4-(((2S,3R,4R,5S,6S)-3,5-dihydroxy-4-methoxy-6-methyltetrahydro-2H-pyran-2-yl)oxy)-3-iodo-5,6-dimethoxy-2-methylbenzothioate 17

Synthetic procedure same as for the preparation of 13. Isolated as white solid. LCMS (analytical method B or C) Rt=8.93 min, LC/HRMS M/Z observed for [M+2H]+=1016.8810 ¹H NMR (500 MHz, Chloroform-d) δ 7.50 (d, J=8.2 Hz, 3H), 7.26 (s, 4H), 6.68 (s, 2H), 6.25 (s, 3H), 5.83-5.75 (m, 2H), 5.73 (s, 2H), 5.64 (d, J=14.1 Hz, 2H), 5.06 (t, J=12.7 Hz, 4H), 4.75-4.52 (m, 6H), 4.48 (s, 2H), 4.32 (s, 3H), 4.20 (dd, J=9.4, 6.1 Hz, 2H), 4.05 (s, 4H), 3.89 (s, 4H), 3.84 (d, J=1.6 Hz, 5H), 3.77 (dd, J=15.6, 9.1 Hz, 4H), 3.73-3.60 (m, 7H), 3.58 (s, 4H), 3.49 (t, J=7.2 Hz, 4H), 3.43-3.22 (m, 13H), 3.18 (d, J=17.2 Hz, 2H), 2.98 (s, 3H), 2.37 (s, 7H), 2.33-2.13 (m, 8H), 2.10 (s, 7H), 1.90 (s, 3H), 1.59 (s, 19H), 1.50-1.37 (m, 10H), 1.35-1.13 (m, 22H), 0.93 (d, J=6.8 Hz, 7H).

### Example 5

### Synthesis of a Calicheamicin Construct Comprising a Bis-Val-Cit Dipeptide Linker

A drug-linker compound according to Formula 15

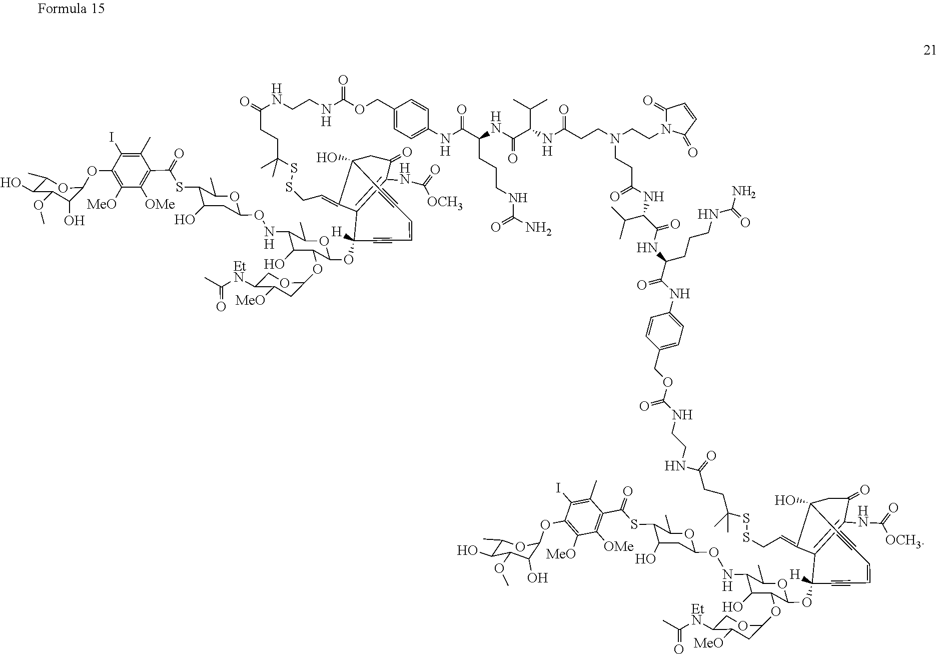

was synthesized as set forth immediately below.

Synthesis Part 1: Linker Formation.

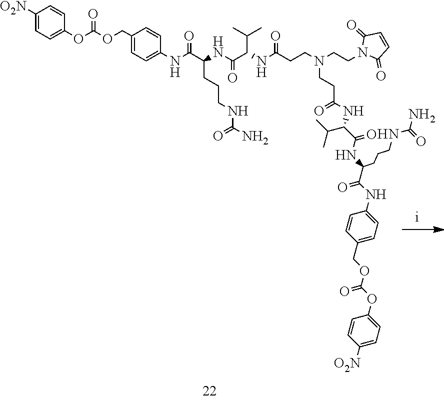 [SMILES: CC(C)[C@H](NC(=O)CCN(CCC(=O)N[C@H](C(=O)N[C@@H](CCCNC(N)=O)C(=O)Nc1ccc(COC(=O)Oc2ccc([N+](=O)[O-])cc2)cc1)C(C)C)CCN1C(=O)C=CC1=O)C(=O)N[C@@H](CCCNC(N)=O)C(=O)Nc1ccc(COC(=O)Oc2ccc([N+](=O)[O-])cc2)cc1]

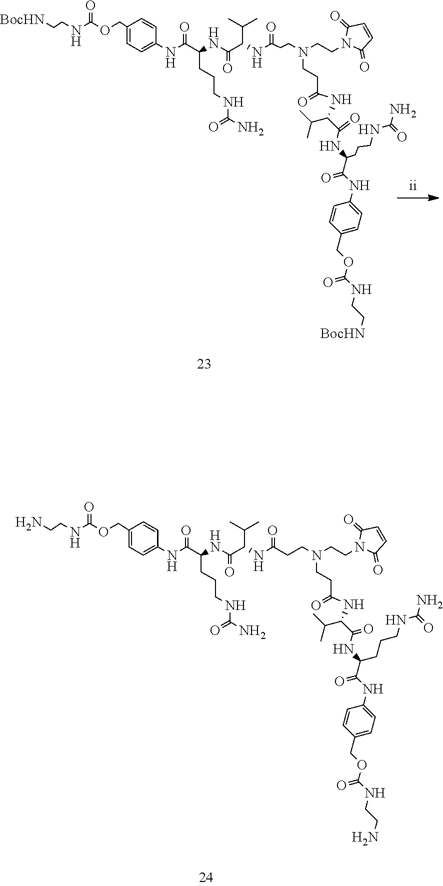 [SMILES: CC(C)[C@H](NC(=O)CCN(CCC(=O)N[C@H](C(=O)N[C@@H](CCCNC(N)=O)C(=O)Nc1ccc(COC(=O)NCCNC(=O)OC(C)(C)C)cc1)C(C)C)CCN1C(=O)C=CC1=O)C(=O)N[C@@H](CCCNC(N)=O)C(=O)Nc1ccc(COC(=O)NCCNC(=O)OC(C)(C)C)cc1 ; CC(C)[C@H](NC(=O)CCN(CCC(=O)N[C@H](C(=O)N[C@@H](CCCNC(N)=O)C(=O)Nc1ccc(COC(=O)NCCN)cc1)C(C)C)CCN1C(=O)C=CC1=O)C(=O)N[C@@H](CCCNC(N)=O)C(=O)Nc1ccc(COC(=O)NCCN)cc1]

### ((((2S,5S,15S,18S)-10-(2-(2,5-dioxo-2,5-dihydro-1H-pyrrol-1-yl)ethyl)-5,15-diisopropyl-4,7,13,16-tetraoxo-2,18-bis(3-ureidopropyl)-3,6,10,14,17-pentaazanonadecanedioyl)bis(azanediyl))bis(4,1-phenylene))bis(methylene) bis(4-nitrophenyl) bis(carbonate) 22

Synthesis of 4-nitrophenyl carbonate 22 was accomplished similarly to carbonate 18.

### Bis tert-butyl carboxylate of ((((2S,5S,15S,18S)-10-(2-(2,5-dioxo-2,5-dihydro-1H-pyrrol-1-yl)ethyl)-5,15-diisopropyl-4,7,13,16-tetraoxo-2,18-bis(3-ureidopropyl)-3,6,10,14,17-pentaazanonadecanedioyl)bis(azanediyl))bis(4,1-phenylene))bis(methylene) bis((2-aminoethyl)carbamate) 23

Synthesis was performed using the same synthetic procedure as for the preparation of linker 15 and 19. Isolated yield 13 mg (51%), LCMS (analytical method A): Rt=1.68 min, [M+H]⁺=1379.85.

### ((((2S,5S,15S,18S)-10-(2-(2,5-dioxo-2,5-dihydro-1H-pyrrol-1-yl)ethyl)-5,15-diisopropyl-4,7,13,16-tetraoxo-2,18-bis(3-ureidopropyl)-3,6,10,14,17-pentaazanonadecanedioyl)bis(azanediyl))bis(4,1-phenylene))bis(methylene) bis((2-aminoethyl)carbamate) 24

Synthetic procedure same as for the preparation of linker 16 and 20. LCMS (analytical method A): Rt=1.52 min, [M+H]⁺=1179.67.

Synthesis Part 2. Linker-Drug Fabrication

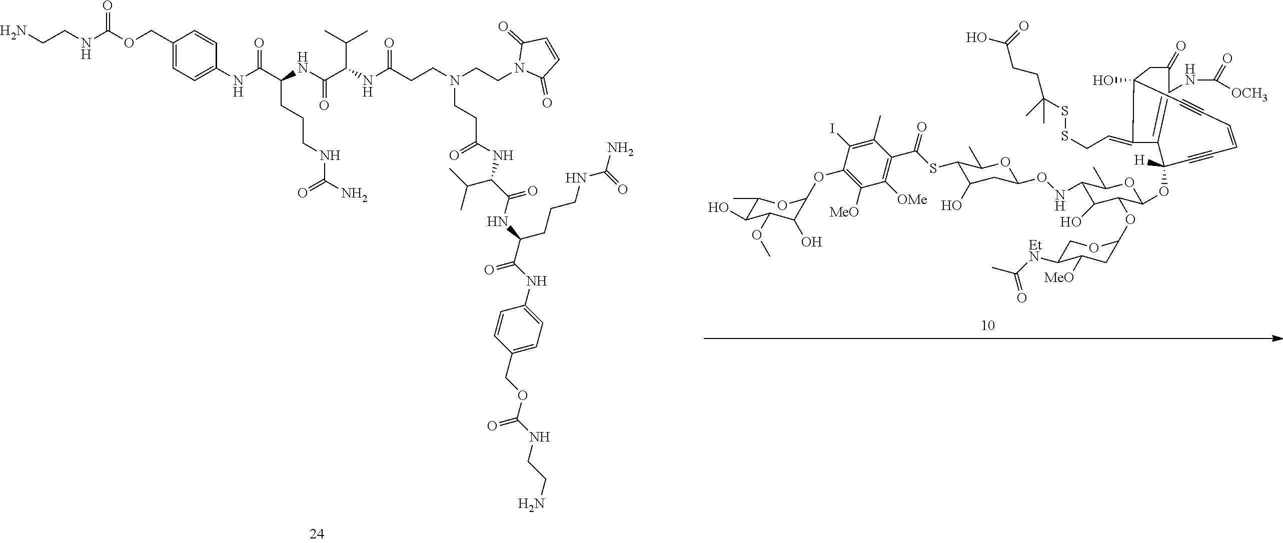

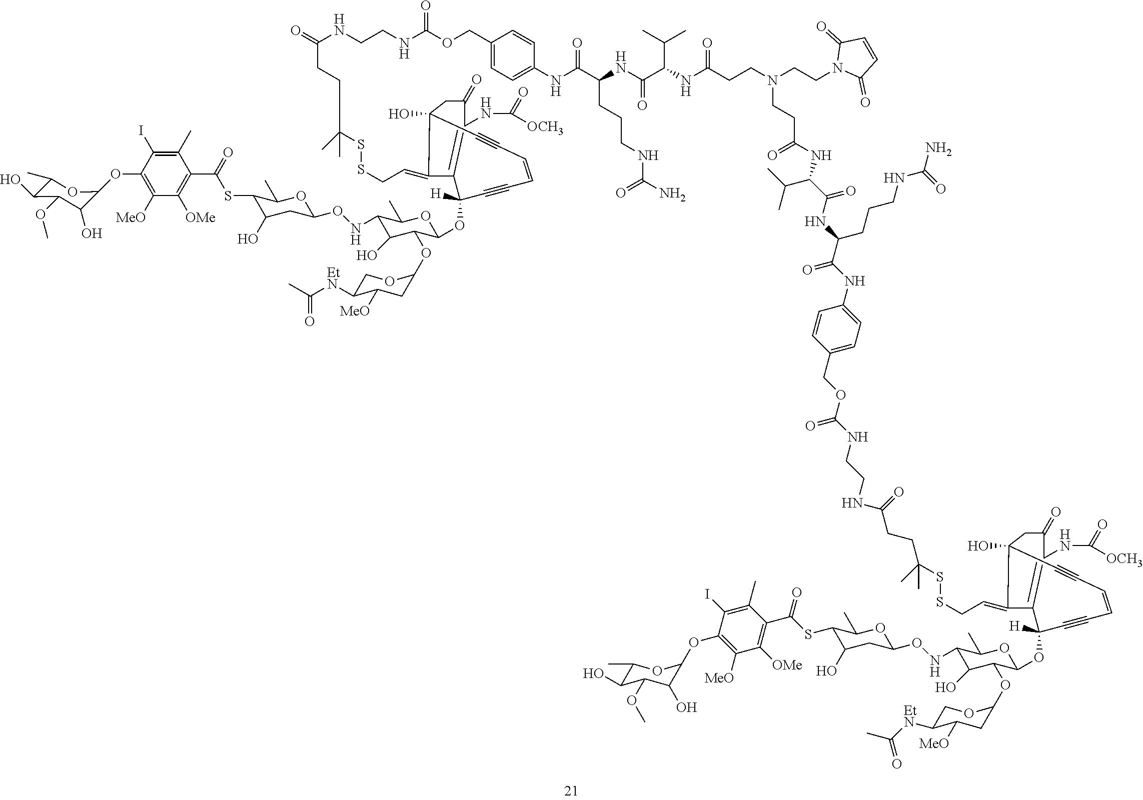

Maleimido Bis-Val-Cit-PABA-Calicheamicin gamma 1 derivative 21.

Synthetic procedure same as for the preparation of 13 and 17. Isolated as white solid. LCMS (analytical method B or C) Rt=7.80 min, LC/HRMS M/Z observed for [M+3H]⁺=1366.7897.

### Example 6

### Synthesis of Calicheamicin Linker-Drugs Comprising Val-Cit Dipeptide Linkers with Variable PEG Spacers

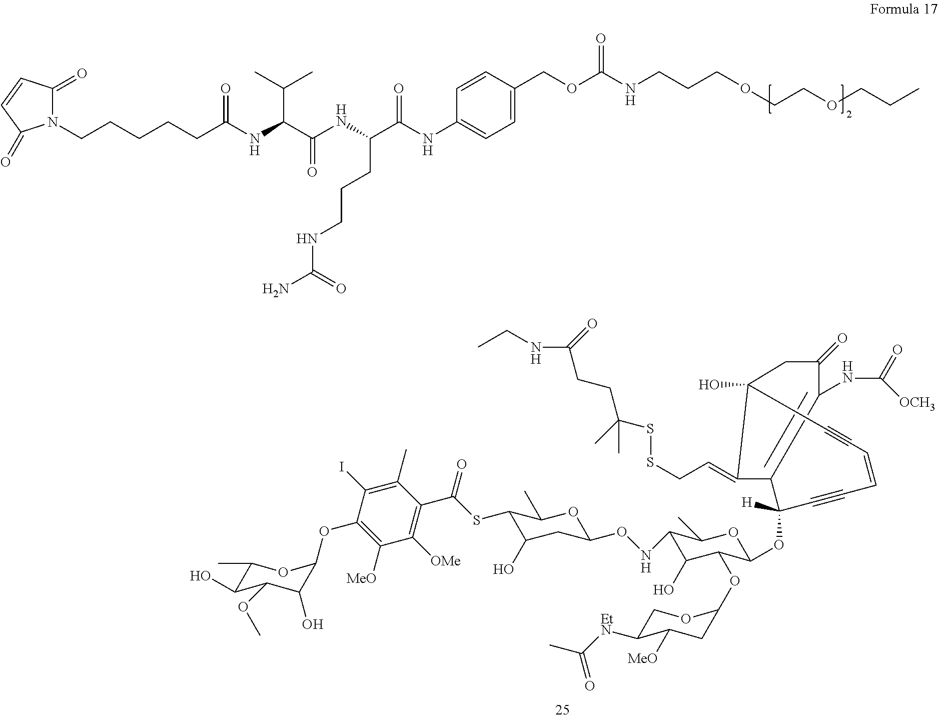

Was synthesized as set forth immediately below

Synthesis Part 1: Linker Formation.

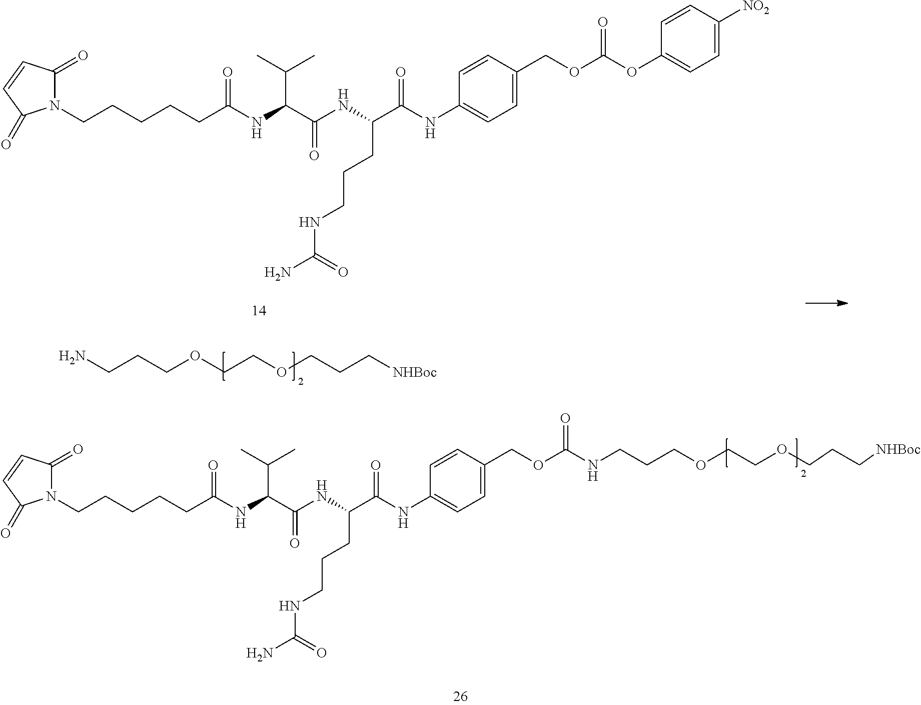

### 4-((S)-2-((S)-2-(6-(2,5-dioxo-2,5-dihydro-1H-pyrrol-1-yl)hexanamido)-3-methylbutanamido)-5-ureidopentanamido)benzyl (2,2-dimethyl-4-oxo-3,9,12,15-tetraoxa-5-azaoctadecan-18-yl)carbamate (26)

Synthesis was performed using the same synthetic procedure as for the preparation of linker 15 LCMS (analytical method A): Rt=1.81 min, [M+H]⁺=919.36.

### 4-((S)-2-((S)-2-(6-(2,5-dioxo-2,5-dihydro-1H-pyrrol-1-yl)hexanamido)-3-methylbutanamido)-5-ureidopentanamido)benzyl (3-(2-(2-(3-aminopropoxy)ethoxy)ethoxy)propyl)carbamate (27)

Synthetic procedure same as for the preparation of linker 16. LCMS (analytical method A): Rt=1.46 min, M/Z observed for [M+H]⁺=819.36.

Synthesis Part 2. Drug-Linker Fabrication

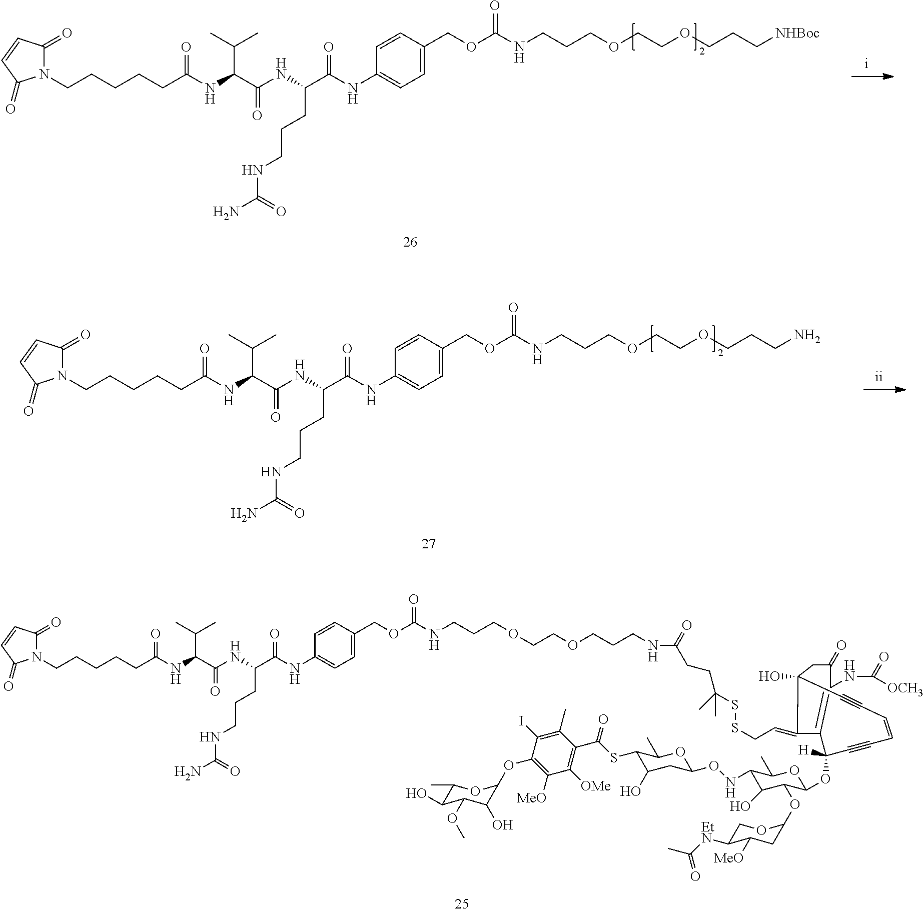

### S-((2R,3S,4S,6S)-6-(((2R,3S,4R,5R,6R)-6-(((2S,5Z,9R,13Z)-13-(1-(4-((S)-2-((S)-2-(6-(2,5-dioxo-2,5-dihydro-1H-pyrrol-1-yl)hexanamido)-3-methylbutanamido)-5-ureidopentanamido)phenyl)-22,22-dimethyl-3,19-dioxo-2,8,11,14-tetraoxa-23,24-dithia-4,18-diazahexacosan-26-ylidene)-9-hydroxy-12-((methoxycarbonyl)amino)-11-oxobicyclo[7.3.1]trideca-1(12),5-dien-3,7-diyn-2-yl)oxy)-5-(((2S,4S,5S)-5-(N-ethylacetamido)-4-methoxytetrahydro-2H-pyran-2-yl)oxy)-4-hydroxy-2-methyltetrahydro-2H-pyran-3-yl)amino)oxy)-4-hydroxy-2-methyltetrahydro-2H-pyran-3-yl) 4-(((2S,3R,4R,5S,6S)-3,5-dihydroxy-4-methoxy-6-methyltetrahydro-2H-pyran-2-yl)oxy)-3-iodo-5,6-dimethoxy-2-methylbenzothioate (25)

Synthetic procedure same as for the preparation of 13 and 17. Isolated as white solid. LCMS (analytical method B or C) Rt=8.62 min, LC/HRMS M/Z observed for [M+2H]⁺=1139.9088.

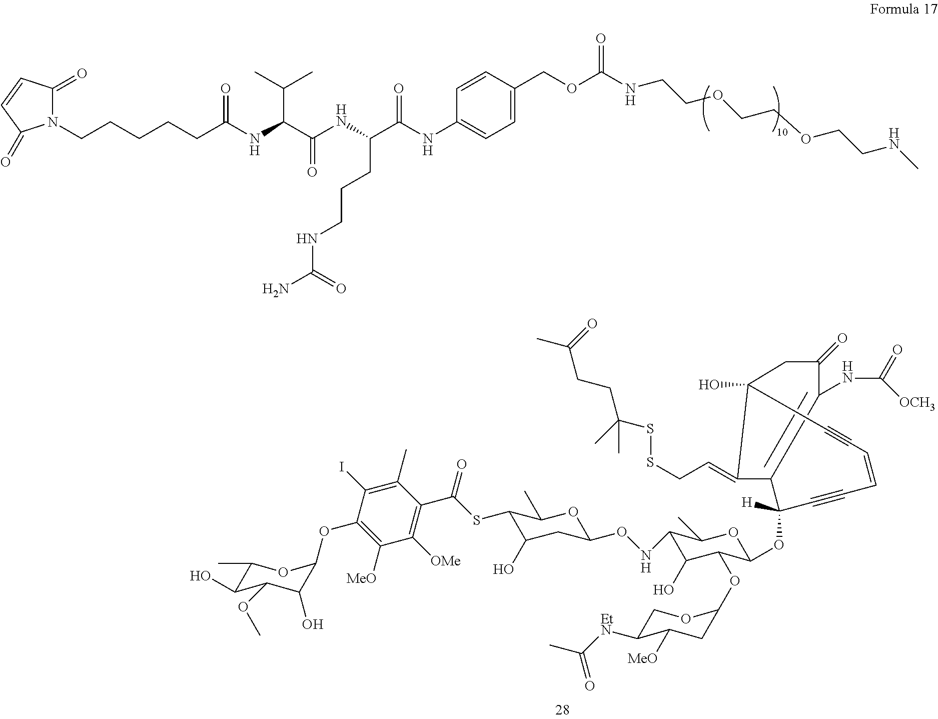

Was synthesized as set forth immediately below.

Synthesis Part 1: Linker Formation.

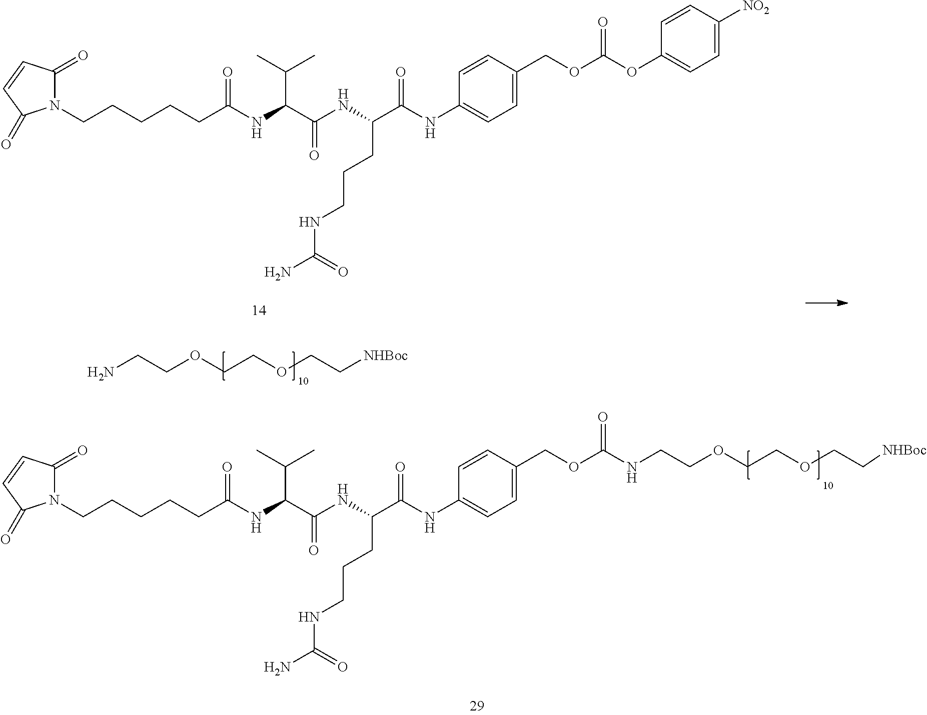

tert-butyl (4-((S)-2-((S)-2-(6-(2,5-dioxo-2,5-dihydro-1H-pyrrol-1-yl)hexanamido)-3-methylbutanamido)-5-ureidopentanamido)benzyl) (3,6,9,12,15,18,21,24,27,30,33-undecaoxapentatriacontane-1,35-diyl)dicarbamate (29).

Synthesis was performed using the same synthetic procedure as for the preparation of linker 15 LCMS (analytical method A): Rt=1.80 min, M/Z observed for [M+H]⁺=1243.69.

### 4-((S)-2-((S)-2-(6-(2,5-dioxo-2,5-dihydro-1H-pyrrol-1-yl)hexanamido)-3-methylbutanamido)-5-ureidopentanamido)benzyl (35-amino-3,6,9,12,15,18,21,24,27,30,33-undecaoxapentatriacontyl)carbamate (30)

Synthetic procedure same as for the preparation of linker 16. LCMS (analytical method A): Rt=1.50 min, M/Z observed for [M+H]⁺=1143.50.

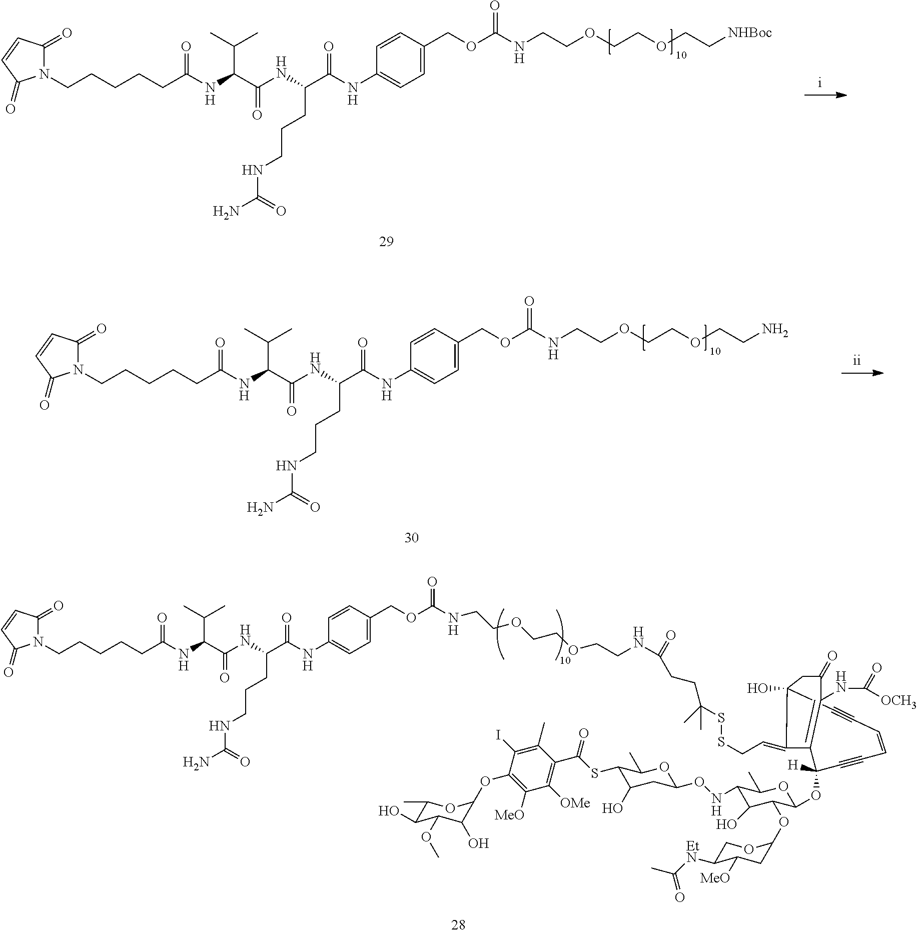

Synthesis Part 2. Drug-Linker Fabrication

### S-((2R,3S,4S,6S)-6-((((2R,3S,4R,5R,6R)-6-(((2S,5Z,9R,13Z)-13-(1-(4-((S)-2-((S)-2-(6-(2,5-dioxo-2,5-dihydro-1H-pyrrol-1-yl)hexanamido)-3-methylbutanamido)-5-ureidopentanamido)phenyl)-44,44-dimethyl-3,41-dioxo-2,7,10,13,16,19,22,25,28,31,34,37-dodecaoxa-45,46-dithia-4,40-diazaoctatetracontan-48-ylidene)-9-hydroxy-12-((methoxycarbonyl)amino)-11-oxobicyclo [7.3.1]trideca-1 (12),5-dien-3,7-diyn-2-yl)oxy)-5-(((2S,4S,5S)-5-(N-ethylacetamido)-4-methoxytetrahydro-2H-pyran-2-yl)oxy)-4-hydroxy-2-methyltetrahydro-2H-pyran-3-yl)amino)oxy)-4-hydroxy-2-methyltetrahydro-2H-pyran-3-yl) 4-(((2S,3R,4R,5S,6S)-3,5-dihydroxy-4-methoxy-6-methyltetrahydro-2H-pyran-2-yl)oxy)-3-iodo-5,6-dimethoxy-2-methylbenzothioate (28)

Synthetic procedure same as for the preparation of 13 and 17. Isolated as white solid. LCMS (analytical method B or C) Rt=8.61 min, LC/HRMS M/Z observed for [M+2H]⁺=1302.0007.

### Example 7

### Dipeptide Linker—Calicheamicin Constructs are Efficiently Cleaved In Vitro

A Cathepsin B assay was performed in order to demonstrate that the dipeptide linker-drug constructs made in the previous Examples were susceptible to enzymatic cleavage. Initially linker drug constructs from Examples 3 and 4 were treated with IM N-acetyl cysteine solution to quench maleimide functionality prior to the cathepsin B treatment. Excess of N-acetyl cysteine is used to activate the Cathepsin B.

More specifically quenched linker-drugs in 1:1 acetonitrile:water solution (20 ul) were diluted with 20 mM HisC1 pH 6.0 to a final acetonitrile content of 10% v/v (80 ul). Cathepsin B enzyme was added to result in 2.5 mol %, 5 mol % or 10 mol % enzyme relative to the linker-drug. Reactions were vortexed gently and kept at room temperature. At the time points of 15, 30, 60 and 90 min 3 ul of the reaction mixture were placed into the total recovery vial containing 5 uL Tris pH 9, 2 uL 100 mM dihydroxy ascorbic acid (DHAA), 15 ul water. The samples were analyzed using Analytical Method B set forth above. Conversion was calculated based on the decrease of the starting material peak area. The results are shown in FIG. 2A-2C.

The Val-Cit calicheamicin construct was generally cleaved more rapidly than the Val-Ala dipeptidyl construct though both constructs were effectively cleaved by Cathepsin B. Release of the expected payload amine was confirmed by mass spectrometry characterization showing [MH]+ ion of 1520.53. Under ionization conditions approximating those found in a cell the product of disulfide bond cleavage and rearrangement was also observed (FIG. 1) as [MH]+ ion of 1334.39.

The aforementioned results clearly demonstrate that the linker cleavage process provides for clean release of the calicheamicin payload. This data indicates the exemplary dipeptidyl drug linkers possess desirable therapeutic characteristics and may be effectively incorporated in the disclosed antibody drug conjugates.

### Example 8

### Calicheamicin—Linker Constructs Exhibit Therapeutically Effective Cytotoxicity In Vitro

Further assays were run to demonstrate that calicheamicin-linker constructs such as those described above retained cell killing capability and could function as part of an antibody drug conjugate. 293 T cells were used along with MES SA and MES SA/Dx cells which comprise uterine sarcoma lines purchased from ATCC. The MES SA/Dx cell line was generated from MES SA cells cultured with increasing amounts of doxorubicin resulting in 100-fold resistance to doxorubicin and upregulation of MDR1. Besides doxorubicin MES-SA/Dx cells exhibit marked cross-resistance to a number of other chemotherapeutic agents (including daunorubicin, dactinomycin, vincristine, taxol, colchicine) as well as moderate cross-resistance to mitomycin C and melphalan.

The cells were cultured in a T75 flask to ˜50-80% confluency and harvested with trypsin into a single cell suspension. Five hundred (500) cells per well were seeded in tissue culture plates in 50 μL/well culture media and incubated at 37° C. for 18-24 hours. Compounds were diluted as 400× final desired concentrations in DMSO. Serial dilutions in DMSO were then diluted in culture media for a final DMSO concentration of 0.25% and 50 μL/well of the final dilution was added to the cells (Vf=100 μL). Upon plating and treatment, cells were returned to the incubator for an additional 72 hours. CellTiter-Glo reagent was prepared per manufacturer's instructions and added at 100 μL/well to the cultures. CellTiter-Glo allows for relative enumeration of metabolically active cells by quantifying intracellular ATP concentrations. After 5 minutes of incubation with CellTiter-Glo at ambient room temperature, 125 μL/well of the Cell Titer Glo/cell lysate solution was transferred into black assay plates, which were then read in a luminometer within 30 minutes. Luminescence readings obtained from cultures that did not receive any treatment other than 0.25% DMSO were set as 100% control and all other luminescence values were normalized to these controls (e.g., Normalized RLU, relative luminescence unit).

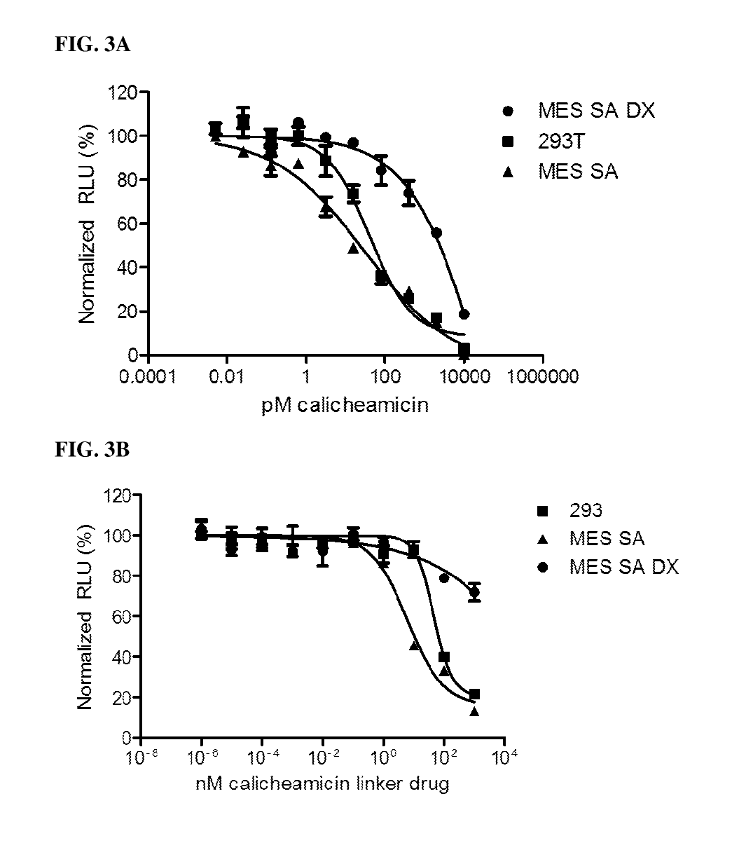

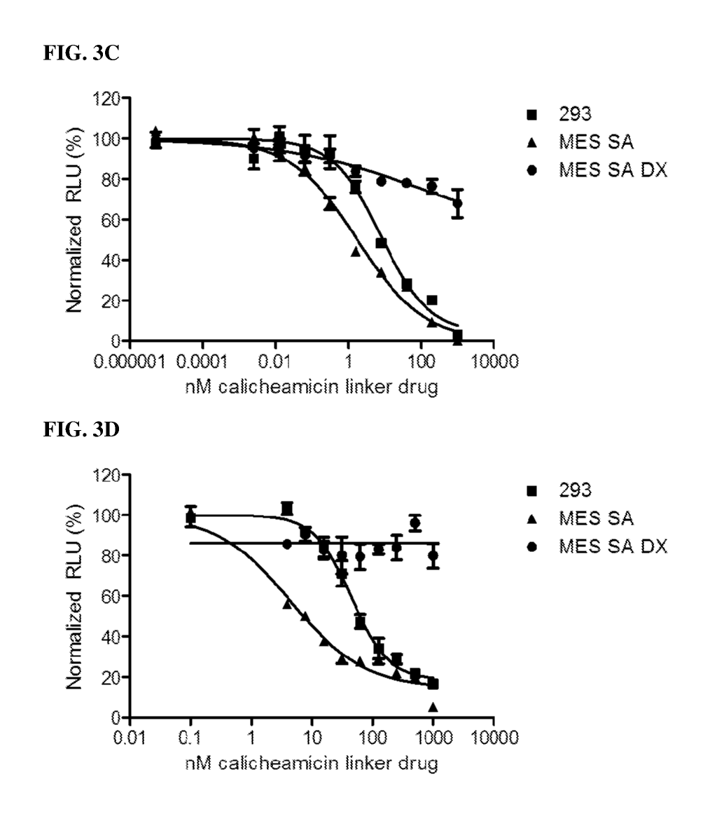

The results of the assays are shown in FIGS. 3A-3D and selected IC50 values derived from the same data presented in Table 5 below. More particularly, FIGS. 3A-3D depict concentration dependent in vitro cell killing curves for calicheamicin (FIG. 3A), Val-Cit calicheamicin (Formula 3) from Example 3 above (FIG. 3B), Val-Ala calicheamicin (Formula 4) from Example 4 above (FIG. 3C) and calicheamicin comprising an oxime linker (Formula 1) from Example 1 above. Cell killing capacity was determined for MES cells, MES SA/DX cells and 293T control cells for each of the respective compounds.

As shown by the curves set forth in FIGS. 3A-3D calicheamicin and each of the linker drug compounds demonstrated pharmaceutically acceptable activity and killed MES SA cells and 293T control cells at relatively low concentrations. In this regard naked calicheamicin showed activity at concentrations approximately an order of magnitude lower than that afforded by the linker-drug constructs. Moreover, as to be expected the MES SA/DX cells were more resistant and required higher toxin concentrations of both naked toxin and drug-linker construct to induce cell death.

With regard to Table 5 derived IC50 values indicate that the naked calicheamicin and a cytotoxin control have activities in the picomolar range while the calicheamicin-linker constructs have activities in the nanomolar range. It will be appreciated that the reduction in cytotoxicity provided by the addition of a linker moiety is desirable in that it results in reduced non-localized toxicity in the event that the drug linker somehow disassociates from the targeting moiety. As such, the data set forth in FIGS. 3A-3D indicates that the disclosed calicheamicin-linkers are favorable candidates for inclusion in antibody drug conjugates.

TABLE 5
Derived IC50 Values for Calicheamicin
and Calicheamicin Linker Constructs
SMALL MOLECULE IVK (10 nM start, 5X dilutions)

| | Toxin | N acetyl calicheamicin | N acetyl calicheamicin |
|---|---|---|---|
| IC50 (pM) | Control | (lot A) | (lot B) |
| 293 T | 43.75 | 40.93 | 52 |
| MES SA | 49.63 | 30.82 | 69.29 |
| MES SA | 1454 | 30575 | 17121 |
| DX | | | |

LINKER DRUG IVK (1000 nM start, 10X dilutions)

| IC50 (nM) | Val-Cit calicheamicin | buffer |
|---|---|---|
| 293 T | 45.99 | >1000 |
| MES SA | 6.299 | >1000 |
| MES SA | >1000 | >1000 |
| DX | | |

### Example 9

### Conjugation of Calicheamicin-Linker Constructs to a Cell Binding Agent

In order to further characterize the calicheamicin-linker constructs of the instant invention dipeptidyl drug-linker compounds fabricated as set forth in Examples 3 and 4 above were conjugated to site-specific anti-SEZ6 antibodies using a selective reduction process comprising a stabilizing agent (e.g., L-arginine) and a mild reducing agent (e.g., glutathione). As discussed above, selective conjugation preferentially conjugates the calicheamicin-linker constructs to an engineered free cysteine on the antibody with a little non-specific conjugation.

In this respect the target conjugation site for the hSC17ss1 construct is the unpaired cysteine on each light chain at position 214 (C214). To effect conjugation of these engineered sites preparations of hSC17ss1 were partially reduced in a buffer containing IM L-arginine/8 mM glutathione, reduced (GSH)/5 mM EDTA, pH 8.0 for a minimum of two hours at room temperature. The preparations were then buffer exchanged into a 20 mM Tris/3.2 mM EDTA, pH 7.0 buffer using a 30 kDa membrane (Millipore Amicon Ultra). The resulting partially reduced preparations has free thiol concentrations between 1.9 and 2.3, and all preparations were then conjugated to Val-Ala calicheamicin (hSC17ss1-va) and Val-Cit calicheamicin (hSC17ss1-vc) via maleimido moieties overnight at 4° C. The reaction was then quenched with the addition of 1.2 molar excess of NAC using a 10 mM stock solution prepared in water. After a minimum quench time of 20 minutes, preparations of antibody-calicheamicin were then diafiltered into 20 mM histidine chloride, pH 6.0 by diafiltration using a 30 kDa membrane (Millipore Amicon Ultra).

### Example 10

### Characterization of Calicheamicin ADCs

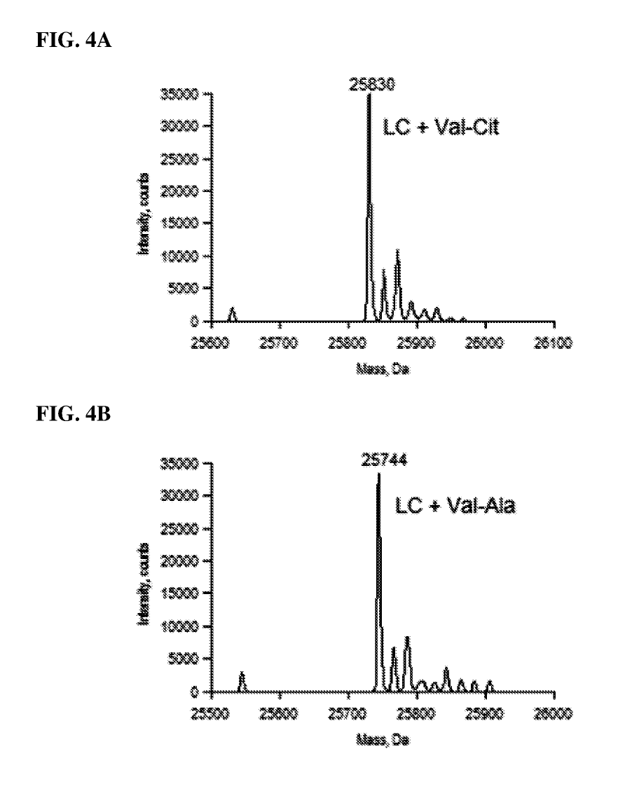

The non-reduced masses of calicheamicin antibody-drug conjugates were determined by AB Sciex 5600 Triple Time-of-Flight Mass Spectrometer (HR Triple TOF MS) and by Bruker maXis II Ultra High Resolution Time-of-Flight Mass Spectrometer (UHR-TOF MS). Both were equipped with electrospray ionization (ESI) sources, which were directly coupled to ultra-high performance liquid chromatography (UHPLC) systems. Samples were first diluted to 1 mg/mL then analyzed in their non-reduced form. The proteins are separated on a reverse phase column (Poroshell 300 SB-C3, 5 um, 1.0×75 mm, Agilent P/N 661750-909; Acquity BEH300 C4, 1.7 um, 2.1×50 mm, Waters P/N 186004495) with a denaturing mobile phase system. Mobile Phase A is 0.1% (v/v) formic acid in water. Mobile phase B is 0.1% (v/v) formic acid in 80% (v/v) 2-propanol, 10% (v/v) acetonitrile, 10% (v/v) water (mobile phase B). The MS spectra (e.g., FIGS. 4A and 4B) of each protein are averaged and then deconvoluted to obtain the average mass and monoisotopic mass. Summarized in Table 5 below are the theoretical and observed average and monoisotopic masses of SC17ss1 LC with the corresponding conjugated calicheamicin linker-drug.

TABLE 6

| | | Theoretical | Observed |
|---|---|---|---|
| | | Average | Average |
| LC-Linker-Drug | Dipeptide | Mass (Da) | Mass (Da) |
| SC17ss1-LD19.4 | Val-Cit | 25830 | 25830 |
| (Formula 4′) | | | |
| SC17-ss1-LD19.5 | Val-Ala | 25743 | 25744 |
| (Formula 5′) | | | |
| N149ss1-LD19.4 | Val-Cit | 25702 | 25702 |
| SC27ss1-LD19.4 | Val-Cit | 25409 | 25409 |
| SC57ss1-LD19.4 | Val-Cit | 25525 | 25525 |
| | | Theoretical | Observed |
| | | Monoisotopic | Monoisotopic |
| | | Mass (Da) | Mass (Da) |
| hIgG1ss1-LD19.11 | Val-Cit-PEG | 26562.40 | 26562.27 |
| (Formula 17′) | | | |
| hIgG1ss1-LD19.15 | Val-Cit-PEG | 26238.22 | 26238.04 |
| (Formula 16′) | | | |
| N149ss1-LD19.11 | Val-Cit-PEG | 26170.58 | 26170.25 |
| N149ss1-LD19.15 | Val-Cit-PEG | 25846.40 | 25845.95 |

This data indicates that the calicheamicin-linker constructs were successfully conjugated to the free cysteine of the engineered anti-SEZ6 antibodies.

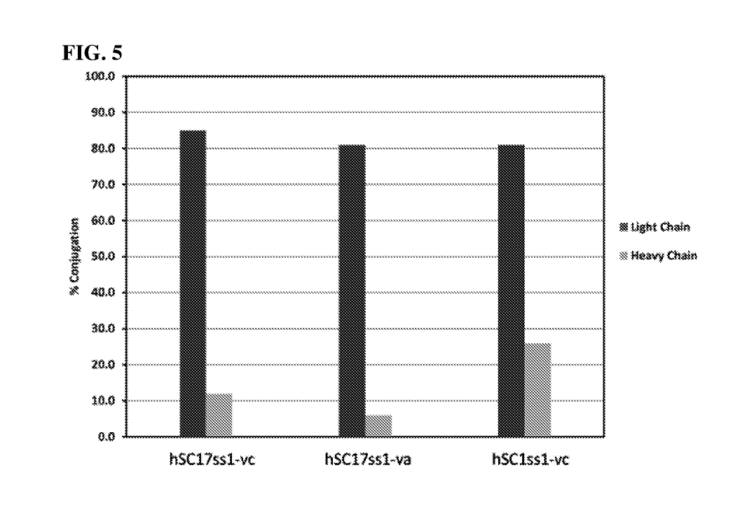

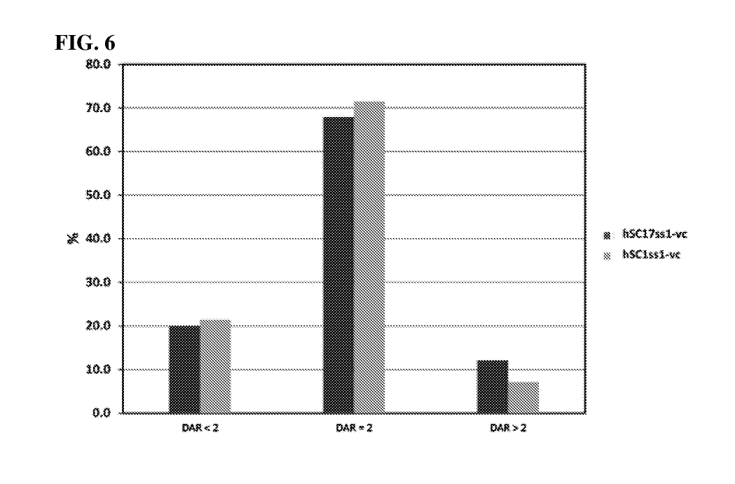

The antibody-drug preparations from the previous example (along with another preparation, hSC1ss1-vc, which immunospecifically binds to CD46 and was conjugated in substantially the same manner as the other preparations) were further characterized by reverse phase (RP-HPLC) analysis to quantify heavy vs light chain conjugation sites. More specifically, as shown in FIG. 5 RP-HPLC was used to determine the percentage of on-target light-chain conjugation for hSC17ss1-vc (Formula 4′), hSC17ss1-va (Formula 5)′, and hSC1ss1-vc (Formula 4′) (FIG. 5). The analysis was conducted using an Aeris WIDEPORE 3.6 μm C4 column (Phenomenex) with 0.1% (v/v) trifluoroacetic acid (TFA) in water as mobile phase A, and 0.1% (v/v) TFA in 90% (v/v) acetonitrile as mobile phase B. Samples were fully reduced with DTT prior to analysis, and then injected onto the column, where a gradient of 30-70% mobile phase B was applied over 15 minutes. UV signal at 214 nm was collected and then used to calculate the extent of heavy and light chain conjugation.

Percent conjugation on the heavy and light chains was determined by integrating the area under the RP-HPLC curve of the previously established peaks (light chain, light chain+1 drug, heavy chain, heavy chain+1 drug, heavy chain+2 drugs, etc.) and calculating the % conjugated for each chain separately. As shown in FIG. 5 percent conjugation on light chains of the hSC17 site specific is >80% for conjugation with both the Val-Cit and Val-Ala calicheamicin constructs. hSC1ss1 site specific conjugated to Val-Cit calicheamicin also yielded light chain conjugation >80%. Percent conjugation on the heavy chains of the samples described above is <15% for the hSC17 site specific conjugates and <30% for the hSC1ss1 site specific conjugation which is expected due to the higher DAR of 2.3 obtained for this sample compared to DARs of 1.9 and 1.8 obtained for the hSC17 site specific Val-Cit and Val-Ala conjugates respectively. In all cases, the conjugation parameters can be further optimized to increase percent conjugation on the light chains while decreasing percent conjugations on the heavy chains.

The same hSC17ss1-vc, hSC17ss1-va, and hSC1ss1-vc preparations were also analyzed using a hydrophobic interaction chromatography (HIC) HPLC based method to determine the amount of DAR=2 species relative to the unwanted DAR>2 species for the ADC. In this regard HIC was conducted using a PolyPropyl A column (PolyLC) with 1.5M ammonium sulfate and 25 mM potassium phosphate in water as mobile phase A, and 0.25% w/v CHAPS and 25 mM potassium phosphate in water as mobile phase B. Samples were injected directly onto the column, where a gradient of 0-100% mobile phase B was applied over 15 minutes. UV signal at 280 nm was collected, and the chromatogram analyzed for unconjugated antibody and higher DAR species. DAR calculations were performed by integrating the area under the HIC curve of the previously established peaks (DAR=0, DAR=1, DAR=2, DAR=4, etc) and calculating the % of each peak. The resulting DAR distribution for hSC17ss1-vc and hSC1ss1-vc are shown in FIG. 6. The DAR distributions as determined by HIC of the hSC17 site-specific conjugate preparations (data not shown for hSC17ss1-va) indicate that all three conjugates yielded >65% DAR=2. The conjugate preparations also yielded less than 25% DAR <2 and less than 15% DAR >2.

The same procedures were used to analyse subsequent conjugations of N149, SC27 and SC57 (IgG1 site-specific antibodies to various determinants) with the following results summarized in Table 7. DAR calculations were performed either using HIC method described above or Size exclusion chromatography method described below.

Size-Exclusion Chromatography (SEC) was used to characterize size heterogeneity of calicheamicin antibody-drug conjugates. The analysis employed an Acquity 1.7-μm, 4.6×300 mm UPLC BEH200 SEC column with 25 mM Sodium Phosphate, pH 6.5, 500 mM L-Arginine, and 10% Isopropanol (IPA) in water as mobile phase. Samples were injected neat and mobile phase was applied isocratically at 0.2 mL/min for 22 min. UV signal at 280 nM was collected and peak area was used to calculate the extent of aggregation and fragmentation of the ADC.

TABLE 7

| | % | % | % | Average | |
|---|---|---|---|---|---|
| | Aggregate | Monomer | Fragments | DAR (HIC) | |
| ADC | (SEC) | (SEC) | (SEC) | or (RP) | |
| SC17.SS1.LD19.4 | 1.1 | 97.3 | 1.6 | 1.9 | (RP) |
| (Formula 4′) | | | | | |
| SC17.SS1.LD19.5 | 0.6 | 97.1 | 2.4 | 1.8 | (RP) |
| (Formula 5) | | | | | |
| SC17.SS1.LD19.6 | 14.4 | 85.6 | NA | 1.8 | (RP) |
| (Formula 14′) | | | | | |
| SC27.SS1.LD19.4 | 7.5 | 92.5 | NA | 2.1 | (RP) |
| (Formula 4′) | | | | | |
| SC57.SS1.LD19.4 | 9.6 | 90.4 | NA | 1.8 | (RP) |
| N149.SS1.LD19.4 | 2.7 | 97.3 | NA | 2.2 | (RP) |
| N149.SS1.LD19.6 | 3.6 | 96.4 | NA | 1.8 | (RP) |
| N149.SS1.LD19.11 | 2.7 | 96.3 | 1 | 2.3 | (HIC) |
| (Formula 17′) | | | | | |
| N149.SS1.LD19.15 | 3.5 | 95.5 | 1 | 1.8 | (HIC) |
| (Formula 16′) | | | | | |
| hIgG1.SS1.LD19.4 | 14.6 | 85.4 | NA | 2.0 | (RP) |
| hIgG1.SS1.LD19.6 | 8.3 | 91.7 | NA | 2.1 | (RP) |
| hIgG1.SS1.LD19.11 | 3.3 | 95.8 | 0.9 | 2.1 | (HIC) |
| hIgG1.SS1.LD19.15 | 4.4 | 94.7 | 0.9 | 1.9 | (HIC) |

The relatively tight average DAR and low rates of aggregation or fragmentation strongly suggests that the resulting preparations will exhibit a favorable therapeutic index and relatively low non-specific toxicity.

### Example 11

### Calicheamicin ADCs Kill Antigen Expressing Cells In Vitro

To determine whether the anti-SEZ6 ADCs of the invention are able to internalize and mediate the delivery of cytotoxic agents to live tumor cells, an in vitro cell killing assay was performed using selected anti-SEZ6 ADCs such as those provided in Example 9.

Cells were cultured and plated generally as described in Example 8 above. One day later, the tumor cells were exposed to humanized anti-SEZ6 ADCs (hSC17ss1-va, hSC17ss1-vc and hSC17ss1-ox comprising the oxime drug-linker of Formula 1) at various concentrations ranging from 0 pM to 1000 pM. After incubation for 96 hours viable cells were enumerated using CellTiter-Glo® (Promega) as described in Example 7 above. Raw luminescence counts using cultures containing untreated cells were set as 100% reference values and all other counts were calculated as a percentage of the reference value.

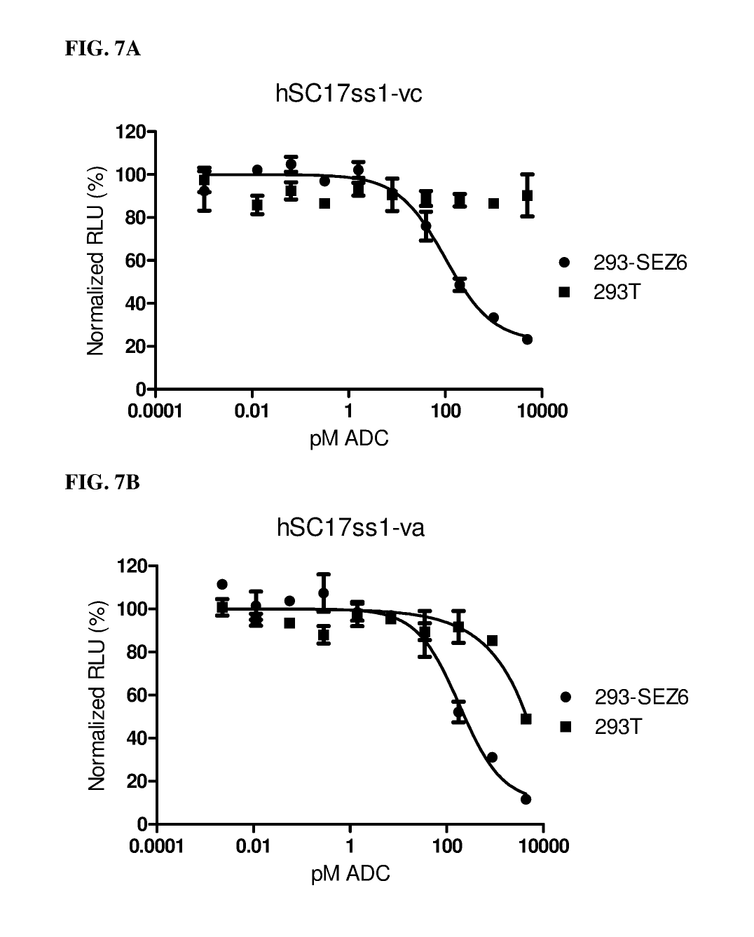

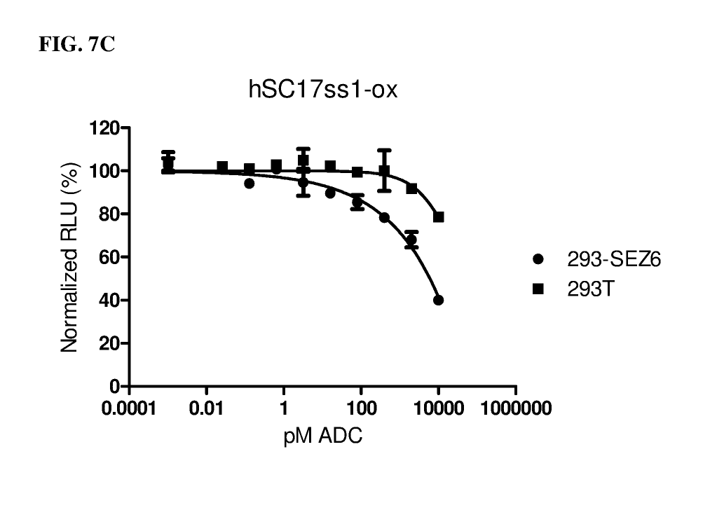

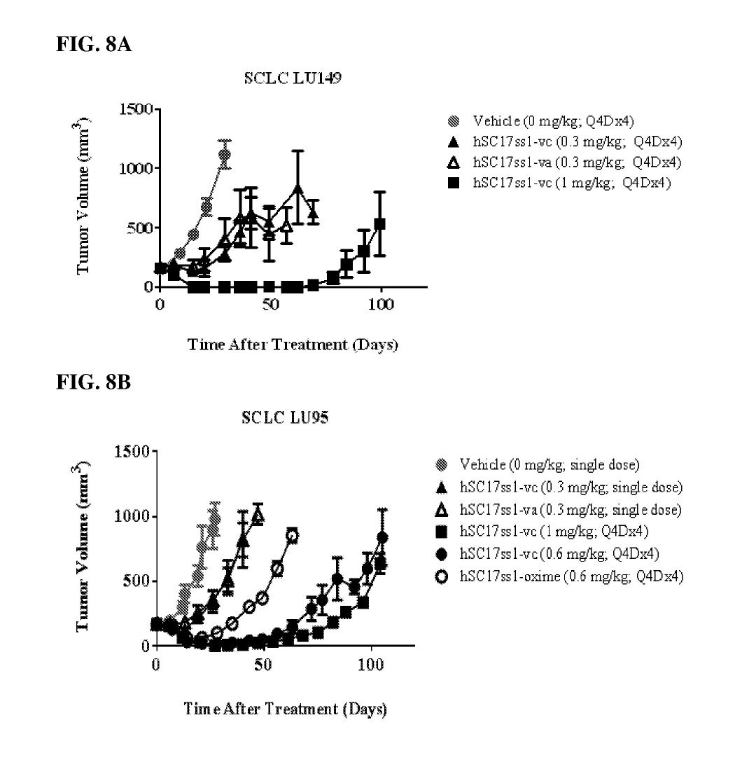

Results for the in vitro assays are shown in FIGS. 7A-7C appended hereto. More particularly FIG. 7A shows the ability of hSC17ss1-vc to eliminate antigen expressing cells while FIG. 7B shows the same for hSC17ss1-va and FIG. 7C for hSC17ss1-ox. In each case the 293T-SEZ6 cells transduced to overexpress SEZ6 were more killed at substantially lower ADC concentrations than the parental line (293T) indicating specificity for the ADC for SEZ6. The data presented in FIGS. 7A-7C demonstrates the ability of anti-SEZ6 ADCs to internalize and deliver cytotoxic calicheamicin payloads thereby supporting use of the disclosed calicheamicin-linker constructs as ADC components.

The same procedures using appropriate target expressing cell lines were used to determine cell killing ability of subsequent calicheamicin conjugates of N149, SC27 and SC57 with the following results summarized in Table 8.

TABLE 8

| | | | IC50 in non-target | |
|---|---|---|---|---|
| | IC50 in target expressing | | expressing cell line, | |
| ADC | cell line, pM (cell line) | | pM (cell line) | |
| SC17.LD19.4 | 1000-5000 | (293-SC17) | >70000 | (293T) |
| (Formula 4′) | | | | |
| SC17.LD19.5 | 1000-2000 | (293-SC17) | >40000 | (293T) |
| (Formula 5′) | | | | |
| SC17.LD19.6 | 2000 | (293-SC17) | >40000 | (293T) |
| (oxime) | | | | |
| SC27.LD19.4 | 60 | (293-SC27) | >40000 | (293T) |
| SC57.LD19.4 | 200 | (293-SC57) | >40000 | (293T) |

| | | | |
|---|---|---|---|
| hIgG1.LD19.4 | 7890 | (MES SA) | N/A |
| hN149.LD19.4 | 23.01 | (MES SA) | N/A |
| hN149.LD19.11 | 3.7 | (MES SA) | N/A |
| (Formula 17′) | | | |
| hN149.LD19.15 | 15.33 | (MES SA) | N/A |
| (Formula 16′) | | | |
| hIgG1.LD19.11 | 3398 | (MES SA) | N/A |
| hIgG1.LD19.15 | 1656 | (MES SA) | N/A |

Overall the target specific ADCs kill the target expressing cells with high efficiency showing relatively low IC50 values. Such values, combined with the lack of killing of non-target expressing cells are indicative of therapeutically useful compounds.

### Example 12

### Calicheamicin ADCs Kill Antigen Expressing Cells In Vivo

In vivo experiments were conducted to confirm the cell killing properties of the calicheamicin ADCs hSC17ss1-vc and hSC17ss1-va demonstrated in Example 11 immediately above. To this end the site-specific SC17-targeted ADCs prepared as set forth in the previous Examples were tested for in vivo therapeutic effects in immunocompromised NODSCID mice bearing subcutaneous patient-derived xenograft (PDX) small cell lung cancer (SCLC) tumors having endogenous SEZ6 cell surface protein expression. More particularly the anti-SEZ6 ADCs were each tested in three different SCLC models.

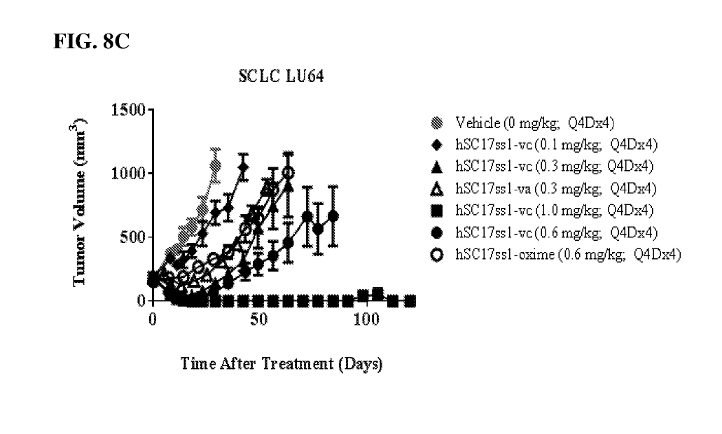

SCLC-PDX lines, LU64, LU95, and LU149 were each injected as a dissociated cell inoculum under the skin near the mammary fat pad region, and measured weekly with calipers (ellipsoid volume=a×b²/2, where a is the long diameter, and b is the short diameter of an ellipse). After tumors grew to an average size of 200 mm³ (range, 100-300 mm³), the mice were randomized into treatment groups (n=5 mice per group) of equal tumor volume averages. Mice (5 per group) were treated with either vehicle (5% glucose in sterile water), or hSC17ss1-vc and hSC17ss1-va calicheamicin preparations (0.1-1 mg/kg) via an intraperitoneal injection (300 μL volume) once every 4 days for total doses (Q4D×4), and therapeutic effects assessed by weekly tumor volume (with calipers as above) and weight measurements. Endpoint criteria for individual mice or treatment groups included health assessment (any sign of sickness), weight loss (more than 20% weight loss from study start), and tumor burden (tumor volumes >1000 mm³). Efficacy was monitored by weekly tumor volume measurements (mm³) until groups reached an average of approximately 800-1000 mm³. Tumor volumes were calculated as an average with standard error mean for all mice in treatment group and were plotted versus time (days) since initial treatment. The results of the treatments are depicted in FIGS. 8A-8C where mean tumor volumes with standard error mean (SEM) in 5 mice per treatment group are shown.

Specifically hSC17ss1-vc, hSC17ss1-va, and hSC17ss1-oxime (Formula 14′) ADCs were evaluated in mice bearing SCLC PDX-LU149 (FIG. 8A), PDX-LU95 (FIG. 8B) or PDX-LU64 (FIG. 8C) at selected dosages to determine their ability to retard tumor growth. The data presented in the subject FIGS. demonstrated that hSC17ss1-vc and hSC17ss1-va ADCs had similar (hSC17ss1-vc compared with hSC17ss1-va) or varied (hSC17ss1-oxime compared with hSC17ss1-vc) therapeutic effects at medium dose levels (0.3-0.6 mg/kg; single dose or Q4D×4 dosing regimen). Furthermore, it was also shown that appropriate dose levels such as those used in the present Example (e.g., 1 mg/kg; Q4D×4) can achieve durable responses for 50 days or longer in SCLC PDX-bearing mice.

In these models and at the doses given, hSC17ss1-vc and hSC17ss1-va ADC preparations had comparable in vivo efficacy when tested in 3 mouse models of SCLC PDX. The hSC17ss1-oxime ADC preparations had some therapeutic effect, but less than the hSC17ss1-vc when evaluated at the same dose in 2 SCLC PDX models. In vivo efficacy of SC17-binding ADCs in mice bearing SCLC-PDX tumors was dose level dependent and potent at higher dose levels. Taken together this data indicates that SC 17 calicheamicin ADCs offer comparable and potent in vivo therapeutic efficacy.

### Example 13

### Mouse Tolerability Study

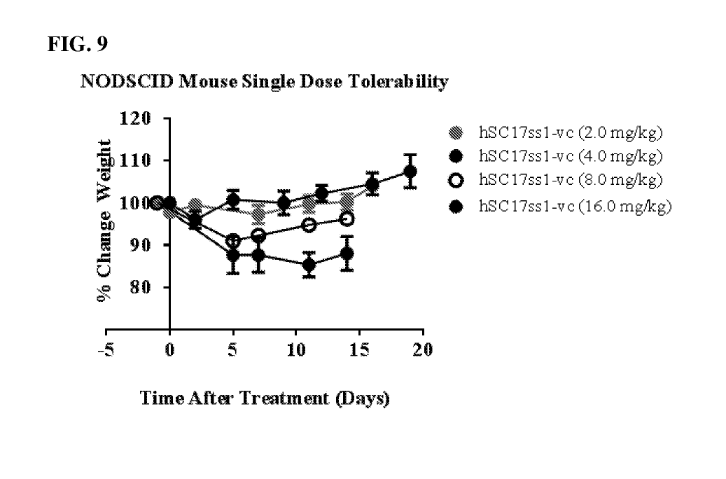

In vivo tolerability of the hSC17ss1-vc calicheamicin ADC prepared as set forth in the previous Examples was tested in immunocompromised NODSCID mice. Naïve 5-7 week-old mice were weighed (21-28 g) and -randomized into treatment groups (n=3-4 mice per group) of equal average animal weights. Mice were treated with a single dose of hSC17ss1-vc calicheamicin preparation (2-16 mg/kg) via an intravenous injection (100 μL volume), and mice weight measurements were monitored 2-3 times weekly for 2 to 3 weeks. Endpoint criteria for individual mice included weight loss (more than 10% from study start) and assessment of physical health (posture, activity, temperature, breathing rate, or any other sign of sickness). The results are shown in FIG. 9 where percent (%) weight change from study start is monitored over time. The data presented in FIG. 9 demonstrated that hSC17ss1-vc was well tolerated at a single dose of 8 mg/kg or lower in immunocompromised NODSCID mice. Recovery from an initial weight loss at Day 5 after treatment occurred with 8 mg/kg dose level; however, animals treated with the 16 mg/kg dose level did not recover and were taken down due to poor health (endpoint criteria).

### Example 14

### Pharmacokinetics in Cynomolgus Monkey Pharmacokinetics (PK) of hSC17ss1-vc and hSC27ss1-vc calicheamicin

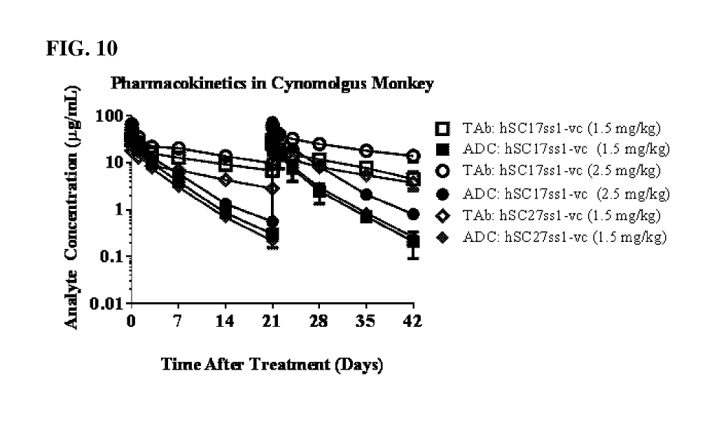

ADC prepared as set forth in the previous Examples was evaluated in Cynomolgus Monkeys. Cynomolgus monkeys (n=3 males per group) were treated with 1.5 mg/kg hSC17ss1-vc, 2.5 mg/kg hSC17ss1-vc, or 1.5 mg/kg hSC27ss1-vc via a 20 minute intravenous infusion once every 3 weeks for a total of 2 doses (Q3W×2). Pharmacokinetics was assessed to confirm exposures associated with toxicities (see Example 15). Plasma samples were collected at various time points after each dose, and total antibody (TAb) and ADC analyte concentrations were assessed by sandwich ELISA assay-type methods. TAb and ADC concentration versus time data are shown in FIG. 10.

The data presented in FIG. 10 (Formula 4′) demonstrate that PK of calicheamicin ADCs is dose-linear. MAb exposures are greater than ADC exposures as expected. Little to no accumulation of the ADC was observed. Taken together, PK of calicheamicin ADCs in Cynomolgus Monkey is consistent with expected PK for an antibody and/or ADC.

### Example 15

### Monkey Toxicology Study

### Study Design:

Cynomolgus monkeys (3/dose level) were administered SC17ss1LD19.4 at 1.5 and 2.5 mg/kg dose levels 3 weeks apart for a total of 2 doses. Animals were necropsied 3 weeks following the last dose. Endpoints included clinical observations, body weight, hematology, clinical chemistry, coagulation, urinalysis, organ weights, gross pathology, and histopathology.

### Results:

SC17ss1LD19.4 at 1.5 mg/kg/dose

The administration of SC17ss1LD19.4 at 1.5 mg/kg/dose administered as 2 doses every 3 weeks by intravenous continuous infusion was tolerated. Test article related changes were present in clinical chemistry and histopathology. No test article related changes were present in gross pathology, organ weights, hematology, coagulation and urinalysis.

Clinical chemistry changes were generally dose-related and characterized by elevations in AST. Histopathology changes were slightly and inconsistently dose-related with changes in the kidney, skin, esophagus, tongue, urinary bladder and thymus.

SC17ss1LD19.4 at 2.5 mg/kg/dose

The administration of SC17ss1LD19.4 at 2.5 mg/kg/dose administered as 2 doses every 3 weeks by intravenous continuous infusion was tolerated. Test article related changes were present in organ weights, hematology, clinical chemistry and histopathology. No test article related changes were present in gross pathology, coagulation and urinalysis.

Organ weight changes were characterized by decreased thymus and increased testis weights. Hematology changes were characterized by reductions in platelet and reticulocyte counts. Clinical chemistry changes were slightly dose-related and characterized by elevations in AST, ALT, total protein, globulin, and reductions in albumin. Histopathology changes were slightly and inconsistently dose-related with changes in the kidney, epithelium (skin, esophagus, tongue, urinary bladder), thymus, and testis.

Overall the toxicity profile of the tested compounds indicates that they may be well tolerated in mammals and therapeutically useful.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. A compound, or a pharmaceutically acceptable salt thereof, having the Formula (I):
Ab-[W-(L³)_{z1}-M-(L⁴)_{z2}-P-D]_{z3} (I),
wherein:
Ab is a targeting agent;
W is a connecting group;
M is a cleavable moiety;
L³ and L⁴ are independently a linker;
P is a disulfide protecting group;
D is a calicheamicin or analog thereof;
z1 and z2 are independently an integer from 0 to 10; and
z3 is an integer from 1 to 10.

2. The compound of claim 1, wherein D comprises Formula (Ia): 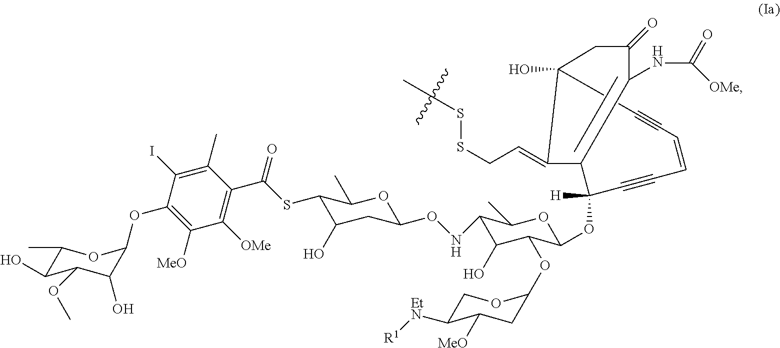
wherein:
R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —Cl₃, —CN, —C(O)R^{1E}, —OR^{1A}, —NR^{1B}R^{1C}, —C(O)OR^{1A}, —C(O)NR^{1B}R^{1C}, —SR^{1D}, —SOₙ₁R^{1B} or —SOᵥ₁NR^{1B}R^{1C};
R^{1A}, R^{1B}, R^{1C}, R^{1D} and R^{1E} are independently hydrogen, halogen, —CF₃, —CCl₃, —CBr₃, —Cl₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{1B} and R^{1C} substituents bonded to the same nitrogen atom may optionally be joined to form a substituted or unsubstituted heterocycloalkyl or substituted or unsubstituted heteroaryl;
n1 is an integer from 0 to 4; and
v1 is 1 or 2.

3-4. (canceled)

5. The compound of claim 1, wherein the targeting agent is selected from the group consisting of a chimeric antibody, a CDR grafted antibody, a humanized antibody a human antibody, an anti-SEZ6 antibody and an immunoreactive fragment thereof.

6. (canceled)

7. The compound of claim 1, wherein W is covalently attached to a cysteine residue within the targeting agent.

8. The compound of claim 7, wherein the cysteine residue is at Kabat position C214.

9. (canceled)

10. The compound of claim 1, or a pharmaceutically acceptable salt thereof, having the Formula (II): 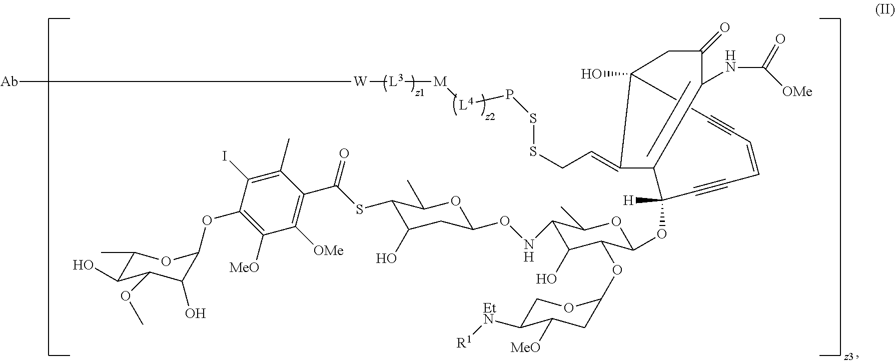
wherein:
Ab is an antibody;
L³ is a bond, —O—, —S—, —NR^{3B}—, —C(O)—, —C(O)O—, —S(O)—, —S(O)₂—, —C(O)NR^{3B}—, —NR^{3B}C(O)—, —NR^{3B}C(O)NH—, —NHC(O)NR^{3B}—, substituted or unsubstituted alkylene, or substituted or unsubstituted heteroalkylene;
L⁴ is a bond, —O—, —S—, —NR^{4B}—, —C(O)—, —C(O)O—, —S(O)—, —S(O)₂—, —C(O)NR^{4B}—, —NR^{4B}C(O)—, —NR^{4B}C(O)NH—, —NHC(O)NR^{4B}—, substituted or unsubstituted alkylene or substituted or unsubstituted heteroalkylene;
R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —Cl₃, —CN, —C(O)R^{1E}, —OR^{1A}, —NR^{1B}R^{1C}, —C(O)OR^{1A}, —C(O)NR^{1B}R^{1C}, —SR^{1D}, —SOₙ₁R^{1B} or —SOᵥ₁NR^{1B}R^{1C};
P is —O—, —C(O)—, —S—, —NR^{2B}—, —C(O)—, —C(O)O—, —S(O)—, —S(O)₂—, —C(O)NR^{2B}—, —NR^{2B}C(O)—, —NR^{2B}C(O)NH—, —NHC(O)NR^{2B}—, substituted or unsubstituted alkyl, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
M is —O—, —S—, —NR^{5B}—, —C(O)—, —C(O)O—, —S(O)—, —S(O)₂—, —C(O)NR^{5B}—, —NR^{5B}C(O)—, —NR^{5B}C(O)NH—, —NHC(O)NR^{5B}—, —[NR^{5B}C(R^{5E})(R^{5F})C(O)]ₙ₂—, substituted or unsubstituted alkylene, substituted or unsubstituted heteroalkylene, substituted or unsubstituted cycloalkylene, substituted or unsubstituted heterocycloalkylene, substituted or unsubstituted arylene substituted or unsubstituted heteroarylene or M^{1A}-M^{1B}-M^{1C};
W is —O—, —S—, —NR^{6B}—, —C(O)—, —C(O)O—, —S(O)—, —S(O)₂—, —C(O)NR^{6B}, —NR^{6B}C(O)—, —NR^{6B}C(O)NH—, —NHC(O)NR^{6B}—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene or W^{1A}—W^{1B}—W^{1C};
M^{1A} is bonded to L³ and M^{1C} is bonded to L⁴;
M^{1A} is a bond, —O—, —S—, —NR^{5AB}—, —C(O)—, —C(O)O—, —S(O)—, —S(O)₂—, —C(O)NR^{5AB}—, —NR^{5AB}C(O)—, —NR^{5AB}C(O)NH—, —NHC(O)NR^{5AB}—, —[NR^{5AB}CR^{5AE}R^{5AF}C(O)]ₙ₃—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
M^{1B} is a bond, —O—, —S—, —NR^{5BB}—, —C(O)—, —C(O)O—, —S(O)—, —S(O)₂—, —C(O)NR^{5BB}—, —NR^{5BB}C(O)—, —NR^{5BB}C(O)NH—, —NHC(O)NR^{5BB}—, —[NR^{5BB}C(R^{5BE})(R^{5BF})C(O)]ₙ₄—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
M^{1C} is a bond, —O—, —S—, —NR^{5CB}—, —C(O)—, —C(O)O—, —S(O)—, —S(O)₂—, —C(O)NR^{5CB}—, NR^{5CB}C(O)—, —NR^{5CB}C(O)NH—, —NHC(O)NR^{5CB}—, —[NR^{5CB}CR^{5CE}R^{5CF}C(O)]ₙ₅—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
W^{1A} is bonded to Ab and W^{1C} is bonded to L³;
W^{1A} is a bond, —O—, —S—, —NR^{6AB}—, —C(O)—, C(O)O—, —S(O)—, —S(O)₂—, —C(O)NR^{6AB}—, —NR^{6AB}C(O)—, —NR^{6AB}C(O)NH—, —NHC(O)NR^{6AB}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
W^{1B} is a bond, —O—, —S—, —NR^{6BB}—, —C(O)—, —C(O)O—, —S(O)—, —S(O)₂—, —C(O)NR^{6BB}—, —NR^{6BB}C(O)—, —NR^{6BB}C(O)NH—, —NHC(O)NR^{6BB}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
W^{1C} is a bond, —O—, —S—, —NR^{6CB}—, —C(O)—, —C(O)O—, —S(O)—, —S(O)₂—, —C(O)NR^{6CB}—, —NR^{6CB}C(O)—, —NR^{6CB}C(O)NH—, —NHC(O)NR^{6CB}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R^{1A}, R^{1B}, R^{1C}, R^{1D}, R^{1E}, R^{2B}, R^{3B}, R^{4B}, R^{5B}, R^{5E}, R^{5F}, R^{5AB}, R^{5AE}, R^{5AF}, R^{5BB}, R^{5BE}, R^{5BF}, R^{5CB}, R^{5CE}, R^{5CF}, R^{6B}, R^{6AB}, R^{6BB} and R^{6CB} are independently hydrogen, halogen, —CF₃, —Cl₃, —CBr₃, —Cl₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OCl₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{1B} and R^{1C} substituents bonded to the same nitrogen atom may optionally be joined to form a substituted or unsubstituted heterocycloalkyl or substituted or unsubstituted heteroaryl;
n1 is an integer from 0 to 4;
v1 is 1 or 2;
n2, n3, n4 and n5 are independently and integer from 1 to 10;
z1 and z2 are independently an integer from 0 to 10; and
z3 is an integer from 1 to 10.

11-18. (canceled)

19. The compound of claim 10, wherein W is 5- or 6-membered substituted or unsubstituted heterocycloalkylene.

20. The compound of claim 19, wherein W has the formula:

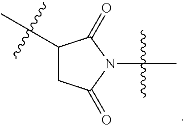 [SMILES: *C1CC(=O)N(*)C1=O]

21. The compound of claim 10, wherein M comprises a peptide.

22. (canceled)

23. The compound of claim 10, wherein M^{1A} and M^{1B} are independently amino acids.

24. The compound of claim 10, wherein at least one of M^{1A} or M^{1B} is selected from the group consisting of valine (val), alanine (ala), and citrulline (cit).

25-26. (canceled)

27. The compound of claim 10, wherein at least one of M^{1A}, M^{1B} or M^{1C} is substituted arylene.

28. The compound of claim 10, wherein at least one of M^{1A}, M^{1B} or M^{1C} has Formula (III): 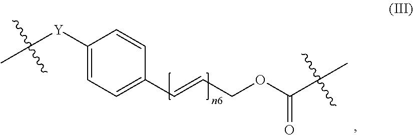
wherein:
Y is —NH—, —O—, —C(O)NH— or —C(O)O—; and
n6 is an integer from 0 to 3.

29. The compound of claim 10, wherein —[W-(L³)Z₁-M-(L⁴)_{z2}-P-D] is:

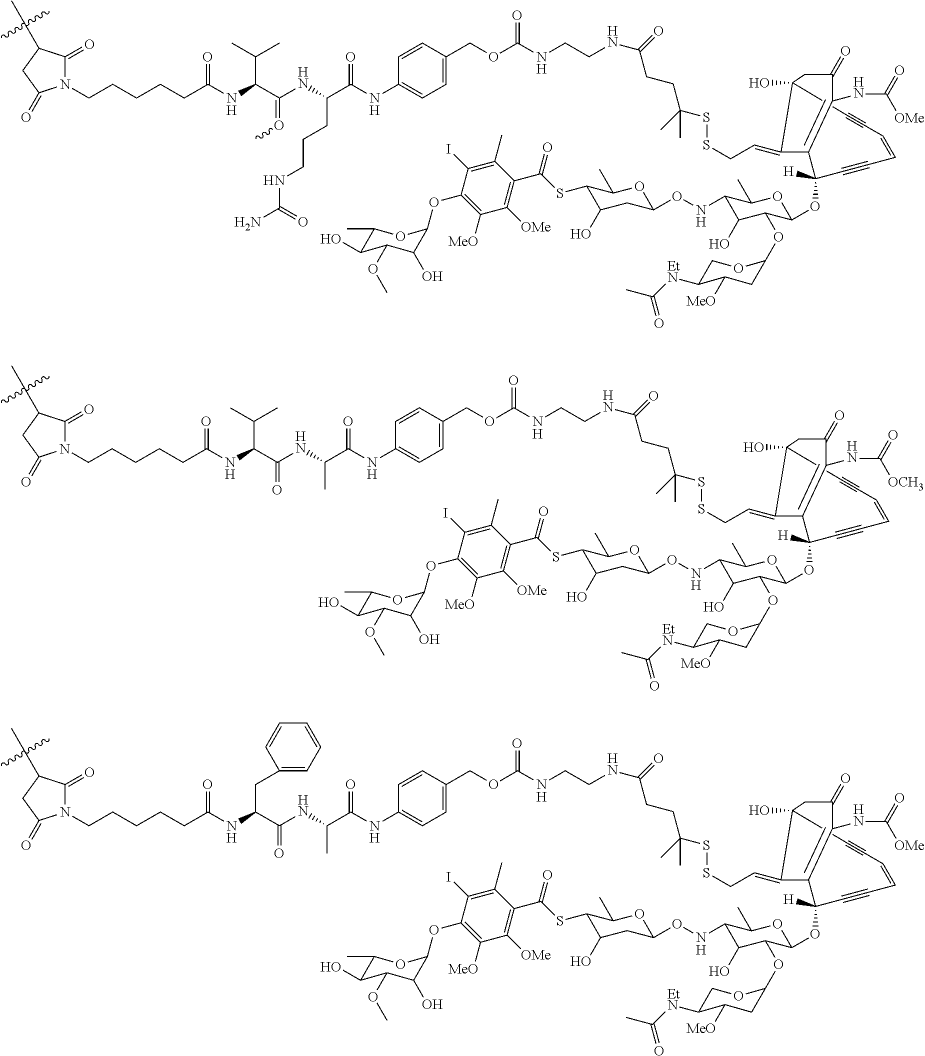

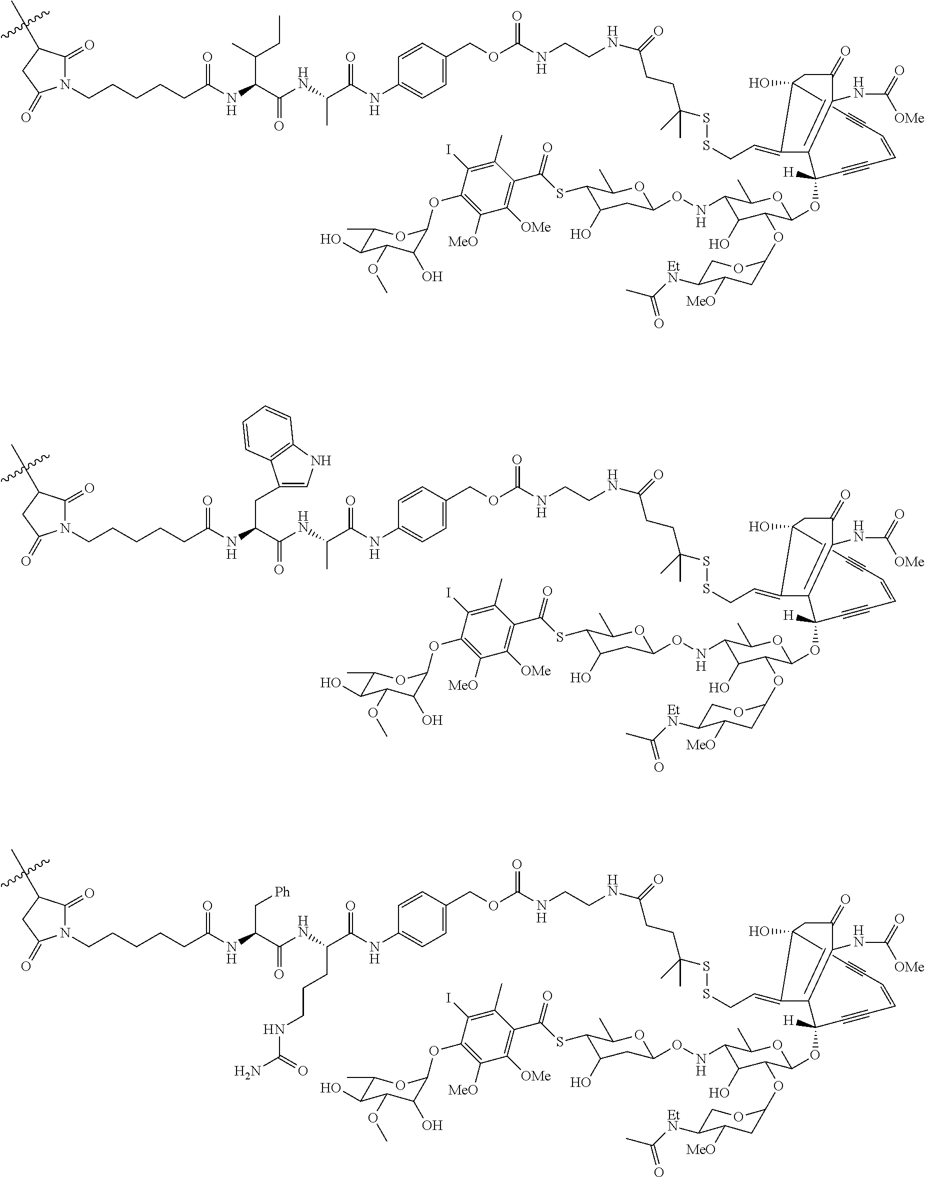

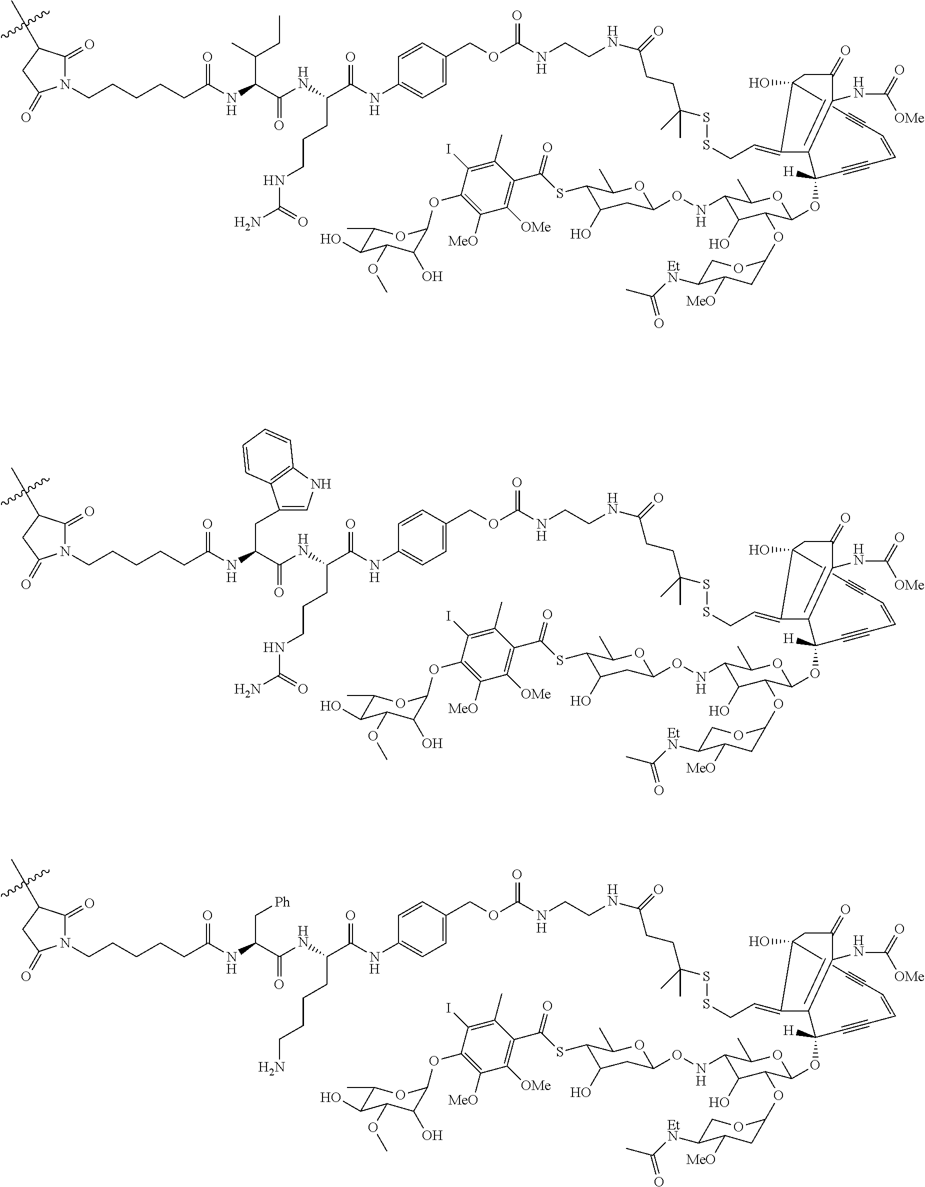

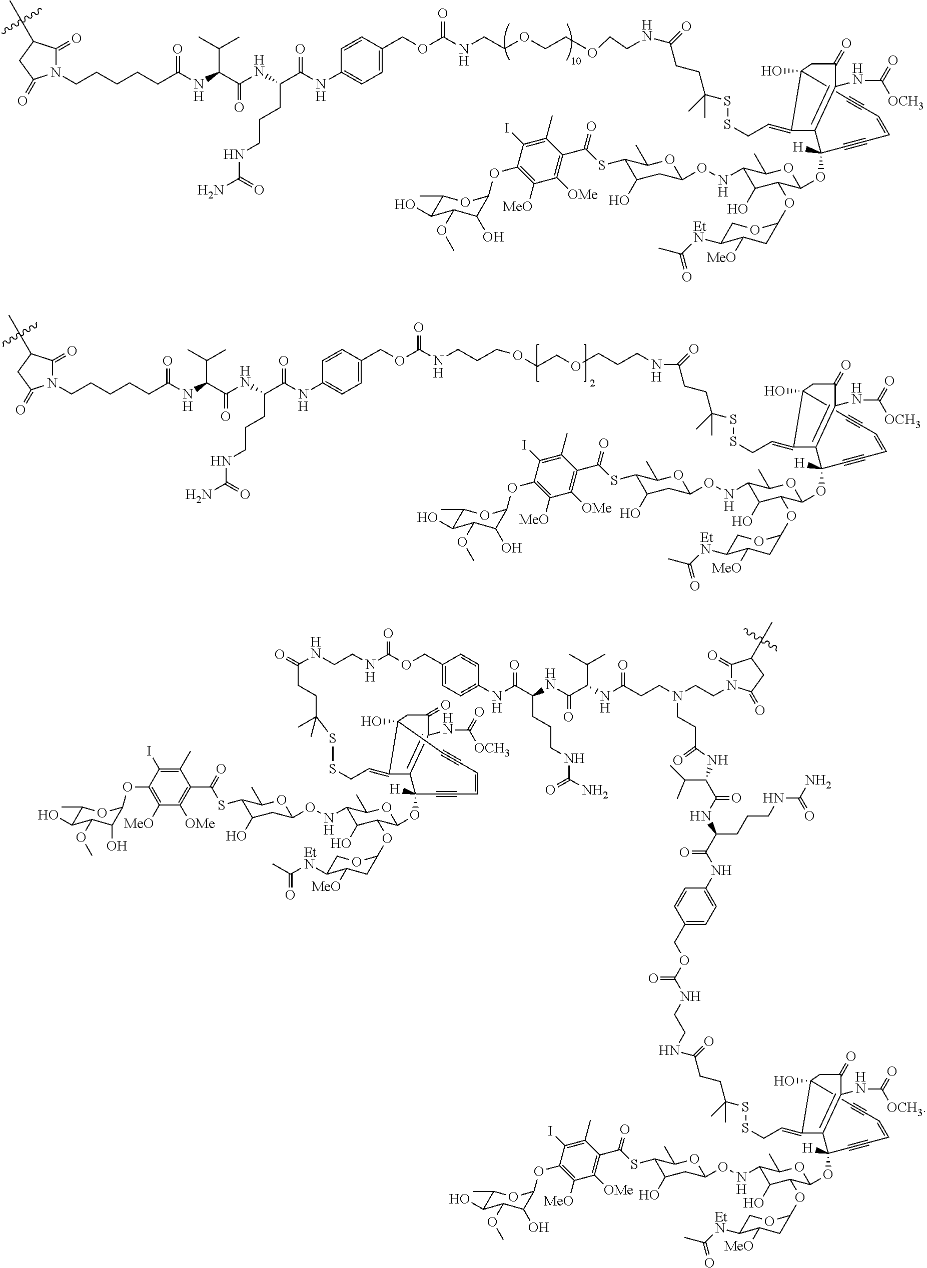

30. (canceled)

31. A pharmaceutical composition comprising a compound of claim 1.

32. A method for treating a patient that has cancer comprising the step of selecting said patient administering to said patient a therapeutically effective amount of the compound of claim 1.

33-34. (canceled)

35. A method for delivering a calicheamicin cytotoxin to a cell comprising contacting the cell with an compound of claim 1.

36. A method of preparing an antibody drug conjugate comprising contacting a calicheamicin construct with a cysteine or lysine of an antibody, the calicheamicin construct having the formula W¹-(L³)_{z1}-M-(L⁴)_{z2}-P-D, wherein W¹ is a functional group reactive with a lysine side chain or cysteine side chain, M is a cleavable moiety, L³ and L⁴ are independently a linker, P is a disulfide protecting group and D is a calicheamicin or analog thereof.

37-41. (canceled)

42. A compound having the Formula (IV): 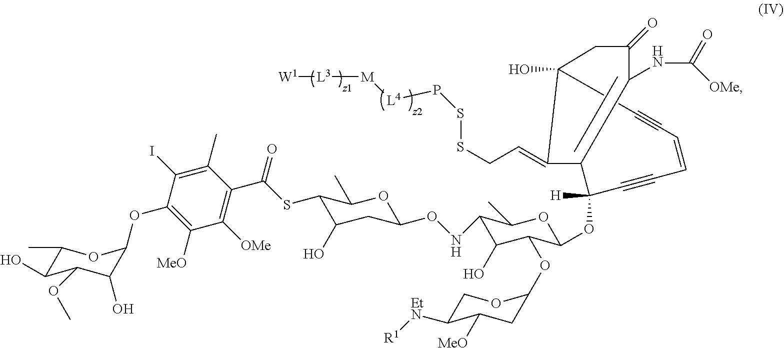
L³ is a bond, —O—, —S—, —NR^{3B}—, —C(O)—, —C(O)O—, —S(O)—, —S(O)₂—, —C(O)NR^{3B}—, —NR^{3B}C(O)—, —NR^{3B}C(O)NH—, —NHC(O)NR^{3B}—, substituted or unsubstituted alkylene or substituted or unsubstituted heteroalkylene;
L⁴ is a bond, —O—, —S—, —NR^{4B}—, —C(O)—, —C(O)O—, —S(O)—, —S(O)₂—, —C(O)NR^{4B}—, —NR^{4B}C(O)—, —NR^{4B}C(O)NH—, —NHC(O)NR^{4B}—, substituted or unsubstituted alkylene or substituted or unsubstituted heteroalkylene;
R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —CF₃, —CCl₃, —CBr₃, —CI₃, —CN, —C(O)R^{1E}, —OR^{1A}, —NR^{1B}R^{1C}, —C(O)OR^{1A}, —C(O)NR^{1B}R^{1C}, —SR^{1D}, —SOₙ₁R^{1B} or —SOᵥ₁NR^{1B}R^{1C};
P is —O—, —S—, —NR^{2B}—, —C(O)—, —C(O)O—, —S(O)—, —S(O)₂—, —C(O)NR^{2B}—, —NR^{2B}C(O)—, —NR^{2B}C(O)NH—, —NHC(O)NR^{2B}—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
M is —O—, —S—, —NR^{5B}—, —C(O)—, —C(O)O—, —S(O)—, —S(O)₂—, —C(O)NR^{5B}—, —NR^{5B}C(O)—, —NR^{5B}C(O)NH—, —NHC(O)NR^{5B}—, —[NR^{5B}C(R^{5E})(R^{5F})C(O)]ₙ₂—, substituted or unsubstituted alkylene, substituted or unsubstituted heteroalkylene, substituted or unsubstituted cycloalkylene, substituted or unsubstituted heterocycloalkylene, substituted or unsubstituted arylene substituted or unsubstituted heteroarylene or M^{1A}-M^{1B}-M^{1C};
W¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, —N₃, —CF₃, —CCl₃, —CBr₃, —Cl₃, —CN, —C(O)R^{7E}, —OR^{7A}, —NR^{7B}R^{7C}, —C(O)OR^{7A}, —C(O)NR^{7B}R^{7C}, —NO₂, —SR^{7D}, SOₙ₇R^{7B}, —SOᵥ₇NR^{7B}R^{7C}, —NHNR^{7B}R^{7C}, —ONR^{7B}R^{7C}, —NHC(O)NHNR^{7B}R^{7C};
M^{1A} is bonded to L³ and M^{1C} is bonded to L⁴;
M^{1A} is a bond, —O—, —S—, —NR^{5AB}—, —C(O)—, —C(O)O—, —S(O)—, —S(O)₂—, —C(O)NR^{5AB}—, —NR^{5AB}C(O)—, —NR^{5AB}C(O)NH—, —NHC(O)NR^{5AB}—, —[NR^{5AB}CR^{5AE}R^{5AF}C(O)]ₙ₃—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
M^{1B} is a bond, —O—, —S—, —NR^{5BB}—, —C(O)—, —C(O)O—, —S(O)—, —S(O)₂—, —C(O)NR^{5BB}—, —NR^{5BB}C(O)—, —NR^{5BB}C(O)NH—, —NHC(O)NR^{5BB}—, —[NR^{5BB}C(R⁵BE)(R⁵BF)C(O)]ₙ₄—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
M^{1C} is a bond, —O—, —S—, —NR^{5CB}—, —C(O)—, —C(O)O—, —S(O)—, —S(O)₂—, —C(O)NR^{5CB}CB, —NR^{5CB}C(O)—, —NR^{5CB}C(O)NH—, —NHC(O)NR^{5CB}—, —[NR^{5CB}CR^{5CE}R^{5CF}C(O)]ₙ₅—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R^{1A}, R^{1B}, R^{1C}, R^{1D}, R^{1E}, R^{2B}, R^{3B}, R^{4B}, R^{5B}, R^{5E}, R^{5F}, R^{5AB}, R^{5AE}, R^{5AF}, R^{5BB}, R^{5BE}, R^{5BF}, R^{5CB}, R^{5CE}, R^{5CF}, R^{6B}, R^{7A}, R^{7B}, R^{7C}, R^{7D}, R^{7E}, are independently hydrogen, halogen, —CF₃, —CCl₃, —CBr₃, —Cl₃, —OH, —NH₂, —COOH, —CONH₂, —N(O)₂, —SH, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCCl₃, —OCBr₃, —OC₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{1B} and R^{1C} substituents bonded to the same nitrogen atom may optionally be joined to form a substituted or unsubstituted heterocycloalkyl or substituted or unsubstituted heteroaryl;
n1 and n7 are independently an integer from 0 to 4;
v1 and v7 are independently 1 or 2; and
n2, n3, n4 and n5 are independently and integer from 1 to 10.

43. The compound of claim 42, wherein the compound is: 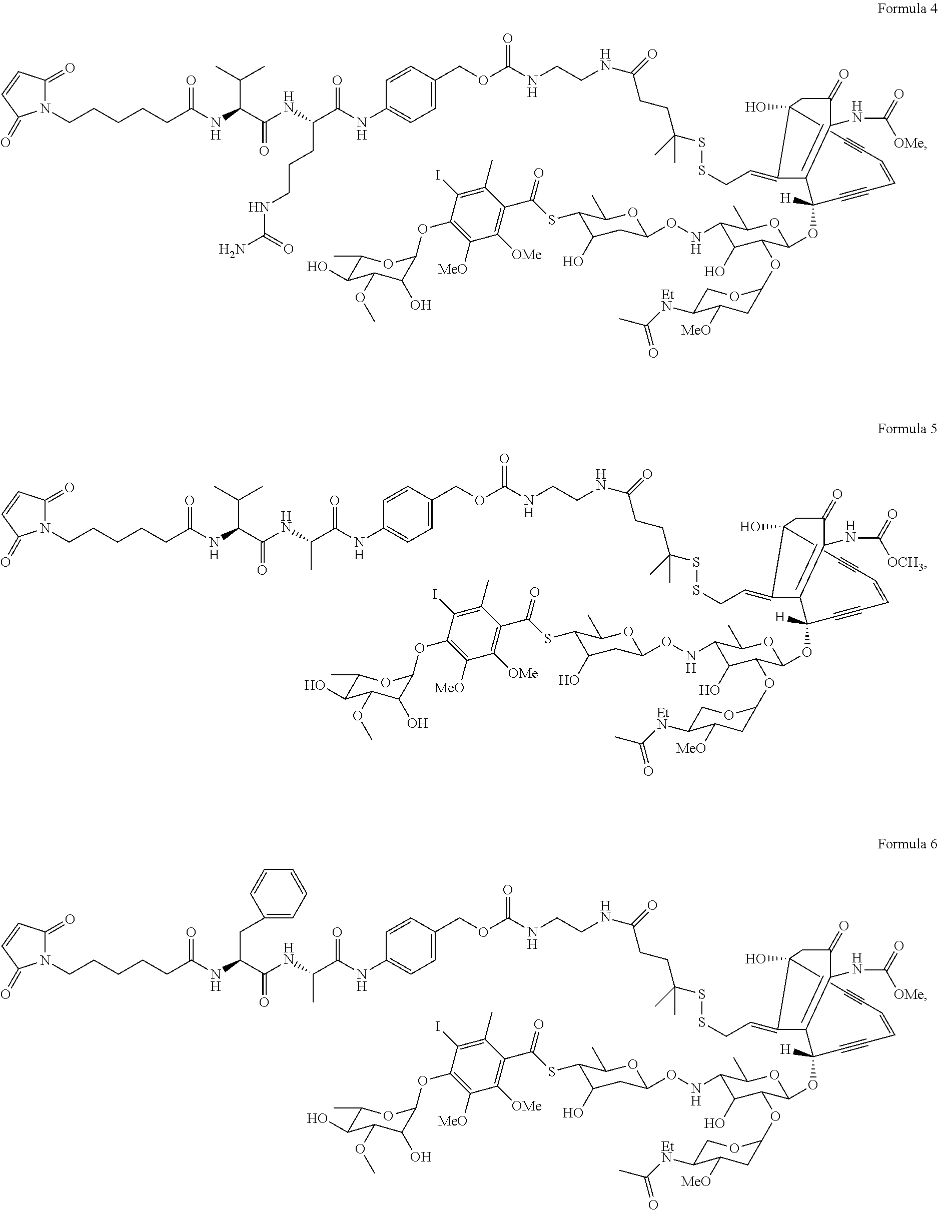 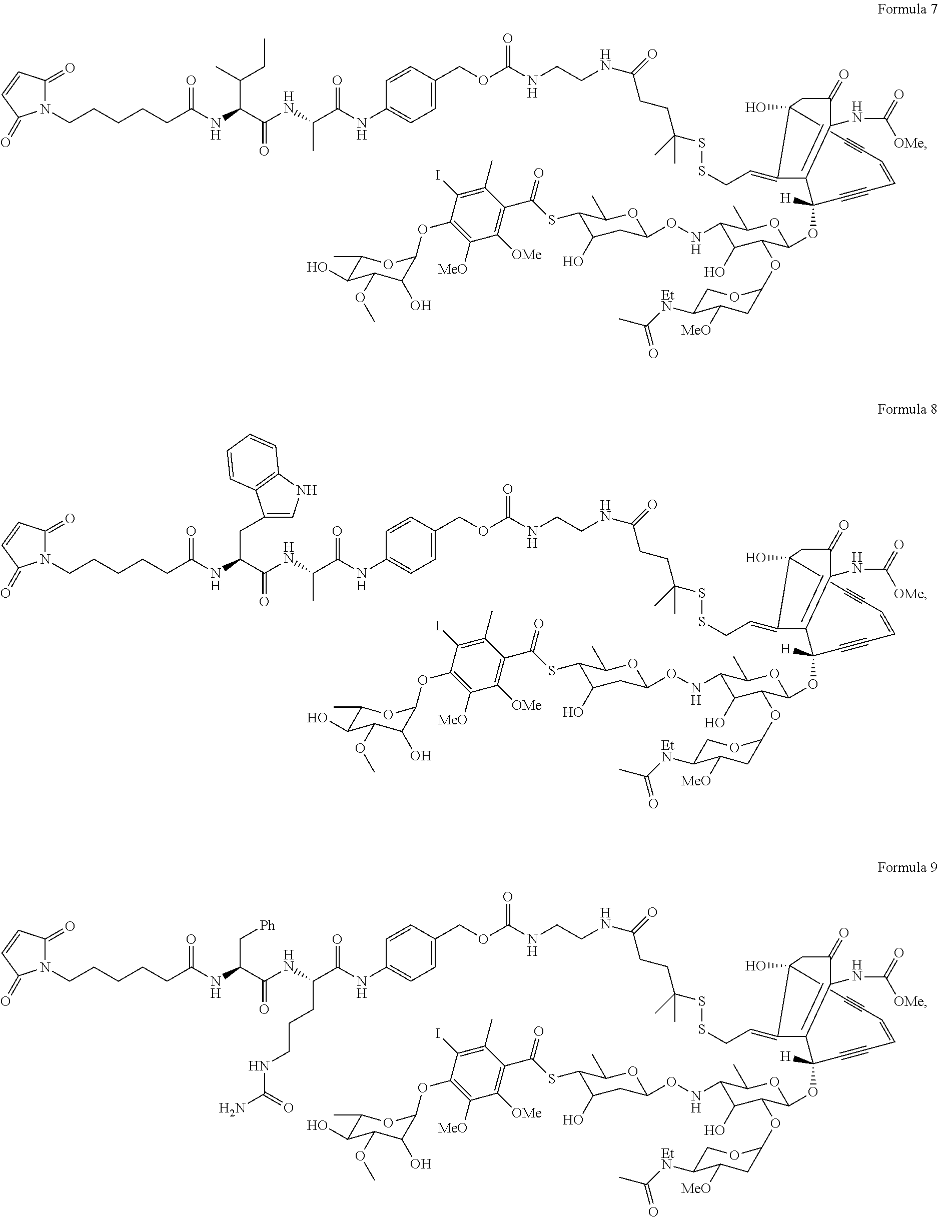 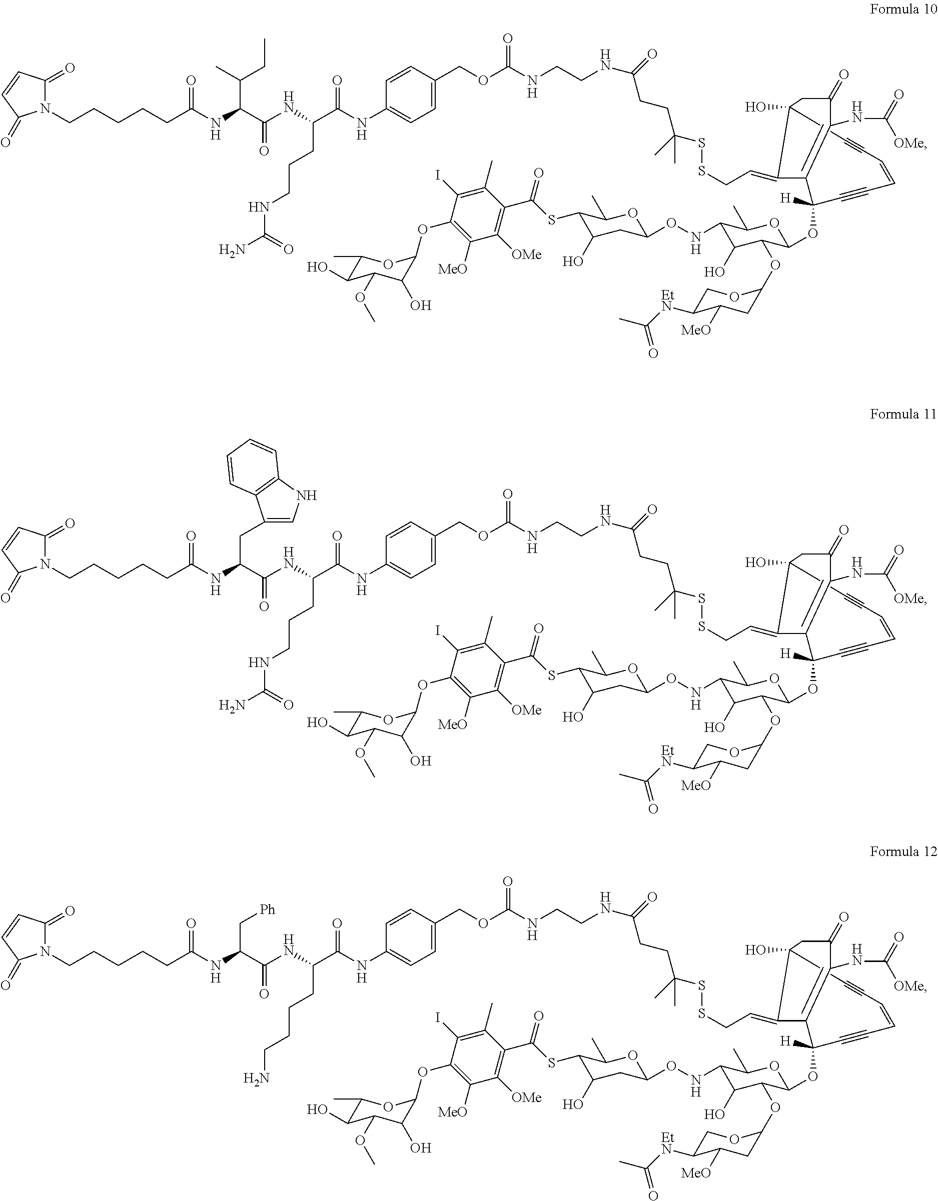 [chemical structure]


SEQUENCE LISTING (5 sequences)

SEQ ID NO: 1 - protein, 107 aa, Homo sapiens [sequence omitted]

SEQ ID NO: 2 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 4 aa, Artificial Sequence
  Synthetic polypeptides
ALAL

SEQ ID NO: 4 - protein, 4 aa, Artificial Sequence
  Synthetic polypeptides
  MOD_RES (1)..(1): bAla
ALAL

SEQ ID NO: 5 - protein, 4 aa, Artificial Sequence
  Synthetic polypeptides
GFLG